(19) United States
(12) Patent Application Publication
(10) Pub. No.: US 2025/0059535 A1
(43) Pub. Date: Feb. 20, 2025

(54) OPA1 ANTISENSE OLIGOMERS FOR TREATMENT OF CONDITIONS AND DISEASES

(71) Applicant: Stoke Therapeutics, Inc., Bedford, MA (US)

(72) Inventors: Isabel Aznarez, Boston, MA (US); Kiat Huat Lim, Billerica, MA (US); Jacob Kach, Jamaica Plain, MA (US)

(21) Appl. No.: 18/658,390

(22) Filed: May 8, 2024

Related U.S. Application Data
(63) Continuation of application No. PCT/US2022/049318, filed on Nov. 8, 2022.
(60) Provisional application No. 63/277,405, filed on Nov. 9, 2021.
(60) Provisional application No. 63/277,767, filed on Nov. 10, 2021.

(51) Int. Cl. C12N 15/113

(52) U.S. Cl. CPC C12N 15/113; C12N 2310/14; C12N 2310/531; C12N 2320/33

(57) ABSTRACT

Agents that target a processed mRNA, e.g., the 5′ UTR of the processed mRNA, can modulate protein expression, e.g., via modulation of translation of the processed mRNA. Alternative splicing events in genes can lead to non-productive mRNA transcripts which in turn can lead to aberrant protein expression. Agents that target the alternative splicing events in genes can modulate the expression level of proteins. Therapeutic agents, which can modulate protein expression by targeting a processed mRNA and/or alternative splicing events, can promote functional protein expression in patients and/or inhibit aberrant protein expression. Such therapeutic agents can be used to treat a condition or disease associated with protein deficiency and/or mitochondrial function deficit.

### CROSS-REFERENCE

This application is a continuation application of International Application No. PCT/US2022/049318, filed Nov. 8, 2022, which claims the benefit of U.S. Provisional Patent Application No. 63/277,405, filed Nov. 9, 2021, and U.S. Provisional Patent Application No. 63/277,767, filed Nov. 10, 2021, each of which is incorporated herein by reference in its entirety.

### REFERENCE TO A SEQUENCE LISTING

This application contains a Sequence Listing which has been submitted electronically in XML format. The Sequence Listing XML is incorporated herein by reference. Said XML file, created on Jan. 19, 2023, is named 47991-738_601_SL.xml and is 4,398,518 bytes in size.

### BACKGROUND

Alternative splicing events in genes can lead to non-productive mRNA transcripts which in turn can lead to aberrant or reduced protein expression, and therapeutic agents which can target the alternative splicing events in genes can modulate the expression level of functional proteins in patients and/or inhibit aberrant protein expression. Such therapeutic agents can be used to treat a condition or disease caused by the protein deficiency.

Autosomal dominant optic atrophy (ADOA) is one of the most commonly diagnosed optic neuropathies. This optic nerve disease is associated with structural and functional mitochondrial deficits that lead to degeneration of the retinal ganglion cells and progressive, irreversible loss of vision. A majority of ADOA patients carry mutations in OPA1 and most mutations lead to haploinsufficiency (Lenaers G. et al. Orphanet J Rare Dis 2012). OPA1 encodes a mitochondrial GTPase with a critical role in mitochondrial fusion, ATP synthesis and apoptosis. Currently, there is no approved disease-modifying treatment for ADOA patients and there is a need for such treatments.

### SUMMARY

In some aspects, provided herein is a method of increasing expression of an OPA1 protein in a cell having a processed mRNA that encodes the OPA1 protein and that comprises a translation regulatory element that inhibits translation of the processed mRNA, the method comprising contacting an agent or a vector encoding the agent to the cell, wherein the agent modulates a structure of the translation regulatory element, thereby increasing expression of the OPA1 protein in the cell.

In some aspects, provided herein is a method of increasing expression of an OPA1 protein in a cell having a processed mRNA that encodes the OPA1 protein and that comprises a translation regulatory element that inhibits translation of the processed mRNA, the method comprising contacting an agent or a vector encoding the agent to the cell, wherein the agent (a) binds to a targeted portion of the processed mRNA; (b) modulates interaction of the translation regulatory element with a factor involved in translation of the processed mRNA; or (c) a combination of (a) and (b), thereby increasing expression of the OPA1 protein in the cell.

In some aspects, provided herein is a method of modulating expression of an OPA1 protein in a cell, the method comprising contacting an agent or a vector encoding the agent to the cell, wherein the agent comprises an antisense oligomer with at least 80% sequence identity to a sequence selected from the group consisting of SEQ ID NOs: 608-1253.

In some aspects, provided herein is a composition comprising an agent or a vector encoding the agent, wherein the agent comprises an antisense oligomer with at least 80% sequence identity to a sequence selected from the group consisting of SEQ ID NOs: 608-1253.

In some aspects, provided herein is a composition comprising a vector encoding an agent, wherein the agent comprises a polynucleic acid comprising a sequence with at least 80% sequence identity to a sequence selected from the group consisting of SEQ ID NOs: 608-1253.

In some aspects, provided herein is a composition comprising an agent, wherein the agent comprises an antisense oligomer that binds to a targeted portion of a processed mRNA that encodes OPA1 protein, wherein the targeted portion of the processed mRNA comprises at least one nucleotide of the main start codon of the processed mRNA or is within the 5′ UTR of the processed mRNA.

In some aspects, provided herein is a composition comprising a vector encoding an agent, wherein the agent comprises a polynucleic acid comprising a sequence that binds to a targeted portion of a processed mRNA that encodes OPA1 protein, wherein the targeted portion of the processed mRNA comprises at least one nucleotide of the main start codon of the processed mRNA or is within the 5′ UTR of the processed mRNA.

In some aspects, provided herein is a composition comprising an agent, wherein the agent modulates structure of a translation regulatory element of a processed mRNA that encodes an OPA1 protein, thereby increasing expression of the OPA1 protein, and wherein the translation regulatory element inhibits translation of the processed mRNA.

In some aspects, provided herein is a composition comprising a vector encoding an agent, wherein the agent modulates structure of a translation regulatory element of a processed mRNA that encodes an OPA1 protein, thereby increasing expression of the OPA1 protein, and wherein the translation regulatory element inhibits translation of the processed mRNA.

In some aspects, provided herein is a composition comprising an agent, wherein the agent increases translation of a processed mRNA in a cell, wherein the processed mRNA encodes OPA1 protein and comprises a translation regulatory element that inhibits the translation of the processed mRNA, wherein the agent modulates a structure of the translation regulatory element, thereby increasing translation efficiency and/or the rate of translation of the processed mRNA, wherein the agent (a) binds to a targeted portion of the processed mRNA; (b) modulates interaction of the translation regulatory element with a factor involved in translation of the processed mRNA; or (c) a combination of (a) and (b).

In some aspects, provided herein is a composition comprising a vector encoding an agent, wherein the agent increases translation of a processed mRNA in a cell, wherein the processed mRNA encodes OPA1 protein and comprises a translation regulatory element that inhibits the translation of the processed mRNA, wherein the agent modulates a structure of the translation regulatory element, thereby increasing translation efficiency and/or the rate of translation of the processed mRNA, wherein the agent (a) binds to a targeted portion of the processed mRNA; (b) modulates interaction of the translation regulatory element with a factor involved in translation of the processed mRNA; or (c) a combination of (a) and (b).

In some aspects, provided herein is a method of increasing expression of a target protein in a cell having a processed mRNA that encodes the target protein and comprises a translation regulatory element that inhibits translation of the processed mRNA, the method comprising delivering into the cell: (1) a first agent or a first nucleic acid sequence encoding the first agent, and (2) a second agent or a second nucleic acid sequence encoding the second agent, wherein the first agent modulates splicing of a pre-mRNA that is transcribed from an target gene that encodes the target protein, and wherein the second agent modulates a structure of the translation regulatory element of the processed mRNA that encodes the target protein, thereby increasing the expression of the target protein in the cell.

In some aspects, provided herein is a method of increasing expression of a target protein in a cell having a processed mRNA that encodes the target protein and comprises a translation regulatory element that inhibits translation of the processed mRNA, the method comprising delivering into the cell: (1) a first agent or a first nucleic acid sequence encoding the first agent, and (2) a second agent or a second nucleic acid sequence encoding the second agent, wherein the first agent modulates splicing of a pre-mRNA that is transcribed from an target gene that encodes the target protein, and wherein the second agent (a) binds to a targeted portion of the processed mRNA; (b) modulates interaction of the translation regulatory element with a factor involved in translation of the processed mRNA; or (c) a combination of (a) and (b), thereby increasing the expression of the target protein in the cell.

In some aspects, provided herein is a method of modulating expression of a target protein in a cell, wherein contacting to the cell: (1) a first agent or a first nucleic acid sequence encoding the first agent, and (2) a second agent or a second nucleic acid sequence encoding the second agent, wherein the first agent comprises a first antisense oligomer with at least 80% sequence identity to a sequence selected from the group consisting of SEQ ID NOs: 6-275 and 280-299, and the second agent comprises a second antisense oligomer that binds to 5′ UTR of a processed mRNA that encodes the target protein, and wherein the target protein is an OPA1 protein.

In some aspects, provided herein is a pharmaceutical composition, comprising (1) a first therapeutic agent or a first nucleic acid sequence encoding the first therapeutic agent, and (2) a second therapeutic agent or a second nucleic acid sequence encoding the second therapeutic agent, wherein the first therapeutic agent comprises a first antisense oligomer with at least 80% sequence identity to a sequence selected from the group consisting of SEQ ID NOs: 6-275 and 280-299, and the second therapeutic agent comprises a second antisense oligomer that binds to 5′ UTR of a processed mRNA that encodes an OPA1 protein.

In some aspects, provided herein is a composition, comprising an antisense oligomer with at least 80% sequence identity to a sequence selected from the group consisting of SEQ ID NOs: 608-1253, wherein the antisense oligomer comprises a backbone modification, a sugar moiety modification, or a combination thereof.

In some aspects, provided herein is a pharmaceutical composition comprising the composition disclosed herein, and a pharmaceutically acceptable carrier or excipient.

In some aspects, provided herein is a pharmaceutical composition, comprising a therapeutic agent or a vector encoding the therapeutic agent, and a pharmaceutically acceptable carrier or excipient, wherein the therapeutic agent modulates structure of a translation regulatory element of a processed mRNA that encodes an OPA1 protein, thereby increasing expression of the OPA1 protein, and wherein the translation regulatory element inhibits translation of the processed mRNA.

In some aspects, provided herein is a pharmaceutical composition, comprising a therapeutic agent or a vector encoding the therapeutic agent, and a pharmaceutically acceptable carrier or excipient, wherein the therapeutic agent (a) binds to a targeted portion of a processed mRNA that encodes an OPA1 protein and that comprises a translation regulatory element; (b) modulates interaction of the translation regulatory element with a factor involved in translation of the processed mRNA; or (c) a combination of (a) and (b), thereby increasing the expression of the OPA1 protein in the cell, and wherein the translation regulatory element inhibits translation of the processed mRNA.

In some aspects, provided herein is a pharmaceutical composition comprising the first therapeutic agent of the method disclosed herein, the second therapeutic agent, and a pharmaceutically acceptable excipient.

In some aspects, provided herein is a kit comprising the first therapeutic agent of the method disclosed herein in a first contained and the second therapeutic agent of the method disclosed herein in a second container.

In some aspects, provided herein is a pharmaceutical composition comprising a first vector encoding the first therapeutic agent of the method disclosed herein, a second vector encoding the second therapeutic agent, and a pharmaceutically acceptable excipient.

In some aspects, provided herein is a kit comprising a first vector the first therapeutic agent of the method disclosed herein in a first contained and a first vector the second therapeutic agent of the method disclosed herein in a second container.

In some aspects, provided herein is a pharmaceutical composition comprising a vector encoding the first therapeutic agent of the method disclosed herein and the second therapeutic agent, and a pharmaceutically acceptable excipient.

In some aspects, provided herein is a pharmaceutical composition, comprising: (1) a first therapeutic agent or a first nucleic acid sequence encoding the first therapeutic agent, (2) a second therapeutic agent or a second nucleic acid sequence encoding the second therapeutic agent, and (3) a pharmaceutically acceptable carrier or excipient, wherein the first therapeutic agent modulates splicing of a pre-mRNA that is transcribed from a target gene that encodes the target protein, and wherein the second therapeutic agent (a) binds to a targeted portion of a processed mRNA that encodes an OPA1 protein and that comprises a translation regulatory element; (b) modulates interaction of the translation regulatory element with a factor involved in translation of the processed mRNA; or (c) a combination of (a) and (b), and wherein the translation regulatory element inhibits translation of the processed mRNA.

In some aspects, provided herein is a pharmaceutical composition, comprising: (1) a first therapeutic agent or a first nucleic acid sequence encoding the first therapeutic agent, (2) a second therapeutic agent or a second nucleic acid sequence encoding the second therapeutic agent, and (3) a pharmaceutically acceptable carrier or excipient, wherein the first therapeutic agent modulates splicing of a pre-mRNA that is transcribed from a target gene that encodes the target protein, and wherein the second therapeutic agent modulates a structure of a translation regulatory element of a processed mRNA that encodes the target protein, wherein the translation regulatory element inhibits translation of the processed mRNA.

In some aspects, provided herein is a method of treating or reducing the likelihood of developing a disease or condition in a subject in need thereof by modulating expression of an OPA1 protein in a cell of the subject, comprising contacting to cells of the subject the therapeutic agent disclosed herein, or the first therapeutic agent and the second therapeutic agent disclosed herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 is a bar graph depicting the fold change of OPA1 protein level in the cells treated with exemplary ASOs according to some embodiments of the present disclosure.

FIGS. 2A, 2B, and 3 show secondary structures of the 5′ UTR region and main start codon of OPA1 transcript ENST00000361908 based on computational simulations using two different software tools. FIG. 2 discloses SEQ ID NOS 1313-1314, respectively in order of appearance. FIG. 3 discloses SEQ ID NO: 1313.

FIGS. 4-5 show the computationally predicted secondary structures of the region covering the 5′ UTR and main start codon of the OPA1 processed mRNA upon binding with exemplary ASOs ASO-U35 and ASO-U34, respectively. FIG. 4 discloses SEQ ID NO: 1313.

FIG. 5 discloses SEQ ID NO: 1313.

FIG. 6 shows the computationally predicted secondary structure of the region covering the 5′ UTR and main start codon of the OPA1 processed mRNA upon binding with exemplary ASO ASO-U33. FIG. 6 discloses SEQ ID NO: 1315.

FIGS. 7-9 show the computationally predicted secondary structures of the region covering the 5′ UTR and main start codon of the OPA1 processed mRNA upon binding with exemplary ASOs ASO-U37, ASO-U35, and ASO-U33, respectively. FIG. 7 discloses SEQ ID NO: 1313. FIG. 8 discloses SEQ ID NO: 1313. FIG. 9 discloses SEQ ID NO: 1313.

FIG. 10 shows the computationally predicted secondary structure of the region covering the 5′ UTR and main start codon of the OPA1 processed mRNA upon binding with exemplary ASO ASO-U39. FIG. 10 discloses SEQ ID NO: 1313.

FIGS. 11A-11C depict a schematic representation of a target mRNA that contains a nonsense-mediated mRNA decay-inducing exon (NMD exon mRNA) and therapeutic agent-mediated exclusion of the nonsense-mediated mRNA decay-inducing exon to increase expression of the full-length target protein or functional RNA. FIG. 11A shows a cell divided into nuclear and cytoplasmic compartments. In the nucleus, a pre-mRNA transcript of a target gene undergoes splicing to generate mRNA, and this mRNA is exported to the cytoplasm and translated into target protein. For this target gene, some fraction of the mRNA contains a nonsense-mediated mRNA decay-inducing exon (NMD exon mRNA) that is degraded in the cytoplasm, thus leading to no target protein production. FIG. 11B shows an example of the same cell divided into nuclear and cytoplasmic compartments. Treatment with a therapeutic agent, such as an antisense oligomer (ASO), promotes the exclusion of the nonsense-mediated mRNA decay-inducing exon and results in an increase in mRNA, which is in turn translated into higher levels of target protein. FIG. 11C shows an example schematic of a novel NMD exon inclusion event (Exon X) identified by the center rectangle which leads to the introduction of a premature termination codon (PTC), resulting in a non-productive mRNA transcript degraded by nonsense-mediated decay (NMD).

FIG. 12 depicts identification of an exemplary nonsense-mediated mRNA decay (NMD)-inducing exon in the OPA1 gene. The identification of the NMD-inducing exon in the OPA1 gene using RNA sequencing is shown, visualized in the UCSC genome browser. The upper panel shows a graphic representation of the OPA1 gene to scale. Peaks corresponding to RNA sequencing reads were identified in intron GRCh38/hg38: chr3 193626204 to 193631611, shown in the middle panel. Bioinformatic analysis identified an exon-like sequence (bottom panel, sequence highlighted in uppercase; GRCh38/hg38: chr3 193628509 to 193628616) flanked by 3′ and 5′ splice sites. Inclusion of this exon leads to the introduction of a premature termination codon rendering the transcript a target of NMD. FIG. 12 discloses SEQ ID NO: 300.

FIG. 13 depicts identification of an exemplary nonsense-mediated mRNA decay (NMD)-inducing exon in the OPA1 gene. The identification of the NMD-inducing exon in the OPA1 gene using RNA sequencing is shown, visualized in the UCSC genome browser. The upper panel shows a graphic representation of the OPA1 gene to scale. Peaks corresponding to RNA sequencing reads were identified in intron GRCh38/hg38: chr3 193593374 to 193614710, shown in the middle panel. Bioinformatic analysis identified an exon-like sequence (bottom panel, sequence highlighted in uppercase; GRCh38/hg38: chr3 193603500 to 193603557) flanked by 3′ and 5′ splice sites. Inclusion of this exon leads to the introduction of a premature termination codon rendering the transcript a target of NMD. FIG. 13 discloses SEQ ID NO: 301.

FIG. 14 depicts confirmation of NMD-inducing exon via puromycin or cycloheximide treatment in various cell lines, as well as the confirmation of NMD-inducing exon in brain and retina samples. RT-PCR analysis using total RNA from water-treated, DMSO-treated, puromycin-treated, or cycloheximide-treated cells confirmed the presence of a band corresponding to the NMD-inducing exon 7x (GRCh38/hg38: chr3 193628509 to 193628616) of OPA1 gene.

FIG. 15 depicts an exemplary ASO walk around OPA1 exon 7x (GRCh38/hg38: chr3 193628509 193628616) region. A graphic representation of an ASO walk performed for around OPA1 exon 7x (GRCh38/hg38: chr3 193628509 193628616) region targeting sequences upstream of the 3′ splice site, across the 3′splice site, exon 7x, across the 5′ splice site, and downstream of the 5′ splice site is shown. ASOs were designed to cover these regions by shifting 5 nucleotides at a time or 3 nucleotides across the splice site regions. FIG. 15 discloses SEQ ID NOS 302-304, respectively, in order of appearance.

FIGS. 16A-16C depict data for an OPA1 exon 7x (GRCh38/hg38: chr3 193628509 193628616) region ASO walk evaluated by TaqMan RT-qPCR. Graphs of fold-change of the OPA1 productive mRNA product relative to Sham are plotted. FIG. 16A shows relative Opal mRNA expression relative to the sham control for microwalk starting from IVS6X-86 to IVS6X-1. FIG. 16B shows relative Opal mRNA expression relative to the sham control for microwalk starting from IVS6X/EX7X+3 to EX7X/IVS7X-3. FIG. 16C shows relative Opal mRNA expression relative to the sham control for microwalk starting from IVS7X+1 to IVS7X+86.

FIG. 17 depicts an OPA1 exon 7x (GRCh38/hg38: chr3 193628509 193628616) region ASO walk evaluated by TaqMan RT-qPCR. Graphs of fold-change of the OPA1 productive mRNA product relative to Sham are plotted.

FIG. 18 illustrates expression of OPA1 transcripts containing the NMD exon in HEK293 cells treated with increasing amounts of cycloheximide.

FIG. 19A illustrates RT-PCR data from the posterior segment of the eye of Chlorocebus sabaeus (green monkey) at postnatal data P93 (3 months) and postnatal day P942 (2.6 years). FIG. 19A confirms expression of OPA1 transcripts containing the NMD exon in these cells.

FIG. 19B illustrates quantification of the NMD exon abundance from FIG. 19A.

FIG. 20A illustrates RT-PCR of the productive and non-productive OPA1 mRNA after treatment of HEK293 cells with various ASOs and cycloheximide.

FIG. 20B illustrates quantification of the data in FIG. 20A.

FIG. 21 illustrates expression of productive OPA1 mRNA by quantitative PCR in HEK293 cells treated with various ASOs and not treated with cycloheximide.

FIG. 22A illustrates RT-PCR for non-productive OPA1 mRNAs in HEK293 cells after treatment with ASO-14 and cycloheximide.

FIG. 22B illustrates quantification of productive OPA1 mRNAs in HEK293 cells after treatment with ASO-14 in the absence of cycloheximide.

FIG. 22C illustrates protein expression of OPA1 in HEK293 cells after treatment with ASO-14 in the absence of cycloheximide.

FIG. 23A illustrates mRNA and protein levels of OPA1 gene in OPA1 haploinsufficient (OPA1+/−) HEK293 cells.

FIG. 23B illustrates OPA1 protein expression in the OPA1 haploinsufficient (OPA1+/−) HEK293 cells after treatment with ASO-14.

FIG. 23C illustrates quantification of OPA1 protein expression in the OPA1 haploinsufficient (OPA1+/−) HEK293 cells after treatment with ASO-14.

FIG. 24A illustrates study design for the in vivo rabbit experiment of Example 2.14.

FIG. 24B illustrates levels of productive and non-productive OPA1 mRNA and protein.

FIG. 24C illustrates quantification of the data from FIG. 24B.

FIG. 25 illustrates exemplary OPA1 ASOs of this disclosure. The right two columns in the chart illustrate the chemical modifications of the exemplary ASOs. Each nucleotide of all the ASOs has 2′-O-methoxyethyl (2′MOE) modification (“MOE”) unless otherwise noted, for instance, letters of larger font size (e.g., G) are locked nucleic acids (“LNA”), underlined letters (e.g., C) are 5′ methyl-cytosines that have 2′-MOE moiety (“5MeC-MOE”), and some ASOs are noted as phosphorodiamidate morpholino oligomers (“PMO”). FIG. 25 discloses SEQ ID NOS 6-148, 148, 148, 149, 149, 149, 150, 150, 150-151, 151, 151, 123, 152, 152, 152-153, 153, 153-154, 154, 154, 144-146, 93, 81-82, 36, 155, 155-156, 156-157, 157-161, 125, 162, 126, 163-166, 92, 167-179, 156, 180, 157, 159, 181, 160, 182, 161, 183-275, and 305-607 respectively, in order of column.

FIG. 26A illustrates RT-PCR results for OPA1 mRNAs using probes spanning exon 7 and exon 8 in HEK293 cells after treatment with ASO-14 and cycloheximide.

FIG. 26B illustrates quantification of OPA1 mRNAs in HEK293 cells after treatment with ASO-14 in the absence of cycloheximide based on qPCR using probes spanning exons 6 and 8, probes spanning exons 7 and 8, or probes spanning exons 23 and 24.

FIG. 26C illustrates sequencing data on the relative amount of various OPA1 mRNA transcripts in HEK293 cells transfected with ASO-14.

FIG. 27A illustrates RT-PCR results for OPA1 mRNAs using probes spanning exon 6 and exon 8 in HEK293 cells after treatment with various exemplary OPA1 ASOs.

FIG. 27B illustrates relative ratio of OPA1 mRNA transcripts having exons 6, 7, and 8 in tandem (“6-7-8”) over the total amount of “6-7-8” transcripts and transcripts having exons 6 and 8 in tandem (“6-8”), in HEK293 cells after treatment with various exemplary OPA1 ASOs.

FIGS. 27C and 27D illustrate quantification of OPA1 mRNAs using probes spanning exons 6 and 8, and probes spanning exons 7 and 8, respectively, in HEK293 cells after treatment with various exemplary OPA1 ASOs.

FIG. 28A illustrates RT-PCR results for OPA1 mRNAs using probes spanning exon 6 and exon 8 (“Exon 6-8 PCR”), or probes spanning exon 7x and exon 8 (“Exon 7x-8 PCR”), in HEK293 cells after treatment with various exemplary OPA1 ASOs and treatment with cycloheximide.

FIG. 28B illustrates expression level of OPA1 protein in HEK293 cells after treatment with various exemplary OPA1 ASOs.

FIG. 28C illustrates dose response in OPA1 mRNAs using probes spanning exon 6 and exon 8 in HEK293 cells after treatment with various exemplary OPA1 ASOs.

FIGS. 28D and 28E illustrate quantification of the dose response in OPA1 mRNAs using probes spanning exons 6 and 8, probes spanning exons 7 and 8, probes spanning exons 23 and 24, respectively, in HEK293 cells after treatment with various exemplary OPA1 ASOs. FIG. 28D summarizes the Ct values for the qPCR reactions, and FIG. 28E summarizes the relative amounts.

FIG. 28F illustrates dose response in expression level of OPA1 protein in HEK293 cells after treatment with various exemplary OPA1 ASOs.

FIGS. 29A-29D illustrate RT-PCR results for OPA1 mRNAs using probes spanning exon 6 and exon 8 (“Exon 6-8”), or probes spanning exon 7x and exon 8 (“Exon 7-8”), in HEK293 cells after treatment with various exemplary OPA1 ASO 18-mers and treatment with or without cycloheximide.

FIGS. 30A-30B illustrate RT-PCR results for OPA1 mRNAs using probes spanning exon 6 and exon 8 (“Exon 6-8”), or probes spanning exon 7x and exon 8 (“Exon 7x-8”), in HEK293 cells after treatment with various exemplary OPA1 ASO 18-mers and treatment with or without cycloheximide.

FIGS. 31A-31D illustrate RT-PCR results for OPA1 mRNAs using probes spanning exon 6 and exon 8 (“Exon 6-8”), or probes spanning exon 7x and exon 8 (“Exon 7-8”), in HEK293 cells after treatment with various exemplary OPA1 ASO 16-mers and treatment with or without cycloheximide.

FIGS. 32A-32C illustrate RT-PCR results for OPA1 mRNAs using probes spanning exon 6 and exon 8 (“Exon 6-8”), or probes spanning exon 7x and exon 8 (“Exon 7x-8”), in HEK293 cells after treatment with various exemplary OPA1 ASO 15-mers and treatment with or without cycloheximide.

FIGS. 33A-33B illustrate dose response in OPA1 mRNAs having Exon 6 and Exon 8 (“6-8”), having Exon 7 and Exon 8 (“7-8”), or having Exon 7x and Exon 8 (“7x-8”) in HEK293 cells after treatment with different concentrations of various exemplary OPA1 ASOs.

FIG. 34A is a histogram that demonstrates ATP level was reduced in mock-treated OPAT+/−HEK293 cells as compared to OPAT+/+HEK293 cells, and ASO-14 treatment of OPAT+/−HEK293 cells increased the ATP level in the cells.

FIGS. 34B-34C demonstrate the OPA1 protein was increased by ASO-14 in OPAT+/+HEK293 cells. FIG. 34B shows the immunoblot gel images of OPA1 and j-actin proteins, and FIG. 34C is a histogram that summarizes quantification of the immunoblot results.

FIGS. 35A-35B show histograms that demonstrate mRNA (FIG. 35A) and protein expression (FIG. 35B) of OPA1 gene were reduced in fibroblast cells from diagnosed patients that have haploinsufficient mutation in OPA1 gene as compared to wildtype (WT) fibroblast cells. FIG. 35C shows a representative immunoblot image of OPA protein expression level in diseased fibroblast cells.

FIGS. 36A, 36B, and 36D show histograms that demonstrate exemplary antisense oligomer, ASO-14, decreased OPA1 NMD exon inclusion (FIG. 36A), increased OPA1 total mRNA level (FIG. 36B), and protein level (FIG. 36D) in wildtype (WT) fibroblast cells and fibroblast cells from diagnosed patients that have haploinsufficient mutation in OPA1 gene. FIG. 36C shows representative immunoblot images of OPA1 protein and loading control β-Tubulin under all types of conditions.

FIGS. 37A-37E demonstrate that patient fibroblast cells (cell lines F35 and F36) show deficiencies in mitochondrial bioenergetics. FIG. 37A shows representative time courses of the oxygen consumption rate of WT cells, F35 cells, and F36 cells at baseline level and when challenged sequentially with oligomycin, FCCP, rotenone and antimycin A. FIGS. 37B-37E show histograms demonstrating that patient fibroblast cells, F35 and F36 cells had reduced basal oxygen consumption rate (FIG. 37B), ATP-linked respiration (FIG. 37C), maximal respiration (FIG. 37D), and spare respiratory capacity (FIG. 37E), as compared to WT fibroblast cells.

FIGS. 38A-38D show histograms demonstrating that treatment of ASO-14 at 20 nM, 40 nM, and 60 nM increased basal oxygen consumption rate (FIG. 38A), ATP-linked respiration (FIG. 38B), maximal respiration (FIG. 38C), and spare respiratory capacity (FIG. 38D) of F35 patient cells in a dose-dependent manner.

FIGS. 39A-39D show histograms demonstrating that treatment of ASO-14 at 20 nM, 40 nM, and 60 nM increased basal oxygen consumption rate (FIG. 39A), ATP-linked respiration (FIG. 39B), maximal respiration (FIG. 39C), and spare respiratory capacity (FIG. 39D) of F36 patient cells in a dose-dependent manner.

FIG. 40 shows a diagram illustrating 5′ UTR region of a series of various OPA1 mRNA transcripts, with overlaying gray boxes indicating the location of start codons of three upstream open reading frames and the main start codon, which is shown to have the best Kozak sequence surrounding it. FIG. 40 discloses “MWRLRRAAVAC” as SEQ ID NO: 1316.

FIG. 41 shows a diagram illustrating 5′ UTR region of a series of various OPA1 mRNA transcripts, with (1) overlaying gray boxes indicating the location of start codons of three upstream open reading frames and the main start codon, and (2) blow-up view of the sequences of G-quadruplex motifs that are predicted to be present in the 5′ UTR according to G4IPDB. See Mishra S K, et al. G4IPDB: A database for G-quadruplex structure forming nucleic acid interacting proteins. Sci Rep. 2016 Dec. 1; 6:38144. doi: 10.1038/srep38144. FIG. 41 discloses SEQ ID NOS: 1317-1319 and “MWRLRRAAVAC” as SEQ ID NO: 1316, respectively in order of appearance.

### DETAILED DESCRIPTION

Agents that modulate transcription of pre-mRNA from a gene, splicing of the pre-mRNA into a mature mRNA (or “processed mRNA”), and/or translation of the mature mRNA can modulate expression of protein that is encoded by the gene. Provided herein, in some aspects, are methods, compositions, and kits relating to an agent that modulates protein expression by modulating mRNA splicing and/or translation. In some aspects, provided herein are methods, compositions, and kits relating to a vector encoding an agent that modulates protein expression by modulating mRNA splicing and/or translation.

In some aspects, provided herein are methods, compositions, and kits relating to an agent, e.g., a therapeutic agent, that can modulate a translation regulatory element present within a processed mRNA, e.g., by modulating a structure of the translation regulatory element, or modulating interaction of the translation regulatory element with a factor involved in translation of the processed mRNA, and thus can modulate translation of the processed mRNA into a protein encoded by the processed mRNA. In some aspects, provided herein are methods, compositions, and kits relating to an agent, e.g., a therapeutic agent, that can bind to at least a portion of the 5′ untranslated region (“5′ UTR”) of a processed mRNA. In some cases, the agent disclosed herein modulates a translation regulatory element present within the 5′ UTR of the processed mRNA, thereby modulating translation of the processed mRNA and modulating expression of a protein from the processed mRNA. Modulation of one or more translation regulatory element present in a processed mRNA according to some embodiments of the present disclosure can thus modulate expression of a target protein translated from the processed mRNA, which in consequence can provide therapeutic intervention for diseases or conditions that are associated with aberrant level or activity of the target protein.

In some aspects, provided herein are methods, compositions, and kits relating to an agent, e.g., a therapeutic agent, that can modulate alternative splicing of a pre-mRNA, or a vector encoding the agent. Alternative splicing events in some genes, e.g., OPA1 gene, can lead to non-productive mRNA transcripts which in turn can lead to aberrant protein expression. Agents which can target the alternative splicing events in those genes can modulate the expression level of functional target proteins in patients and/or inhibit aberrant protein expression. Such agents can thus be used to treat a disease or condition that is associated with aberrant level or activity of the target protein.

One of the alternative splicing events that can lead to non-productive mRNA transcripts is the inclusion of an extra exon in the mRNA transcript that can induce nonsense-mediated mRNA decay. In some aspects, the present disclosure provides compositions, methods, and kits for modulating alternative splicing of a target pre-mRNA, e.g., OPA1 pre-mRNA, to increase the production of protein-coding processed mRNA, and thus, translated functional target protein.

In some aspects, provided herein are compositions, methods, and kits relating to a combination therapy that utilizes an agent that modulates a translation regulatory element of a processed mRNA that encoding a target protein, e.g., OPA1 protein, and an agent that modulates splicing of a pre-mRNA that is transcribed from a gene that encodes the target protein, e.g., OPA1 gene. In some aspects, provided herein are compositions, methods, and kits relating to a combination therapy that utilizes an agent that target at least a portion of the 5′ UTR of a processed mRNA that encoding a target protein, e.g., OPA1 protein, and an agent that modulates splicing of a pre-mRNA that is transcribed from a gene that encodes the target protein, e.g., OPA1 gene.

### Translation Regulatory

Without wishing to be bound by a certain theory, eukaryotic mRNAs can be translated by the scanning mechanism, which begins with assembly of a 43S preinitiation complex (PIC), containing methionyl-initiator tRNA (Met-tRNAi) in a ternary complex (TC) with guanosine triphosphate (GTP)-bound eukaryotic initiation factor 2 (eIF2). The 43S PIC assembly is stimulated by eIFs 1, 1A, 3, and 5. Its subsequent attachment to a processed mRNA at the m7G-capped 5′ end is facilitated by the eIF4F complex-composed of cap-binding protein eIF4E, eIF4G, and RNA helicase eIF4A- and by poly(A)-binding protein (PABP). The PIC can scan the mRNA 5′ untranslated region (UTR) for an AUG nucleotide triplet start codon using complementarity with the anticodon of Met-tRNAi. AUG recognition can evoke hydrolysis of the GTP bound to eIF2 to produce a stable 48S PIC. Release of eIF2-GDP is followed by joining of the large (60S) ribosome subunit, catalyzed by eIF5B, to produce an 80S initiation complex ready to begin protein synthesis.

RNA molecules can fold into intricate shapes that can provide an additional layer of control of gene expression beyond that of their sequence. The 5′ UTR is the region of a processed mRNA that is directly upstream from the main initiation codon. The terms “main initiation codon” or “main start codon,” as used herein, can refer to a start codon that initiate translation of the main open reading frame of a processed mRNA. The term “open reading frame,” as used herein, can refer to a continuous stretch of codons that may begin with a start codon and ends at a stop codon. The “main open reading frame” of a processed mRNA, as used herein, is the open reading frame that encodes the protein that processed mRNA and the gene, from which the processed mRNA is transcribed, are primary responsible for expressing. For instance, the main open reading frame of an OPA1 processed mRNA is the open reading frame on the OPA1 processed mRNA that encodes the OPA1 protein. Similarly, the main open reading frame of an ALB processed mRNA that is transcribed from ALB gene (a gene encoding albumin) is the open reading frame on the ALB processed mRNA that encodes albumin.

The 5′ UTR region can modulate translation of a processed mRNA by differing mechanisms in viruses, prokaryotes and eukaryotes. In some processed mRNAs, 5′ UTRs can be highly structured and some of the secondary structures or higher order structures formed by the 5′ UTR can block entry of the ribosome. For instance, in some processed mRNAs, there can be one or more secondary structures or higher order structures formed within the 5′ UTR or formed by a portion of the sequence of the 5′ UTR and a sequence at other part of the processed mRNA. In some cases, secondary structures like stem, loop (e.g., hairpin loop), and/or stem-loop can be formed within the 5′ UTR. In some cases, there can be G-quadruplex motif present within the 5′ UTR. As used herein, the term “G-quadruplex” can refer to an RNA structure formed in G-rich regions by the stacking of at least two G-tetrads, each of them forming a square-shaped structure by non-Watson-Crick interactions between two or more layers of paired G-quartets. In some cases, G-quadruplex can be extremely stable, for instance, stable in vitro with melting temperatures that are higher than physiological temperature, especially in the presence of potassium ions (K+), which are specifically chelated inside G-quartets. Description of G-quadruplex can be found in Beaudoin J D & Perreault J P. Nucleic Acids Res. 2010 November; 38(20):7022-36. doi: 10.1093/nar/gkg557, Todd A. K., et al. Nucleic Acids Res. 33, 2901-2907 (2005), Huppert J L & Balasubramanian S. Nucleic Acids Res. 2005 May 24; 33(9):2908-16. doi: 10.1093/nar/gki609, each of which is incorporated herein by reference in its entirety. In some cases, the formation of G-quadruplex can be the most stable RNA structure that could block ribosome scanning. In some cases, G-quadruplex has a sequence according to the formula Gx-N1-7-Gx-N1-7-Gx-N1-7-Gx, where x≥3 and N is A, C, G or U. In some cases, a G-rich sequence that may form a G-quadruplex comprises a sequence GGGAGCCGGGCUGGGGCUCACACGGGGG (SEQ ID NO: 1310). In some cases, some of the sequences or secondary or higher order structures of the 5′ UTR region of a processed mRNA can recruit one or more RNA-binding proteins (RBPs), which can be involved in modulation of the translation of the processed mRNA.

Upstream open reading frames and upstream start codon can also modulate protein expression by suppressing translation of the processed mRNA encoding the protein. The terms “upstream open reading frame” or “uORF,” as used herein, can refer to an open reading frame located in the 5′ UTR of a processed mRNA, e.g., an open reading frame that is upstream of the main start codon of the processed mRNA. The terms “upstream start codon,” or “upstream initiation codon,” as used herein, can refer to a start codon located in the 5′ UTR of a processed mRNA, e.g., a start codon upstream of the main start codon. An upstream start codon can suppress translation of the processed mRNA that starts from the main start codon. The nature of ribosome scanning during translation of a processed mRNA, its 5′ to 3′ directionality, can dictate that the initiation codon is frequently the AUG triplet closest to the 5′ end, encountered first by the scanning PIC. In some embodiments, some of the first AUG nucleotide triplets can be skipped when it is flanked by an unfavorable sequence—a process termed “leaky scanning”—to use a downstream AUG. A favorable sequence context in mammals is the “Kozak consensus,” 5′ (A/G)CCAUGG 3′. When an upstream start codon (e.g., upstream AUG, or uAUG) is in-frame with a downstream AUG without an intervening stop codon, leaky scanning may occur at some frequency to allow production of two protein isomers differing only by an N-terminal extension, with the longer form often targeted to a particular cellular compartment. If, in some cases, the uAUG is followed by a stop codon in the same open reading frame (ORF), then translation of the upstream ORF (uORF) can attenuate translation of the downstream main ORF (mORF), because re-initiation of the translation is generally inefficient. Some uORFs inhibit downstream translation primarily because ribosomes can stall during their translation and create a roadblock to scanning PICs that bypass the uORF start codon. “Near-cognate” triplets, e.g., NUG (N is any nucleotide) triplets or A(A/G)G triplets, differing from AUG by a single base, can be selected by the scanning PIC but with lower frequencies, owing to the mismatch with the anticodon of tRNAi and attendant destabilization of the 48S PIC. Start codons disclosed herein can include AUG and near-cognate triplets.

Non-limiting examples of a translation regulatory elements disclosed herein can include a secondary structure of a processed mRNA, e.g., a secondary structure in the 5′ UTR, e.g., a stem, a loop, or a stem-loop, a G-quadruple motif, and an upstream start codon. In some cases, a translation regulatory element of a processed mRNA disclosed herein modulates translation of the processed mRNA by modulating translation efficiency and/or rate of translation of the processed mRNA. In some embodiments, a translation regulatory element disclosed herein inhibits translation of the processed mRNA. For instance, a translation regulatory element can block ribosome scanning during translation, thus suppressing translation efficiency and/or rate of translation. In some cases, an upstream start codon can initiate translation of the upstream open reading frame that is led by it, which prevents the scanning ribosome from re-initiation to translate the main open reading frame, thus suppressing expression of the protein that is encoded by the main open reading frame of the processed mRNA.

Targeting Processed mRNA

In some embodiments, the methods and compositions of the present disclosure relate to modulation of translation of processed mRNA transcribed from a target gene, e.g., OPA1 gene. In some embodiments, an agent provided herein targets a processed mRNA that encodes a target protein (e.g., OPA1 protein) and comprises a translation regulatory element that inhibits translation of the processed mRNA. In some cases, the agent modulates a structure of the translation regulatory element, thereby increasing expression of the target protein in a cell. In some cases, the agent (a) binds to a targeted portion of the processed mRNA; (b) modulates interaction of the translation regulatory element with a factor involved in translation of the processed mRNA; or (c) a combination of (a) and (b), thereby increasing expression of the target protein (e.g., OPA1 protein) in the cell.

In some cases, the resulting increase in expression of OPA1 protein induced by the agent that targets a processed mRNA that encodes OPA1 protein can alleviate symptoms of a condition or disease associated with OPA1 deficiency, such as an eye disease or condition, Optic atrophy type 1, autosomal dominant optic atrophy (ADOA), ADOA-plus syndrome; a mitochondrial disorder; glaucoma; normal tension glaucoma; Charcot-Marie-tooth disease; mitochondria dysfunction; diabetic retinopathy; age-related macular degeneration; retinal ganglion cell death; mitochondrial fission-mediated mitochondrial dysfunction; progressive external ophthalmoplegia; deafness; ataxia; motor neuropathy; sensory neuropathy; myopathy; Behr syndrome; brain dysfunction; encephalopathy; peripheral neuropathy; fatal infantile mitochondrial encephalomyopathy; hypertrophic cardiomyopathy; spastic ataxic syndrome; sensory motor peripheral neuropathy; hypotonia; gastrointestinal dysmotility and dysphagia; optic atrophy; optic atrophy plus syndrome; Mitochondrial DNA depletion syndrome 14; late-onset cardiomyopathy; diabetic cardiomyopathy; Alzheimer's Disease; focal segmental glomerulosclerosis; kidney disease; Huntington's Disease; cognitive function decline in healthy aging; Prion diseases; late onset dementia and parkinsonism; mitochondrial myopathy; Leigh syndrome; Friedreich's ataxia; Parkinson's disease; MELAS (Mitochondrial encephalomyopathy, lactic acidosis, and stroke-like episodes); pyruvate dehydrogenase complex deficiency; chronic kidney disease; Leber's hereditary optic neuropathy; obesity; age-related systemic neurodegeneration; skeletal muscle atrophy; heart and brain ischemic damage; massive liver apoptosis; NARP (neuropathy, ataxia, retinitis pigmentosa); MERRF (myoclonic epilepsy and ragged red fibers); Pearsons/Kems-Sayre syndrome; MIDD (maternally inherited diabetes and deafness); mitochondrial trifunctional protein deficiency; Fuchs corneal endothelial dystrophy; macular telangiectasia; retinitis pigmentosa; Leber congenital amaurosis; inherited maculopathy; Stargardt disease; or Sorsby fundus dystrophy.

In some embodiments, the translation regulatory element is in a 5′ untranslated region (5′ UTR) of the processed mRNA. In some cases, the translation regulatory element comprises at least a portion of a 5′ UTR of the processed mRNA. In some cases, the translation regulatory element comprises a secondary mRNA structure that involves base-pairing with at least one nucleotide of the main start codon of the processed mRNA. In some of these cases, an agent provided herein inhibits the base-pairing with the at least one nucleotide of the main start codon of the processed mRNA. For instance, the agent can unstructured at least one, two, or three of the three nucleotides of the main start codon that tend to be involved in base pairing in a secondary mRNA structure, e.g., a stem, a loop, or a stem-loop. In some embodiments, the mRNA secondary structure comprises a stem, a stem loop, a Guanine quadruplex, or any combination thereof. In some cases, the agent does not bind to the main start codon. For instance, the agent binds to a portion of the processed mRNA that is different from the main start codon, e.g., at least 10, 20, 30, 40, 50, 60, 70, 80, 90, 100, 120, 140, 160, 180, 200, 220, or 230 nucleotides upstream of the main start codon, or at least 10, 20, 30, 40, 50, 60, 70, 80, 90, 100, 120, 140, 160, 180, 200, 250, 300, 400, 500, 600, 800, 1000, or even more nucleotides downstream of the main start codon. In other cases, the agent binds to at least one, two, or three nucleotides of the main start codon. In some cases, the agent inhibits or reduces formation of a secondary mRNA structure comprising the at least one nucleotide of the main start codon of the processed mRNA. For instance, the agent inhibits or reduces base-pairing of the at least one nucleotide of the main start codon of the processed mRNA with another nucleotide of the processed mRNA. In some cases, the another nucleotide is another nucleotide of the 5′ UTR of the processed mRNA.

In some cases, the target protein is OPA1 protein, and the agent inhibits the base-pairing with the at least one nucleotide of the main start codon of the processed mRNA. In some of these cases, the agent binds to a targeted portion of the processed mRNA that is at most 60 nucleotides upstream of the main start codon of the processed mRNA. In some of these cases, the agent binds to a targeted portion of the processed mRNA that is at least 42 nucleotides upstream of the main start codon of the processed mRNA. In some of these cases, the agent binds to a targeted portion of the processed mRNA that is at most 116 nucleotides upstream of the main start codon of the processed mRNA. In some of these cases, the agent binds to a targeted portion of the processed mRNA that is at least 108 nucleotides upstream of the main start codon of the processed mRNA. In some of these cases, the main start codon of the OPA1 mature mRNA is defined by chromosomal coordinates GRCh38 chr3:193,593,378-193,593,380.

In some embodiments, the translation regulatory element comprises at least part of an upstream open reading frame (uORF). In some of these cases, an agent provided herein promotes formation of a secondary mRNA structure that involves the at least part of the uORF. In some of these cases, the translation regulatory element comprises an upstream start codon. In some of these cases, the agent promotes formation of a secondary mRNA structure that involves base-pairing with at least one nucleotide of the upstream start codon. In some cases, the agent does not bind to the upstream start codon. In other cases, the agent binds to the upstream start codon. In some of these embodiments, regardless of whether the agent binds to the upstream start codon, the agent promotes or increases formation of a secondary mRNA structure comprising the at least one nucleotide of the upstream start codon. In some cases, the agent promotes or increases base-pairing of the at least one nucleotide of the upstream start codon with another nucleotide of the processed mRNA, optionally the another nucleotide is another nucleotide of the 5′ UTR of the processed mRNA.

In some embodiments, the target protein is OPA1 protein, and the agent promotes formation of a secondary mRNA structure that involves the at least part of the uORF. In some of these embodiments, the agent binds to a targeted portion of the processed mRNA that is at most 60 nucleotides upstream of the main start codon of the processed mRNA. In some of these embodiments, the agent binds to a targeted portion of the processed mRNA that is at least 52 nucleotides upstream of the main start codon of the processed mRNA. In some of these embodiments, the upstream start codon is defined by genomic coordinates GRCh38 chr3: 193,593,226-193,593,228.

In some embodiments, the translation regulatory element comprises a Guanine quadruplex formed by a G-rich sequence of the processed mRNA. In some of these embodiments, the agent inhibits formation of the Guanine quadruplex. In some embodiments, the G-rich sequence comprises at least a portion of 5′ untranslated region (5′ UTR) of the processed mRNA. In some embodiments, the G-rich sequence is present in the 5′ untranslated region (5′ UTR) of the processed mRNA. In some embodiments, the G-rich sequence comprises a sequence according to the formula Gx-N1-7-Gx-N1-7-Gx-N1-7-Gx, where x≥3 and N is A, C, G or U. In some embodiments, the G-rich sequence comprises a sequence GGGAGCCGGGCUGGGGCUCACACGGGGG (SEQ ID NO: 1310). In some cases, at least one, two, three or all four of the Gx sequences in the 5′ UTR of the processed mRNA are structured, present in a secondary structure, or base-paired with another nucleotide, optionally the another nucleotide is a C or a U. In some of these embodiments, the agent provided herein relaxes, promotes deformation of, or inhibits or reduces formation of the Guanine quadruplex. In some of these embodiments, the agent relaxes, promotes deformation, or inhibits or reduces base-pairing or a structure of at least one, two, three or all four of the Gx sequences of the Guanine quadruplex.

In some embodiments, the target protein is OPA1 protein, and the agent inhibits formation of the Guanine quadruplex, and/or relaxes, promotes deformation, or inhibits or reduces base-pairing or a structure of at least one, two, three or all four of the Gx sequences of the Guanine quadruplex. In some of these embodiments, the agent binds to a targeted portion of the processed mRNA that is at most 60 nucleotides upstream of the main start codon of the processed mRNA. In some of these embodiments, the targeted portion of the processed mRNA is at most 35 nucleotides upstream of the main start codon of the processed mRNA. In some of these embodiments, the targeted portion of the processed mRNA is at least 17 nucleotides upstream of the main start codon of the processed mRNA. In some of these embodiments, the targeted portion of the processed mRNA is at most 60 nucleotides upstream of the main start codon of the processed mRNA and at least 17 nucleotides upstream of the main start codon of the processed mRNA.

In some aspects, an agent provided herein targets a targeted portion within the 5′ UTR of the processed mRNA. In some embodiments, the targeted portion of the processed mRNA comprises at least one nucleotide upstream of the codon immediately downstream from the main start codon of the processed mRNA. In some cases, the targeted portion of the processed mRNA comprises at least one nucleotide that is at most 234 nucleotides upstream of the first nucleotide of the main start codon the processed mRNA. In some cases, the targeted portion of the processed mRNA comprises at least one nucleotide that is at most 234, 220, 200, 180, 160, 140, 120, 100, 80, 90, 70, 60, 50, 40, 30, 20, or 10 nucleotides upstream of the first nucleotide of the main start codon the processed mRNA. In some cases, the targeted portion of the processed mRNA is at least 20, 30, 40, 50, 60, 70, 80, 90, 100, 110, 120, 150, 160, 180, or 200 nucleotides upstream of the main start codon of the processed mRNA. In some cases, the targeted portion of the processed mRNA is about 20, 30, 40, 50, 60, 70, 80, 90, 100, 110, 120, 150, 160, 180, 200, or 220 nucleotides upstream of the main start codon. In some cases, the targeted portion of the processed mRNA is about 110 nucleotides upstream of the main start codon.

In some embodiments, the target protein is an OPA1 protein, the processed mRNA is a processed mRNA encoding OPA1 protein. In some of these embodiments, the targeted portion of the processed mRNA has a sequence with at least 80%, 82%, 84%, 85%, 86%, 88%, 90%, 91%, 92%, 93%, 94%, 9%, 96%, 97%, 98%, or 99%, or 100% sequence identity to any sequence listed in Table 1.4. In some of these embodiments, the targeted portion of the processed mRNA has a sequence with at least 80%, 82%, 84%, 85%, 86%, 88%, 90%, 91%, 92%, 93%, 94%, 9%, 96%, 97%, 98%, or 99%, or 100% sequence identity to at least 8 contiguous nucleotides of a sequence selected from the group consisting of SEQ ID NOs: 1263-1271. In some cases, the processed mRNA has a sequence with at least 80%, 82%, 84%, 85%, 86%, 88%, 90%, 91%, 92%, 93%, 94%, 9%, 96%, 97%, 98%, or 99%, or 100% sequence identity to any sequence listed in Table 1.3. In some cases, the processed mRNA has a sequence with at least 80%, 82%, 84%, 85%, 86%, 88%, 90%, 91%, 92%, 93%, 94%, 9%, 96%, 97%, 98%, or 99%, or 100% sequence identity to a sequence selected from the group consisting of SEQ ID NOs: 1254-1262. In some cases, the agent comprises an antisense oligomer that has at least 80%, 82%, 84%, 85%, 86%, 88%, 90%, 91%, 92%, 93%, 94%, 9%, 96%, 97%, 98%, or 99%, or 100% sequence identity to any sequence in Table 8. In some cases, the agent comprises an antisense oligomer that has at least 80%, 82%, 84%, 85%, 86%, 88%, 90%, 91%, 92%, 93%, 94%, 9%, 96%, 97%, 98%, or 99%, or 100% sequence identity to any sequence selected from the group consisting of SEQ ID NOs: 608-1253. In some cases, the agent comprises an antisense oligomer that has about 100% sequence identity to a sequence selected from the group consisting of SEQ ID NOs: 608-1253. In some cases, the antisense oligomer has at least 80%, 82%, 84%, 85%, 86%, 88%, 90%, 91%, 92%, 93%, 94%, 9%, 96%, 97%, 98%, or 99%, or 100% sequence identity to a sequence selected from the group consisting of SEQ ID NOs: 827-847, 932-937, 953, 968, 988-1023. In some cases, the antisense oligomer has about 100% sequence identity to a sequence selected from the group consisting of SEQ ID NOs: 827-847, 932-937, 953, 968, 988-1023.

In some embodiments, one or more of the translation regulatory elements disclosed herein inhibit the translation of the processed mRNA by inhibiting translation efficiency and/or rate of translation of the processed mRNA. In some embodiments, the agent provided herein that targets a processed mRNA increases the expression of the OPA1 protein in the cell by increasing the translation efficiency and/or rate of translation of the processed mRNA.

In some embodiments, the agent disclosed herein modulates (e.g., promotes or inhibits) binding of factors that regulate translation. Such factors are known in the art, and also described at Patricia R. Araujo et al. Before It Gets Started: Regulating Translation at the 5′ UTR, International Journal of Genomics, vol. 2012, Article ID 475731, 8 pages, 2012, which is incorporated herein by reference in its entirety.

In some embodiments, the translation efficiency and/or rate of translation of the processed mRNA that encodes the OPA1 protein in the cell contacted with the agent or the vector encoding the agent is increased compared to the translation efficiency and/or rate of translation of the processed mRNA in a control cell not contacted with the agent or the vector encoding the agent.

In some cases, the translation efficiency and/or rate of translation of the processed mRNA that encodes the OPA1 protein in the cell contacted with the agent disclosed herein or the vector encoding the agent is increased by about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the translation efficiency and/or rate of translation of the processed mRNA in a control cell not contacted with the agent or the vector encoding the agent.

In some cases, the translation efficiency and/or rate of translation of the processed mRNA that encodes the OPA1 protein in the cell contacted with the agent disclosed herein or the vector encoding the agent is increased by about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to in the absence of the agent.

In some embodiments, the processed mRNA transcript targeted by the disclosed methods, compositions, or kits is a mutant processed mRNA transcript. In some embodiments, the processed mRNA transcript is not a mutant processed mRNA transcript. In some embodiments, the processed mRNA is processed from a pre-mRNA that is a mutant pre-mRNA. In some embodiments, the processed mRNA is processed from a pre-mRNA that is not a mutant pre-mRNA.

mRNA Splicing

Intervening sequences in RNA sequences or introns are removed by a large and highly dynamic RNA-protein complex termed the spliceosome, which orchestrates complex interactions between primary transcripts, small nuclear RNAs (snRNAs) and a large number of proteins. Spliceosomes assemble ad hoc on each intron in an ordered manner, starting with recognition of the 5′ splice site (5′ss) by U1 snRNA or the 3′splice site (3′ss) by the U2 pathway, which involves binding of the U2 auxiliary factor (U2AF) to the 3′ss region to facilitate U2 binding to the branch point sequence (BPS). U2AF is a stable heterodimer composed of a U2AF2-encoded 65-kD subunit (U2AF65), which binds the polypyrimidine tract (PPT), and a U2AF1-encoded 35-kD subunit (U2AF35), which interacts with highly conserved AG dinucleotides at 3′ss and stabilizes U2AF65 binding. In addition to the BPS/PPT unit and 3′ss/5′ss, accurate splicing requires auxiliary sequences or structures that activate or repress splice site recognition, known as intronic or exonic splicing enhancers or silencers. These elements allow genuine splice sites to be recognized among a vast excess of cryptic or pseudo-sites in the genome of higher eukaryotes, which have the same sequences but outnumber authentic sites by an order of magnitude. Although they often have a regulatory function, the exact mechanisms of their activation or repression are poorly understood.

The decision of whether to splice or not to splice can be typically modeled as a stochastic rather than deterministic process, such that even the most defined splicing signals can sometimes splice incorrectly. However, under normal conditions, pre-mRNA splicing proceeds at surprisingly high fidelity. This is attributed in part to the activity of adjacent cis-acting auxiliary exonic and intronic splicing regulatory elements (ESRs or ISRs). Typically, these functional elements are classified as either exonic or intronic splicing enhancers (ESEs or ISEs) or silencers (ESSs or ISSs) based on their ability to stimulate or inhibit splicing, respectively. Although there is now evidence that some auxiliary cis-acting elements may act by influencing the kinetics of spliceosome assembly, such as the arrangement of the complex between U1 snRNP and the 5′ss, it seems very likely that many elements function in concert with trans-acting RNA-binding proteins (RBPs). For example, the serine- and arginine-rich family of RBPs (SR proteins) is a conserved family of proteins that have a key role in defining exons. SR proteins promote exon recognition by recruiting components of the pre-spliceosome to adjacent splice sites or by antagonizing the effects of ESSs in the vicinity. The repressive effects of ESSs can be mediated by members of the heterogeneous nuclear ribonucleoprotein (hnRNP) family and can alter recruitment of core splicing factors to adjacent splice sites. In addition to their roles in splicing regulation, silencer elements are suggested to have a role in repression of pseudo-exons, sets of decoy intronic splice sites with the typical spacing of an exon but without a functional open reading frame. ESEs and ESSs, in cooperation with their cognate trans-acting RBPs, represent important components in a set of splicing controls that specify how, where and when mRNAs are assembled from their precursors.

Alternative splicing is a regulated process during gene expression that can result in multiple isoforms of mature mRNA transcripts that are processed from a single primary mRNA transcript that is transcribed from a single gene, and the resultant multiple proteins that are translated from at least some of the multiple mature mRNA isoforms. In this process, particular exons of a gene may be included within or excluded from the final, processed mRNA produced from that gene. Consequently, the proteins translated from alternatively splices mRNAs will contain differences in their amino acid sequence and, in some cases, in their biological functions.

As described herein, an “alternatively spliced exon” can refer to an exon of a gene that can be either included or excluded naturally from a mature mRNA transcript, thus resulting in different protein products that are translated from the different mature mRNA transcripts. The inclusion or skipping of an alternatively spliced exon can take place naturally in a cell, either randomly, or in a regulated manner, e.g., subject to regulation by external physiological or pathological stimuli, or intracellular signaling. In some cases, the production of alternatively spliced mRNAs, e.g., the splicing of the alternatively spliced exon, is regulated by a system of trans-acting proteins that bind to cis-acting sites on the primary transcript itself. In some cases, an alternatively spliced exon is a coding exon, e.g., an exon that, when included in the mature mRNA transcript, is translated into an amino acid sequence as part of the protein product translated from the mature mRNA transcript. In some cases, the inclusion of an alternatively spliced exon in the mature mRNA transcript would maintain the canonical open reading frame as compared to a mature mRNA transcript without the alternatively spliced exon, e.g., the number of nucleotides in the alternatively spliced exon is divisible by 3.

The sequences marking the exon-intron boundaries are degenerate signals of varying strengths that can occur at high frequency within human genes. In multi-exon genes, different pairs of splice sites can be linked together in many different combinations, creating a diverse array of transcripts from a single gene. This is commonly referred to as alternative pre-mRNA splicing. Although most mRNA isoforms produced by alternative splicing can be exported from the nucleus and translated into functional polypeptides, different mRNA isoforms from a single gene can vary greatly in their translation efficiency. Those mRNA isoforms with premature termination codons (PTCs) at least 50 bp upstream of an exon junction complex are likely to be targeted for degradation by the nonsense-mediated mRNA decay (NMD) pathway. Mutations in traditional (BPS/PPT/3′ss/5′ss) and auxiliary splicing motifs can cause aberrant splicing, such as exon skipping or cryptic (or pseudo-) exon inclusion or splice-site activation, and contribute significantly to human morbidity and mortality. Both aberrant and alternative splicing patterns can be influenced by natural DNA variants in exons and introns.

Given that exon-intron boundaries can occur at any of the three positions of a codon, it is clear that only a subset of alternative splicing events can maintain the canonical open reading frame. For example, only exons that are evenly divisible by 3 can be skipped or included in the mRNA without any alteration of reading frame. Splicing events that do not have compatible phases will induce a frame-shift. Unless reversed by downstream events, frame-shifts can certainly lead to one or more PTCs, probably resulting in subsequent degradation by NMD. NMD is a translation-coupled mechanism that eliminates mRNAs containing PTCs. NMD can function as a surveillance pathway that exists in all eukaryotes. NMD can reduce errors in gene expression by eliminating mRNA transcripts that contain premature stop codons. Translation of these aberrant mRNAs could, in some cases, lead to deleterious gain-of-function or dominant-negative activity of the resulting proteins. NMD targets not only transcripts with PTCs but also a broad array of mRNA isoforms expressed from many endogenous genes, suggesting that NMD is a master regulator that drives both fine and coarse adjustments in steady-state RNA levels in the cell.

An NMD-inducing exon (“NIE” or “NMD exon”) is an exon or a pseudo-exon that is a region within an intron and can activate the NMD pathway if included in a mature RNA transcript. In constitutive splicing events, the intron containing an NMD exon is usually spliced out, but the intron or a portion thereof (e.g., NMD exon) may be retained during alternative or aberrant splicing events. Mature mRNA transcripts containing such an NMD exon may be non-productive due to frame shifts which induce the NMD pathway. Inclusion of a NMD exon in mature RNA transcripts may downregulate gene expression. mRNA transcripts containing an NMD exon may be referred to as “NIE-containing mRNA” or “NMD exon mRNA” in the current disclosure.

Cryptic (or pseudo-splice sites) have the same splicing recognition sequences as genuine splice sites but are not used in splicing reactions. They outnumber genuine splice sites in the human genome by an order of a magnitude and are normally repressed by thus far poorly understood molecular mechanisms. Cryptic 5′ splice sites have the consensus NNN/GUNNNN or NNN/GCNNNN where N is any nucleotide and/is the exon-intron boundary. Cryptic 3′ splice sites have the consensus NAG/N. Their activation is positively influenced by surrounding nucleotides that make them more similar to the optimal consensus of authentic splice sites, namely MAG/GURAGU and YAG/G, respectively, where M is C or A, R is G or A, and Y is C or U.

Splice sites and their regulatory sequences can be readily identified by a skilled person using suitable algorithms publicly available, listed for example in Kralovicova, J. and Vorechovsky, I. (2007) Global control of aberrant splice site activation by auxiliary splicing sequences: evidence for a gradient in exon and intron definition. Nucleic Acids Res., 35, 6399-6413 (www.ncbi.nlm.nih.gov/pmc/articles/PMC2095810/pdf/gkm680.pdf).

The cryptic splice sites or splicing regulatory sequences may compete for RNA-binding proteins, such as U2AF, with a splice site of the NMD exon. In some embodiments, an agent may bind to a cryptic splice site or splicing regulatory sequence to prevent binding of RNA-binding proteins and thereby favor binding of RNA-binding proteins to the NMD exon splice sites.

In some embodiments, the cryptic splice site may not comprise the 5′ or 3′ splice site of the NMD exon. In some embodiments, the cryptic splice site may be at least 10 nucleotides, at least 20 nucleotides, at least 50 nucleotides, at least 100 nucleotides or at least 200 nucleotides upstream of the NMD exon 5′ splice site. In some embodiments, the cryptic splice site may be at least 10 nucleotides, at least 20 nucleotides, at least 50 nucleotides, at least 100 nucleotides, at least 200 nucleotides downstream of the NMD exon 3′ splice site.

### Target Transcripts

In some embodiments, the methods and compositions of the present disclosure exploit the presence of NMD exon in the pre-mRNA transcribed from the OPA1 gene. Splicing of the identified OPA1 NMD exon pre-mRNA species to produce functional mature OPA1 mRNA may be induced using an agent such as an ASO that stimulates exon skipping of an NMD exon. Induction of exon skipping may result in inhibition of an NMD pathway. The resulting mature OPA1 mRNA can be translated normally without activating NMD pathway, thereby increasing the amount of OPA1 protein in the patient's cells and alleviating symptoms of a condition or disease associated with OPA1 deficiency, such as an eye disease or condition, Optic atrophy type 1, autosomal dominant optic atrophy (ADOA), ADOA-plus syndrome; a mitochondrial disorder; glaucoma; normal tension glaucoma; Charcot-Marie-Tooth disease; mitochondria dysfunction; diabetic retinopathy; age-related macular degeneration; retinal ganglion cell death; mitochondrial fission-mediated mitochondrial dysfunction; progressive external ophthalmoplegia; deafness; ataxia; motor neuropathy; sensory neuropathy; myopathy; Behr syndrome; brain dysfunction; encephalopathy; peripheral neuropathy; fatal infantile mitochondrial encephalomyopathy; hypertrophic cardiomyopathy; spastic ataxic syndrome; sensory motor peripheral neuropathy; hypotonia; gastrointestinal dysmotility and dysphagia; optic atrophy; optic atrophy plus syndrome; Mitochondrial DNA depletion syndrome 14; late-onset cardiomyopathy; diabetic cardiomyopathy; Alzheimer's Disease; focal segmental glomerulosclerosis; kidney disease; Huntington's Disease; cognitive function decline in healthy aging; Prion diseases; late onset dementia and parkinsonism; mitochondrial myopathy; Leigh syndrome; Friedreich's ataxia; Parkinson's disease; MELAS (Mitochondrial encephalomyopathy, lactic acidosis, and stroke-like episodes); pyruvate dehydrogenase complex deficiency; chronic kidney disease; Leber's hereditary optic neuropathy; obesity; age-related systemic neurodegeneration; skeletal muscle atrophy; heart and brain ischemic damage; massive liver apoptosis; NARP (neuropathy, ataxia, retinitis pigmentosa); MERRF (myoclonic epilepsy and ragged red fibers); Pearsons/Kerns-Sayre syndrome; MIDD (maternally inherited diabetes and deafness); mitochondrial trifunctional protein deficiency; Fuchs corneal endothelial dystrophy; macular telangiectasia; retinitis pigmentosa; Leber congenital amaurosis; inherited maculopathy; Stargardt disease; or Sorsby fundus dystrophy.

In some embodiments, the methods and compositions of the present disclosure exploit the alternative splicing of the pre-mRNA transcribed from the OPA1 gene. In some cases, splicing of a coding exon, e.g., an alternatively spliced exon, e.g., OPA1 exon 7 (or an exon encoded by genomic region spanning from GRCh38/hg38: chr3 193626092 to 193626202), can modulate the level of OPA1 protein expressed from the OPA1 gene. As described herein, the term “OPA1 exon 7” or grammatically equivalents thereof, is used interchangeably with the term “exon (GRCh38/hg38: chr3 193626092 to 193626202)” or “an exon encoded by genomic region spanning from GRCh38/hg38: chr3 193626092 to 193626202.” Without wishing to be bound by a certain theory, the presence or absence of an amino acid sequence encoded by exon 7 or exon (GRCh38/hg38: chr3 193626092 to 193626202) can modulate the stability of the OPA1 protein. For instance, in some cases, the OPA1 protein encoded by a mature mRNA transcript that lacks exon 7 can have fewer proteolytic cleavage sites as compared to an OPA1 protein encoded by a corresponding mature mRNA transcript that has contains exon 7. In some cases, the OPA1 protein an OPA1 protein encoded by a corresponding mature mRNA transcript that has contains encoded by a mature mRNA transcript that lacks exon 7 is a functional protein. The OPA1 protein encoded by a mature mRNA transcript that lacks exon 7 can be at least partially functional as compared to an OPA1 protein encoded by a corresponding mature mRNA transcript that has contains exon 7. In some cases, the OPA1 protein encoded by a mature mRNA transcript that lacks exon 7 is at least partially functional as compared to a full-length wild-type OPA1 protein. In some cases, increase of OPA1 protein encoded by a mature mRNA transcript that lacks exon 7 in a cell can result in more functional OPA1 protein in the cell, due to the higher stability of the OPA1 protein lacking exon 7 and its at least partial functional equivalence.

In other embodiments, a coding exon of OPA1 pre-mRNA other than exon 7 is targeted by an agent disclosed herein, which promotes exclusion of the coding exon other than exon 7. In these other embodiments, the agent that promotes exclusion of the coding exon other than exon 7 increases expression of OPA1 protein encoded by a mature mRNA transcript that lacks the excluded exon.

Alternative splicing of the OPA1 pre-mRNA species, e.g., skipping of a coding exon, e.g., an alternatively spliced exon, e.g., exon 7, to produce functional mature OPA1 protein may be induced using an agent such as an ASO that stimulates the exon skipping. Induction of exon skipping may result in modulation of levels of different alternatively spliced mRNA transcripts. The resulting mature OPA1 mRNA can be translated into different OPA1 proteins, thereby modulating the amount of OPA1 protein in the patient's cells and alleviating symptoms of a condition or disease associated with OPA1 deficiency, such as an eye disease or condition, Optic atrophy type 1, autosomal dominant optic atrophy (ADOA), ADOA-plus syndrome; a mitochondrial disorder; glaucoma; normal tension glaucoma; Charcot-Marie-tooth disease; mitochondria dysfunction; diabetic retinopathy; age-related macular degeneration; retinal ganglion cell death; mitochondrial fission-mediated mitochondrial dysfunction; progressive external ophthalmoplegia; deafness; ataxia; motor neuropathy; sensory neuropathy; myopathy; Behr syndrome; brain dysfunction; encephalopathy; peripheral neuropathy; fatal infantile mitochondrial encephalomyopathy; hypertrophic cardiomyopathy; spastic ataxic syndrome; sensory motor peripheral neuropathy; hypotonia; gastrointestinal dysmotility and dysphagia; optic atrophy; optic atrophy plus syndrome; Mitochondrial DNA depletion syndrome 14; late-onset cardiomyopathy; diabetic cardiomyopathy; Alzheimer's Disease; focal segmental glomerulosclerosis; kidney disease; Huntington's Disease; cognitive function decline in healthy aging; Prion diseases; late onset dementia and parkinsonism; mitochondrial myopathy; Leigh syndrome; Friedreich's ataxia; Parkinson's disease; MELAS (Mitochondrial encephalomyopathy, lactic acidosis, and stroke-like episodes); pyruvate dehydrogenase complex deficiency; chronic kidney disease; Leber's hereditary optic neuropathy; obesity; age-related systemic neurodegeneration; skeletal muscle atrophy; heart and brain ischemic damage; massive liver apoptosis; NARP (neuropathy, ataxia, retinitis pigmentosa); MERRF (myoclonic epilepsy and ragged red fibers); Pearsons/Kems-Sayre syndrome; MIDD (maternally inherited diabetes and deafness); mitochondrial trifunctional protein deficiency; Fuchs corneal endothelial dystrophy; macular telangiectasia; retinitis pigmentosa; Leber congenital amaurosis; inherited maculopathy; Stargardt disease; or Sorsby fundus dystrophy.

In some embodiments, the diseases or conditions that can be treated or ameliorated using the method or composition disclosed herein are not directly associated with the target protein (gene) that the therapeutic agent targets. In some embodiments, a therapeutic agent provided herein can target a protein (gene) that is not directly associated with a disease or condition, but the modulation of expression of the target protein (gene) can treat or ameliorate the disease or condition.

In various embodiments, the present disclosure provides a therapeutic agent which can target OPA1 mRNA transcripts to modulate splicing or protein expression level. The therapeutic agent can be a small molecule, polynucleotide, or polypeptide. In some embodiments, the therapeutic agent is an ASO. Various regions or sequences on the OPA1 pre-mRNA can be targeted by a therapeutic agent, such as an ASO. In some embodiments, the ASO targets an OPA1 pre-mRNA transcript containing an NMD exon. In some embodiments, the ASO targets a sequence within an NMD exon of an OPA1 pre-mRNA transcript. In some embodiments, the ASO targets a sequence upstream (or 5′) from the 5′ end of an NMD exon (3′ss) of an OPA1 pre-mRNA transcript. In some embodiments, the ASO targets a sequence downstream (or 3′) from the 3′ end of an NMD exon (5′ss) of an OPA1 pre-mRNA transcript. In some embodiments, the ASO targets a sequence that is within an intron flanking on the 5′ end of the NMD exon of an OPA1 pre-mRNA transcript. In some embodiments, the ASO targets a sequence that is within an intron flanking the 3′ end of the NMD exon of an OPA1 pre-mRNA transcript. In some embodiments, the ASO targets a sequence comprising an NMD exon-intron boundary of an OPA1 pre-mRNA transcript. An NMD exon-intron boundary can refer to the junction of an intron sequence and an NMD exon region. The intron sequence can flank the 5′ end of the NMD exon, or the 3′ end of the NMD exon. In some embodiments, the ASO targets a sequence within an exon of an OPA1 pre-mRNA transcript. In some embodiments, the ASO targets a sequence within an intron of an OPA1 pre-mRNA transcript. In some embodiments, the ASO targets a sequence comprising both a portion of an intron and a portion of an exon of an OPA1 pre-mRNA transcript.

In some embodiments, the ASO targets a sequence about 4 to about 300 nucleotides upstream (or 5′) from the 5′ end of the NMD exon. In some embodiments, the ASO targets a sequence about 1 to about 20 nucleotides, about 20 to about 50 nucleotides, about 50 to about 100 nucleotides, about 100 to about 150 nucleotides, about 150 to about 200 nucleotides, about 200 to about 250 nucleotides, or about 250 to about 300 nucleotides upstream (or 5′) from the 5′ end of the NMD exon region. In some embodiments, the ASO may target a sequence more than 300 nucleotides upstream from the 5′ end of the NMD exon. In some embodiments, the ASO targets a sequence about 4 to about 300 nucleotides downstream (or 3′) from the 3′ end of the NMD exon. In some embodiments, the ASO targets a sequence about 1 to about 20 nucleotides, about 20 to about 50 nucleotides, about 50 to about 100 nucleotides, about 100 to about 150 nucleotides, about 150 to about 200 nucleotides, about 200 to about 250 nucleotides, or about 250 to about 300 nucleotides downstream from the 3′ end of the NMD exon. In some embodiments, the ASO targets a sequence more than 300 nucleotides downstream from the 3′ end of the NMD exon.

In some embodiments, the OPA1 NMD exon-containing pre-mRNA transcript is encoded by a genetic sequence with at least about 80%, 85%, 90%, 95%, 96%, 97%, 98%, 99% or 100% sequence identity to SEQ ID NO. 1. In some embodiments, the OPA1 NMD exon pre-mRNA transcript comprises a sequence with at least about 80%, 85%, 90%, 95%, 96%, 97%, 98%, 99% or 100% sequence identity to any one of SEQ ID NOs: 2-5.

In some embodiments, the OPA1 NMD exon-containing pre-mRNA transcript (or NMD exon mRNA) comprises a sequence with at least about 80%, 85%, 90%, 95%, 97%, or 100% sequence identity to any one of SEQ ID NOs: 2-5. In some embodiments, OPA1 NMD exon-containing pre-mRNA transcript (or NMD exon mRNA) is encoded by a sequence with at least about 80%, 85%, 90%, 95%, 97%, or 100% sequence identity to any one of SEQ ID NOs: 2-5. In some embodiments, the targeted portion of the NMD exon mRNA comprises a sequence with at least 80%, 85%, 90%, 95%, 97%, or 100% sequence identity to a region comprising at least 8 contiguous nucleic acids of any one of SEQ ID NOs: 2-5.

In some embodiments, the ASO targets exon 6x of an OPA1 NMD exon-containing pre-mRNA comprising NIE exon 6, exon 7x of an OPA1 NMD exon-containing pre-mRNA comprising NIE exon 7, or exon 28x of an OPA1 NMD exon-containing pre-mRNA comprising NIE exon 28. In some embodiments, the ASO targets exon (GRCh38/hg38: chr3 193628509 193628616) of OPA1 pre-mRNA; or exon (GRCh38/hg38: chr3 193603500 193603557) of OPA1. In some embodiments, the ASO targets an NMD exon of OPA1 pre-mRNA other than NMD exon (GRCh38/hg38: chr3 193628509 193628616).

In some embodiments, the ASO targets a sequence about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream (or 5′) from the 5′ end of exon 6x of OPA1, exon 7x of OPA1, or exon 28x of OPA1. In some embodiments, the ASO targets a sequence about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream (or 5′) from GRCh38/hg38: chr3 193628509 of OPA1; or GRCh38/hg38: chr3 193603500 of OPA1.

In some embodiments, the ASO targets a sequence at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream (or 5′) from the 5′ end of exon 6x of OPA1, exon 7x of OPA1, or exon 28x of OPA1. In some embodiments, the ASO targets a sequence at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream (or 5′) from GRCh38/hg38: chr3 193628509 of OPA1; or GRCh38/hg38: chr3 193603500 of OPA1.

In some embodiments, the ASO targets a sequence about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream (or 3′) from the 3′ end of exon 6x of OPA1, exon 7x of OPA1, or exon 28x of OPAL. In some embodiments, the ASO targets a sequence about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream (or 3′) from GRCh38/hg38: chr3 193628616 of OPA1; or GRCh38/hg38: chr3 193603557 of OPAL.

In some embodiments, the ASO targets a sequence at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream (or 3′) from the 3′ end of exon 6x of OPA1, exon 7x of OPA1, or exon 28x of OPAL. In some embodiments, the ASO targets a sequence at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream (or 3′) from GRCh38/hg38: chr3 193628616 of OPA1; or GRCh38/hg38: chr3 193603557 of OPAL.

In some embodiments, the ASO has a sequence complementary to the targeted portion of the NMD exon mRNA according to any one of SEQ ID NOs: 2-5, or 279.

In some embodiments, the ASO targets a sequence upstream from the 5′ end of an NMD exon. For example, ASOs targeting a sequence upstream from the 5′ end of an NMD exon (exon 6x of OPA1, exon 7x of OPA1, or exon 28x of OPA1) comprises a sequence that is at least about 80%, 85%, 90%, 95%, 97%, or 100% complimentary to at least 8 contiguous nucleic acids of SEQ ID NO: 2 or 3. For example, ASOs targeting a sequence upstream from the 5′ end of an NMD exon (e.g., exon (GRCh38/hg38: chr3 193628509 to 193628616) of OPA1; or exon (GRCh38/hg38: chr3 193603500 193603557) of OPA1) can comprise a sequence with at least 80%, 85%, 90%, 95%, 97%, or 100% sequence identity to SEQ ID NO: 4 or 5.

In some embodiments, the ASOs target a sequence containing an exon-intron boundary (or junction). For example, ASOs targeting a sequence containing an exon-intron boundary can comprise a sequence that is at least about 80%, 85%, 90%, 95%, 97%, or 100% complimentary to at least 8 contiguous nucleic acids of any one of SEQ ID NOs: 2-5. In some embodiments, the ASOs target a sequence downstream from the 3′ end of an NMD exon. For example, ASOs targeting a sequence downstream from the 3′ end of an NMD exon (e.g., exon 6x of OPA1, exon 7x of OPA1, or exon 28x of OPA1) can comprise a sequence with at least 80%, 85%, 90%, 95%, 97%, or 100% sequence identity to SEQ ID NO: 2 or 3, or at least 8 contiguous nucleic acids of SEQ ID NO: 2 or 3. For example, ASOs targeting a sequence downstream from the 3′ end of an NMD exon (e.g., exon (GRCh38/hg38: chr3 193628509 to 193628616) of OPA1; or exon (GRCh38/hg38: chr3 193603500 to 193603557) of OPA1) can comprise a sequence with at least 80%, 85%, 90%, 95%, 97%, or 100% sequence identity to SEQ ID NO: 4 or 5, or at least 8 contiguous nucleic acids of SEQ ID NO: 4 or 5. In some embodiments, ASOs target a sequence within an NMD exon.

In some embodiments, the ASO targets exon 6x of an OPA1 NMD exon-containing pre-mRNA comprising NIE exon 6, exon 7x of an OPA1 NMD exon-containing pre-mRNA comprising NIE exon 7, or exon 28x of an OPA1 NMD exon-containing pre-mRNA comprising NIE exon 28. In some embodiments, the ASO targets a sequence downstream (or 3′) from the 5′ end of exon 6x, exon 7x, or exon 28x of an OPA1 pre-mRNA. In some embodiments, the ASO targets a sequence upstream (or 5′) from the 3′ end of exon 6x, exon 7x, or exon 28x of an OPA1 pre-mRNA.

In some embodiments, the targeted portion of the OPA1 NMD exon-containing pre-mRNA is in intron 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50. In some embodiments, hybridization of an ASO to the targeted portion of the NMD exon pre-mRNA results in exon skipping of at least one of NMD exon within intron 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, and subsequently increases OPA1 protein production. In some embodiments, the targeted portion of the OPA1 NMD exon-containing pre-mRNA is in intron 6 of OPA1, or intron 28 of OPAL. In some embodiments, the targeted portion of the OPA1 NMD exon-containing pre-mRNA is intron (GRCh38/hg38: chr3 193626203 to 193631611) of OPA1; or intron (GRCh38/hg38: chr3 193593374 to 193614710) of OPAL.

In some embodiments, the methods and compositions of the present disclosure are used to increase the expression of OPA1 by inducing exon skipping of a pseudo-exon of an OPA1 NMD exon-containing pre-mRNA. In some embodiments, the pseudo-exon is a sequence within any of introns 1-50. In some embodiments, the pseudo-exon is a sequence within any of introns 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50. In some embodiments, the pseudo-exon can be an OPA1 intron or a portion thereof. In some embodiments, the pseudo-exon is within intron 6 of OPA1, or intron 28 of OPAL. In some embodiments, the pseudo-exon is within intron (GRCh38/hg38: chr3 193626203 to 193631611) of OPA1; or intron (GRCh38/hg38: chr3 193593374 to 193614710) of OPA1.

In some embodiments, the ASO targets an OPA1 pre-mRNA transcript to induce exon skipping of a coding exon, e.g., an alternatively spliced exon. In some embodiments, the ASO targets a sequence within a coding exon, e.g., an alternatively spliced exon, of an OPA1 pre-mRNA transcript. In some embodiments, the ASO targets a sequence upstream (or 5′) from the 5′ end of a coding exon (3′ss) of an OPA1 pre-mRNA transcript. In some embodiments, the ASO targets a sequence downstream (or 3′) from the 3′ end of a coding exon (5′ss) of an OPA1 pre-mRNA transcript. In some embodiments, the ASO targets a sequence that is within an intron flanking on the 5′ end of the coding exon of an OPA1 pre-mRNA transcript. In some embodiments, the ASO targets a sequence that is within an intron flanking the 3′ end of the coding exon of an OPA1 pre-mRNA transcript. In some embodiments, the ASO targets a sequence comprising an exon-intron boundary of an OPA1 pre-mRNA transcript. An exon-intron boundary can refer to the junction of an intron sequence and an exon sequence. The intron sequence can flank the 5′ end of the coding exon, or the 3′ end of the coding exon. In some embodiments, the ASO targets a sequence within an exon of an OPA1 pre-mRNA transcript. In some embodiments, the ASO targets a sequence within an intron of an OPA1 pre-mRNA transcript. In some embodiments, the ASO targets a sequence comprising both a portion of an intron and a portion of an exon of an OPA1 pre-mRNA transcript.

In some embodiments, the ASO targets a sequence about 4 to about 300 nucleotides upstream (or 5′) from the 5′ end of the coding exon, e.g., alternatively spliced exon. In some embodiments, the ASO targets a sequence about 1 to about 20 nucleotides, about 20 to about 50 nucleotides, about 50 to about 100 nucleotides, about 100 to about 150 nucleotides, about 150 to about 200 nucleotides, about 200 to about 250 nucleotides, or about 250 to about 300 nucleotides upstream (or 5′) from the 5′ end of the coding exon region. In some embodiments, the ASO may target a sequence more than 300 nucleotides upstream from the 5′ end of the coding exon. In some embodiments, the ASO targets a sequence about 4 to about 300 nucleotides downstream (or 3′) from the 3′ end of the coding exon. In some embodiments, the ASO targets a sequence about 1 to about 20 nucleotides, about 20 to about 50 nucleotides, about 50 to about 100 nucleotides, about 100 to about 150 nucleotides, about 150 to about 200 nucleotides, about 200 to about 250 nucleotides, or about 250 to about 300 nucleotides downstream from the 3′ end of the coding exon. In some embodiments, the ASO targets a sequence more than 300 nucleotides downstream from the 3′ end of the coding exon.

In some embodiments, the OPA1 pre-mRNA transcript is encoded by a genetic sequence with at least about 80%, 85%, 90%, 95%, 96%, 97%, 98%, 99% or 100% sequence identity to SEQ ID NO. 1. In some embodiments, the OPA1 pre-mRNA transcript comprises a sequence with at least about 80%, 85%, 90%, 95%, 96%, 97%, 98%, 99% or 100% sequence identity to any one of SEQ ID NOs: 2-5.

In some embodiments, the OPA1 pre-mRNA transcript (or NMD exon mRNA) comprises a sequence with at least about 80%, 85%, 90%, 95%, 97%, or 100% sequence identity to any one of SEQ ID NOs: 2-5. In some embodiments, OPA1 pre-mRNA transcript (or NMD exon mRNA) is encoded by a sequence with at least about 80%, 85%, 90%, 95%, 97%, or 100% sequence identity to any one of SEQ ID NOs: 2-5. In some embodiments, the targeted portion of the OPA1 pre-mRNA comprises a sequence with at least 80%, 85%, 90%, 95%, 97%, or 100% sequence identity to a region comprising at least 8 contiguous nucleic acids of any one of SEQ ID NOs: 2-5.

In some embodiments, the ASO targets exon 7 of an OPA1 pre-mRNA, i.e., the ASO targets exon (GRCh38/hg38: chr3 193626092 to 193626202) of OPA1 pre-mRNA.

In some embodiments, the ASO targets a coding exon of an OPA1 pre-mRNA other than exon 7, i.e., the ASO targets an exon of OPA1 pre-mRNA other than exon defined by (GRCh38/hg38: chr3 193626092 to 193626202).

In some embodiments, the ASO targets a sequence about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream (or 5′) from the 5′ end of exon 7 of OPA1. In some embodiments, the ASO targets a sequence about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream (or 5′) from GRCh38/hg38: chr3 193626092 of OPA1.

In some embodiments, the ASO targets a sequence at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream (or 5′) from the 5′ end of exon 7 of OPA1. In some embodiments, the ASO targets a sequence at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream (or 5′) from GRCh38/hg38: 193626092 of OPA1.

In some embodiments, the ASO targets a sequence about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream (or 3′) from the 3′ end of exon 7 of OPA1. In some embodiments, the ASO targets a sequence about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream (or 3′) from GRCh38/hg38: chr3 193626202 of OPA1.

In some embodiments, the ASO targets a sequence at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream (or 3′) from the 3′ end of exon 7 of OPA1. In some embodiments, the ASO targets a sequence at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream (or 3′) from GRCh38/hg38: chr3 193626202 of OPA1.

In some embodiments, the ASO has a sequence complementary to the targeted portion of the NMD exon mRNA according to any one of SEQ ID NOs: 2-5, or 277.

In some embodiments, the ASO targets a sequence upstream from the 5′ end of a coding exon, e.g., an alternatively spliced exon. For example, ASOs targeting a sequence upstream from the 5′ end of a coding exon (e.g., exon 7 of OPA1) comprises a sequence that is at least about 80%, 85%, 90%, 95%, 97%, or 100% complimentary to at least 8 contiguous nucleic acids of SEQ ID NO: 2 or 3. For example, ASOs targeting a sequence upstream from the 5′ end of a coding exon (e.g., exon (GRCh38/hg38: 193626092 to 193626202) of OPA1) can comprise a sequence with at least 80%, 85%, 90%, 95%, 97%, or 100% sequence identity to SEQ ID NO: 4 or 5.

In some embodiments, the ASOs target a sequence containing an exon-intron boundary (or junction). For example, ASOs targeting a sequence containing an exon-intron boundary can comprise a sequence that is at least about 80%, 85%, 90%, 95%, 97%, or 100% complimentary to at least 8 contiguous nucleic acids of any one of SEQ ID NOs: 2-5. In some embodiments, the ASOs target a sequence downstream from the 3′ end of a coding exon, e.g., an alternatively spliced exon. For example, ASOs targeting a sequence downstream from the 3′ end of a coding exon (e.g., exon 7 of OPA1) can comprise a sequence with at least 80%, 85%, 90%, 95%, 97%, or 100% sequence identity to SEQ ID NO: 2 or 3, or at least 8 contiguous nucleic acids of SEQ ID NO: 2 or 3. For example, ASOs targeting a sequence downstream from the 3′ end of a coding exon (e.g., exon 7 of OPA1) can comprise a sequence with at least 80%, 85%, 90%, 95%, 97%, or 100% sequence identity to SEQ ID NO: 4 or 5, or at least 8 contiguous nucleic acids of SEQ ID NO: 4 or 5. In some embodiments, ASOs target a sequence within a coding exon, e.g., an alternatively spliced exon.

### Exon Inclusion

As used herein, a “NMD exon-containing pre-mRNA” is a pre-mRNA transcript that contains at least one pseudo-exon. Alternative or aberrant splicing can result in inclusion of the at least one pseudo-exon in the mature mRNA transcripts. The terms “mature mRNA,” “processed mRNA,” and “fully-spliced mRNA,” are used interchangeably herein to describe a fully processed mRNA that has completed splicing events in a cell. Inclusion of the at least one pseudo-exon can be non-productive mRNA and lead to NMD of the mature mRNA. NMD exon-containing mature mRNA may sometimes lead to aberrant protein expression.

In some embodiments, the included pseudo-exon is the most abundant pseudo-exon in a population of NMD exon-containing pre-mRNAs transcribed from the gene encoding the target protein in a cell. In some embodiments, the included pseudo-exon is the most abundant pseudo-exon in a population of NMD exon-containing pre-mRNAs transcribed from the gene encoding the target protein in a cell, wherein the population of NMD exon-containing pre-mRNAs comprises two or more included pseudo-exons. In some embodiments, an antisense oligomer targeted to the most abundant pseudo-exon in the population of NMD exon-containing pre-mRNAs encoding the target protein induces exon skipping of one or two or more pseudo-exons in the population, including the pseudo-exon to which the antisense oligomer is targeted or binds. In some embodiments, the targeted region is in a pseudo-exon that is the most abundant pseudo-exon in a NMD exon-containing pre-mRNA encoding the OPA1 protein.

The degree of exon inclusion can be expressed as percent exon inclusion, e.g., the percentage of transcripts in which a given pseudo-exon is included. In brief, percent exon inclusion can be calculated as the percentage of the amount of RNA transcripts with the exon inclusion, over the sum of the average of the amount of RNA transcripts with exon inclusion plus the average of the amount of RNA transcripts with exon exclusion.

In some embodiments, an included pseudo-exon is an exon that is identified as an included pseudo-exon based on a determination of at least about 5%, at least about 10%, at least about 15%, at least about 20%, at least about 25%, at least about 30%, at least about 35%, at least about 40%, at least about 45%, or at least about 50%, inclusion. In embodiments, a included pseudo-exon is an exon that is identified as a included pseudo-exon based on a determination of about 5% to about 100%, about 5% to about 95%, about 5% to about 90%, about 5% to about 85%, about 5% to about 80%, about 5% to about 75%, about 5% to about 70%, about 5% to about 65%, about 5% to about 60%, about 5% to about 55%, about 5% to about 50%, about 5% to about 45%, about 5% to about 40%, about 5% to about 35%, about 5% to about 30%, about 5% to about 25%, about 5% to about 20%, about 5% to about 15%, about 10% to about 100%, about 10% to about 95%, about 10% to about 90%, about 10% to about 85%, about 10% to about 80%, about 10% to about 75%, about 10% to about 70%, about 10% to about 65%, about 10% to about 60%, about 10% to about 55%, about 10% to about 50%, about 10% to about 45%, about 10% to about 40%, about 10% to about 35%, about 10% to about 30%, about 10% to about 25%, about 10% to about 20%, about 15% to about 100%, about 15% to about 95%, about 15% to about 90%, about 15% to about 85%, about 15% to about 80%, about 15% to about 75%, about 15% to about 70%, about 15% to about 65%, about 15% to about 60%, about 15% to about 55%, about 15% to about 50%, about 15% to about 45%, about 15% to about 40%, about 15% to about 35%, about 15% to about 30%, about 15% to about 25%, about 20% to about 100%, about 20% to about 95%, about 20% to about 90%, about 20% to about 85%, about 20% to about 80%, about 20% to about 75%, about 20% to about 70%, about 20% to about 65%, about 20% to about 60%, about 20% to about 55%, about 20% to about 50%, about 20% to about 45%, about 20% to about 40%, about 20% to about 35%, about 20% to about 30%, about 25% to about 100%, about 25% to about 95%, about 25% to about 90%, about 25% to about 85%, about 25% to about 80%, about 25% to about 75%, about 25% to about 70%, about 25% to about 65%, about 25% to about 60%, about 25% to about 55%, about 25% to about 50%, about 25% to about 45%, about 25% to about 40%, or about 25% to about 35%, inclusion. ENCODE data (described by, e.g., Tilgner, et al., 2012, “Deep sequencing of subcellular RNA fractions shows splicing to be predominantly co-transcriptional in the human genome but inefficient for lncRNAs,” Genome Research 22(9):1616-25) can be used to aid in identifying exon inclusion.

In some embodiments, contacting cells with an ASO that is complementary to a targeted portion of an OPA1 pre-mRNA transcript results in an increase in the amount of OPA1 protein produced by at least 10, 20, 30, 40, 50, 60, 80, 100, 150, 200, 250, 300, 350, 400, 450, 500, or 1000%, compared to the amount of the protein produced by a cell in the absence of the ASO/absence of treatment. In some embodiments, the total amount of OPA1 protein produced by the cell to which the antisense oligomer is contacted is increased about 20% to about 300%, about 50% to about 300%, about 100% to about 300%, about 150% to about 300%, about 20% to about 50%, about 20% to about 100%, about 20% to about 150%, about 20% to about 200%, about 20% to about 250%, about 50% to about 100%, about 50% to about 150%, about 50% to about 200%, about 50% to about 250%, about 100% to about 150%, about 100% to about 200%, about 100% to about 250%, about 150% to about 200%, about 150% to about 250%, about 200% to about 250%, at least about 10%, at least about 20%, at least about 50%, at least about 100%, at least about 150%, at least about 200%, at least about 250%, or at least about 300%, compared to the amount of target protein produced by a control compound. In some embodiments, the total amount of OPA1 protein produced by the cell to which the antisense oligomer is contacted is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the amount of target protein produced by a control compound. A control compound can be, for example, an oligonucleotide that is not complementary to a targeted portion of the pre-mRNA.

In some embodiments, contacting cells with an ASO that is complementary to a targeted portion of an OPA1 pre-mRNA transcript results in an increase in the amount of mRNA encoding OPA1, including the mature mRNA encoding the target protein. In some embodiments, the amount of mRNA encoding OPA1 protein, or the mature mRNA encoding the OPA1 protein, is increased by at least 10, 20, 30, 40, 50, 60, 80, 100, 150, 200, 250, 300, 350, 400, 450, 500, or 1000%, compared to the amount of the protein produced by a cell in the absence of the ASO/absence of treatment. In some embodiments, the total amount of the mRNA encoding OPA1 protein, or the mature mRNA encoding OPA1 protein produced in the cell to which the antisense oligomer is contacted is increased about 20% to about 300%, about 50% to about 300%, about 100% to about 300%, about 150% to about 300%, about 20% to about 50%, about 20% to about 100%, about 20% to about 150%, about 20% to about 200%, about 20% to about 250%, about 50% to about 100%, about 50% to about 150%, about 50% to about 200%, about 50% to about 250%, about 100% to about 150%, about 100% to about 200%, about 100% to about 250%, about 150% to about 200%, about 150% to about 250%, about 200% to about 250%, at least about 10%, at least about 20%, at least about 50%, at least about 100%, at least about 150%, at least about 200%, at least about 250%, or at least about 300%, compared to the amount of mature RNA produced in an untreated cell, e.g., an untreated cell or a cell treated with a control compound. In some embodiments, the total amount of the mRNA encoding OPA1 protein, or the mature mRNA encoding OPA1 protein produced in the cell to which the antisense oligomer is contacted is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold compared to the amount of mature RNA produced in an untreated cell, e.g., an untreated cell or a cell treated with a control compound. A control compound can be, for example, an oligonucleotide that is not complementary to a targeted portion of the OPA1 NMD exon-containing pre-mRNA.

The NMD exon can be in any length. In some embodiments, the NMD exon comprises a full sequence of an intron, in which case, it can be referred to as intron retention. In some embodiments, the NMD exon can be a portion of the intron. In some embodiments, the NMD exon can be a 5′ end portion of an intron including a 5′ss sequence. In some embodiments, the NMD exon can be a 3′ end portion of an intron including a 3′ss sequence. In some embodiments, the NMD exon can be a portion within an intron without inclusion of a 5′ss sequence. In some embodiments, the NMD exon can be a portion within an intron without inclusion of a 3′ss sequence. In some embodiments, the NMD exon can be a portion within an intron without inclusion of either a 5′ss or a 3′ss sequence. In some embodiments, the NMD exon can be from 5 nucleotides to 10 nucleotides in length, from 10 nucleotides to 15 nucleotides in length, from 15 nucleotides to 20 nucleotides in length, from 20 nucleotides to 25 nucleotides in length, from 25 nucleotides to 30 nucleotides in length, from 30 nucleotides to 35 nucleotides in length, from 35 nucleotides to 40 nucleotides in length, from 40 nucleotides to 45 nucleotides in length, from 45 nucleotides to 50 nucleotides in length, from 50 nucleotides to 55 nucleotides in length, from 55 nucleotides to 60 nucleotides in length, from 60 nucleotides to 65 nucleotides in length, from 65 nucleotides to 70 nucleotides in length, from 70 nucleotides to 75 nucleotides in length, from 75 nucleotides to 80 nucleotides in length, from 80 nucleotides to 85 nucleotides in length, from 85 nucleotides to 90 nucleotides in length, from 90 nucleotides to 95 nucleotides in length, or from 95 nucleotides to 100 nucleotides in length. In some embodiments, the NMD exon can be at least 10 nucleotides, at least 20 nucleotides, at least 30 nucleotides, at least 40 nucleotides, at least 50 nucleotides, at least 60 nucleoids, at least 70 nucleotides, at least 80 nucleotides in length, at least 90 nucleotides, or at least 100 nucleotides in length. In some embodiments, the NMD exon can be from 100 to 200 nucleotides in length, from 200 to 300 nucleotides in length, from 300 to 400 nucleotides in length, from 400 to 500 nucleotides in length, from 500 to 600 nucleotides in length, from 600 to 700 nucleotides in length, from 700 to 800 nucleotides in length, from 800 to 900 nucleotides in length, from 900 to 1,000 nucleotides in length. In some embodiments, the NMD exon may be longer than 1,000 nucleotides in length.

Inclusion of a pseudo-exon can lead to a frameshift and the introduction of a premature termination codon (PIC) in the mature mRNA transcript rendering the transcript a target of NMD. Mature mRNA transcript containing NMD exon can be non-productive mRNA transcript which does not lead to protein expression. The PIC can be present in any position downstream of an NMD exon. In some embodiments, the PIC can be present in any exon downstream of an NMD exon. In some embodiments, the PIC can be present within the NMD exon. For example, inclusion of exon 6x of OPA1, exon 7x of OPA1, or exon 28x of OPA1, in an mRNA transcript encoded by the OPA1 gene can induce a PIC in the mRNA transcript. For example, inclusion of exon (GRCh38/hg38: chr3 193628509 193628616) of OPA1; or exon (GRCh38/hg38: chr3 193603500 193603557) of OPA1 in an mRNA transcript encoded by the OPA1.

In some aspects, provided herein is a method of modulating expression of an OPA1 protein by promoting inclusion of a coding exon. The method can comprise contacting an agent to a cell having an OPA1 pre-mRNA, wherein the agent comprises an oligonucleotide that binds to: (a) a targeted portion of the pre-mRNA within an intronic region immediately upstream of a 5′ end of the coding exon of the pre-mRNA; or (b) a targeted portion of the pre-mRNA within an intronic region immediately downstream of a 3′ end of the coding exon of the pre-mRNA; whereby the agent increases a level of a processed mRNA that is processed from the pre-mRNA and that contains the coding exon in the cell. In some cases, the coding exon to be included is an alternatively spliced exon. In some cases, the method promotes inclusion of the coding exon in the processed mRNA during splicing of the pre-mRNA in the cell.

In some of these embodiments for inclusion of coding exon, the target portion of the pre-mRNA is within a region spanning from 100 to 50, from 100 to 60, from 100 to 70, from 100 to 80, or from 100 to 90 nucleotides upstream of a 5′ end of the coding exon. In some cases, the target portion of the pre-mRNA is within a region spanning from 40 to 100, from 50 to 100, from 60 to 100, from 70 to 100, from 80 to 100, or from 90 to 100 nucleotides downstream of a 3′ end of the coding exon. In some cases, the coding exon is exon 7 of OPAL. In some cases, the coding exon comprises a sequence with at least 80%, at least 90%, or 100% sequence identity to SEQ ID NO: 277. In some cases, the coding exon comprises SEQ ID NO: 277. The targeted portion of the pre-mRNA can be within a region spanning from 100 to 50, from 100 to 60, from 100 to 70, from 100 to 80, or from 100 to 90 nucleotides upstream of genomic site GRCh38/hg38: chr3 193626092. In some cases, the targeted portion of the pre-mRNA is within a region spanning from 40 to 100, from 50 to 100, from 60 to 100, from 70 to 100, from 80 to 100, or from 90 to 100 nucleotides downstream of genomic site GRCh38/hg38: chr3 193626202.

In some cases, the inclusion of the coding exon in the processed mRNA in the cell contacted with the agent is increased by about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to in the absence of the agent.

### Exclusion of Both NMD Exon and Coding Exon

In some embodiments, provided herein is a method of modulating expression of a target protein by targeting a pre-mRNA and modulating exclusion of both a coding exon and a nonsense-mediated RNA decay-inducing exon (NMD exon) from the pre-mRNA. In some cases, the method comprises contacting an agent to the cell, and the agent promotes exclusion of both the coding exon and the NMD exon from the pre-mRNA, thereby increasing level of a processed mRNA that is processed from the pre-mRNA and lacks both the coding exon and the NMD exon. In some cases, the agent binds to a targeted portion of the pre-mRNA, or modulates binding of a factor involved in splicing of the coding exon, the NMD exon, or both. In some cases, the agent interferes with binding of the factor involved in splicing of the coding exon, the NMD exon, or both, to a region of the targeted portion. In some cases, the NMD exon is within an intronic region adjacent to the coding exon. In some cases, the NMD exon is within an intronic region immediately upstream of the coding exon. In some cases, the NMD exon is within an intronic region immediately downstream of the coding exon. In some cases, the coding exon is an alternatively spliced exon.

In some cases, the targeted portion of the pre-mRNA is proximal to the coding exon. The targeted portion of the pre-mRNA can be located in an intronic region immediately upstream of the coding exon. The targeted portion of the pre-mRNA can be located in an intronic region immediately downstream of the coding exon. In some cases, the targeted portion of the pre-mRNA can be located within the coding exon. In some cases, the targeted portion of the pre-mRNA is within a region spanning from 49 to 1, from 39 to 1, from 29 to 1, or from 19 to 1 nucleotides upstream of 5′ end of the coding exon. In some cases, the targeted portion of the pre-mRNA is within a region spanning from 100 nucleotides upstream of the coding exon to 100 nucleotides downstream of the coding exon. In some cases, the targeted portion comprises about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more consecutive nucleotides of the coding exon.

In some cases, the targeted portion of the pre-mRNA is proximal to the NMD exon. In some cases, the targeted portion of the pre-mRNA is located in an intronic region immediately upstream of the NMD exon. In some cases, the targeted portion of the pre-mRNA is located in an intronic region immediately downstream of the NMD exon. In some cases, the targeted portion of the pre-mRNA is located within the NMD exon. In some cases, the targeted portion of the pre-mRNA is within a region spanning from 100 nucleotides upstream of the NMD exon to 100 nucleotides downstream of the NMD exon.

In some embodiments, the method described herein is applicable to modulation of expression of OPA1 protein by modulating exclusion of both exon 7 and an NMD exon (e.g., exon 7x) of OPA1 pre-mRNA that contains both exon 7 and exon 7x. In some cases, the coding exon comprises a sequence with at least 80%, at least 90%, or 100% sequence identity to SEQ ID NO: 277. In some cases, the coding exon comprises SEQ ID NO: 277. In some cases, the targeted portion of the pre-mRNA is immediately upstream of the coding exon GRCh38/hg38: chr3 193626092 to 193626202. In some cases, the targeted portion of the pre-mRNA is immediately downstream of the coding exon GRCh38/hg38: chr3 193626092 to 193626202. In some cases, the targeted portion of the pre-mRNA is within a region spanning from 49 to 1, from 39 to 1, from 29 to 1, or from 19 to 1 nucleotides upstream of GRCh38/hg38: chr3 193626092. In some cases, the targeted portion of the pre-mRNA is within a region spanning from 100 nucleotides upstream of genomic site GRCh38/hg38: chr3 193626092 to 100 nucleotides downstream of genomic site GRCh38/hg38: chr3 193626202. In some cases, the targeted portion of the pre-mRNA is within the coding exon GRCh38/hg38: chr3 193626092 to 193626202. In some cases, the targeted portion of the pre-mRNA comprises an exon-intron junction of the coding exon GRCh38/hg38: chr3 193626092 to 193626202. In some cases, the NMD exon comprises a sequence with at least 80%, at least 90%, or 100% sequence identity to SEQ ID NO: 279. In some cases, the NMD exon comprises SEQ ID NO: 279. In some cases, the targeted portion of the pre-mRNA is immediately upstream of the NMD exon GRCh38/hg38: chr3 193628509 to 193628616. In some cases, the targeted portion of the pre-mRNA is immediately downstream of the NMD exon GRCh38/hg38: chr3 193628509 to 193628616. In some cases, the targeted portion of the pre-mRNA is within a region spanning from 100 nucleotides upstream of genomic site GRCh38/hg38: chr3 193628509 to 100 nucleotides downstream of genomic site GRCh38/hg38: chr3 193628616.

In some cases, the targeted portion of the pre-mRNA is within the NMD exon GRCh38/hg38: chr3 193628509 to 193628616. In some cases, the targeted portion of the pre-mRNA comprises an exon-intron junction of the NMD exon GRCh38/hg38: chr3 193628509 to 193628616. In some cases, the targeted portion comprises about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more consecutive nucleotides of the NMD exon.

In some cases, the exclusion of the coding exon from the pre-mRNA in the cell contacted with the agent is increased by about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to in the absence of contacting with the agent. In some cases, the exclusion of the NMD exon from the pre-mRNA in the cell contacted with the agent is increased by about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to in the absence of contacting with the agent. In some cases, the method results in an increase in the level of the processed mRNA in the cell. The level of the processed mRNA in the cell contacted with the agent can be increased by about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to in the absence of contacting with the agent.

In some cases, the method results in an increase in expression of the OPA1 protein in the cell. A level of the OPA1 protein expressed from the processed mRNA in the cell contacted with the agent can be increased by about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to in the absence of contacting with the agent.

In some cases, a level of the OPA1 protein expressed from the processed mRNA in the cell contacted with the agent is increased by at least about 1.5-fold compared to in the absence of contacting with the agent.

In some cases, the OPA1 protein expressed from the processed mRNA that lacks exon 7 and exon 7x is a functional OPA1 protein. The OPA1 protein expressed from the processed mRNA that lacks exon 7 and exon 7x can be at least partially functional as compared to a wild-type OPA1 protein. The OPA1 protein expressed from the processed mRNA that lacks exon 7 and exon 7x can be at least partially functional as compared to a full-length wild-type OPA1 protein.

### Protein Expression

In some embodiments, the methods described herein are used to increase the production of a functional target protein, e.g., OPA1 protein. As used herein, the term “functional” refers to the amount of activity or function of a target protein, e.g., OPA1 protein, that is necessary to eliminate any one or more symptoms of a treated condition or disease, e.g., Optic atrophy type 1. In some embodiments, the methods are used to increase the production of a partially functional target protein, e.g., OPA1 protein. As used herein, the term “partially functional” refers to any amount of activity or function of the target protein, e.g., OPA1 protein, that is less than the amount of activity or function that is necessary to eliminate or prevent any one or more symptoms of a disease or condition. In some embodiments, a partially functional protein or RNA will have at least 10%, at least 20%, at least 30%, at least 40%, at least 50%, at least 60%, at least 70%, at least 75%, at least 80%, at least 85%, at least 90%, or at least 95% less activity relative to the fully functional protein or RNA.

In some embodiments, the method is a method of increasing the expression of a target protein, protein by cells of a subject having a target gene that encodes the target protein, wherein the subject has a disease or condition caused by a deficient amount of activity of the target protein, and wherein the deficient amount of the target protein is caused by haploinsufficiency of the target protein. In such an embodiment, the subject has a first allele encoding a functional target protein, and a second allele from which the target protein is not produced. In another such embodiment, the subject has a first allele encoding a functional target protein, and a second allele encoding a nonfunctional target protein. In another such embodiment, the subject has a first allele encoding a functional target protein, and a second allele encoding a partially functional target protein. In any of these embodiments, the agent binds to a targeted portion of the target pre-mRNA transcribed from the second allele, thereby inducing exon skipping of the pseudo-exon from the pre-mRNA, and causing an increase in the level of mature mRNA encoding functional target protein, and an increase in the expression of the target protein in the cells of the subject. Alternatively, or additionally, the agent can bind to a targeted portion of the target processed mRNA that encodes the target protein, thereby increasing translation of the target processed mRNA and increasing expression of the target protein.

In some embodiments, the method is a method of increasing the expression of the OPA1, protein by cells of a subject having an OPA1 gene, wherein the subject has a disease or condition, e.g., Optic atrophy type 1, caused by a deficient amount of activity of OPA1 protein, and wherein the deficient amount of the OPA1 protein is caused by haploinsufficiency of the OPA1 protein. In such an embodiment, the subject has a first allele encoding a functional OPA1 protein, and a second allele from which the OPA1 protein is not produced. In another such embodiment, the subject has a first allele encoding a functional OPA1 protein, and a second allele encoding a nonfunctional OPA1 protein. In another such embodiment, the subject has a first allele encoding a functional OPA1 protein, and a second allele encoding a partially functional OPA1 protein. In any of these embodiments, the antisense oligomer binds to a targeted portion of the OPA1 pre-mRNA transcribed from the second allele, thereby inducing exon skipping of the pseudo-exon from the pre-mRNA, and causing an increase in the level of mature mRNA encoding functional OPA1 protein, and an increase in the expression of the OPA1 protein in the cells of the subject. Alternatively, or additionally, the agent can bind to a targeted portion of the OPA1 processed mRNA that encodes the OPA1 protein, thereby increasing translation of the OPA1 processed mRNA and increasing expression of the OPA1 protein.

In some embodiments, the method is a method of increasing the expression of the target protein by cells of a subject, wherein the subject has a disease or condition caused by a deficient amount of activity of target protein, and wherein the deficient amount of the target protein is caused by autosomal recessive inheritance.

In some embodiments, the method is a method of increasing the expression of the OPA1 protein by cells of a subject, wherein the subject has a disease or condition caused by a deficient amount of activity of OPA1 protein, and wherein the deficient amount of the OPA1 protein is caused by autosomal recessive inheritance.

In some embodiments, the method is a method of increasing the expression of the target protein by cells of a subject, wherein the subject has a disease or condition caused by a deficient amount of activity of target protein, and wherein the deficient amount of the target protein is caused by autosomal dominant inheritance.

In some embodiments, the method is a method of increasing the expression of the OPA1 protein by cells of a subject having an OPA1 pre-mRNA, wherein the subject has a disease or condition, e.g., Optic atrophy type 1, caused by a deficient amount of activity of OPA1, protein, and wherein the deficient amount of the OPA1 protein is caused by autosomal dominant inheritance.

In related embodiments, the method is a method of using an ASO to increase the expression of a protein or functional RNA. In some embodiments, an ASO may be used to increase the expression of target protein in cells of a subject having a target pre-mRNA, wherein the subject has a deficiency in the amount or function of a target protein.

In related embodiments, the method is a method of using an ASO to increase the expression of a protein or functional RNA. In some embodiments, an ASO may be used to increase the expression of OPA1 protein in cells of a subject having an OPA1 pre-mRNA, wherein the subject has a deficiency, e.g., Optic atrophy type 1; in the amount or function of an OPA1 protein.

In some embodiments, the pre-mRNA transcript that encodes the protein that is causative of the disease or condition is targeted by the agent, e.g., the oligonucleotides, described herein. In some cases, it is the NMD exon-containing pre-mRNA transcript targeted by the agent, e.g., the oligonucleotides, described herein. In some cases, the agent, e.g., the oligonucleotides, described herein, are designed to target a coding exon of the pre-mRNA. In some cases, the agent, e.g., the oligonucleotides, described herein can induce skipping of the NMD exon, a coding exon, or both. In some embodiments, a NMD exon-containing pre-mRNA transcript that encodes a protein that is not causative of the disease is targeted by the ASOs. For example, a disease that is the result of a mutation or deficiency of a first protein in a particular pathway may be ameliorated by targeting a pre-mRNA that encodes a second protein, thereby increasing production of the second protein. In some embodiments, the function of the second protein is able to compensate for the mutation or deficiency of the first protein (which is causative of the disease or condition).

In some embodiments, the subject has:
(a) a first mutant allele from which
   (i) the OPA1 protein is produced at a reduced level compared to production from a wild-type allele,
   (ii) the OPA1 protein is produced in a form having reduced function compared to an equivalent wild-type protein, or
   (iii) the OPA1 protein or functional RNA is not produced; and
(b) a second mutant allele from which
   (i) the OPA1 protein is produced at a reduced level compared to production from a wild-type allele,
   (ii) the OPA1 protein is produced in a form having reduced function compared to an equivalent wild-type protein, or
   (iii) the OPA1 protein is not produced, and
      wherein the NMD exon-containing pre-mRNA is transcribed from the first allele and/or the second allele. In these embodiments, the ASO binds to a targeted portion of the NMD exon-containing pre-mRNA transcribed from the first allele or the second allele, thereby inducing exon skipping of the pseudo-exon from the NMD exon-containing pre-mRNA, and causing an increase in the level of mRNA encoding OPA1 protein and an increase in the expression of the target protein or functional RNA in the cells of the subject. In these embodiments, the target protein or functional RNA having an increase in expression level resulting from the exon skipping of the pseudo-exon from the NMD exon-containing pre-mRNA may be either in a form having reduced function compared to the equivalent wild-type protein (partially-functional), or having full function compared to the equivalent wild-type protein (fully-functional).

In some embodiments, the subject has:
(a) a first mutant allele from which
   (i) the OPA1 protein is produced at a reduced level compared to production from a wild-type allele,
   (ii) the OPA1 protein is produced in a form having reduced function compared to an equivalent wild-type protein, or
   (iii) the OPA1 protein or functional RNA is not produced; and
(b) a second mutant allele from which
   (i) the OPA1 protein is produced at a reduced level compared to production from a wild-type allele,
   (ii) the OPA1 protein is produced in a form having reduced function compared to an equivalent wild-type protein, or
   (iii) the OPA1 protein is not produced, and
      wherein the OPA1 pre-mRNA is transcribed from the first allele and/or the second allele. In these embodiments, the ASO binds to a targeted portion of the OPA1 pre-mRNA transcribed from the first allele or the second allele, thereby inducing exon skipping of a coding exon from the OPA1 pre-mRNA, and causing an increase in the expression of the target OPA1 protein in the cells of the subject. In these embodiments, the target OPA1 protein having an increase in expression level resulting from the exon skipping of the coding exon from the OPA1 pre-mRNA may be either in a form having reduced function compared to the equivalent full-length wild-type protein (partially-functional), or having full function compared to the equivalent full-length wild-type protein (fully-functional).

In some embodiments, the level of mRNA encoding OPA1 protein is increased 1.1 to 10-fold, when compared to the amount of mRNA encoding OPA1 protein that is produced in a control cell, e.g., one that is not treated with the antisense oligomer or one that is treated with an antisense oligomer that does not bind to the targeted portion of the OPA1 pre-mRNA.

In some embodiments, a level of the OPA1 protein expressed in the cell contacted with the agent disclosed herein or the vector encoding the agent is increased compared to the level of the OPA1 protein in a control cell not contacted with the agent or the vector encoding the agent. In some cases, a level of the OPA1 protein expressed in the cell contacted with the agent or the vector encoding the agent is increased by about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the level of the OPA1 protein in a control cell not contacted with the agent or the vector encoding the agent.

In some cases, a level of the OPA1 protein expressed in the cell contacted with the agent disclosed herein or the vector encoding the agent is increased by about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to in the absence of the agent.

In some embodiments, the OPA1 protein translated from the processed mRNA is a functional OPA1 protein. In some embodiments, the OPA1 protein translated from the processed mRNA is fully functional. In some embodiments, the OPA1 protein translated from the processed mRNA is a wild-type OPA1 protein. In some embodiments, the OPA1 protein translated from the processed mRNA is a full-length OPA1 protein. In some embodiments, the OPA1 protein translated from the processed mRNA has at least 80%, 82%, 84%, 85%, 86%, 88%, 90%, 91%, 92%, 93%, 94%, 9%, 96%, 97%, 98%, or 99%, or 100% sequence identity to any sequence listed in Table 2. In some embodiments, the OPA1 protein translated from the processed mRNA has at least 80%, 82%, 84%, 85%, 86%, 88%, 90%, 91%, 92%, 93%, 94%, 9%, 96%, 97%, 98%, or 99%, or 100% sequence identity to the sequence selected from the group consisting of SEQ ID NOs: 1272-1280.

In some embodiments, a subject treated using the methods of the present disclosure expresses a partially functional OPA1 protein from one allele, wherein the partially functional OPA1 protein may be caused by a frameshift mutation, a nonsense mutation, a missense mutation, or a partial gene deletion. In some embodiments, a subject treated using the methods of the disclosure expresses a nonfunctional OPA1 protein from one allele, wherein the nonfunctional OPA1 protein may be caused by a frameshift mutation, a nonsense mutation, a missense mutation, a partial gene deletion, in one allele. In some embodiments, a subject treated using the methods of the disclosure has an OPA1 whole gene deletion, in one allele.

### Therapeutic Agents

In various embodiments of the present disclosure, compositions and methods comprising a therapeutic agent are provided to modulate protein expression level of a target protein, e.g., OPA1 protein. In some embodiments, provided herein are compositions and methods to modulate translation of target processed mRNA, e.g., OPA1 processed mRNA. In some embodiments, provided herein are compositions and methods to modulate alternative splicing of target pre-mRNA, e.g., OPA1 pre-mRNA. In some embodiments, provided herein are compositions and methods to induce exon skipping in the splicing of target pre-mRNA, e.g., OPA1 pre-mRNA e.g., to induce skipping of a pseudo-exon during splicing of target pre-mRNA, e.g., OPA1 pre-mRNA.

A therapeutic agent disclosed herein can be a translation modulator, e.g., an agent disclosed herein that modulates translation of a processed mRNA that encodes a target protein. A therapeutic agent disclosed herein can be a NIE repressor agent. A therapeutic agent may comprise a polynucleic acid polymer.

According to one aspect of the present disclosure, provided herein is a method of treatment or prevention of a condition or disease associated with a functional OPA1 protein deficiency, comprising administering a NIE repressor agent to a subject to increase levels of functional OPA1 protein, wherein the agent binds to a region of the pre-mRNA transcript to decrease inclusion of the NMD exon in the mature transcript. For example, provided herein is a method of treatment or prevention of a condition associated with a functional OPA1 protein deficiency, comprising administering a NIE repressor agent to a subject to increase levels of functional OPA1 protein, wherein the agent binds to a region of an intron containing an NMD exon (e.g., exon 6x of OPA1, exon 7x of OPA1, or exon 28x of OPA1) of the pre-mRNA transcript or to a NMD exon-activating regulatory sequence in the same intron. For example, provided herein is a method of treatment or prevention of a condition associated with a functional OPA1 protein deficiency, comprising administering a NIE repressor agent to a subject to increase levels of functional OPA1 protein, wherein the agent binds to a region of an intron containing an NMD exon (e.g., exon (GRCh38/hg38: chr3 193628509 193628616) of OPA1; or exon (GRCh38/hg38: chr3 193603500 193603557) of OPA1) of the pre-mRNA transcript or to a NMD exon-activating regulatory sequence in the same intron. In some embodiments, the method comprises administering a NIE repressor agent to a subject to increase levels of functional OPA1 protein, wherein the agent binds to a region of an intron containing an NMD exon (e.g., exon of OPA1 other than exon 7x defined by (GRCh38/hg38: chr3 193628509 193628616) or exon defined by (GRCh38/hg38: chr3 193603500 193603557)) of the pre-mRNA transcript or to a NMD exon-activating regulatory sequence in the same intron. In some embodiments, the therapeutic agent promotes exclusion of an NMD exon of OPA1 pre-mRNA other than exon 7x defined by (GRCh38/hg38: chr3 193628509 193628616) or exon defined by (GRCh38/hg38: chr3 193603500 193603557). In some embodiments, the composition disclosed herein includes an agent that promotes exclusion of an NMD exon of OPA1 pre-mRNA other than exon 7x defined by (GRCh38/hg38: chr3 193628509 193628616) or exon defined by (GRCh38/hg38: chr3 193603500 193603557).

Where reference is made to reducing NMD exon inclusion in the mature mRNA, the reduction may be complete, e.g., 100%, or may be partial. The reduction may be clinically significant. The reduction/correction may be relative to the level of NMD exon inclusion in the subject without treatment, or relative to the amount of NMD exon inclusion in a population of similar subjects. The reduction/correction may be at least 10% less NMD exon inclusion relative to the average subject, or the subject prior to treatment. The reduction may be at least 20% less NMD exon inclusion relative to an average subject, or the subject prior to treatment. The reduction may be at least 40% less NMD exon inclusion relative to an average subject, or the subject prior to treatment. The reduction may be at least 50% less NMD exon inclusion relative to an average subject, or the subject prior to treatment. The reduction may be at least 60% less NMD exon inclusion relative to an average subject, or the subject prior to treatment. The reduction may be at least 80% less NMD exon inclusion relative to an average subject, or the subject prior to treatment. The reduction may be at least 90% less NMD exon inclusion relative to an average subject, or the subject prior to treatment.

According to one aspect of the present disclosure, provided herein is a method of treatment or prevention of a condition or disease associated with a functional OPA1 protein deficiency, comprising administering an agent to a subject to increase levels of functional OPA1 protein, wherein the agent binds to a region of the pre-mRNA transcript to decrease inclusion of a coding exon (e.g., exon 7) in the mature transcript. For example, provided herein is a method of treatment or prevention of a condition associated with a functional OPA1 protein deficiency, comprising administering an agent to a subject to increase levels of functional OPA1 protein, wherein the agent binds to a region containing a coding exon (e.g., exon 7 of OPA1) of the pre-mRNA transcript. For example, provided herein is a method of treatment or prevention of a condition associated with a functional OPA1 protein deficiency, comprising administering an agent to a subject to increase levels of functional OPA1 protein, wherein the agent binds to a region containing a coding exon (e.g., exon (GRCh38/hg38: chr3 193626092 to 193626202) of OPA1) of the pre-mRNA transcript. In some embodiments, the method comprises administering an agent to a subject to increase levels of functional OPA1 protein, wherein the agent binds to a region containing a coding exon (e.g., exon of OPA1 other than exon 7 defined by (GRCh38/hg38: chr3 193626092 to 193626202)) of the pre-mRNA transcript. In some embodiments, the therapeutic agent promotes exclusion of a coding exon of OPA1 pre-mRNA other than exon 7 defined by (GRCh38/hg38: chr3 193626092 to 193626202). In some embodiments, the composition disclosed herein includes an agent that promotes exclusion of a coding exon of OPA1 pre-mRNA other than exon 7 defined by (GRCh38/hg38: chr3 193626092 to 193626202).

Where reference is made to increasing active OPA1 protein levels, the increase may be clinically significant. The increase may be relative to the level of active OPA1 protein in the subject without treatment, or relative to the amount of active OPA1 protein in a population of similar subjects. The increase may be at least 10% more active OPA1 protein relative to the average subject, or the subject prior to treatment. The increase may be at least 20% more active OPA1 protein relative to the average subject, or the subject prior to treatment. The increase may be at least 40% more active OPA1 protein relative to the average subject, or the subject prior to treatment. The increase may be at least 50% more active OPA1 protein relative to the average subject, or the subject prior to treatment. The increase may be at least 80% more active OPA1 protein relative to the average subject, or the subject prior to treatment. The increase may be at least 100% more active OPA1 protein relative to the average subject, or the subject prior to treatment. The increase may be at least 200% more active OPA1 protein relative to the average subject, or the subject prior to treatment. The increase may be at least 500% more active OPA1 protein relative to the average subject, or the subject prior to treatment.

In embodiments wherein the NIE repressor agent comprises a polynucleic acid polymer, the polynucleic acid polymer may be about 50 nucleotides in length. The polynucleic acid polymer may be about 45 nucleotides in length. The polynucleic acid polymer may be about 40 nucleotides in length. The polynucleic acid polymer may be about 35 nucleotides in length. The polynucleic acid polymer may be about 30 nucleotides in length. The polynucleic acid polymer may be about 24 nucleotides in length. The polynucleic acid polymer may be about 25 nucleotides in length. The polynucleic acid polymer may be about 20 nucleotides in length. The polynucleic acid polymer may be about 19 nucleotides in length. The polynucleic acid polymer may be about 18 nucleotides in length. The polynucleic acid polymer may be about 17 nucleotides in length. The polynucleic acid polymer may be about 16 nucleotides in length. The polynucleic acid polymer may be about 15 nucleotides in length. The polynucleic acid polymer may be about 14 nucleotides in length. The polynucleic acid polymer may be about 13 nucleotides in length. The polynucleic acid polymer may be about 12 nucleotides in length. The polynucleic acid polymer may be about 11 nucleotides in length. The polynucleic acid polymer may be about 10 nucleotides in length. The polynucleic acid polymer may be between about 10 and about 50 nucleotides in length. The polynucleic acid polymer may be between about 10 and about 45 nucleotides in length. The polynucleic acid polymer may be between about 10 and about 40 nucleotides in length. The polynucleic acid polymer may be between about 10 and about 35 nucleotides in length. The polynucleic acid polymer may be between about 10 and about 30 nucleotides in length. The polynucleic acid polymer may be between about 10 and about 25 nucleotides in length. The polynucleic acid polymer may be between about 10 and about 20 nucleotides in length. The polynucleic acid polymer may be between about 15 and about 25 nucleotides in length. The polynucleic acid polymer may be between about 15 and about 30 nucleotides in length. The polynucleic acid polymer may be between about 12 and about 30 nucleotides in length.

The sequence of the polynucleic acid polymer can be at least 50%, 55%, 60%, 65%, 70%, 75%, 80%, 85%, 90%, 91%, 92%, 93%, 94%, 95%, 96%, 97%, 98%, 99%, or 99.5% complementary to a target sequence of an mRNA transcript, e.g., a pre-mRNA transcript or a processed mRNA transcript. The sequence of the polynucleic acid polymer can be 100% complementary to a target sequence of a mRNA transcript (e.g., a pre-mRNA transcript or a processed mRNA transcript).

The sequence of the polynucleic acid polymer may have 4 or fewer mismatches to a target sequence of the mRNA transcript. The sequence of the polynucleic acid polymer may have 3 or fewer mismatches to a target sequence of the mRNA transcript. The sequence of the polynucleic acid polymer may have 2 or fewer mismatches to a target sequence of the mRNA transcript. The sequence of the polynucleic acid polymer may have 1 or fewer mismatches to a target sequence of the mRNA transcript. The sequence of the polynucleic acid polymer may have no mismatches to a target sequence of the mRNA transcript.

The polynucleic acid polymer may specifically hybridize to a target sequence of the mRNA transcript. For example, the polynucleic acid polymer may have 91%, 92%, 93%, 94%, 95%, 96%, 97%, 98%, 99%, 99.5% or 100% sequence complementarity to a target sequence of the mRNA transcript. The hybridization may be under high stringent hybridization conditions.

The polynucleic acid polymer comprising a sequence with at least 50%, 55%, 60%, 65%, 70%, 75%, 80%, 85%, 90%, 91%, 92%, 93%, 94%, 95%, 96%, 97%, 98%, 99%, or 99.5% sequence identity to a sequence selected from the group consisting of SEQ ID NOs: 2-5. The polynucleic acid polymer may comprise a sequence with 100% sequence identity to a sequence selected from the group consisting of SEQ ID NOs: 2-5.

Where reference is made to a polynucleic acid polymer sequence, the skilled person will understand that one or more substitutions may be tolerated, optionally two substitutions may be tolerated in the sequence, such that it maintains the ability to hybridize to the target sequence; or where the substitution is in a target sequence, the ability to be recognized as the target sequence. References to sequence identity may be determined by BLAST sequence alignment using standard/default parameters. For example, the sequence may have 99% identity and still function according to the present disclosure. In other embodiments, the sequence may have 98% identity and still function according to the present disclosure. In another embodiment, the sequence may have 95% identity and still function according to the present disclosure. In another embodiment, the sequence may have 90% identity and still function according to the present disclosure.

In some cases, an agent, e.g., a therapeutic agent, disclosed herein comprises a modified snRNA, such as a modified human or murine snRNA. In some cases, an agent, e.g., a therapeutic agent, comprises a vector, such as a viral vector, that encodes a modified snRNA. In some embodiments, the modified snRNA is a modified U1 snRNA (see, e.g., Alanis et al., Human Molecular Genetics, 2012, Vol. 21, No. 11 2389-2398). In some embodiments, the modified snRNA is a modified U7 snRNA (see, e.g., Gadgil et al., J Gene Med. 2021; 23:e3321). Modified U7 snRNAs can be made by any method known in the art including the methods described in Meyer, K.; Schumperli, Daniel (2012), Antisense Derivatives of U7 Small Nuclear RNA as Modulators of Pre-mRNA Splicing. In: Stamm, Stefan; Smith, Christopher W. J.; Lthrmann, Reinhard (eds.) Alternative pre-mRNA Splicing: Theory and Protocols (pp. 481-494), Chichester: John Wiley & Sons 10.1002/9783527636778.ch45, incorporated by reference herein in its entirety. In some embodiments, a modified U7 (smOPT) does not compete with WT U7 (Stefanovic et al., 1995).

In some embodiments, the modified snRNA comprises an smOPT modification. For example, the modified snRNA can comprise a sequence AAUUUUUGGAG (SEQ ID NO: 1311). For example, the sequence AAUUUUUGGAG (SEQ ID NO: 1311) can replace a sequence AAUUUGUCUAG (SEQ ID NO: 1312) in a wild-type U7 snRNA to generate the modified U& snRNA (smOPT). In some embodiments, a smOPT modification of a U7 snRNA renders the particle functionally inactive in histone pre-mRNA processing (Stefanovic et al., 1995). In some embodiments, a modified U7 (smOPT) is expressed stably in the nucleus and at higher levels than WT U7 (Stefanovic et al., 1995). In some embodiments, the snRNA comprises a U1 snRNP-targeted sequence. In some embodiments, the snRNA comprises a U7 snRNP-targeted sequence. In some embodiments, the snRNA comprises a modified U7 snRNP-targeted sequence and wherein the modified U7 snRNP-targeted sequence comprises smOPT. In some embodiments, the modified snRNA has been modified to comprise a single-stranded nucleotide sequence that hybridizes to a pre-mRNA, such as an ASCE-containing pre-mRNA. For example, the modified snRNA can be modified to comprise a single-stranded nucleotide sequence that hybridizes to an OPA1 pre-mRNA. In some embodiments, the modified snRNA has been modified to comprise a single-stranded nucleotide sequence that hybridizes to an OPA1 processed mRNA, e.g., 5′ UTR of OPA1 processed mRNA. In some embodiments, the modified snRNA has been modified to comprise a single-stranded nucleotide sequence that comprises one or two or more sequences of the ASOs disclosed herein. In some embodiments, the modified snRNA has been modified to comprise a single-stranded nucleotide sequence that hybridizes to a targeted portion of an OPA1 processed mRNA, or a targeted portion of an OPA1 pre-mRNA. In some embodiments, the modified snRNA has been modified to comprise a single-stranded nucleotide sequence that comprises two or more sequences that hybridize to two or more target regions of an OPA1 processed mRNA, or two or more target regions of an OPA1 pre-mRNA. For example, a modified snRNA can be modified to comprise a single-stranded nucleotide sequence that hybridizes to at least 8 contiguous nucleic acids of an OPA1 processed mRNA or an OPA1 pre-mRNA. In some embodiments, the modified snRNA has been modified to comprise a single-stranded nucleotide sequence that hybridizes to any of the target regions of an OPA1 processed mRNA or an OPA1 pre-mRNA disclosed herein. In some embodiments, the modified snRNA has been modified to comprise a single-stranded nucleotide sequence that comprises two or more sequences that hybridize to two or more target regions of an OPA1 processed mRNA or an OPA1 pre-mRNA.

### Antisense Oligomers

Provided herein is a composition comprising an antisense oligomer that induces exon skipping by binding to a targeted portion of an OPA1 processed mRNA or an OPA1 pre-mRNA, e.g., an OPA1 NMD exon-containing pre-mRNA. As used herein, the terms “ASO” and “antisense oligomer” are used interchangeably and refer to an oligomer such as a polynucleotide, comprising nucleobases that hybridizes to a target nucleic acid (e.g., an OPA1 processed mRNA or an OPA1 pre-mRNA, e.g., an OPA1 NMD exon-containing pre-mRNA) sequence by Watson-Crick base pairing or wobble base pairing (G-U). The ASO may have exact sequence complementary to the target sequence or near complementarity (e.g., sufficient complementarity to bind the target sequence and enhancing splicing at a splice site). ASOs are designed so that they bind (hybridize) to a target nucleic acid (e.g., a targeted portion of a mRNA transcript) and remain hybridized under physiological conditions. Typically, if they hybridize to a site other than the intended (targeted) nucleic acid sequence, they hybridize to a limited number of sequences that are not a target nucleic acid (to a few sites other than a target nucleic acid). Design of an ASO can take into consideration the occurrence of the nucleic acid sequence of the targeted portion of the mRNA transcript (the processed mRNA or the pre-mRNA) or a sufficiently similar nucleic acid sequence in other locations in the genome or cellular mRNA or transcriptome, such that the likelihood the ASO will bind other sites and cause “off-target” effects is limited. Any antisense oligomers known in the art (for example, in PCT Application No. PCT/US2014/054151, published as WO 2015/035091, titled “Reducing Nonsense-Mediated mRNA Decay,” incorporated by reference herein), can be used to practice the methods described herein.

In some embodiments, ASOs “specifically hybridize” to or are “specific” to a target nucleic acid or a targeted portion of an OPA1 processed mRNA or an OPA1 pre-mRNA, e.g., a NMD exon-containing pre-mRNA. Typically, such hybridization occurs with a Tₘ substantially greater than 37° C., preferably at least 50° C., and typically between 60° C. to approximately 90° C. Such hybridization preferably corresponds to stringent hybridization conditions. At a given ionic strength and pH, the Tₘ is the temperature at which 50% of a target sequence hybridizes to a complementary oligonucleotide.

Oligomers, such as oligonucleotides, are “complementary” to one another when hybridization occurs in an antiparallel configuration between two single-stranded polynucleotides. A double-stranded polynucleotide can be “complementary” to another polynucleotide, if hybridization can occur between one of the strands of the first polynucleotide and the second. Complementarity (the degree to which one polynucleotide is complementary with another) is quantifiable in terms of the proportion (e.g., the percentage) of bases in opposing strands that are expected to form hydrogen bonds with each other, according to generally accepted base-pairing rules. The sequence of an antisense oligomer (ASO) need not be 100% complementary to that of its target nucleic acid to hybridize. In certain embodiments, ASOs can comprise at least 70%, at least 75%, at least 80%, at least 85%, at least 90%, at least 95%, at least 96%, at least 97%, at least 98%, or at least 99% sequence complementarity to a target region within the target nucleic acid sequence to which they are targeted. For example, an ASO in which 18 of 20 nucleobases of the oligomeric compound are complementary to a target region, and would therefore specifically hybridize, would represent 90 percent complementarity. In this example, the remaining non-complementary nucleobases may be clustered together or interspersed with complementary nucleobases and need not be contiguous to each other or to complementary nucleobases. Percent complementarity of an ASO with a region of a target nucleic acid can be determined routinely using BLAST programs (basic local alignment search tools) and PowerBLAST programs known in the art (Altschul, et al., J. Mol. Biol., 1990, 215, 403-410; Zhang and Madden, Genome Res., 1997, 7, 649-656).

An ASO need not hybridize to all nucleobases in a target sequence and the nucleobases to which it does hybridize may be contiguous or noncontiguous. ASOs may hybridize over one or more segments of a pre-mRNA transcript, such that intervening or adjacent segments are not involved in the hybridization event (e.g., a loop structure or hairpin structure may be formed). In certain embodiments, an ASO hybridizes to noncontiguous nucleobases in a target pre-mRNA transcript. For example, an ASO can hybridize to nucleobases in a pre-mRNA transcript that are separated by one or more nucleobase(s) to which the ASO does not hybridize.

The ASOs described herein comprise nucleobases that are complementary to nucleobases present in a target portion of an OPA1 processed mRNA or an OPA1 pre-mRNA, e.g., a NMD exon-containing pre-mRNA. The term ASO embodies oligonucleotides and any other oligomeric molecule that comprises nucleobases capable of hybridizing to a complementary nucleobase on a target mRNA but does not comprise a sugar moiety, such as a peptide nucleic acid (PNA). The ASOs may comprise naturally-occurring nucleotides, nucleotide analogs, modified nucleotides, or any combination of two or three of the preceding. The term “naturally occurring nucleotides” includes deoxyribonucleotides and ribonucleotides. The term “modified nucleotides” includes nucleotides with modified or substituted sugar groups and/or having a modified backbone. In some embodiments, all of the nucleotides of the ASO are modified nucleotides. Chemical modifications of ASOs or components of ASOs that are compatible with the methods and compositions described herein will be evident to one of skill in the art and can be found, for example, in U.S. Pat. Nos. 6,147,200, 8,258,109, 5,656,612, U.S. Patent Publication No. 2012/0190728, and Dias and Stein, Mol. Cancer Ther. 2002, 347-355, herein incorporated by reference in their entirety.

One or more nucleobases of an ASO may be any naturally occurring, unmodified nucleobase such as adenine, guanine, cytosine, thymine and uracil, or any synthetic or modified nucleobase that is sufficiently similar to an unmodified nucleobase such that it is capable of hydrogen bonding with a nucleobase present on a target pre-mRNA. Examples of modified nucleobases include, without limitation, hypoxanthine, xanthine, 7-methylguanine, 5, 6-dihydrouracil, 5-methylcytosine, and 5-hydroxymethoylcytosine.

The ASOs described herein also comprise a backbone structure that connects the components of an oligomer. The term “backbone structure” and “oligomer linkages” may be used interchangeably and refer to the connection between monomers of the ASO. In naturally occurring oligonucleotides, the backbone comprises a 3′-5′ phosphodiester linkage connecting sugar moieties of the oligomer. The backbone structure or oligomer linkages of the ASOs described herein may include (but are not limited to) phosphorothioate, phosphorodithioate, phosphoroselenoate, phosphorodiselenoate, phosphoroanilothioate, phosphoraniladate, phosphoramidate, and the like. See, e.g., LaPlanche, et al., Nucleic Acids Res. 14:9081 (1986); Stec, et al., J. Am. Chem. Soc. 106:6077 (1984), Stein, et al., Nucleic Acids Res. 16:3209 (1988), Zon, et al., Anti-Cancer Drug Design 6:539 (1991); Zon, et al., Oligonucleotides and Analogues: A Practical Approach, pp. 87-108 (F. Eckstein, Ed., Oxford University Press, Oxford England (1991)); Stec, et al., U.S. Pat. No. 5,151,510; Uhlmann and Peyman, Chemical Reviews 90:543 (1990). In some embodiments, the backbone structure of the ASO does not contain phosphorous but rather contains peptide bonds, for example in a peptide nucleic acid (PNA), or linking groups including carbamate, amides, and linear and cyclic hydrocarbon groups. In some embodiments, the backbone modification is a phosphorothioate linkage. In some embodiments, the backbone modification is a phosphoramidate linkage.

In some embodiments, the stereochemistry at each of the phosphorus internucleotide linkages of the ASO backbone is random. In some embodiments, the stereochemistry at each of the phosphorus internucleotide linkages of the ASO backbone is controlled and is not random. For example, U.S. Pat. App. Pub. No. 2014/0194610, “Methods for the Synthesis of Functionalized Nucleic Acids,” incorporated herein by reference, describes methods for independently selecting the handedness of chirality at each phosphorous atom in a nucleic acid oligomer. In some embodiments, an ASO used in the methods of the disclosure, including, but not limited to, any of the ASOs set forth herein in Tables 5 and 6, comprises an ASO having phosphorus internucleotide linkages that are not random. In some embodiments, a composition used in the methods of the disclosure comprises a pure diastereomeric ASO. In some embodiments, a composition used in the methods of the disclosure comprises an ASO that has diastereomeric purity of at least about 90%, at least about 91%, at least about 92%, at least about 93%, at least about 94%, at least about 95%, at least about 96%, at least about 97%, at least about 98%, at least about 99%, about 100%, about 90% to about 100%, about 91% to about 100%, about 92% to about 100%, about 93% to about 100%, about 94% to about 100%, about 95% to about 100%, about 96% to about 100%, about 97% to about 100%, about 98% to about 100%, or about 99% to about 100%.

In some embodiments, the ASO has a nonrandom mixture of Rp and Sp configurations at its phosphorus internucleotide linkages. For example, it has been suggested that a mix of Rp and Sp is required in antisense oligonucleotides to achieve a balance between good activity and nuclease stability (Wan, et al., 2014, “Synthesis, biophysical properties and biological activity of second generation antisense oligonucleotides containing chiral phosphorothioate linkages,” Nucleic Acids Research 42(22): 13456-13468, incorporated herein by reference). In some embodiments, an ASO used in the methods of the disclosure, including, but not limited to, any of the ASOs set forth herein in SEQ ID NOs: 2-5, comprises about 5-100% Rp, at least about 5% Rp, at least about 10% Rp, at least about 15% Rp, at least about 20% Rp, at least about 25% Rp, at least about 30% Rp, at least about 35% Rp, at least about 40% Rp, at least about 45% Rp, at least about 50% Rp, at least about 55% Rp, at least about 60% Rp, at least about 65% Rp, at least about 70% Rp, at least about 75% Rp, at least about 80% Rp, at least about 85% Rp, at least about 90% Rp, or at least about 95% Rp, with the remainder Sp, or about 100% Rp. In some embodiments, an ASO used in the methods of the disclosure, including, but not limited to, any of the ASOs set forth herein comprise a sequence with at least about 80%, 85%, 90%, 95%, 97%, or 100% sequence identity to a region comprising at least 8 contiguous nucleic acids of any one of SEQ ID NOs: 2-5, comprises about 10% to about 100% Rp, about 15% to about 100% Rp, about 20% to about 100% Rp, about 25% to about 100% Rp, about 30% to about 100% Rp, about 35% to about 100% Rp, about 40% to about 100% Rp, about 45% to about 100% Rp, about 50% to about 100% Rp, about 55% to about 100% Rp, about 60% to about 100% Rp, about 65% to about 100% Rp, about 70% to about 100% Rp, about 75% to about 100% Rp, about 80% to about 100% Rp, about 85% to about 100% Rp, about 90% to about 100% Rp, or about 95% to about 100% Rp, about 20% to about 80% Rp, about 25% to about 75% Rp, about 30% to about 70% Rp, about 40% to about 60% Rp, or about 45% to about 55% Rp, with the remainder Sp.

In some embodiments, an ASO used in the methods of the disclosure, including, but not limited to, any of the ASOs set forth herein comprise a sequence with at least about 80%, 85%, 90%, 95%, 97%, or 100% sequence identity to a region comprising at least 8 contiguous nucleic acids of any one of SEQ ID NOs: 2-5, comprises about 5-100% Sp, at least about 5% Sp, at least about 10% Sp, at least about 15% Sp, at least about 20% Sp, at least about 25% Sp, at least about 30% Sp, at least about 35% Sp, at least about 40% Sp, at least about 45% Sp, at least about 50% Sp, at least about 55% Sp, at least about 60% Sp, at least about 65% Sp, at least about 70% Sp, at least about 75% Sp, at least about 80% Sp, at least about 85% Sp, at least about 90% Sp, or at least about 95% Sp, with the remainder Rp, or about 100% Sp. In embodiments, an ASO used in the methods of the disclosure, including, but not limited to, any of the ASOs set forth herein comprise a sequence with at least about 80%, 85%, 90%, 95%, 97%, or 100% sequence identity to a region comprising at least 8 contiguous nucleic acids of any one of SEQ ID NOs: 2-5, comprises about 10% to about 100% Sp, about 15% to about 100% Sp, about 20% to about 100% Sp, about 25% to about 100% Sp, about 30% to about 100% Sp, about 35% to about 100% Sp, about 40% to about 100% Sp, about 45% to about 100% Sp, about 50% to about 100% Sp, about 55% to about 100% Sp, about 60% to about 100% Sp, about 65% to about 100% Sp, about 70% to about 100% Sp, about 75% to about 100% Sp, about 80% to about 100% Sp, about 85% to about 100% Sp, about 90% to about 100% Sp, or about 95% to about 100% Sp, about 20% to about 80% Sp, about 25% to about 75% Sp, about 30% to about 70% Sp, about 40% to about 60% Sp, or about 45% to about 55% Sp, with the remainder Rp.

Any of the ASOs described herein may contain a sugar moiety that comprises ribose or deoxyribose, as present in naturally occurring nucleotides, or a modified sugar moiety or sugar analog, including a morpholine ring. Non-limiting examples of modified sugar moieties include 2′ substitutions such as 2′-O-methyl (2′-O-Me), 2′-O-methoxyethyl (2′MOE), 2′-O-aminoethyl, 2′-O—N-methyl-acetamide (2′-NMA); 2′F; N3′→P5′ phosphoramidate, 2′dimethylaminooxyethoxy, 2′dimethylaminoethoxyethoxy, 2′-guanidinidium, 2′-O-guanidinium ethyl, carbamate modified sugars, and bicyclic modified sugars. In some embodiments, the sugar moiety modification is selected from 2′-O-Me, 2′F, and 2′MOE. In some embodiments, the sugar moiety modification is an extra bridge bond, such as in a locked nucleic acid (LNA). In some embodiments the sugar analog contains a morpholine ring, such as phosphorodiamidate morpholino (PMO). In some embodiments, the sugar moiety comprises a ribofuranosyl or 2′deoxyribofuransyl modification. In some embodiments, the sugar moiety comprises 2′4′-constrained 2′O-methyloxyethyl (cMOE) modifications. In some embodiments, the sugar moiety comprises cEt 2′, 4′ constrained 2′-O ethyl BNA modifications. In some embodiments, the sugar moiety comprises tricycloDNA (tcDNA) modifications. In some embodiments, the sugar moiety comprises ethylene nucleic acid (ENA) modifications. In some embodiments, the sugar moiety comprises MCE modifications. Modifications are known in the art and described in the literature, e.g., by Jarver, et al., 2014, “A Chemical View of Oligonucleotides for Exon Skipping and Related Drug Applications,” Nucleic Acid Therapeutics 24(1): 37-47, incorporated by reference for this purpose herein.

In some embodiments, each monomer of the ASO is modified in the same way, for example each linkage of the backbone of the ASO comprises a phosphorothioate linkage or each ribose sugar moiety comprises a 2′O-methyl modification. Such modifications that are present on each of the monomer components of an ASO are referred to as “uniform modifications.” In some examples, a combination of different modifications may be desired, for example, an ASO may comprise a combination of phosphorodiamidate linkages and sugar moieties comprising morpholine rings (morpholinos). Combinations of different modifications to an ASO are referred to as “mixed modifications” or “mixed chemistries.”

In some embodiments, the ASO comprises one or more backbone modifications. In some embodiments, the ASO comprises one or more sugar moiety modification. In some embodiments, the ASO comprises one or more backbone modifications and one or more sugar moiety modifications. In some embodiments, the ASO comprises a 2′MOE modification and a phosphorothioate backbone. In some embodiments, the ASO comprises a phosphorodiamidate morpholino (PMO). In some embodiments, the ASO comprises a peptide nucleic acid (PNA). Any of the ASOs or any component of an ASO (e.g., a nucleobase, sugar moiety, backbone) described herein may be modified in order to achieve desired properties or activities of the ASO or reduce undesired properties or activities of the ASO. For example, an ASO or one or more components of any ASO may be modified to enhance binding affinity to a target sequence on a pre-mRNA transcript; reduce binding to any non-target sequence; reduce degradation by cellular nucleases (i.e., RNase H); improve uptake of the ASO into a cell and/or into the nucleus of a cell; alter the pharmacokinetics or pharmacodynamics of the ASO; and/or modulate the half-life of the ASO.

In some embodiments, the ASOs are comprised of 2′-O-(2-methoxyethyl) (MOE) phosphorothioate-modified nucleotides. In some embodiments, the ASOs are comprised of 2′NMA phosphorothioate-modified nucleotides. ASOs comprised of such nucleotides are especially well-suited to the methods disclosed herein; oligomers having such modifications have been shown to have significantly enhanced resistance to nuclease degradation and increased bioavailability, making them suitable, for example, for oral delivery in some embodiments described herein. See e.g., Geary, et al., J Pharmacol Exp Ther. 2001; 296(3):890-7; Geary, et al., J Pharmacol Exp Ther. 2001; 296(3):898-904.

Methods of synthesizing ASOs will be known to one of skill in the art. Alternatively or in addition, ASOs may be obtained from a commercial source.

Unless specified otherwise, the left-hand end of single-stranded nucleic acid (e.g., pre-mRNA transcript, oligonucleotide, ASO, etc.) sequences is the 5′ end and the left-hand direction of single or double-stranded nucleic acid sequences is referred to as the 5′ direction. Similarly, the right-hand end or direction of a nucleic acid sequence (single or double stranded) is the 3′ end or direction. Generally, a region or sequence that is 5′ to a reference point in a nucleic acid is referred to as “upstream,” and a region or sequence that is 3′ to a reference point in a nucleic acid is referred to as “downstream.” Generally, the 5′ direction or end of an mRNA is where the initiation or start codon is located, while the 3′ end or direction is where the termination codon is located. In some aspects, nucleotides that are upstream of a reference point in a nucleic acid may be designated by a negative number, while nucleotides that are downstream of a reference point may be designated by a positive number. For example, a reference point (e.g., an exon-exon junction in mRNA) may be designated as the “zero” site, and a nucleotide that is directly adjacent and upstream of the reference point is designated “minus one,” e.g., “−1,” while a nucleotide that is directly adjacent and downstream of the reference point is designated “plus one,” e.g., “+1.”

In some embodiments, the ASOs are complementary to (and bind to) a targeted portion of an OPA1 pre-mRNA, e.g., an OPA1 NMD exon-containing pre-mRNA, that is downstream (in the 3′ direction) of the 5′ splice site (or 3′ end of the NMD exon) of the included exon in an OPA1 pre-mRNA (e.g., the direction designated by positive numbers relative to the 5′ splice site). In some embodiments, the ASOs are complementary to a targeted portion of the OPA1 pre-mRNA, e.g., the OPA1 NMD exon-containing pre-mRNA that is within the region about +1 to about +500 relative to the 5′ splice site (or 3′ end) of the included exon. In some embodiments, the ASOs may be complementary to a targeted portion of an OPA1 pre-mRNA, e.g., an OPA1 NMD exon-containing pre-mRNA, that is within the region between nucleotides +6 and +40,000 relative to the 5′ splice site (or 3′ end) of the included exon. In some aspects, the ASOs are complementary to a targeted portion that is within the region about +1 to about +40,000, about +1 to about +30,000, about +1 to about +20,000, about +1 to about +15,000, about +1 to about +10,000, about +1 to about +5,000, about +1 to about +4,000, about +1 to about +3,000, about +1 to about +2,000, about +1 to about +1,000, about +1 to about +500, about +1 to about +490, about +1 to about +480, about +1 to about +470, about +1 to about +460, about +1 to about +450, about +1 to about +440, about +1 to about +430, about +1 to about +420, about +1 to about +410, about +1 to about +400, about +1 to about +390, about +1 to about +380, about +1 to about +370, about +1 to about +360, about +1 to about +350, about +1 to about +340, about +1 to about +330, about +1 to about +320, about +1 to about +310, about +1 to about +300, about +1 to about +290, about +1 to about +280, about +1 to about +270, about +1 to about +260, about +1 to about +250, about +1 to about +240, about +1 to about +230, about +1 to about +220, about +1 to about +210, about +1 to about +200, about +1 to about +190, about +1 to about +180, about +1 to about +170, about +1 to about +160, about +1 to about +150, about +1 to about +140, about +1 to about +130, about +1 to about +120, about +1 to about +110, about +1 to about +100, about +1 to about +90, about +1 to about +80, about +1 to about +70, about +1 to about +60, about +1 to about +50, about +1 to about +40, about +1 to about +30, or about +1 to about +20 relative to 5′ splice site (or 3′ end) of the included exon. In some aspects, the ASOs are complementary to a targeted portion that is within the region from about +1 to about +100, from about +100 to about +200, from about +200 to about +300, from about +300 to about +400, or from about +400 to about +500 relative to 5′ splice site (or 3′ end) of the included exon.

In some embodiments, the ASOs are complementary to (and bind to) a targeted portion of an OPA1 pre-mRNA, e.g., an OPA1 NMD exon-containing pre-mRNA, that is upstream (in the 5′ direction) of the 5′ splice site (or 3′ end) of the included exon in an OPA1 pre-mRNA, e.g., an OPA1 NMD exon-containing pre-mRNA (e.g., the direction designated by negative numbers relative to the 5′ splice site). In some embodiments, the ASOs are complementary to a targeted portion of the OPA1 pre-mRNA, e.g., the OPA1 NMD exon-containing pre-mRNA, that is within the region about −4 to about −270 relative to the 5′ splice site (or 3′end) of the included exon. In some embodiments, the ASOs may be complementary to a targeted portion of an OPA1 pre-mRNA, e.g., an OPA1 NMD exon-containing pre-mRNA, that is within the region between nucleotides −1 and −40,000 relative to the 5′ splice site (or 3′ end) of the included exon. In some aspects, the ASOs are complementary to a targeted portion that is within the region about −1 to about −40,000, about −1 to about −30,000, about −1 to about −20,000, about −1 to about −15,000, about −1 to about −10,000, about −1 to about −5,000, about −1 to about −4,000, about −1 to about −3,000, about −1 to about −2,000, about −1 to about −1,000, about −1 to about −500, about −1 to about −490, about −1 to about −480, about −1 to about −470, about −1 to about −460, about −1 to about −450, about −1 to about −440, about −1 to about −430, about −1 to about −420, about −1 to about −410, about −1 to about −400, about −1 to about −390, about −1 to about −380, about −1 to about −370, about −1 to about −360, about −1 to about −350, about −1 to about −340, about −1 to about −330, about −1 to about −320, about −1 to about −310, about −1 to about −300, about −1 to about −290, about −1 to about −280, about −1 to about −270, about −1 to about −260, about −1 to about −250, about −1 to about −240, about −1 to about −230, about −1 to about −220, about −1 to about −210, about −1 to about −200, about −1 to about −190, about −1 to about −180, about −1 to about −170, about −1 to about −160, about −1 to about −150, about −1 to about −140, about −1 to about −130, about −1 to about −120, about −1 to about −110, about −1 to about −100, about −1 to about −90, about −1 to about −80, about −1 to about −70, about −1 to about −60, about −1 to about −50, about −1 to about −40, about −1 to about −30, or about −1 to about −20 relative to 5′ splice site (or 3′ end) of the included exon.

In some embodiments, the ASOs are complementary to a targeted region of an OPA1 pre-mRNA, e.g., an OPA1 NMD exon-containing pre-mRNA, that is upstream (in the 5′ direction) of the 3′ splice site (or 5′ end) of the included exon in an OPA1 pre-mRNA (e.g., in the direction designated by negative numbers). In some embodiments, the ASOs are complementary to a targeted portion of the OPA1 pre-mRNA, e.g., the OPA1 NMD exon-containing pre-mRNA, that is within the region about −1 to about −500 relative to the 3′ splice site (or 5′ end) of the included exon. In some embodiments, the ASOs are complementary to a targeted portion of the OPA1 pre-mRNA that is within the region −1 to −40,000 relative to the 3′ splice site of the included exon. In some aspects, the ASOs are complementary to a targeted portion that is within the region about −1 to about −40,000, about −1 to about −30,000, −1 to about −20,000, about −1 to about −15,000, about −1 to about −10,000, about −1 to about −5,000, about −1 to about −4,000, about −1 to about −3,000, about −1 to about −2,000, about −1 to about −1,000, about −1 to about −500, about −1 to about −490, about −1 to about −480, about −1 to about −470, about −1 to about −460, about −1 to about −450, about −1 to about −440, about −1 to about −430, about −1 to about −420, about −1 to about −410, about −1 to about −400, about −1 to about −390, about −1 to about −380, about −1 to about −370, about −1 to about −360, about −1 to about −350, about −1 to about −340, about −1 to about −330, about −1 to about −320, about −1 to about −310, about −1 to about −300, about −1 to about −290, about −1 to about −280, about −1 to about −270, about −1 to about −260, about −1 to about −250, about −1 to about −240, about −1 to about −230, about −1 to about −220, about −1 to about −210, about −1 to about −200, about −1 to about −190, about −1 to about −180, about −1 to about −170, about −1 to about −160, about −1 to about −150, about −1 to about −140, about −1 to about −130, about −1 to about −120, about −1 to about −110, about −1 to about −100, about −1 to about −90, about −1 to about −80, about −1 to about −70, about −1 to about −60, about −1 to about −50, about −1 to about −40, about −1 to about −30, or about −1 to about −20 relative to 3′ splice site of the included exon. In some aspects, the ASOs are complementary to a targeted portion that is within the region from about −1 to about −100, from about −100 to about −200, from about −200 to about −300, from about −300 to about −400, or from about −400 to about −500 relative to 3′ splice site of the included exon.

In some embodiments, the ASOs are complementary to a targeted region of an OPA1 pre-mRNA, e.g., an OPA1 NMD exon-containing pre-mRNA, that is downstream (in the 3′ direction) of the 3′ splice site (5′ end) of the included exon in an OPA1 pre-mRNA, e.g., an OPA1 NMD exon-containing pre-mRNA (e.g., in the direction designated by positive numbers). In some embodiments, the ASOs are complementary to a targeted portion of the OPA1 pre-mRNA that is within the region of about +1 to about +40,000 relative to the 3′ splice site of the included exon. In some aspects, the ASOs are complementary to a targeted portion that is within the region about +1 to about +40,000, about +1 to about +30,000, about +1 to about +20,000, about +1 to about +15,000, about +1 to about +10,000, about +1 to about +5,000, about +1 to about +4,000, about +1 to about +3,000, about +1 to about +2,000, about +1 to about +1,000, about +1 to about +500, about +1 to about +490, about +1 to about +480, about +1 to about +470, about +1 to about +460, about +1 to about +450, about +1 to about +440, about +1 to about +430, about +1 to about +420, about +1 to about +410, about +1 to about +400, about +1 to about +390, about +1 to about +380, about +1 to about +370, about +1 to about +360, about +1 to about +350, about +1 to about +340, about +1 to about +330, about +1 to about +320, about +1 to about +310, about +1 to about +300, about +1 to about +290, about +1 to about +280, about +1 to about +270, about +1 to about +260, about +1 to about +250, about +1 to about +240, about +1 to about +230, about +1 to about +220, about +1 to about +210, about +1 to about +200, about +1 to about +190, about +1 to about +180, about +1 to about +170, about +1 to about +160, about +1 to about +150, about +1 to about +140, about +1 to about +130, about +1 to about +120, about +1 to about +110, about +1 to about +100, about +1 to about +90, about +1 to about +80, about +1 to about +70, about +1 to about +60, about +1 to about +50, about +1 to about +40, about +1 to about +30, or about +1 to about +20, or about +1 to about +10 relative to 3′ splice site of the included exon.

In some embodiments, the targeted portion of the OPA1 pre-mRNA, e.g., the OPA1 NMD exon-containing pre-mRNA, is within the region +100 relative to the 5′ splice site (3′ end) of the included exon to −100 relative to the 3′ splice site (5′ end) of the included exon. In some embodiments, the targeted portion of the OPA1 NMD exon-containing pre-mRNA is within the NMD exon. In some embodiments, the target portion of the OPA1 NMD exon-containing pre-mRNA comprises a pseudo-exon and intron boundary.

The ASOs may be of any length suitable for specific binding and effective enhancement of splicing. In some embodiments, the ASOs consist of 8 to 50 nucleobases. For example, the ASO may be 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, or 50 nucleobases in length. In some embodiments, the ASOs consist of more than 50 nucleobases. In some embodiments, the ASO is from 8 to 50 nucleobases, 8 to 40 nucleobases, 8 to 35 nucleobases, 8 to 30 nucleobases, 8 to 25 nucleobases, 8 to 20 nucleobases, 8 to 15 nucleobases, 9 to 50 nucleobases, 9 to 40 nucleobases, 9 to 35 nucleobases, 9 to 30 nucleobases, 9 to 25 nucleobases, 9 to 20 nucleobases, 9 to 15 nucleobases, 10 to 50 nucleobases, 10 to 40 nucleobases, 10 to 35 nucleobases, 10 to 30 nucleobases, 10 to 25 nucleobases, 10 to 20 nucleobases, 10 to 15 nucleobases, 11 to 50 nucleobases, 11 to 40 nucleobases, 11 to 35 nucleobases, 11 to 30 nucleobases, 11 to 25 nucleobases, 11 to 20 nucleobases, 11 to 15 nucleobases, 12 to 50 nucleobases, 12 to 40 nucleobases, 12 to 35 nucleobases, 12 to 30 nucleobases, 12 to 25 nucleobases, 12 to 20 nucleobases, 12 to 15 nucleobases, 13 to 50 nucleobases, 13 to 40 nucleobases, 13 to 35 nucleobases, 13 to 30 nucleobases, 13 to 25 nucleobases, 13 to 20 nucleobases, 14 to 50 nucleobases, 14 to 40 nucleobases, 14 to 35 nucleobases, 14 to 30 nucleobases, 14 to 25 nucleobases, 14 to 20 nucleobases, 15 to 50 nucleobases, 15 to 40 nucleobases, 15 to 35 nucleobases, 15 to 30 nucleobases, 15 to 25 nucleobases, 15 to 20 nucleobases, 20 to 50 nucleobases, 20 to 40 nucleobases, 20 to 35 nucleobases, 20 to 30 nucleobases, 20 to 25 nucleobases, 25 to 50 nucleobases, 25 to 40 nucleobases, 25 to 35 nucleobases, or 25 to 30 nucleobases in length. In some embodiments, the ASOs are 18 nucleotides in length. In some embodiments, the ASOs are 15 nucleotides in length. In some embodiments, the ASOs are 25 nucleotides in length.

In some embodiments, two or more ASOs with different chemistries but complementary to the same targeted portion of the processed mRNA or the pre-mRNA are used. In some embodiments, two or more ASOs that are complementary to different targeted portions of the processed mRNA or the pre-mRNA are used.

In some embodiments, the antisense oligonucleotides of the disclosure are chemically linked to one or more moieties or conjugates, e.g., a targeting moiety or other conjugate that enhances the activity or cellular uptake of the oligonucleotide. Such moieties include, but are not limited to, a lipid moiety, e.g., as a cholesterol moiety, a cholesteryl moiety, an aliphatic chain, e.g., dodecandiol or undecyl residues, a polyamine or a polyethylene glycol chain, or adamantane acetic acid. Oligonucleotides comprising lipophilic moieties and preparation methods have been described in the published literature. In embodiments, the antisense oligonucleotide is conjugated with a moiety including, but not limited to, an abasic nucleotide, a polyether, a polyamine, a polyamide, a peptides, a carbohydrate, e.g., N-acetylgalactosamine (GalNAc), N-Ac-Glucosamine (GluNAc), or mannose (e.g., mannose-6-phosphate), a lipid, or a polyhydrocarbon compound. Conjugates can be linked to one or more of any nucleotides comprising the antisense oligonucleotide at any of several positions on the sugar, base or phosphate group, as understood in the art and described in the literature, e.g., using a linker. Linkers can include a bivalent or trivalent branched linker. In embodiments, the conjugate is attached to the 3′ end of the antisense oligonucleotide. Methods of preparing oligonucleotide conjugates are described, e.g., in U.S. Pat. No. 8,450,467, “Carbohydrate conjugates as delivery agents for oligonucleotides,” incorporated by reference herein.

In some embodiments, an agent disclosed herein comprises a cell-penetrating peptide conjugated to an antisense oligomer, e.g., an ASO disclosed herein. The terms “cell-penetrating peptide” or “CPP” are used interchangeably and can refer to cationic cell-penetrating peptides, also called transport peptides, carrier peptides, or peptide transduction domains. The peptides can have the capability of inducing cell penetration within at least 70%, 80%, 90%, or 95%, or 100% of cells of a given cell culture population and allow macromolecular translocation within multiple tissues in vivo upon systemic administration. Non-limiting examples of the cell-penetrating peptide that can be used in an agent disclosed herein are listed in Table 9. The synthesis, structures, and delivery characteristics of morpholino oligomers are detailed in U.S. Patent Publication No. US 2010/0016215, and Jearawiriyapaisarn et al. (2008), Mol Ther. 2008 September; 16(9): 1624-1629, both of which are incorporated herein in their entireties.

In some embodiments, the agent disclosed herein comprises a cell-penetrating peptide conjugated to a phosphoramidate morpholino oligomer, wherein the phosphoramidate morpholino oligomer has the sequence of any of the ASOs disclosed herein. The terms “morpholino oligomer” or “PMO” (phosphoramidate- or phosphorodiamidate morpholino oligomer) refer to an oligonucleotide composed of morpholino subunit structures, where (i) the structures are linked together by phosphorus-containing linkages, one to three atoms long, preferably two atoms long, and preferably uncharged or cationic, joining the morpholino nitrogen of one subunit to a 5′ exocyclic carbon of an adjacent subunit, and (ii) each morpholino ring bears a purine or pyrimidine base-pairing moiety effective to bind, by base specific hydrogen bonding, to a base in a polynucleotide. Variations can be made to the phosphorodiamidate linkage as long as they do not interfere with binding or activity. For example, the oxygen attached to phosphorus may be substituted with sulfur (thiophosphorodiamidate). The 5′ oxygen may be substituted with amino or lower alkyl substituted amino. The pendant nitrogen attached to phosphorus may be unsubstituted, monosubstituted, or disubstituted with (optionally substituted) lower alkyl. See also the discussion of cationic linkages below. The purine or pyrimidine base pairing moiety is typically adenine, cytosine, guanine, uracil, thymine or inosine. The synthesis, structures, and binding characteristics of morpholino oligomers are detailed in U.S. Pat. Nos. 5,698,685, 5,217,866, 5,142,047, 5,034,506, 5,166,315, 5,521,063, and 5,506,337, and PCT Publication. No. WO 2008036127, all of which are incorporated herein by reference.

In some embodiments, the nucleic acid to be targeted by an ASO is an OPA1 processed mRNA in a cell. In some embodiments, the nucleic acid to be targeted by an ASO is an OPA1 pre-mRNA, e.g., NMD exon-containing pre-mRNA expressed in a cell, such as a eukaryotic cell. In some embodiments, the term “cell” may refer to a population of cells. In some embodiments, the cell is in a subject. In some embodiments, the cell is isolated from a subject. In some embodiments, the cell is ex vivo. In some embodiments, the cell is a condition or disease-relevant cell or a cell line. In some embodiments, the cell is in vitro (e.g., in cell culture).

### Pharmaceutical Compositions

Pharmaceutical compositions or formulations comprising the agent, e.g., antisense oligonucleotide, of the described compositions and for use in any of the described methods can be prepared according to conventional techniques well known in the pharmaceutical industry and described in the published literature. In embodiments, a pharmaceutical composition or formulation for treating a subject comprises an effective amount of any antisense oligomer as described herein, or a pharmaceutically acceptable salt, solvate, hydrate or ester thereof. The pharmaceutical formulation comprising an antisense oligomer may further comprise a pharmaceutically acceptable excipient, diluent or carrier.

Pharmaceutically acceptable salts are suitable for use in contact with the tissues of humans and lower animals without undue toxicity, irritation, allergic response, etc., and are commensurate with a reasonable benefit/risk ratio. (See, e.g., S. M. Berge, et al., J. Pharmaceutical Sciences, 66: 1-19 (1977), incorporated herein by reference for this purpose. The salts can be prepared in situ during the final isolation and purification of the compounds, or separately by reacting the free base form with a suitable organic acid. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other documented methodologies such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

In some embodiments, the compositions are formulated into any of many possible dosage forms such as, but not limited to, tablets, capsules, gel capsules, liquid syrups, soft gels, suppositories, and enemas. In embodiments, the compositions are formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers. In embodiments, a pharmaceutical formulation or composition of the present disclosure includes, but is not limited to, a solution, emulsion, microemulsion, foam or liposome-containing formulation (e.g., cationic or noncationic liposomes).

The pharmaceutical composition or formulation described herein may comprise one or more penetration enhancers, carriers, excipients or other active or inactive ingredients as appropriate and well known to those of skill in the art or described in the published literature. In embodiments, liposomes also include sterically stabilized liposomes, e.g., liposomes comprising one or more specialized lipids. These specialized lipids result in liposomes with enhanced circulation lifetimes. In embodiments, a sterically stabilized liposome comprises one or more glycolipids or is derivatized with one or more hydrophilic polymers, such as a polyethylene glycol (PEG) moiety. In some embodiments, a surfactant is included in the pharmaceutical formulation or compositions. The use of surfactants in drug products, formulations and emulsions is well known in the art. In embodiments, the present disclosure employs a penetration enhancer to effect the efficient delivery of the antisense oligonucleotide, e.g., to aid diffusion across cell membranes and/or enhance the permeability of a lipophilic drug. In some embodiments, the penetration enhancers are a surfactant, fatty acid, bile salt, chelating agent, or non-chelating nonsurfactant.

In some embodiments, the pharmaceutical formulation comprises multiple antisense oligonucleotides. In embodiments, the antisense oligonucleotide is administered in combination with another drug or therapeutic agent.

### Combination Therapies

In some aspects, provided herein are methods, compositions, and kits relating to a combination therapy. In some embodiments, a combination therapy disclosed herein involves utilizes an agent that modulates a translation regulatory element of a processed mRNA that encoding a target protein, e.g., OPA1 protein, and an agent that modulates splicing of a pre-mRNA that is transcribed from a gene that encodes the target protein, e.g., OPA1 gene. In some aspects, provided herein are compositions, methods, and kits relating to a combination therapy that utilizes an agent that target at least a portion of the 5′ UTR of a processed mRNA that encoding a target protein, e.g., OPA1 protein, and an agent that modulates splicing of a pre-mRNA that is transcribed from a gene that encodes the target protein, e.g., OPA1 gene.

In some cases, provided herein is a method of increasing expression of a target protein in a cell having a processed mRNA that encodes the target protein and comprises a translation regulatory element that inhibits translation of the processed mRNA, the method comprising delivering into the cell: (1) a first agent or a first nucleic acid sequence encoding the first agent, and (2) a second agent or a second nucleic acid sequence encoding the second agent, wherein the first agent modulates splicing of a pre-mRNA that is transcribed from an target gene that encodes the target protein, and wherein the second agent modulates a structure of the translation regulatory element of the processed mRNA that encodes the target protein, thereby increasing the expression of the target protein in the cell. In some embodiments, provided herein is a method of treatment that comprising administering to a subject in need thereof (1) a first agent or a first nucleic acid sequence encoding the first agent, and (2) a second agent or a second nucleic acid sequence encoding the second agent, wherein the first agent modulates splicing of a pre-mRNA that is transcribed from an target gene that encodes the target protein, and wherein the second agent modulates a structure of the translation regulatory element of the processed mRNA that encodes the target protein, thereby increasing the expression of the target protein in the cells of the subject.

In some cases, provided herein is a method of increasing expression of a target protein in a cell having a processed mRNA that encodes the target protein and comprises a translation regulatory element that inhibits translation of the processed mRNA, the method comprising delivering into the cell: (1) a first agent or a first nucleic acid sequence encoding the first agent, and (2) a second agent or a second nucleic acid sequence encoding the second agent, wherein the first agent modulates splicing of a pre-mRNA that is transcribed from an target gene that encodes the target protein, and wherein the second agent (a) binds to a targeted portion of the processed mRNA; (b) modulates interaction of the translation regulatory element with a factor involved in translation of the processed mRNA; or (c) a combination of (a) and (b), thereby increasing the expression of the target protein in the cell. In some embodiments, provided herein is a method of treatment that comprising administering to a subject in need thereof (1) a first agent or a first nucleic acid sequence encoding the first agent, and (2) a second agent or a second nucleic acid sequence encoding the second agent, wherein the first agent modulates splicing of a pre-mRNA that is transcribed from an target gene that encodes the target protein, and wherein the second agent (a) binds to a targeted portion of the processed mRNA; (b) modulates interaction of the translation regulatory element with a factor involved in translation of the processed mRNA; or (c) a combination of (a) and (b), thereby increasing the expression of the target protein in the cells of the subject.

In some embodiments, provided herein is a method of modulating expression of a target protein in a cell, wherein contacting to the cell: (1) a first agent or a first nucleic acid sequence encoding the first agent, and (2) a second agent or a second nucleic acid sequence encoding the second agent, wherein the first agent comprises a first antisense oligomer with at least 80% sequence identity to a sequence selected from the group consisting of SEQ ID NOs: 6-275 and 280-299, and the second agent comprises a second antisense oligomer that binds to 5′ UTR of a processed mRNA that encodes the target protein, and wherein the target protein is an OPA1 protein. In some cases, provided herein is a method of treatment that comprising administering to a subject in need thereof (1) a first agent or a first nucleic acid sequence encoding the first agent, and (2) a second agent or a second nucleic acid sequence encoding the second agent, wherein the first agent comprises a first antisense oligomer with at least 80% sequence identity to a sequence selected from the group consisting of SEQ ID NOs: 6-275 and 280-299, and the second agent comprises a second antisense oligomer that binds to 5′ UTR of a processed mRNA that encodes the target protein, and wherein the target protein is an OPA1 protein.

In some aspects, provided herein is a pharmaceutical composition, comprising (1) a first therapeutic agent or a first nucleic acid sequence encoding the first therapeutic agent, and (2) a second therapeutic agent or a second nucleic acid sequence encoding the second therapeutic agent, wherein the first therapeutic agent comprises a first antisense oligomer with at least 80% sequence identity to a sequence selected from the group consisting of SEQ ID NOs: 6-275 and 280-299, and the second therapeutic agent comprises a second antisense oligomer that binds to 5′ UTR of a processed mRNA that encodes an OPA1 protein.

In some aspects, provided herein is a pharmaceutical composition, comprising: (1) a first therapeutic agent or a first nucleic acid sequence encoding the first therapeutic agent, (2) a second therapeutic agent or a second nucleic acid sequence encoding the second therapeutic agent, and (3) a pharmaceutically acceptable carrier or excipient, wherein the first therapeutic agent modulates splicing of a pre-mRNA that is transcribed from a target gene that encodes the target protein, and wherein the second therapeutic agent (a) binds to a targeted portion of a processed mRNA that encodes an OPA1 protein and that comprises a translation regulatory element; (b) modulates interaction of the translation regulatory element with a factor involved in translation of the processed mRNA; or (c) a combination of (a) and (b), and wherein the translation regulatory element inhibits translation of the processed mRNA.

In some aspects, provided herein is a pharmaceutical composition, comprising: (1) a first therapeutic agent or a first nucleic acid sequence encoding the first therapeutic agent, (2) a second therapeutic agent or a second nucleic acid sequence encoding the second therapeutic agent, and (3) a pharmaceutically acceptable carrier or excipient, wherein the first therapeutic agent modulates splicing of a pre-mRNA that is transcribed from a target gene that encodes the target protein, and wherein the second therapeutic agent modulates a structure of a translation regulatory element of a processed mRNA that encodes the target protein, wherein the translation regulatory element inhibits translation of the processed mRNA.

In some of these cases, the first antisense oligomer has at least 80%, 90%, or 100% sequence identity to a sequence selected from the group consisting of SEQ ID NOs: 36, 236, 242, 250, 280-283, 288, and 290-292. In some of these cases, the first antisense oligomer has at least 80%, 90%, or 100% sequence identity to a sequence selected from the group consisting of SEQ ID NOs: 227-242, 250, 280-283, 288, and 290-292. In some of these the first antisense oligomer has at least 80%, 90%, or 100% sequence identity to SEQ ID NO: 267. In some of these the first antisense oligomer has at least 80%, 90%, or 100% sequence identity to a sequence selected from the group consisting of SEQ ID NOs: 236, 242, 250, 280-283, 288, and 290-292. In some of these the second antisense oligomer has at least 80% sequence identity to a sequence selected from the group consisting of SEQ ID NOs: 608-1253. In some of these the second antisense oligomer has at least 80%, 90%, or 100% sequence identity to a sequence selected from the group consisting of SEQ ID NOs: 827-847, 932-937, 953, 968, 988-1023.

In some embodiments, the agent disclosed in the present disclosure can be used in combination with one or more additional therapeutic agents. In some embodiments, the one or more additional therapeutic agents can comprise a small molecule. For example, the one or more additional therapeutic agents can comprise a small molecule described in WO2016128343A1, WO2017053982A1, WO2016196386A1, WO201428459A1, WO201524876A2, WO2013119916A2, and WO2014209841A2, which are incorporated by reference herein in their entirety. In some embodiments, the one or more additional therapeutic agents comprise an ASO that can be used to correct intron retention.

### Treatment of Subjects

Any of the compositions provided herein may be administered to an individual. “Individual” may be used interchangeably with “subject” or “patient.” An individual may be a mammal, for example a human or animal such as a non-human primate, a rodent, a rabbit, a rat, a mouse, a horse, a donkey, a goat, a cat, a dog, a cow, a pig, or a sheep. In embodiments, the individual is a human. In embodiments, the individual is a fetus, an embryo, or a child. In other embodiments, the individual may be another eukaryotic organism, such as a plant. In some embodiments, the compositions provided herein are administered to a cell ex vivo.

In some embodiments, the compositions provided herein are administered to an individual as a method of treating a disease or disorder. In some embodiments, the individual has a genetic disease, such as any of the diseases described herein. In some embodiments, the individual is at risk of having a disease, such as any of the diseases described herein. In some embodiments, the individual is at increased risk of having a disease or disorder caused by insufficient amount of a protein or insufficient activity of a protein. If an individual is “at an increased risk” of having a disease or disorder caused insufficient amount of a protein or insufficient activity of a protein, the method involves preventative or prophylactic treatment. For example, an individual may be at an increased risk of having such a disease or disorder because of family history of the disease. Typically, individuals at an increased risk of having such a disease or disorder benefit from prophylactic treatment (e.g., by preventing or delaying the onset or progression of the disease or disorder). In embodiments, a fetus is treated in utero, e.g., by administering the ASO composition to the fetus directly or indirectly (e.g., via the mother).

In some cases, the subject pharmaceutical composition and method are applicable for treatment of a condition or disease associated with OPA1 deficiency. In some cases, the subject pharmaceutical composition and method are applicable for treatment of an eye disease or condition. In some cases, the subject pharmaceutical composition and method are applicable for treatment of Optic atrophy type 1, autosomal dominant optic atrophy (ADOA), ADOA-plus syndrome; a mitochondrial disorder; glaucoma; normal tension glaucoma; Charcot-Marie-Tooth disease; mitochondria dysfunction; diabetic retinopathy; age-related macular degeneration; retinal ganglion cell death; mitochondrial fission-mediated mitochondrial dysfunction; progressive external ophthalmoplegia; deafness; ataxia; motor neuropathy; sensory neuropathy; myopathy; Behr syndrome; brain dysfunction; encephalopathy; peripheral neuropathy; fatal infantile mitochondrial encephalomyopathy; hypertrophic cardiomyopathy; spastic ataxic syndrome; sensory motor peripheral neuropathy; hypotonia; gastrointestinal dysmotility and dysphagia; optic atrophy; optic atrophy plus syndrome; Mitochondrial DNA depletion syndrome 14; late-onset cardiomyopathy; diabetic cardiomyopathy; Alzheimer's Disease; focal segmental glomerulosclerosis; kidney disease; Huntington's Disease; cognitive function decline in healthy aging; Prion diseases; late onset dementia and parkinsonism; mitochondrial myopathy; Leigh syndrome; Friedreich's ataxia; Parkinson's disease; MELAS (Mitochondrial encephalomyopathy, lactic acidosis, and stroke-like episodes); pyruvate dehydrogenase complex deficiency; chronic kidney disease; Leber's hereditary optic neuropathy; obesity; age-related systemic neurodegeneration; skeletal muscle atrophy; heart and brain ischemic damage; massive liver apoptosis; NARP (neuropathy, ataxia, retinitis pigmentosa); MERRF (myoclonic epilepsy and ragged red fibers); Pearsons/Kems-Sayre syndrome; MIDD (maternally inherited diabetes and deafness); mitochondrial trifunctional protein deficiency; Fuchs corneal endothelial dystrophy; macular telangiectasia; retinitis pigmentosa; Leber congenital amaurosis; inherited maculopathy; Stargardt disease; or Sorsby fundus dystrophy.

In some embodiments, the compositions and methods provided herein are applicable for treatment of mitochondrial disorders, for instance, alleviation of one or more optic symptoms of a primary mitochondrial disorder. In some cases, the compositions and methods provided herein are applicable for treatment of optic neuropathies (e.g., DOA or dominant optic atrophy, LHON or Leber hereditary optic neuropathy), CPEO (chronic progressive external ophthalmoplegia), or pigmentary retinopathy (e.g., NARP (neuropathy, ataxia, retinitis pigmentosa), MELAS (mitochondrial encephalopathy, lactic acidosis and stroke like episodes), MERRF (myoclonic epilepsy and ragged red fibers), Leigh syndrome, Pearsons/Kems-Sayre syndrome, MIDD (maternally inherited diabetes and deafness), or mitochondrial trifunctional protein deficiency).

In some embodiments, the compositions and methods provided herein are applicable for treatment of age-related ophthalmic diseases with associated mitochondrial dysfunction, such as Glaucoma, age-related macular degeneration, diabetic retinopathy, Fuchs corneal endothelial dystrophy, or Macular telangiectasia.

In some embodiments, the compositions and methods provided herein are applicable for treatment of hereditary ophthalmic diseases with associated mitochondrial dysfunction, such as, Retinitis pigmentosa (e.g., CERKL retinitis pigmentosa), Leber congenital amaurosis, or Inherited maculopathies (e.g., Stargardt's disease or Sorsby's fundus dystrophy).

Autosomal dominant optic atrophy (ADOA) is the most common inherited optic nerve disorder and is characterized by retinal ganglion cell loss. In some cases, 65-90% of ADOA cases are caused by mutations in one allele of the OPA1 gene. OPA1 gene encodes an OPA1 protein that is a mitochondrial GTPase, which can have a critical maintenance role in mitochondria structure and function. Most OPA1 mutations can lead to a haploinsufficiency, resulting in about a 50% decrease of normal OPA1 protein levels. Approximately 1 out of 30,000 people are affected globally with a higher incidence of ˜1 out of 10,000 in Denmark due to a founder effect. ADOA can present within the first decade of life. 80% of ADOA patients are symptomatic before 10 years of age. The disease can cause progressive and irreversible vision loss and up to 46% of patients are registered as legally blind.

In some cases, a therapeutic agent comprises an oligonucleotide. In some cases, a therapeutic agent comprises a vector, e.g., a viral vector, expressing an oligonucleotide that binds to the targeted region of a pre-mRNA the encodes the target peptide sequence. The methods provided herein can be adapted to contacting a vector that encodes an agent, e.g., an oligonucleotide, to a cell, so that the agent binds to a pre-mRNA in the cell and modulates the processing of the pre-mRNA. In some cases, the viral vector comprises an adenoviral vector, adeno-associated viral (AAV) vector, lentiviral vector, Herpes Simplex Virus (HSV) viral vector, retroviral vector, or any applicable viral vector. In some cases, a therapeutic agent comprises a gene editing tool that is configured to modify a gene encoding the target peptide sequence such that a gene region that encodes the inefficient translation region is deleted. In some cases, a gene editing tool comprises vector, e.g., viral vector, for gene editing based on CRISPR-Cas9, TALEN, Zinc Finger, or other applicable technologies.

Suitable routes for administration of ASOs of the present disclosure may vary depending on cell type to which delivery of the ASOs is desired. Multiple tissues and organs are affected by ADOA, with the eye being the most significantly affected tissue. The ASOs of the present disclosure may be administered to patients parenterally, for example, by intravitreal injection, intrathecal injection, intracerebroventricular injection, intraperitoneal injection, intramuscular injection, subcutaneous injection, or intravenous injection.

In embodiments, the antisense oligonucleotide is administered with one or more agents capable of promoting penetration of the subject antisense oligonucleotide across the blood-brain barrier by any method known in the art. For example, delivery of agents by administration of an adenovirus vector to motor neurons in muscle tissue is described in U.S. Pat. No. 6,632,427, “Adenoviral-vector-mediated gene transfer into medullary motor neurons,” incorporated herein by reference. Delivery of vectors directly to the brain, e.g., the striatum, the thalamus, the hippocampus, or the substantia nigra, is described, e.g., in U.S. Pat. No. 6,756,523, “Adenovirus vectors for the transfer of foreign genes into cells of the central nervous system particularly in brain,” incorporated herein by reference.

In some embodiments, the antisense oligonucleotides are linked or conjugated with agents that provide desirable pharmaceutical or pharmacodynamic properties. In embodiments, the antisense oligonucleotide is coupled to a substance, known in the art to promote penetration or transport across the blood-brain barrier, e.g., an antibody to the transferrin receptor. In embodiments, the antisense oligonucleotide is linked with a viral vector, e.g., to render the antisense compound more effective or increase transport across the blood-brain barrier. In embodiments, osmotic blood brain barrier disruption is assisted by infusion of sugars, e.g., meso erythritol, xylitol, D(+) galactose, D(+) lactose, D(+) xylose, dulcitol, myo-inositol, L(−) fructose, D(−) mannitol, D(+) glucose, D(+) arabinose, D(−) arabinose, cellobiose, D(+) maltose, D(+) raffinose, L(+) rhamnose, D(+) melibiose, D(−) ribose, adonitol, D(+) arabitol, L(−) arabitol, D(+) fucose, L(−) fucose, D(−) lyxose, L(+) lyxose, and L(−) lyxose, or amino acids, e.g., glutamine, lysine, arginine, asparagine, aspartic acid, cysteine, glutamic acid, glycine, histidine, leucine, methionine, phenylalanine, proline, serine, threonine, tyrosine, valine, and taurine. Methods and materials for enhancing blood brain barrier penetration are described, e.g., in U.S. Pat. No. 9,193,969, “Compositions and methods for selective delivery of oligonucleotide molecules to specific neuron types,” U.S. Pat. No. 4,866,042, “Method for the delivery of genetic material across the blood brain barrier,” U.S. Pat. No. 6,294,520, “Material for passage through the blood-brain barrier,” and U.S. Pat. No. 6,936,589, “Parenteral delivery systems,” each incorporated herein by reference.

In some embodiments, subjects treated using the methods and compositions are evaluated for improvement in condition using any methods known and described in the art.

Methods of Identifying Additional ASOs that Induce Exon Skipping

Also within the scope of the present disclosure are methods for identifying or determining ASOs that induce exon skipping of an OPA1 NMD exon-containing pre-mRNA. For example, a method can comprise identifying or determining ASOs that induce pseudo-exon skipping of an OPA1 NMD exon-containing pre-mRNA. ASOs that specifically hybridize to different nucleotides within the target region of the pre-mRNA may be screened to identify or determine ASOs that improve the rate and/or extent of splicing of the target intron. In some embodiments, the ASO may block or interfere with the binding site(s) of a splicing repressor(s)/silencer. Any method known in the art may be used to identify (determine) an ASO that when hybridized to the target region of the exon results in the desired effect (e.g., pseudo-exon skipping, protein or functional RNA production). These methods also can be used for identifying ASOs that induce exon skipping of the included exon by binding to a targeted region in an intron flanking the included exon, or in a non-included exon. An example of a method that may be used is provided below.

A round of screening, referred to as an ASO “walk” may be performed using ASOs that have been designed to hybridize to a target region of a pre-mRNA. For example, the ASOs used in the ASO walk can be tiled every 5 nucleotides from approximately 100 nucleotides upstream of the 3′ splice site of the included exon (e.g., a portion of sequence of the exon located upstream of the target/included exon) to approximately 100 nucleotides downstream of the 3′ splice site of the target/included exon and/or from approximately 100 nucleotides upstream of the 5′ splice site of the included exon to approximately 100 nucleotides downstream of the 5′ splice site of the target/included exon (e.g., a portion of sequence of the exon located downstream of the target/included exon). For example, a first ASO of 15 nucleotides in length may be designed to specifically hybridize to nucleotides +6 to +20 relative to the 3′ splice site of the target/included exon. A second ASO may be designed to specifically hybridize to nucleotides +11 to +25 relative to the 3′ splice site of the target/included exon. ASOs are designed as such spanning the target region of the pre-mRNA. In embodiments, the ASOs can be tiled more closely, e.g., every 1, 2, 3, or 4 nucleotides. Further, the ASOs can be tiled from 100 nucleotides downstream of the 5′ splice site, to 100 nucleotides upstream of the 3′ splice site. In some embodiments, the ASOs can be tiled from about 1,160 nucleotides upstream of the 3′ splice site, to about 500 nucleotides downstream of the 5′ splice site. In some embodiments, the ASOs can be tiled from about 500 nucleotides upstream of the 3′ splice site, to about 1,920 nucleotides downstream of the 3′ splice site.

One or more ASOs, or a control ASO (an ASO with a scrambled sequence, sequence that is not expected to hybridize to the target region) are delivered, for example by transfection, into a disease-relevant cell line that expresses the target pre-mRNA (e.g., a NMD exon-containing pre-mRNA described herein). The exon skipping effects of each of the ASOs may be assessed by any method known in the art, for example by reverse transcriptase (RT)-PCR using primers that span the splice junction, as described in Example 2.4. A reduction or absence of a longer RT-PCR product produced using the primers spanning the region containing the included exon (e.g., including the flanking exons of the NMD exon) in ASO-treated cells as compared to in control ASO-treated cells indicates that splicing of the target NMD exon has been enhanced. In some embodiments, the exon skipping efficiency (or the splicing efficiency to splice the intron containing the NMD exon), the ratio of spliced to unspliced pre-mRNA, the rate of splicing, or the extent of splicing may be improved using the ASOs described herein. The amount of protein or functional RNA that is encoded by the target pre-mRNA can also be assessed to determine whether each ASO achieved the desired effect (e.g., enhanced functional protein production). Any method known in the art for assessing and/or quantifying protein production, such as Western blotting, flow cytometry, immunofluorescence microscopy, and ELISA, can be used.

A second round of screening, referred to as an ASO “micro-walk” may be performed using ASOs that have been designed to hybridize to a target region of a pre-mRNA. The ASOs used in the ASO micro-walk are tiled every 1 nucleotide to further refine the nucleotide acid sequence of the pre-mRNA that when hybridized with an ASO results in exon skipping (or enhanced splicing of NMD exon).

Regions defined by ASOs that promote splicing of the target intron are explored in greater detail by means of an ASO “micro-walk”, involving ASOs spaced in 1-nt steps, as well as longer ASOs, typically 18-25 nt.

As described for the ASO walk above, the ASO micro-walk is performed by delivering one or more ASOs, or a control ASO (an ASO with a scrambled sequence, sequence that is not expected to hybridize to the target region), for example by transfection, into a disease-relevant cell line that expresses the target pre-mRNA. The splicing-inducing effects of each of the ASOs may be assessed by any method known in the art, for example by reverse transcriptase (RT)-PCR using primers that span the NMD exon, as described herein (see, e.g., Example 2.4). A reduction or absence of a longer RT-PCR product produced using the primers spanning the NMD exon in ASO-treated cells as compared to in control ASO-treated cells indicates that exon skipping (or splicing of the target intron containing an NMD exon) has been enhanced. In some embodiments, the exon skipping efficiency (or the splicing efficiency to splice the intron containing the NMD exon), the ratio of spliced to unspliced pre-mRNA, the rate of splicing, or the extent of splicing may be improved using the ASOs described herein. The amount of protein or functional RNA that is encoded by the target pre-mRNA can also be assessed to determine whether each ASO achieved the desired effect (e.g., enhanced functional protein production). Any method known in the art for assessing and/or quantifying protein production, such as Western blotting, flow cytometry, immunofluorescence microscopy, and ELISA, can be used.

ASOs that when hybridized to a region of a pre-mRNA result in exon skipping (or enhanced splicing of the intron containing a NMD exon) and increased protein production may be tested in vivo using animal models, for example transgenic mouse models in which the full-length human gene has been knocked-in or in humanized mouse models of disease. Suitable routes for administration of ASOs may vary depending on the disease and/or the cell types to which delivery of the ASOs is desired. ASOs may be administered, for example, by intravitreal injection, intrathecal injection, intracerebroventricular injection, intraperitoneal injection, intramuscular injection, subcutaneous injection, or intravenous injection. Following administration, the cells, tissues, and/or organs of the model animals may be assessed to determine the effect of the ASO treatment by for example evaluating splicing (e.g., efficiency, rate, extent) and protein production by methods known in the art and described herein. The animal models may also be any phenotypic or behavioral indication of the disease or disease severity.

Also within the scope of the present disclosure is a method to identify or validate an NMD-inducing exon in the presence of an NMD inhibitor, for example, cycloheximide. An exemplary method is provided in Example 2.2.

Methods of Identifying Additional ASOs that Modulate mRNA Translation.

Also within the scope of the present disclosure are methods for identifying or determining ASOs that modulates translation of OPA1 processed mRNA transcripts. For example, a method can comprise identifying or determining ASOs that modulate translation of OPA1 processed mRNA transcripts. ASOs that specifically hybridize to different nucleotides within the targeted portion of the processed mRNA may be screened to identify or determine ASOs that improve the rate and/or efficiency of translation of the processed mRNA. In some embodiments, the ASO may interfere interaction of one or more translation factors with the processed mRNA. Any method known in the art may be used to identify (determine) an ASO that when hybridized to the targeted portion of the processed mRNA results in the desired effect (e.g., increase in rate and/or efficiency of translation of the processed mRNA). An example of a method that may be used is provided below.

A round of screening, referred to as an ASO “walk” may be performed using ASOs that have been designed to hybridize to a targeted portion of a mRNA transcript, e.g., a processed mRNA transcript. For example, the ASOs used in the ASO walk can be tiled every 5 nucleotides from approximately 100, 200, 300, 400, or 500 nucleotides upstream of a region of interest (e.g., 5′-UTR of the processed mRNA) to approximately 100 nucleotides downstream of the region of interest. For example, a first ASO of 15 nucleotides in length may be designed to specifically hybridize to the first 15 nucleotides at the 5′ end of the processed mRNA, e.g., +1 to +15 relative to the 5′ end of Exon 1. A second ASO may be designed to specifically hybridize to nucleotides +6 to +20 relative to the 5′ end of Exon 1. ASOs are designed as such spanning the targeted portion of the mRNA transcript. In embodiments, the ASOs can be tiled more closely, e.g., every 1, 2, 3, or 4 nucleotides.

One or more ASOs, or a control ASO (an ASO with a scrambled sequence, sequence that is not expected to hybridize to the target region) are delivered, for example by transfection, into a disease-relevant cell line that expresses the target a processed mRNA (e.g., OPA processed mRNA or a processed mRNA comprising 5′-UTR of OPA1 mRNA and a sequence coding for a reporter protein). The translation modulation effects of each of the ASOs may be assessed by any method known in the art, for example by assessing the expression level of the protein encoded by the processed mRNA. In some cases, if the targeted portion involves 5′-UTR of a target processed mRNA, the translation modulation effects of the ASOs may be assessed by an assay, in which the 5′-UTR of the target processed mRNA is linked with a coding sequence for a reporter protein (such as luciferase), and the expression of the reporter protein in cells treated with the ASO or control ASO is examined, as described in Examples 1.3. An increase in the reporter protein level (e.g., luciferase expression level in the luciferase assay as illustrated in Examples 1.3) in the in ASO-treated cells as compared to in control ASO-treated cells indicates that translation of the processed mRNA has been enhanced. In some embodiments, level of the processed mRNA in the cells is monitored and the protein level is normalized by the level of the processed mRNA, so that any effect of the ASO treatment on the level of the processed mRNA may be excluded for the assessment of the effect of the ASO on the translation modulation of the processed mRNA. In some cases, in a reporter protein assay as described above, it is the level of the reporter protein in the cells being examined. Any method known in the art for assessing and/or quantifying protein production, such as Western blotting, flow cytometry, immunofluorescence microscopy, or ELISA, can be used.

A second round of screening, referred to as an ASO “micro-walk” may be performed using ASOs that have been designed to hybridize to a target region of a processed mRNA (e.g., OPA1 processed mRNA or a processed mRNA comprising 5′-UTR of OPA1 mRNA and a sequence coding for a reporter protein). The ASOs used in the ASO micro-walk are tiled every 1 nucleotide to further refine the nucleotide acid sequence of the processed mRNA that when hybridized with an ASO results in increase in rate and/or efficacy of translation of the processed mRNA.

Regions defined by ASOs that promote mRNA translation are explored in greater detail by means of an ASO “micro-walk”, involving ASOs spaced in 1-nt steps, as well as longer ASOs, typically 18-25 nt.

As described for the ASO walk above, the ASO micro-walk is performed by delivering one or more ASOs, or a control ASO (an ASO with a scrambled sequence, sequence that is not expected to hybridize to the target region), for example by transfection, into a disease-relevant cell line that expresses the target processed mRNA (e.g., OPA1 processed mRNA or a processed mRNA comprising 5′-UTR of OPA1 mRNA and a sequence coding for a reporter protein). The translation modulation effects of each of the ASOs may be assessed by any method known in the art, for example by Western blotting, Jess blotting, or bioluminescent quantification if luciferase is used as a reporter protein as described herein (see, e.g., Example 1.1).

ASOs that when hybridized to a region of a processed mRNA result in increased mRNA translation and increased protein production may be tested in vivo using animal models, for example transgenic mouse models in which the full-length human gene has been knocked-in or in humanized mouse models of disease. Suitable routes for administration of ASOs may vary depending on the disease and/or the cell types to which delivery of the ASOs is desired. ASOs may be administered, for example, by intravitreal injection, intrathecal injection, intracerebroventricular injection, intraperitoneal injection, intramuscular injection, subcutaneous injection, or intravenous injection. Following administration, the cells, tissues, and/or organs of the model animals may be assessed to determine the effect of the ASO treatment by for example evaluating splicing (e.g., efficiency, rate, extent) and protein production by methods known in the art and described herein. The animal models may also be any phenotypic or behavioral indication of the disease or disease severity.

### SPECIFIC EMBODIMENTS (A)

Embodiment A1. A method of treating Optic atrophy type 1 in a subject in need thereof, by increasing the expression of a target protein or functional RNA by a cell of the subject, wherein the cell has an mRNA that contains a nonsense-mediated RNA decay-inducing exon (NMD exon mRNA), and wherein the NMD exon mRNA encodes the target protein or functional RNA, the method comprising contacting the cell of the subject with a therapeutic agent that binds to a targeted portion of the NMD exon mRNA encoding the target protein or functional RNA, whereby the nonsense-mediated RNA decay-inducing exon is excluded from the NMD exon mRNA encoding the target protein or functional RNA, thereby increasing the level of mRNA encoding the target protein or functional RNA, and increasing the expression of the target protein or functional RNA in the cell of the subject.

Embodiment A2. The method of embodiment A1, wherein the target protein is OPA1.

Embodiment A3. A method of increasing expression of OPA1 protein by a cell having an mRNA that contains a nonsense-mediated RNA decay-inducing exon (NMD exon mRNA) and encodes OPA1 protein, the method comprising contacting the cell with an agent that binds to a targeted portion of the NMD exon mRNA encoding OPA1 protein, whereby the nonsense-mediated RNA decay-inducing exon is excluded from the NMD exon mRNA encoding OPA1 protein, thereby increasing the level of mRNA encoding OPA1 protein, and increasing the expression of OPA1 protein in the cell.

Embodiment A4. The method of any one of embodiments A1 to A3, wherein the nonsense-mediated RNA decay-inducing exon is spliced out from the NMD exon mRNA encoding the target protein or functional RNA.

Embodiment A5. The method of any one of embodiments A1 to A4, wherein the target protein does not comprise an amino acid sequence encoded by the nonsense-mediated RNA decay-inducing exon.

Embodiment A6. The method of any one of embodiments A1 to A5, wherein the target protein is a full-length target protein.

Embodiment A7. The method of any one of embodiments A1 to A6, wherein the agent is an antisense oligomer (ASO) complementary to the targeted portion of the NMD exon mRNA.

Embodiment A8. The method of any one of embodiments A1 to A7, wherein the mRNA is pre-mRNA.

Embodiment A9. The method of any one of embodiments A1 to A8, wherein the contacting comprises contacting the therapeutic agent to the mRNA, wherein the mRNA is in a nucleus of the cell.

Embodiment A10. The method of any one of embodiments A1 to A9, wherein the target protein or the functional RNA corrects a deficiency in the target protein or functional RNA in the subject.

Embodiment A11. The method of any one of embodiments A1 to A10, wherein the cells are in or from a subject with a condition caused by a deficient amount or activity of an OPA1 protein.

Embodiment A12. The method of any one of embodiments A1 to A11, wherein the deficient amount of the target protein is caused by haploinsufficiency of the target protein, wherein the subject has a first allele encoding a functional target protein, and a second allele from which the target protein is not produced, or a second allele encoding a nonfunctional target protein, and wherein the antisense oligomer binds to a targeted portion of a NMD exon mRNA transcribed from the first allele.

Embodiment A13. The method of any one of embodiments A1 to A11, wherein the subject has a condition caused by a disorder resulting from a deficiency in the amount or function of the target protein, wherein the subject has
(a) a first mutant allele from which
   (i) the target protein is produced at a reduced level compared to production from a wild-type allele,
   (ii) the target protein is produced in a form having reduced function compared to an equivalent wild-type protein, or
   (iii) the target protein is not produced, and
(b) a second mutant allele from which
   (i) the target protein is produced at a reduced level compared to production from a wild-type allele,
   (ii) the target protein is produced in a form having reduced function compared to an equivalent wild-type protein, or
   (iii) the target protein is not produced, and
wherein when the subject has a first mutant allele (a)(iii)., the second mutant allele is (b)(i) or (b)(ii) and wherein when the subject has a second mutant allele (b)(iii), the first mutant allele is (a)(i) or (a)(ii), and wherein the NMD exon mRNA is transcribed from either the first mutant allele that is (a)(i) or (a)(ii), and/or the second allele that is (b)(i) or (b)(ii).

Embodiment A14. The method of embodiment A13, wherein the target protein is produced in a form having reduced function compared to the equivalent wild-type protein.

Embodiment A15. The method of embodiment A13, wherein the target protein is produced in a form that is fully-functional compared to the equivalent wild-type protein.

Embodiment A16. The method of any one of embodiments A1 to A15, wherein the targeted portion of the NMD exon mRNA is within the nonsense-mediated RNA decay-inducing exon.

Embodiment A17. The method of any one of embodiments A1 to A15, wherein the targeted portion of the NMD exon mRNA is either upstream or downstream of the nonsense-mediated RNA decay-inducing exon.

Embodiment A18. The method of any one of embodiments A1 to A17, wherein the NMD exon mRNA comprises a sequence with at least about 80%, 85%, 90%, 95%, 97%, or 100% sequence identity to SEQ ID NO: 2 or 3.

Embodiment A19. The method of any one of embodiments A1 to A17, wherein the NMD exon mRNA is encoded by a genetic sequence with at least about 80%, 85%, 90%, 95%, 97%, or 100% sequence identity to SEQ ID NO: 1.

Embodiment A20. The method of any one of embodiments A1 to A17, wherein the targeted portion of the NMD exon mRNA comprises a sequence with at least 80%, 85%, 90%, 95%, 97%, or 100% sequence identity to a region comprising at least 8 contiguous nucleic acids of SEQ ID NO: 2 or 3.

Embodiment A21. The method of any one of embodiments A1 to A20, wherein the agent is an antisense oligomer (ASO) and wherein the ASO comprises a sequence that is at least about 80%, 85%, 90%, 95%, 97%, or 100% complementary to at least 8 contiguous nucleic acids of SEQ ID NO: 2 or 3.

Embodiment A22. The method of any one of embodiments A1 to A15, wherein the targeted portion of the NMD exon mRNA is within the nonsense-mediated RNA decay-inducing exon 6x of OPA1, exon 7x of OPA1, or exon 28x of OPAL.

Embodiment A23. The method of any one of embodiments A1 to A15, wherein the targeted portion of the NMD exon mRNA is upstream or downstream of the nonsense-mediated RNA decay-inducing exon 6x of OPA1, exon 7x of OPA1, or exon 28x of OPAL.

Embodiment A24. The method of any one of embodiments A1 to A15, wherein the targeted portion of the NMD exon mRNA comprises an exon-intron junction exon 6x of OPA1, exon 7x of OPA1, or exon 28x of OPAL.

Embodiment A25. The method of any one of embodiments A1 to A24, wherein the target protein produced is full-length protein, or wild-type protein.

Embodiment A26. The method of any one of embodiments A1 to A25, wherein the total amount of the mRNA encoding the target protein or functional RNA produced in the cell contacted with the antisense oligomer is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the total amount of the mRNA encoding the target protein or functional RNA produced in a control cell.

Embodiment A27. The method of any one of embodiments A1 to A25, wherein the total amount of the mRNA encoding the target protein or functional RNA produced in the cell contacted with the antisense oligomer is increased about 20% to about 300%, about 50% to about 300%, about 100% to about 300%, about 150% to about 300%, about 20% to about 50%, about 20% to about 100%, about 20% to about 150%, about 20% to about 200%, about 20% to about 250%, about 50% to about 100%, about 50% to about 150%, about 50% to about 200%, about 50% to about 250%, about 100% to about 150%, about 100% to about 200%, about 100% to about 250%, about 150% to about 200%, about 150% to about 250%, about 200% to about 250%, at least about 10%, at least about 20%, at least about 50%, at least about 100%, at least about 150%, at least about 200%, at least about 250%, or at least about 300%, compared to the total amount of the mRNA encoding the target protein or functional RNA produced in a control cell.

Embodiment A28. The method of one any of embodiments A1 to A25, wherein the total amount of target protein produced by the cell contacted with the antisense oligomer is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the total amount of target protein produced by a control cell.

Embodiment A29. The method of one any of embodiments A1 to A25, wherein the total amount of target protein produced by the cell contacted with the antisense oligomer is increased about 20% to about 300%, about 50% to about 300%, about 100% to about 300%, about 150% to about 300%, about 20% to about 50%, about 20% to about 100%, about 20% to about 150%, about 20% to about 200%, about 20% to about 250%, about 50% to about 100%, about 50% to about 150%, about 50% to about 200%, about 50% to about 250%, about 100% to about 150%, about 100% to about 200%, about 100% to about 250%, about 150% to about 200%, about 150% to about 250%, about 200% to about 250%, at least about 10%, at least about 20%, at least about 50%, at least about 100%, at least about 150%, at least about 200%, at least about 250%, or at least about 300%, compared to the total amount of target protein produced by a control cell.

Embodiment A30. The method of any one of embodiments A1 to 29, wherein the agent is an antisense oligomer (ASO) and wherein the antisense oligomer comprises a backbone modification comprising a phosphorothioate linkage or a phosphorodiamidate linkage.

Embodiment A31. The method of any one of embodiments A1 to A30, wherein the agent is an antisense oligomer (ASO) and wherein the antisense oligomer comprises a phosphorodiamidate morpholino, a locked nucleic acid, a peptide nucleic acid, a 2′-O-methyl, a 2′-Fluoro, or a 2′-O-methoxyethyl moiety.

Embodiment A32. The method of any one of embodiments A1 to A31, wherein the agent is an antisense oligomer (ASO) and wherein the antisense oligomer comprises at least one modified sugar moiety.

Embodiment A33. The method of embodiment A32, wherein each sugar moiety is a modified sugar moiety.

Embodiment A34. The method of any one of embodiments A1 to A33, wherein the agent is an antisense oligomer (ASO) and wherein the antisense oligomer consists of from 8 to 50 nucleobases, 8 to 40 nucleobases, 8 to 35 nucleobases, 8 to 30 nucleobases, 8 to 25 nucleobases, 8 to 20 nucleobases, 8 to 15 nucleobases, 9 to 50 nucleobases, 9 to 40 nucleobases, 9 to 35 nucleobases, 9 to 30 nucleobases, 9 to 25 nucleobases, 9 to 20 nucleobases, 9 to 15 nucleobases, 10 to 50 nucleobases, 10 to 40 nucleobases, 10 to 35 nucleobases, 10 to 30 nucleobases, 10 to 25 nucleobases, 10 to 20 nucleobases, 10 to 15 nucleobases, 11 to 50 nucleobases, 11 to 40 nucleobases, 11 to 35 nucleobases, 11 to 30 nucleobases, 11 to 25 nucleobases, 11 to 20 nucleobases, 11 to 15 nucleobases, 12 to 50 nucleobases, 12 to 40 nucleobases, 12 to 35 nucleobases, 12 to 30 nucleobases, 12 to 25 nucleobases, 12 to 20 nucleobases, or 12 to 15 nucleobases.

Embodiment A35. The method of any one of embodiments A1 to A34, wherein the agent is an antisense oligomer (ASO) and wherein the antisense oligomer is at least 80%, at least 85%, at least 90%, at least 95%, at least 98%, at least 99%, or 100%, complementary to the targeted portion of the NMD exon mRNA encoding the protein.

Embodiment A36. The method of any one of embodiments A1 to A35, wherein the method further comprises assessing OPA1 mRNA or protein expression.

Embodiment A37. The method of any one of embodiments A1 to A36, wherein Optic atrophy type 1 is treated and wherein the antisense oligomer binds to a targeted portion of an OPA1 NMD exon mRNA, wherein the targeted portion is within SEQ ID NO: 2 or 3.

Embodiment A38. The method of any one of embodiments A1 to A37, wherein the subject is a human.

Embodiment A39. The method of any one of embodiments A1 to A38, wherein the subject is a non-human animal.

Embodiment A40. The method of any one of embodiments A1 to A39, wherein the subject is a fetus, an embryo, or a child.

Embodiment A41. The method of any one of embodiments A1 to A40, wherein the cells are ex vivo.

Embodiment A42. The method of any one of embodiments A1 to A41, wherein the therapeutic agent is administered by intrathecal injection, intracerebroventricular injection, intraperitoneal injection, intramuscular injection, subcutaneous injection, or intravenous injection of the subject.

Embodiment A43. The method of any of embodiments A1 to A42, wherein the method further comprises administering a second therapeutic agent to the subject.

Embodiment A44. The method of embodiment A43, wherein the second therapeutic agent is a small molecule.

Embodiment A45. The method of embodiment A43, wherein the second therapeutic agent is an ASO.

Embodiment A46. The method of any one of embodiments A43 to A45, wherein the second therapeutic agent corrects intron retention.

Embodiment A47. An antisense oligomer as used in a method of any of embodiments A1 to A46.

Embodiment A48. An antisense oligomer comprising a sequence with at least about 80%, 85%, 90%, 95%, 97%, or 100% sequence identity to a region comprising at least 8 contiguous nucleic acids of SEQ ID NO: 2 or 3.

Embodiment A49. A pharmaceutical composition comprising the antisense oligomer of embodiment A47 or A48 and an excipient.

Embodiment A50. A method of treating a subject in need thereof, comprising administering the pharmaceutical composition of embodiment A49 to the subject, wherein the administering is by intravitreal injection, intrathecal injection, intracerebroventricular injection, intraperitoneal injection, intramuscular injection, subcutaneous injection, or intravenous injection.

Embodiment A51. A composition comprising a therapeutic agent for use in a method of increasing expression of a target protein or a functional RNA by cells to treat Optic atrophy type 1 in a subject in need thereof, associated with a deficient protein or deficient functional RNA, wherein the deficient protein or deficient functional RNA is deficient in amount or activity in the subject, wherein the target protein is:
(a) the deficient protein; or
(b) a compensating protein which functionally augments or replaces the deficient protein or in the subject;
and wherein the functional RNA is:
(c) the deficient RNA; or
(d) a compensating functional RNA which functionally augments or replaces the deficient functional RNA in the subject;
wherein the therapeutic agent enhances exclusion of the nonsense-mediated RNA decay-inducing exon from the NMD exon mRNA encoding the target protein or functional RNA, thereby increasing production or activity of the target protein or the functional RNA in the subject.

Embodiment A52. A composition comprising a therapeutic agent for use in a method of treating a condition associated with OPA1 protein in a subject in need thereof, the method comprising the step of increasing expression of OPA1 protein by cells of the subject, wherein the cells have an mRNA that contains a nonsense-mediated RNA decay-inducing exon (NMD exon mRNA) and encodes OPA1 protein, the method comprising contacting the cells with the therapeutic agent, whereby the nonsense-mediated RNA decay-inducing exon is excluded from the NMD exon mRNA that encodes OPA1 protein, thereby increasing the level of mRNA encoding OPA1 protein, and increasing the expression of OPA1 protein in the cells of the subject.

Embodiment A53. The composition of embodiment A52, wherein the condition is a disease or disorder.

Embodiment A54. The composition of embodiment A53, wherein the disease or disorder is Optic atrophy type 1.

Embodiment A55. The composition of any one of embodiments A52 to 54, wherein the OPA1 protein and NMD exon mRNA are encoded by the OPA1 gene.

Embodiment A56. The composition of any one of embodiments A51 to A55, wherein the nonsense-mediated RNA decay-inducing exon is spliced out from the NMD exon mRNA encoding the OPA1 protein.

Embodiment A57. The composition of any one of embodiments A51 to A56, wherein the OPA1 protein does not comprise an amino acid sequence encoded by the nonsense-mediated RNA decay-inducing exon.

Embodiment A58. The composition of any one of embodiments A51 to A57, wherein the OPA1 protein is a full-length OPA1 protein.

Embodiment A59. The composition of any one of embodiments A51 to A58, wherein the therapeutic agent is an antisense oligomer (ASO) complementary to the targeted portion of the NMD exon mRNA.

Embodiment A60. The composition of any of embodiments A51 to A59, wherein the therapeutic agent is an antisense oligomer (ASO) and wherein the antisense oligomer targets a portion of the NMD exon mRNA that is within the nonsense-mediated RNA decay-inducing exon.

Embodiment A61. The composition of any of embodiments A51 to A59, wherein the therapeutic agent is an antisense oligomer (ASO) and wherein the antisense oligomer targets a portion of the NMD exon mRNA that is upstream or downstream of the nonsense-mediated RNA decay-inducing exon.

Embodiment A62. The composition of any one of embodiments A51 to A61, wherein the target protein is OPA1.

Embodiment A63. The composition of embodiment A62, wherein the NMD exon mRNA comprises a sequence with at least about 80%, 85%, 90%, 95%, 97%, or 100% sequence identity to SEQ ID NO: 2 or 3.

Embodiment A64. The composition of embodiment A62, wherein the NMD exon mRNA is encoded by a genetic sequence with at least about 80%, 85%, 90%, 95%, 97%, or 100% sequence identity to SEQ ID NO: 1.

Embodiment A65. The composition of embodiment A62, wherein the targeted portion of the NMD exon mRNA comprises a sequence with at least 80%, 85%, 90%, 95%, 97%, or 100% sequence identity to a region comprising at least 8 contiguous nucleic acids of SEQ ID NO: 2 or 3.

Embodiment A66. The composition of any one of embodiments A62 to A65, wherein the targeted portion of the NMD exon mRNA is within the nonsense-mediated RNA decay-inducing exon 6x of OPA1, exon 7x of OPA1, or exon 28x of OPAL.

Embodiment A67. The composition of any one of embodiments A62 to A65, wherein the targeted portion of the NMD exon mRNA is upstream or downstream of the nonsense-mediated RNA decay-inducing exon 6x of OPA1, exon 7x of OPA1, or exon 28x of OPAL.

Embodiment A68. The composition of any one of embodiments A62 to A65, wherein the targeted portion of the NMD exon mRNA comprises an exon-intron junction of exon 6x of OPA1, exon 7x of OPA1, or exon 28x of OPAL.

Embodiment A69. The composition of any one of embodiments A62 to A68, wherein the therapeutic agent is an antisense oligomer (ASO) and wherein the ASO comprises a sequence that is at least about 80%, 85%, 90%, 95%, 97%, or 100% complementary to a region comprising at least 8 contiguous nucleic acids of SEQ ID NO: 2 or 3.

Embodiment A70. The composition of any one of embodiments A51 to A69, wherein the mRNA encoding the target protein or functional RNA is a full-length mature mRNA, or a wild-type mature mRNA.

Embodiment A71. The composition of any one of embodiments A51 to A70, wherein the target protein produced is full-length protein, or wild-type protein.

Embodiment A72. The composition of any one of embodiments A51 to A71, wherein the therapeutic agent is an antisense oligomer (ASO) and wherein the antisense oligomer comprises a backbone modification comprising a phosphorothioate linkage or a phosphorodiamidate linkage.

Embodiment A73. The composition of any of embodiments A51 to A72, wherein the therapeutic agent is an antisense oligomer (ASO) and wherein said antisense oligomer is an antisense oligonucleotide.

Embodiment A74. The composition of any of embodiments A51 to A73, wherein the therapeutic agent is an antisense oligomer (ASO) and wherein the antisense oligomer comprises a phosphorodiamidate morpholino, a locked nucleic acid, a peptide nucleic acid, a 2′-O-methyl, a 2′-Fluoro, or a 2′-O-methoxyethyl moiety.

Embodiment A75. The composition of any of embodiments A51 to A74, wherein the therapeutic agent is an antisense oligomer (ASO) and wherein the antisense oligomer comprises at least one modified sugar moiety.

Embodiment A76. The composition of embodiment A75, wherein each sugar moiety is a modified sugar moiety.

Embodiment A77. The composition of any of embodiments A51 to A76, wherein the therapeutic agent is an antisense oligomer (ASO) and wherein the antisense oligomer consists of from 8 to 50 nucleobases, 8 to 40 nucleobases, 8 to 35 nucleobases, 8 to 30 nucleobases, 8 to 25 nucleobases, 8 to 20 nucleobases, 8 to 15 nucleobases, 9 to 50 nucleobases, 9 to 40 nucleobases, 9 to 35 nucleobases, 9 to 30 nucleobases, 9 to 25 nucleobases, 9 to 20 nucleobases, 9 to 15 nucleobases, 10 to 50 nucleobases, 10 to 40 nucleobases, 10 to 35 nucleobases, 10 to 30 nucleobases, 10 to 25 nucleobases, 10 to 20 nucleobases, 10 to 15 nucleobases, 11 to 50 nucleobases, 11 to 40 nucleobases, 11 to 35 nucleobases, 11 to 30 nucleobases, 11 to 25 nucleobases, 11 to 20 nucleobases, 11 to 15 nucleobases, 12 to 50 nucleobases, 12 to 40 nucleobases, 12 to 35 nucleobases, 12 to 30 nucleobases, 12 to 25 nucleobases, 12 to 20 nucleobases, or 12 to 15 nucleobases.

Embodiment A78. A pharmaceutical composition comprising the therapeutic agent of any of the compositions of embodiments A51 to A77, and an excipient.

Embodiment A79. A method of treating a subject in need thereof, comprising administering the pharmaceutical composition of embodiment A78 to the subject, wherein the administering is by intravitreal injection, intrathecal injection, intracerebroventricular injection, intraperitoneal injection, intramuscular injection, subcutaneous injection, or intravenous injection.

Embodiment A80. The method of any of embodiments A51 to A79, wherein the method further comprises administering a second therapeutic agent to the subject.

Embodiment A81. The method of embodiment A80, wherein the second therapeutic agent is a small molecule.

Embodiment A82. The method of embodiment A80, wherein the second therapeutic agent is an ASO.

Embodiment A83. The method of any one of embodiments A80 to A82, wherein the second therapeutic agent corrects intron retention.

Embodiment A84. A pharmaceutical composition comprising: an antisense oligomer that hybridizes to a target sequence of an OPA1 mRNA transcript, wherein the OPA1 mRNA transcript comprises a nonsense-mediated RNA decay-inducing exon, wherein the antisense oligomer induces exclusion of the nonsense-mediated RNA decay-inducing exon from the OPA1 mRNA transcript; and a pharmaceutical acceptable excipient.

Embodiment A85. The pharmaceutical composition of embodiment A84, wherein the OPA1 mRNA transcript is an OPA1 NMD exon mRNA transcript.

Embodiment A86. The pharmaceutical composition of embodiment A84 or A85, wherein the targeted portion of the NMD exon mRNA is within the nonsense-mediated RNA decay-inducing exon 6x of OPA1, exon 7x of OPA1, or exon 28x of OPAL.

Embodiment A87. The pharmaceutical composition of embodiment A84 or A85, wherein the targeted portion of the NMD exon mRNA is upstream or downstream of the nonsense-mediated RNA decay-inducing exon 6x of OPA1, exon 7x of OPA1, or exon 28x of OPAL.

Embodiment A88. The pharmaceutical composition of embodiment A84 or A85, wherein the targeted portion of the NMD exon mRNA comprises an exon-intron junction exon 6x of OPA1, exon 7x of OPA1, or exon 28x of OPAL.

Embodiment A89. The pharmaceutical composition of any one of embodiments A84 to A88, wherein the OPA1 NMD exon mRNA transcript is encoded by a genetic sequence with at least about 80%, 85%, 90%, 95%, 96%, 97%, 98%, 99% or 100% sequence identity to SEQ ID NO: 1.

Embodiment A90. The pharmaceutical composition of embodiment A84 or A88, wherein the OPA1 NMD exon mRNA transcript comprises a sequence with at least about 80%, 85%, 90%, 95%, 96%, 97%, 98%, 99% or 100% sequence identity to SEQ ID NO: 2 or 3.

Embodiment A91. The pharmaceutical composition of embodiment A84, wherein the antisense oligomer comprises a backbone modification comprising a phosphorothioate linkage or a phosphorodiamidate linkage.

Embodiment A92. The pharmaceutical composition of embodiment A84, wherein the antisense oligomer is an antisense oligonucleotide.

Embodiment A93. The pharmaceutical composition of embodiment A84, wherein the antisense oligomer comprises a phosphorodiamidate morpholino, a locked nucleic acid, a peptide nucleic acid, a 2′-O-methyl, a 2′-Fluoro, or a 2′-O-methoxyethyl moiety.

Embodiment A94. The pharmaceutical composition of embodiment A84, wherein the antisense oligomer comprises at least one modified sugar moiety.

Embodiment A95. The pharmaceutical composition of embodiment A84, wherein the antisense oligomer comprises from 8 to 50 nucleobases, 8 to 40 nucleobases, 8 to 35 nucleobases, 8 to 30 nucleobases, 8 to 25 nucleobases, 8 to 20 nucleobases, 8 to 15 nucleobases, 9 to 50 nucleobases, 9 to 40 nucleobases, 9 to 35 nucleobases, 9 to 30 nucleobases, 9 to 25 nucleobases, 9 to 20 nucleobases, 9 to 15 nucleobases, 10 to 50 nucleobases, 10 to 40 nucleobases, 10 to 35 nucleobases, 10 to 30 nucleobases, 10 to 25 nucleobases, 10 to 20 nucleobases, 10 to 15 nucleobases, 11 to 50 nucleobases, 11 to 40 nucleobases, 11 to 35 nucleobases, 11 to 30 nucleobases, 11 to 25 nucleobases, 11 to 20 nucleobases, 11 to 15 nucleobases, 12 to 50 nucleobases, 12 to 40 nucleobases, 12 to 35 nucleobases, 12 to 30 nucleobases, 12 to 25 nucleobases, 12 to 20 nucleobases, or 12 to 15 nucleobases.

Embodiment A96. The pharmaceutical composition of embodiment A84 or A85, wherein the antisense oligomer is at least 80%, at least 85%, at least 90%, at least 95%, at least 98%, at least 99%, or is 100% complementary to a targeted portion of the OPA1 NMD exon mRNA transcript.

Embodiment A97. The pharmaceutical composition of embodiment A84 or A85, wherein the targeted portion of the OPA1 NMD exon mRNA transcript is within SEQ ID NO: 2 or 3.

Embodiment A98. The pharmaceutical composition of embodiment A84, wherein the antisense oligomer comprises a nucleotide sequence that is at least about 80%, 85%, 90%, 91%, 92%, 93%, 94%, 95%, 96%, 97%, 98%, or 99% sequence identity to a region comprising at least 8 contiguous nucleic acids of SEQ ID NO: 2 or 3.

Embodiment A99. The pharmaceutical composition of embodiment A84, wherein the antisense oligomer comprises a nucleotide sequence that is identical a region comprising at least 8 contiguous nucleic acids SEQ ID NO: 2 or 3.

Embodiment A100. The pharmaceutical composition of any one of the embodiments A84 to A99, wherein the pharmaceutical composition is formulated for intravitreal injection, intrathecal injection, intracerebroventricular injection, intraperitoneal injection, intramuscular injection, subcutaneous injection, or intravenous injection.

Embodiment A101. The method of any of embodiments A84 to A100, wherein the method further comprises administering a second therapeutic agent to the subject.

Embodiment A102. The method of embodiment A101, wherein the second therapeutic agent is a small molecule.

Embodiment A103. The method of embodiment A101, wherein the second therapeutic agent is an ASO.

Embodiment A104. The method of any one of embodiments A101 to A103, wherein the second therapeutic agent corrects intron retention.

Embodiment A105. A method of inducing processing of a deficient OPA1 mRNA transcript to facilitate removal of a nonsense-mediated RNA decay-inducing exon to produce a fully processed OPA1 mRNA transcript that encodes a functional form of an OPA1 protein, the method comprising:
(a) contacting an antisense oligomer to a target cell of a subject;
(b) hybridizing the antisense oligomer to the deficient OPA1 mRNA transcript, wherein the deficient OPA1 mRNA transcript is capable of encoding the functional form of an OPA1 protein and comprises at least one nonsense-mediated RNA decay-inducing exon;
(c) removing the at least one nonsense-mediated RNA decay-inducing exon from the deficient OPA1 mRNA transcript to produce the fully processed OPA1 mRNA transcript that encodes the functional form of OPA1 protein; and
(d) translating the functional form of OPA1 protein from the fully processed OPA1 mRNA transcript.

Embodiment A106. A method of treating a subject having a condition caused by a deficient amount or activity of OPA1 protein comprising administering to the subject an antisense oligomer comprising a nucleotide sequence with at least about 80%, 85%, 90%, 91%, 92%, 93%, 94%, 95%, 96%, 97%, 98%, or 99% sequence identity to a region comprising at least 8 contiguous nucleic acids of SEQ ID NO: 2 or 3.

Embodiment A107. A method of treating Optic atrophy type 1 in a subject in need thereof, by increasing the expression of a target protein or functional RNA by a cell of the subject, wherein the cell has an mRNA that contains a nonsense-mediated RNA decay-inducing exon (NMD exon mRNA), and wherein the NMD exon mRNA encodes the target protein or functional RNA, the method comprising contacting the cell of the subject with a therapeutic agent that modulates splicing of the NMD exon mRNA encoding the target protein or functional RNA, whereby the nonsense-mediated RNA decay-inducing exon is excluded from the NMD exon mRNA encoding the target protein or functional RNA, thereby increasing the level of mRNA encoding the target protein or functional RNA, and increasing the expression of the target protein or functional RNA in the cell of the subject.

Embodiment A108. A method of increasing expression of OPA1 protein by a cell having an mRNA that contains a nonsense-mediated RNA decay-inducing exon (NMD exon mRNA) and encodes OPA1 protein, the method comprising contacting the cell with an agent that modulates splicing of the NMD exon mRNA encoding OPA1 protein, whereby the nonsense-mediated RNA decay-inducing exon is excluded from the NMD exon mRNA encoding OPA1 protein, thereby increasing the level of mRNA encoding OPA1 protein, and increasing the expression of OPA1 protein in the cell.

Embodiment A109. The method of embodiment A107 or A108, wherein the agent
(a) binds to a targeted portion of the NMD exon mRNA encoding the target protein or functional RNA;
(b) binds to one or more components of a spliceosome; or
(c) a combination of (a) and (b).

Embodiment B1. A method of modulating expression of a target protein, by a cell having an mRNA that comprises a nonsense-mediated RNA decay-inducing exon (NMD exon) and encodes the target protein, the method comprising contacting a therapeutic agent to the cell, whereby the therapeutic agent modulates splicing of the NMD exon from the mRNA, thereby modulating level of processed mRNA encoding the target protein, and modulating the expression of the target protein in the cell, wherein the target protein is selected from the group consisting of: OPA1 proteins.

Embodiment B2. A method of treating a disease or condition in a subject in need thereof by modulating expression of a target protein in a cell of the subject, comprising: contacting the cell of the subject with a therapeutic agent that modulates splicing of a nonsense-mediated mRNA decay-inducing exon (NMD exon) from an mRNA in the cell, wherein the mRNA comprises the NMD exon and encodes the target protein, thereby modulating level of processed mRNA encoding the target protein, and modulating expression of the target protein in the cell of the subject, wherein the target protein is selected from the group consisting of: OPA1 proteins.

Embodiment B3. The method of embodiment B1 or B2, wherein the therapeutic agent
(a) binds to a targeted portion of the mRNA encoding the target protein;
(b) modulates binding of a factor involved in splicing of the NMD exon; or
(c) a combination of (a) and (b).

Embodiment B4. The method of embodiment B3, wherein the therapeutic agent interferes with binding of the factor involved in splicing of the NMD exon to a region of the targeted portion.

Embodiment B5. The method of embodiment B3 or B4, wherein the targeted portion is proximal to the NMD exon.

Embodiment B6. The method of any one of embodiments B3 to B5, wherein the targeted portion is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of 5′ end of the NMD exon.

Embodiment B7. The method of any one of embodiments B3 to B6, wherein the targeted portion is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides, about 40 nucleotides, about 30 nucleotides, about 20 nucleotides, about 10 nucleotides, about 5 nucleotides, about 4 nucleotides, about 2 nucleotides, about 1 nucleotides upstream of 5′ end of the NMD exon.

Embodiment B8. The method of any one of embodiments B3 to B5, wherein the targeted portion is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of 3′ end of the NMD exon.

Embodiment B9. The method of any one of embodiments B3 to B5 or B8, wherein the targeted portion is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides, about 40 nucleotides, about 30 nucleotides, about 20 nucleotides, about 10 nucleotides, about 5 nucleotides, about 4 nucleotides, about 2 nucleotides, about 1 nucleotides downstream of 3′ end of the NMD exon.

Embodiment B10. The method of any one of embodiments B3 to B5, wherein the targeted portion is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site selected from the group consisting of: GRCh38/hg38: chr3 193628509; and GRCh38/hg38: chr3 193603500.

Embodiment B11. The method of any one of embodiments B3 to B5 or B10, wherein the targeted portion is about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site selected from the group consisting of: GRCh38/hg38: chr3 193628509; and GRCh38/hg38: chr3 193603500.

Embodiment B12. The method of any one of embodiments B3 to B5, wherein the targeted portion is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site selected from the group consisting of: GRCh38/hg38: chr3 193628616; and GRCh38/hg38: chr3 193603557.

Embodiment B13. The method of any one of embodiments B3 to B5 or B12, wherein the targeted portion is about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site selected from the group consisting of: GRCh38/hg38: chr3 193628616; and GRCh38/hg38: chr3 193603557.

Embodiment B14. The method of any one of embodiments B3 to B13, wherein the targeted portion is located in an intronic region between two canonical exonic regions of the mRNA encoding the target protein, and wherein the intronic region contains the NMD exon.

Embodiment B15. The method of any one of embodiments B3 to B14, wherein the targeted portion at least partially overlaps with the NMD exon.

Embodiment B16. The method of any one of embodiments B3 to B15, wherein the targeted portion at least partially overlaps with an intron upstream or downstream of the NMD exon.

Embodiment B17. The method of any one of embodiments B3 to B16, wherein the targeted portion comprises 5′ NMD exon-intron junction or 3′ NMD exon-intron junction.

Embodiment B18. The method of any one of embodiments B3 to B16, wherein the targeted portion is within the NMD exon.

Embodiment B19. The method of any one of embodiments B1 to B18, wherein the targeted portion comprises about 5, 6, 7, 8, 9, 10, 11, 12, 13, 14, 15, 16, 17, 18, 19, 20, 21, 22, 23, 24, 25, 26, 27, 28, 29, 30, or more consecutive nucleotides of the NMD exon.

Embodiment B20. The method of any one of embodiments B1 to B19, wherein the mRNA encoding the target protein comprises a sequence with at least about 80%, 85%, 90%, 95%, 97%, or 100% sequence identity to SEQ ID NO: 4 or 5.

Embodiment B21. The method of any one of embodiments B1 to B20, wherein the mRNA encoding the target protein is encoded by a genetic sequence with at least about 80%, 85%, 90%, 95%, 97%, or 100% sequence identity to SEQ ID NO: 1.

Embodiment B22. The method of any one of embodiments B3 to B21, wherein the targeted portion of the mRNA comprises a sequence with at least 80%, 85%, 90%, 95%, 97%, or 100% sequence identity to a region comprising at least 8 contiguous nucleic acids of SEQ ID NO: 4 or 5.

Embodiment B23. The method of any one of embodiments B1 to B22, wherein the agent is an antisense oligomer (ASO) and wherein the ASO comprises a sequence that is at least about 80%, 85%, 90%, 95%, 97%, or 100% complementary to at least 8 contiguous nucleic acids of SEQ ID Ns: 4 or 5.

Embodiment B24. The method of any one of embodiments B3 to B23, wherein the targeted portion of the mRNA is within the nonsense-mediated RNA decay-inducing exon selected from the group consisting of: GRCh38/hg38: chr3 193628509 193628616; and GRCh38/hg38: chr3 193603500 193603557.

Embodiment B25. The method of any one of embodiments B3 to B23, wherein the targeted portion of the mRNA is upstream or downstream of the nonsense-mediated RNA decay-inducing exon selected from the group consisting of: GRCh38/hg38: chr3 193628509 193628616; and GRCh38/hg38: chr3 193603500 193603557.

Embodiment B26. The method of any one of embodiments B3 to B23, wherein the targeted portion of the mRNA comprises an exon-intron junction of exon selected from the group consisting of: GRCh38/hg38: chr3 193628509 193628616; and GRCh38/hg38: chr3 193603500 193603557.

Embodiment B27. The method of any one of embodiments B1 to B26, wherein the target protein produced is a full-length protein or a wild-type protein.

Embodiment B28. The method of any one of embodiments B1 to B27, wherein the therapeutic agent promotes exclusion of the NMD exon from the pre-mRNA encoding the target protein.

Embodiment B29. The method of embodiment B28, wherein exclusion of the NMD exon from the pre-mRNA encoding the target protein in the cell contacted with the therapeutic agent is increased by about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to exclusion of the NMD exon from the pre-mRNA encoding the target protein in a control cell.

Embodiment B30. The method of embodiment B28 or B29, wherein the therapeutic agent increases the level of the processed mRNA encoding the target protein in the cell.

Embodiment B31. The method of any one of embodiments B28 to B30, wherein the level of the processed mRNA encoding the target protein produced in the cell contacted with the therapeutic agent is increased by about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to a level of the processed mRNA encoding the target protein in a control cell.

Embodiment B32. The method of any one of embodiments B28 to B31, wherein the therapeutic agent increases the expression of the target protein in the cell.

Embodiment B33. The method of any one of embodiments B28 to B32, wherein a level of the target protein produced in the cell contacted with the therapeutic agent is increased by about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to a level of the target protein produced in a control cell.

Embodiment B34. The method of any one of embodiments B2 to B33, wherein the disease or condition is induced by a loss-of-function mutation in the target protein.

Embodiment B35. The method of embodiment B34, wherein the disease or condition is associated with haploinsufficiency of a gene encoding the target protein, and wherein the subject has a first allele encoding a functional target protein, and a second allele from which the target protein is not produced or produced at a reduced level, or a second allele encoding a nonfunctional target protein or a partially functional target protein.

Embodiment B36. The method of any one of embodiments B2 to B35, wherein the disease or condition is selected from the group consisting of. Optic atrophy type 1.

Embodiment B37. The method of any one of embodiments B34 to B36, wherein the therapeutic agent promotes exclusion of the NMD exon from the pre-mRNA encoding the target protein and increases the expression of the target protein in the cell.

Embodiment B38. The method of any one of embodiments B1 to B27, wherein the therapeutic agent inhibits exclusion of the NMD exon from the pre-mRNA encoding the target protein.

Embodiment B39. The method of embodiment B38, wherein exclusion of the NMD exon from the pre-mRNA encoding the target protein in the cell contacted with the therapeutic agent is decreased by about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to exclusion of the NMD exon from the pre-mRNA encoding the target protein in a control cell.

Embodiment B40. The method of embodiment B38 or B39, wherein the therapeutic agent decreases the level of the processed mRNA encoding the target protein in the cell.

Embodiment B41. The method of any one of embodiments B38 to B40, wherein the level of the processed mRNA encoding the target protein in the cell contacted with the therapeutic agent is decreased by about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to a level of the processed mRNA encoding the target protein in a control cell.

Embodiment B42. The method of any one of embodiments B38 to B41, wherein the therapeutic agent decreases the expression of the target protein in the cell.

Embodiment B43. The method of any one of embodiments B38 to B42, wherein a level of the target protein produced in the cell contacted with the therapeutic agent is decreased by about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to a level of the target protein produced in a control cell.

Embodiment B44. The method of any one of embodiments B2 to B27 or B38 to B43, wherein the disease or condition is induced by a gain-of-function mutation in the target protein.

Embodiment B45. The method of embodiment B44, wherein the subject has an allele from which the target protein is produced at an increased level, or an allele encoding a mutant target protein that exhibits increased activity in the cell.

Embodiment B46. The method of embodiment B44 or B45, wherein the therapeutic agent inhibits exclusion of the NMD exon from the pre-mRNA encoding the target protein and decreases the expression of the target protein in the cell.

Embodiment B47. The method of any one of embodiments B1 to B46, wherein the therapeutic agent is an antisense oligomer (ASO) and wherein the antisense oligomer comprises a backbone modification comprising a phosphorothioate linkage or a phosphorodiamidate linkage.

Embodiment B48. The method of any one of embodiments B1 to B47, wherein the therapeutic agent is an antisense oligomer (ASO) and wherein the antisense oligomer comprises a phosphorodiamidate morpholino, a locked nucleic acid, a peptide nucleic acid, a 2′-O-methyl, a 2′-Fluoro, or a 2′-O-methoxyethyl moiety.

Embodiment B49. The method of any one of embodiments B1 to B48, wherein the therapeutic agent is an antisense oligomer (ASO) and wherein the antisense oligomer comprises at least one modified sugar moiety.

Embodiment B50. The method of embodiment B49, wherein each sugar moiety is a modified sugar moiety.

Embodiment B51. The method of any one of embodiments B1 to B50, wherein the therapeutic agent is an antisense oligomer (ASO) and wherein the antisense oligomer consists of from 8 to 50 nucleobases, 8 to 40 nucleobases, 8 to 35 nucleobases, 8 to 30 nucleobases, 8 to 25 nucleobases, 8 to 20 nucleobases, 8 to 15 nucleobases, 9 to 50 nucleobases, 9 to 40 nucleobases, 9 to 35 nucleobases, 9 to 30 nucleobases, 9 to 25 nucleobases, 9 to 20 nucleobases, 9 to 15 nucleobases, 10 to 50 nucleobases, 10 to 40 nucleobases, 10 to 35 nucleobases, 10 to 30 nucleobases, 10 to 25 nucleobases, 10 to 20 nucleobases, 10 to 15 nucleobases, 11 to 50 nucleobases, 11 to 40 nucleobases, 11 to 35 nucleobases, 11 to 30 nucleobases, 11 to 25 nucleobases, 11 to 20 nucleobases, 11 to 15 nucleobases, 12 to 50 nucleobases, 12 to 40 nucleobases, 12 to 35 nucleobases, 12 to 30 nucleobases, 12 to 25 nucleobases, 12 to 20 nucleobases, or 12 to 15 nucleobases.

Embodiment B52. The method of any one of embodiments B3 to B51, wherein the therapeutic agent is an antisense oligomer (ASO) and wherein the antisense oligomer is at least 80%, at least 85%, at least 90%, at least 95%, at least 98%, at least 99%, or 100%, complementary to the targeted portion of the mRNA.

Embodiment B53. The method of any one of embodiments B1 to B52, wherein the method further comprises assessing mRNA level or expression level of the target protein.

Embodiment B54. The method of any one of embodiments B1 to B53, wherein the subject is a human.

Embodiment B55. The method of any one of embodiments B1 to B53, wherein the subject is a non-human animal.

Embodiment B56. The method of any one of embodiments B2 to B54, wherein the subject is a fetus, an embryo, or a child.

Embodiment B57. The method of any one of embodiments B1 to B56, wherein the cells are ex vivo.

Embodiment B58. The method of any one of embodiments B2 to B56, wherein the therapeutic agent is administered by intravitreal injection, intrathecal injection, intracerebroventricular injection, intraperitoneal injection, intramuscular injection, subcutaneous injection, intravitreal, or intravenous injection of the subject.

Embodiment B59. The method of any one of embodiments B2 to B56 or B58, wherein the method further comprises administering a second therapeutic agent to the subject.

Embodiment B60. The method of any one of embodiments B1 to B59, wherein the second therapeutic agent is a small molecule.

Embodiment B61. The method of any one of embodiments B1 to B59, wherein the second therapeutic agent is an antisense oligomer.

Embodiment B62. The method of any one of embodiments B1 to B61, wherein the second therapeutic agent corrects intron retention.

Embodiment B63. The method of any one of embodiments B2 to B62, wherein the disease or condition is Optic atrophy type 1.

### FURTHER SPECIFIC EMBODIMENTS

Embodiment 1. A method of modulating expression of an OPA1 protein in a cell having a pre-mRNA that is transcribed from an OPA1 gene and that comprises a nonsense-mediated RNA decay-inducing exon (NMD exon), the method comprising contacting an agent or a vector encoding the agent to the cell, whereby the agent modulates splicing of the NMD exon from the pre-mRNA, thereby modulating a level of processed mRNA that is processed from the pre-mRNA, and modulating the expression of the OPA1 protein in the cell, wherein the agent comprises an antisense oligomer with at least 80% sequence identity to a sequence selected from the group consisting of SEQ ID NOs: 6-275 and 280-299.

Embodiment 2. The method of embodiment 1, wherein the agent:
(a) binds to a targeted portion of the pre-mRNA;
(b) modulates binding of a factor involved in splicing of the NMD exon; or
(c) a combination of (a) and (b).

Embodiment 3. The method of embodiment 2, wherein the agent interferes with binding of the factor involved in splicing of the NMD exon to a region of the targeted portion.

Embodiment 4. The method of embodiment 2, wherein the targeted portion of the pre-mRNA is proximal to the NMD exon.

Embodiment 5. The method of embodiment 2, wherein the targeted portion of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of 5′ end of the NMD exon.

Embodiment 6. The method of embodiment 2, wherein the targeted portion of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides, about 40 nucleotides, about 30 nucleotides, about 20 nucleotides, about 10 nucleotides, about 5 nucleotides, about 4 nucleotides, about 2 nucleotides, about 1 nucleotides upstream of 5′ end of the NMD exon.

Embodiment 7. The method of embodiment 2, wherein the targeted portion of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of 3′ end of the NMD exon.

Embodiment 8. The method of embodiment 2, wherein the targeted portion of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides, about 40 nucleotides, about 30 nucleotides, about 20 nucleotides, about 10 nucleotides, about 5 nucleotides, about 4 nucleotides, about 2 nucleotides, about 1 nucleotides downstream of 3′ end of the NMD exon.

Embodiment 9. The method of embodiment 2, wherein the targeted portion of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr3 193628509.

Embodiment 10. The method of embodiment 2, wherein the targeted portion of the pre-mRNA is about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site GRCh38/hg38: chr3 193628509.

Embodiment 11. The method of embodiment 2, wherein the targeted portion of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr3 193628616.

Embodiment 12. The method of embodiment 2, wherein the targeted portion of the pre-mRNA is about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site GRCh38/hg38: chr3 193628616.

Embodiment 13. The method of embodiment 2, wherein the targeted portion of the pre-mRNA is located in an intronic region between two canonical exonic regions of the pre-mRNA, and wherein the intronic region contains the NMD exon.

Embodiment 14. The method of embodiment 2, wherein the targeted portion of the pre-mRNA at least partially overlaps with the NMD exon.

Embodiment 15. The method of embodiment 2, wherein the targeted portion of the pre-mRNA at least partially overlaps with an intron upstream or downstream of the NMD exon.

Embodiment 16. The method of embodiment 2, wherein the targeted portion of the pre-mRNA comprises 5′ NMD exon-intron junction or 3′ NMD exon-intron junction.

Embodiment 17. The method of embodiment 2, wherein the targeted portion of the pre-mRNA is within the NMD exon.

Embodiment 18. The method of embodiment 2, wherein the targeted portion of the pre-mRNA comprises about 5, 6, 7, 8, 9, 10, 11, 12, 13, 14, 15, 16, 17, 18, 19, 20, 21, 22, 23, 24, 25, 26, 27, 28, 29, 30, or more consecutive nucleotides of the NMD exon.

Embodiment 19. The method of any one of embodiments 1 to 18, wherein the NMD exon comprises a sequence with at least 80%, at least 90%, or 100% sequence identity to SEQ ID NO: 279.

Embodiment 20. The method of any one of embodiments 1 to 18, wherein the NMD exon comprises a sequence of SEQ ID NO: 279.

Embodiment 21. The method of embodiment 2, wherein the targeted portion of the pre-mRNA is within the nonsense-mediated RNA decay-inducing exon GRCh38/hg38: chr3 193628509 to 193628616.

Embodiment 22. The method of embodiment 2, wherein the targeted portion of the pre-mRNA is upstream or downstream of the nonsense-mediated RNA decay-inducing exon GRCh38/hg38: chr3 193628509 to 193628616.

Embodiment 23. The method of embodiment 2, wherein the targeted portion of the pre-mRNA comprises an exon-intron junction of exon GRCh38/hg38: chr3 193628509 to 193628616.

Embodiment 24. The method of any one of embodiments 1 to 23, wherein the OPA1 protein expressed from the processed mRNA is a full-length OPA1 protein or a wild-type OPA1 protein.

Embodiment 25. The method of any one of embodiments 1 to 23, wherein the OPA1 protein expressed from the processed mRNA is a functional OPA1 protein.

Embodiment 26. The method of any one of embodiments 1 to 23, wherein the OPA1 protein expressed from the processed mRNA is at least partially functional as compared to a wild-type OPA1 protein.

Embodiment 27. The method of any one of embodiments 1 to 23, wherein the OPA1 protein expressed from the processed mRNA is at least partially functional as compared to a full-length wild-type OPA1 protein.

Embodiment 28. The method of any one of embodiments 1 to 23, or 25 to 27, wherein the OPA1 protein expressed from the processed mRNA is an OPA1 protein that lacks an amino acid sequence encoded by a nucleic acid sequence with at least 80% sequence identity to SEQ ID NO: 277.

Embodiment 29. The method of any one of embodiments 1 to 28, wherein the method promotes exclusion of the NMD exon from the pre-mRNA.

Embodiment 30. The method of embodiment 29, wherein the exclusion of the NMD exon from the pre-mRNA in the cell contacted with the agent is increased by about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to in the absence of the agent.

Embodiment 31. The method of any one of embodiments 1 to 30, wherein the method results in an increase in the level of the processed mRNA in the cell.

Embodiment 32. The method of embodiment 31, wherein the level of the processed mRNA in the cell contacted with the agent is increased by about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to in the absence of the agent.

Embodiment 33. The method of any one of embodiments 1 to 32, wherein the method results in an increase in the expression of the OPA1 protein in the cell.

Embodiment 34. The method of embodiment 33, wherein a level of the OPA1 protein expressed from the processed mRNA in the cell contacted with the agent is increased by about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to in the absence of the agent.

Embodiment 35. The method of any one of embodiments 1 to 34, wherein the agent comprises an antisense oligomer with at least 80%, at least 90%, or 100% sequence identity to a sequence selected from the group consisting of SEQ ID NOs: 36, 236, 242, 250, 280-283, 288, and 290-292.

Embodiment 36. The method of any one of embodiments 1 to 34, wherein the agent further comprises a gene editing molecule.

Embodiment 37. The method of embodiment 36, wherein the gene editing molecule comprises CRISPR-Cas9.

Embodiment 38. A method of modulating expression of an OPA1 protein in a cell having a pre-mRNA that is transcribed from an OPA1 gene, wherein the pre-mRNA comprises a coding exon, the method comprising contacting an agent or a vector encoding the agent to the cell, whereby the agent promotes exclusion of the coding exon from the pre-mRNA, thereby increasing a level of a processed mRNA that is processed from the pre-mRNA and that lacks the coding exon in the cell.

Embodiment 39. The method of embodiment 38, wherein the agent:
(a) binds to a targeted portion of the pre-mRNA;
(b) modulates binding of a factor involved in splicing of the coding exon; or
(c) a combination of (a) and (b).

Embodiment 40. The method of embodiment 39, wherein the agent interferes with binding of the factor involved in splicing of the coding exon to a region of the targeted portion.

Embodiment 41. The method of embodiment 39, wherein the targeted portion of the pre-mRNA is proximal to the coding exon.

Embodiment 42. The method of embodiment 39, wherein the targeted portion of the pre-mRNA is located in an intronic region immediately upstream of the coding exon.

Embodiment 43. The method of embodiment 39, wherein the targeted portion of the pre-mRNA is within a region spanning from 100 to 50, from 90 to 50, from 80 to 50, from 70 to 50, from 60 to 50, from 60 to 40, from 60 to 30, from 60 to 20, from 60 to 10, from 49 to 1, from 39 to 1, from 29 to 1, or from 19 to 1 nucleotides upstream of 5′ end of the coding exon.

Embodiment 44. The method of embodiment 39, wherein the targeted portion of the pre-mRNA is within a region spanning from 49 to 1, from 39 to 1, from 29 to 1, or from 19 to 1 nucleotides upstream of 5′ end of the coding exon.

Embodiment 45. The method of embodiment 39, wherein the targeted portion of the pre-mRNA is located in an intronic region immediately downstream of the coding exon.

Embodiment 46. The method of embodiment 39, wherein the targeted portion of the pre-mRNA is within a region spanning from 1 to 49, from 1 to 39, from 1 to 29, from 1 to 19, from 10 to 60, from 20 to 60, from 30 to 60, from 40 to 60, from 50 to 60, from 50 to 70, from 50 to 80, from 50 to 90, or from 50 to 100 nucleotides downstream of 3′ end of the coding exon.

Embodiment 47. The method of embodiment 39, wherein the targeted portion of the pre-mRNA is within a region spanning from 1 to 49, from 1 to 39, from 1 to 29, or from 1 to 19 nucleotides downstream of 3′ end of the coding exon.

Embodiment 48. The method of embodiment 39, wherein the targeted portion of the pre-mRNA at least partially overlaps with the coding exon.

Embodiment 49. The method of embodiment 39, wherein the targeted portion of the pre-mRNA at least partially overlaps with an intron immediately upstream or immediately downstream of the coding exon.

Embodiment 50. The method of embodiment 39, wherein the targeted portion of the pre-mRNA comprises 5′ coding exon-intron junction or 3′ coding exon-intron junction.

Embodiment 51. The method of embodiment 39, wherein the targeted portion is within the coding exon of the pre-mRNA.

Embodiment 52. The method of any one of embodiments 39 to 51, wherein the coding exon is an alternatively spliced exon.

Embodiment 53. The method of any one of embodiments 39 to 52, wherein the coding exon comprises a sequence with at least 80%, at least 90%, or 100% sequence identity to SEQ ID NO: 277.

Embodiment 54. The method of any one of embodiments 39 to 52, wherein the coding exon comprises SEQ ID NO: 277.

Embodiment 55. The method of embodiment 39, wherein the targeted portion of the pre-mRNA is immediately upstream of the coding exon GRCh38/hg38: chr3 193626092 to 193626202.

Embodiment 56. The method of embodiment 39, wherein the targeted portion of the pre-mRNA is within a region spanning from 49 to 1, from 39 to 1, from 29 to 1, or from 19 to 1 nucleotides upstream of genomic site GRCh38/hg38: chr3 193626092.

Embodiment 57. The method of embodiment 39, wherein the targeted portion of the pre-mRNA is immediately downstream of the coding exon GRCh38/hg38: chr3 193626092 to 193626202.

Embodiment 58. The method of embodiment 39, wherein the targeted portion of the pre-mRNA is within a region spanning from 1 to 49, from 1 to 39, from 1 to 29, or from 1 to 19 nucleotides downstream of genomic site GRCh38/hg38: chr3 193626202.

Embodiment 59. The method of embodiment 39, wherein the targeted portion of the pre-mRNA is within the coding exon GRCh38/hg38: chr3 193626092 to 193626202.

Embodiment 60. The method of embodiment 39, wherein the targeted portion of the pre-mRNA comprises an exon-intron junction of exon GRCh38/hg38: chr3 193626092 to 193626202.

Embodiment 61. The method of embodiment 39, wherein the targeted portion comprises about 5, 6, 7, 8, 9, 10, 11, 12, 13, 14, 15, 16, 17, 18, 19, 20, 21, 22, 23, 24, 25, 26, 27, 28, 29, 30, or more consecutive nucleotides of the coding exon.

Embodiment 62. The method of embodiment 39, wherein the targeted portion of the pre-mRNA comprises a sequence with at least 80%, 85%, 90%, 95%, 97%, or 100% sequence identity to a region comprising at least 8 contiguous nucleic acids of SEQ ID NO: 277.

Embodiment 63. The method of any one of embodiments 38 to 62, wherein the exclusion of the coding exon from the pre-mRNA in the cell contacted with the agent is increased by about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to in the absence of the agent.

Embodiment 64. The method of any one of embodiments 38 to 63, wherein the method results in an increase in expression of the OPA1 protein in the cell.

Embodiment 65. The method of embodiment 64, wherein a level of the OPA1 protein expressed from the processed mRNA in the cell contacted with the agent is increased by about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to in the absence of the agent.

Embodiment 66. The method of embodiment 64, wherein a level of the OPA1 protein expressed from the processed mRNA in the cell contacted with the agent is increased by at least about 1.5-fold compared to in the absence of the agent.

Embodiment 67. The method of any one of embodiments 64 to 66, wherein the OPA1 protein expressed from the processed mRNA is a functional OPA1 protein.

Embodiment 68. The method of any one of embodiments 64 to 66, wherein the OPA1 protein expressed from the processed mRNA is at least partially functional as compared to a wild-type OPA1 protein.

Embodiment 69. The method of any one of embodiments 64 to 66, wherein the OPA1 protein expressed from the processed mRNA is at least partially functional as compared to a full-length wild-type OPA1 protein.

Embodiment 70. The method of any one of embodiments 64 to 69, wherein the OPA1 protein expressed from the processed mRNA comprises fewer proteolytic cleavage sites than an OPA1 protein encoded by a corresponding mRNA containing the coding exon.

Embodiment 71. The method of any one of embodiments 38 to 70, wherein the agent promotes exclusion of a nonsense-mediated RNA decay-inducing exon (NMD exon) from the pre-mRNA.

Embodiment 72. The method of embodiment 71, wherein the NMD exon comprises a sequence with at least 80%, at least 90%, or 100% sequence identity to SEQ ID NO: 279.

Embodiment 73. The method of embodiment 71, wherein the NMD exon comprises a sequence of SEQ ID NO: 279.

Embodiment 74. The method of any one of embodiments 64 to 73, wherein the OPA1 protein expressed from the processed mRNA comprises fewer proteolytic cleavage sites than an OPA1 protein encoded by a corresponding mRNA containing the coding exon.

Embodiment 75. The method of any one of embodiments 38 to 74, wherein the agent comprises an antisense oligomer with at least 80%, at least 90%, or 100% sequence identity to a sequence selected from the group consisting of SEQ ID NOs: 227-242, 250, 280-283, 288, and 290-292.

Embodiment 76. The method of any one of embodiments 38 to 74, wherein the agent comprises a gene editing molecule.

Embodiment 77. The method of embodiment 76, wherein the gene editing molecule comprises CRISPR-Cas9.

Embodiment 78. A method of modulating expression of an OPA1 protein in a cell having a pre-mRNA that is transcribed from an OPA1 gene, wherein the pre-mRNA comprises a coding exon, the method comprising contacting an agent or a vector encoding the agent to the cell,
wherein the agent comprises an antisense oligomer that binds to:
(a) a targeted portion of the pre-mRNA within an intronic region immediately upstream of a 5′ end of the coding exon of the pre-mRNA; or
(b) a targeted portion of the pre-mRNA within an intronic region immediately downstream of a 3′ end of the coding exon of the pre-mRNA;
whereby the agent increases a level of a processed mRNA that is processed from the pre-mRNA and that contains the coding exon in the cell.

Embodiment 79. The method of embodiments 78, wherein the coding exon is an alternatively spliced exon.

Embodiment 80. The method of embodiments 78 or 79, wherein the method promotes inclusion of the coding exon in the processed mRNA during splicing of the pre-mRNA in the cell.

Embodiment 81. The method of any one of embodiments 78 to 80, wherein the target portion of the pre-mRNA is within a region spanning from 100 to 50, from 100 to 60, from 100 to 70, from 100 to 80, or from 100 to 90 nucleotides upstream of a 5′ end of the coding exon.

Embodiment 82. The method of any one of embodiments 78 to 80, wherein the target portion of the pre-mRNA is within a region spanning from 40 to 100, from 50 to 100, from 60 to 100, from 70 to 100, from 80 to 100, or from 90 to 100 nucleotides downstream of a 3′ end of the coding exon.

Embodiment 83. The method of any one of embodiments 78 to 80, wherein the coding exon comprises a sequence with at least 80%, at least 90%, or 100% sequence identity to SEQ ID NO: 277.

Embodiment 84. The method of any one of embodiments 78 to 80, wherein the coding exon comprises SEQ ID NO: 277.

Embodiment 85. The method of any one of embodiments 78 to 80, wherein the targeted portion of the pre-mRNA is within a region spanning from 100 to 50, from 100 to 60, from 100 to 70, from 100 to 80, or from 100 to 90 nucleotides upstream of genomic site GRCh38/hg38: chr3 193626092.

Embodiment 86. The method of any one of embodiments 78 to 80, wherein the targeted portion of the pre-mRNA is within a region spanning from 40 to 100, from 50 to 100, from 60 to 100, from 70 to 100, from 80 to 100, or from 90 to 100 nucleotides downstream of genomic site GRCh38/hg38: chr3 193626202.

Embodiment 87. The method of any one of embodiments 78 to 86, wherein the inclusion of the coding exon in the processed mRNA in the cell contacted with the agent is increased by about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to in the absence of the agent.

Embodiment 88. The method of any one of embodiments 78 to 87, wherein the agent comprises an antisense oligomer with at least 80%, at least 90%, or 100% sequence identity to SEQ ID NO: 267.

Embodiment 89. A method of modulating expression of a target protein in a cell having a pre-mRNA transcribed from a gene that encodes the target protein, wherein the pre-mRNA comprises a coding exon and a nonsense-mediated RNA decay-inducing exon (NMD exon), the method comprising contacting an agent or a vector encoding the agent to the cell,
wherein the agent promotes exclusion of both the coding exon and the NMD exon from the pre-mRNA, thereby increasing a level of a processed mRNA that is processed from the pre-mRNA and that lacks both the NMD exon and the coding exon in the cell.

Embodiment 90. The method of embodiment 89, wherein the agent:
(a) binds to a targeted portion of the pre-mRNA;
(b) modulates binding of a factor involved in splicing of the coding exon, the NMD exon, or both; or
(c) a combination of (a) and (b).

Embodiment 91. The method of embodiment 90, wherein the agent interferes with binding of the factor involved in splicing of the coding exon, the NMD exon, or both, to a region of the targeted portion.

Embodiment 92. The method of any one of embodiments 89 to 91, wherein the NMD exon is within an intronic region adjacent to the coding exon.

Embodiment 93. The method of embodiment 92, wherein the NMD exon is within an intronic region immediately upstream of the coding exon.

Embodiment 94. The method of embodiment 92, wherein the NMD exon is within an intronic region immediately downstream of the coding exon.

Embodiment 95. The method of any one of embodiments 90 to 94, wherein the targeted portion of the pre-mRNA is proximal to the coding exon.

Embodiment 96. The method of any one of embodiments 90 to 94, wherein the targeted portion of the pre-mRNA is located in an intronic region immediately upstream of the coding exon.

Embodiment 97. The method of any one of embodiments 90 to 94, wherein the targeted portion of the pre-mRNA is located in an intronic region immediately downstream of the coding exon.

Embodiment 98. The method of any one of embodiments 90 to 94, wherein the targeted portion of the pre-mRNA is located within the coding exon.

Embodiment 99. The method of any one of embodiments 90 to 94, wherein the targeted portion of the pre-mRNA is within a region spanning from 49 to 1, from 39 to 1, from 29 to 1, or from 19 to 1 nucleotides upstream of 5′ end of the coding exon.

Embodiment 100. The method of any one of embodiments 90 to 94, wherein the targeted portion of the pre-mRNA is within a region spanning from 100 nucleotides upstream of the coding exon to 100 nucleotides downstream of the coding exon.

Embodiment 101. The method of any one of embodiments 89 to 100, wherein the coding exon is an alternatively spliced exon.

Embodiment 102. The method of any one of embodiments 89 to 101, wherein the coding exon comprises a sequence with at least 80%, at least 90%, or 100% sequence identity to SEQ ID NO: 277.

Embodiment 103. The method of any one of embodiments 89 to 101, wherein the coding exon comprises SEQ ID NO: 277.

Embodiment 104. The method of embodiment 90, wherein the targeted portion of the pre-mRNA is immediately upstream of the coding exon GRCh38/hg38: chr3 193626092 to 193626202.

Embodiment 105. The method of embodiment 90, wherein the targeted portion of the pre-mRNA is immediately downstream of the coding exon GRCh38/hg38: chr3 193626092 to 193626202.

Embodiment 106. The method of any one of embodiments 90 to 94, wherein the targeted portion of the pre-mRNA is within a region spanning from 49 to 1, from 39 to 1, from 29 to 1, or from 19 to 1 nucleotides upstream of GRCh38/hg38: chr3 193626092.

Embodiment 107. The method of embodiment 90, wherein the targeted portion of the pre-mRNA is within a region spanning from 100 nucleotides upstream of genomic site GRCh38/hg38: chr3 193626092. to 100 nucleotides downstream of genomic site GRCh38/hg38: chr3 193626202.

Embodiment 108. The method of embodiment 90, wherein the targeted portion of the pre-mRNA is within the coding exon GRCh38/hg38: chr3 193626092 to 193626202.

Embodiment 109. The method of embodiment 90, wherein the targeted portion of the pre-mRNA comprises an exon-intron junction of the coding exon GRCh38/hg38: chr3 193626092 to 193626202.

Embodiment 110. The method of embodiment 90, wherein the targeted portion comprises about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more consecutive nucleotides of the coding exon.

Embodiment 111. The method of embodiment 90, wherein the targeted portion of the pre-mRNA is proximal to the NMD exon.

Embodiment 112. The method of embodiment 90, wherein the targeted portion of the pre-mRNA is located in an intronic region immediately upstream of the NMD exon.

Embodiment 113. The method of embodiment 90, wherein the targeted portion of the pre-mRNA is located in an intronic region immediately downstream of the NMD exon.

Embodiment 114. The method of embodiment 90, wherein the targeted portion of the pre-mRNA is located within the NMD exon.

Embodiment 115. The method of embodiment 90, wherein the targeted portion of the pre-mRNA is within a region spanning from 100 nucleotides upstream of the NMD exon to 100 nucleotides downstream of the NMD exon.

Embodiment 116. The method of any one of embodiments 89 to 115, wherein the NMD exon comprises a sequence with at least 80%, at least 90%, or 100% sequence identity to SEQ ID NO: 279.

Embodiment 117. The method of embodiment 89, wherein the NMD exon comprises SEQ ID NO: 279.

Embodiment 118. The method of embodiment 90, wherein the targeted portion of the pre-mRNA is immediately upstream of the NMD exon GRCh38/hg38: chr3 193628509 to 193628616.

Embodiment 119. The method of embodiment 90, wherein the targeted portion of the pre-mRNA is immediately downstream of the NMD exon GRCh38/hg38: chr3 193628509 to 193628616.

Embodiment 120. The method of embodiment 90, wherein the targeted portion of the pre-mRNA is within a region spanning from 100 nucleotides upstream of genomic site GRCh38/hg38: chr3 193628509 to 100 nucleotides downstream of genomic site GRCh38/hg38: chr3 193628616.

Embodiment 121. The method of embodiment 90, wherein the targeted portion of the pre-mRNA is within the NMD exon GRCh38/hg38: chr3 193628509 to 193628616.

Embodiment 122. The method of embodiment 90, wherein the targeted portion of the pre-mRNA comprises an exon-intron junction of the NMD exon GRCh38/hg38: chr3 193628509 to 193628616.

Embodiment 123. The method of embodiment 90, wherein the targeted portion comprises about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more consecutive nucleotides of the NMD exon.

Embodiment 124. The method of any one of embodiments 89 to 123, wherein the exclusion of the coding exon from the pre-mRNA in the cell contacted with the agent is increased by about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to in the absence of the agent.

Embodiment 125. The method of any one of embodiments 89 to 124, wherein the exclusion of the NMD exon from the pre-mRNA in the cell contacted with the agent is increased by about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to in the absence of the agent.

Embodiment 126. The method of any one of embodiments 89 to 125, wherein the agent results in an increase in the level of the processed mRNA in the cell.

Embodiment 127. The method of embodiment 126, wherein the level of the processed mRNA in the cell contacted with the agent is increased by about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to in the absence of the agent.

Embodiment 128. The method of any one of embodiments 89 to 127, wherein the method results in an increase in expression of the target protein in the cell.

Embodiment 129. The method of embodiment 128, wherein a level of the target protein expressed from the processed mRNA in the cell contacted with the agent is increased by about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to in the absence of the agent.

Embodiment 130. The method of any one of embodiments 89 to 128, wherein the target protein is an OPA1 protein.

Embodiment 131. The method of embodiment 130, wherein a level of the OPA1 protein expressed from the processed mRNA in the cell contacted with the agent is increased by at least about 1.5-fold compared to in the absence of the agent.

Embodiment 132. The method of embodiment 130, wherein the OPA1 protein expressed from the processed mRNA is a functional OPA1 protein.

Embodiment 133. The method of embodiment 130, wherein the OPA1 protein expressed from the processed mRNA is at least partially functional as compared to a wild-type OPA1 protein.

Embodiment 134. The method of embodiment 130, wherein the OPA1 protein expressed from the processed mRNA is at least partially functional as compared to a full-length wild-type OPA1 protein.

Embodiment 135. The method of any one of embodiments 89 to 127, wherein the agent comprises an antisense oligomer with at least 80%, at least 90%, or 100% sequence identity to a sequence selected from the group consisting of SEQ ID NOs: 236, 242, 250, 280-283, 288, and 290-292.

Embodiment 136. The method of any one of embodiments 78 to 135, wherein the agent comprises a gene editing molecule.

Embodiment 137. The method of embodiment 136, wherein the gene editing molecule comprises CRISPR-Cas9.

Embodiment 138. The method of any one of embodiments 1 to 75 or 78 to 135, wherein the agent is an antisense oligomer (ASO) and wherein the antisense oligomer comprises a backbone modification comprising a phosphorothioate linkage or a phosphorodiamidate linkage.

Embodiment 139. The method of any one of embodiments 1 to 75 or 78 to 138, wherein the agent is an antisense oligomer (ASO) and wherein the antisense oligomer comprises a phosphorodiamidate morpholino, a locked nucleic acid, a peptide nucleic acid, a 2′-O-methyl moiety, a 2′-Fluoro moiety, or a 2′-O-methoxyethyl moiety.

Embodiment 140. The method of any one of embodiments 1 to 75 or 78 to 139, wherein the therapeutic agent is an antisense oligomer (ASO) and wherein the antisense oligomer comprises at least one modified sugar moiety.

Embodiment 141. The method of embodiment 140, wherein each sugar moiety is a modified sugar moiety.

Embodiment 142. The method of any one of embodiments 1 to 75 or 78 to 141, wherein the agent is an antisense oligomer (ASO) and wherein the antisense oligomer consists of from 8 to 50 nucleobases, 8 to 40 nucleobases, 8 to 35 nucleobases, 8 to 30 nucleobases, 8 to 25 nucleobases, 8 to 20 nucleobases, 8 to 15 nucleobases, 9 to 50 nucleobases, 9 to 40 nucleobases, 9 to 35 nucleobases, 9 to 30 nucleobases, 9 to 25 nucleobases, 9 to 20 nucleobases, 9 to 15 nucleobases, 10 to 50 nucleobases, 10 to 40 nucleobases, 10 to 35 nucleobases, 10 to 30 nucleobases, 10 to 25 nucleobases, 10 to 20 nucleobases, 10 to 15 nucleobases, 11 to 50 nucleobases, 11 to 40 nucleobases, 11 to 35 nucleobases, 11 to 30 nucleobases, 11 to 25 nucleobases, 11 to 20 nucleobases, 11 to 15 nucleobases, 12 to 50 nucleobases, 12 to 40 nucleobases, 12 to 35 nucleobases, 12 to 30 nucleobases, 12 to 25 nucleobases, 12 to 20 nucleobases, or 12 to 15 nucleobases.

Embodiment 143. The method of any one of embodiments 1 to 142, wherein the vector comprises a viral vector encoding the agent.

Embodiment 144. The method of embodiment 143, wherein the viral vector comprises an adenoviral vector, adeno-associated viral (AAV) vector, lentiviral vector, Herpes Simplex Virus (HSV) viral vector, or retroviral vector.

Embodiment 145. The method of any one of embodiments 1 to 144, wherein the method further comprises assessing mRNA level or expression level of the OPA1 protein.

Embodiment 146. The method of any one of embodiments 1 to 145, wherein the agent is a therapeutic agent.

Embodiment 147. A pharmaceutical composition comprising the therapeutic agent of embodiment 146 or a vector encoding the therapeutic agent of embodiment 146, and a pharmaceutically acceptable excipient.

Embodiment 148. A pharmaceutical composition, comprising a therapeutic agent or a vector encoding a therapeutic agent, and a pharmaceutically acceptable excipient, wherein the therapeutic agent comprises an antisense oligomer with at least 80% sequence identity to a sequence selected from the group consisting of SEQ ID NOs: 6-275 and 280-299.

Embodiment 149. The pharmaceutical composition of embodiment 148, wherein the therapeutic agent comprises an antisense oligomer with at least 80%, at least 90%, or 100% sequence identity to a sequence selected from the group consisting of SEQ ID NOs: 227-242 and 250.

Embodiment 150. The pharmaceutical composition of embodiment 148, wherein the therapeutic agent comprises an antisense oligomer with at least 80%, at least 90%, or 100% sequence identity to SEQ ID NO: 267.

Embodiment 151. The pharmaceutical composition of embodiment 148, wherein the therapeutic agent comprises an antisense oligomer with at least 80%, at least 90%, or 100% sequence identity to a sequence selected from the group consisting of SEQ ID NOs: 36, 236, 242, 250, and 280-299.

Embodiment 152. A composition, comprising an antisense oligomer with at least 80% sequence identity to a sequence selected from the group consisting of SEQ ID NOs: 6-275 and 280-299, wherein the antisense oligomer comprises a backbone modification, a sugar moiety modification, or a combination thereof.

Embodiment 153. The composition of embodiment 152, wherein the antisense oligomer has at least 80%, at least 90%, or 100% sequence identity to a sequence selected from the group consisting of SEQ ID NOs: 227-242 and 250.

Embodiment 154. The composition of embodiment 152, wherein the antisense oligomer has at least 80%, at least 90%, or 100% sequence identity to SEQ ID NO: 267.

Embodiment 155. The composition of embodiment 152, wherein the antisense oligomer has at least 80%, at least 90%, or 100% sequence identity to a sequence selected from the group consisting of SEQ ID NOs: 36, 236, 242, 250, and 280-299.

Embodiment 156. A pharmaceutical composition, comprising a therapeutic agent or a vector encoding the therapeutic agent, and a pharmaceutically acceptable excipient, wherein the therapeutic agent promotes exclusion of a coding exon from a pre-mRNA, thereby increasing a level of a processed mRNA that is processed from the pre-mRNA and that lacks the coding exon in a cell, wherein the pre-mRNA is transcribed from an OPA1 gene and that comprises the coding exon.

Embodiment 157. A pharmaceutical composition, comprising a therapeutic agent or a vector encoding the therapeutic agent, and a pharmaceutically acceptable excipient, wherein the therapeutic agent comprises an antisense oligomer that binds to a pre-mRNA that is transcribed from an OPA1 gene in a cell, wherein the antisense oligomer binds to:
(a) a targeted portion of the pre-mRNA within an intronic region immediately upstream of a 5′ end of the coding exon of the pre-mRNA; or
(b) a targeted portion of the pre-mRNA within an intronic region immediately downstream of a 3′ end of the coding exon of the pre-mRNA;
whereby the therapeutic agent increases a level of a processed mRNA that is processed from the pre-mRNA and that contains the coding exon in the cell.

Embodiment 158. A pharmaceutical composition, comprising a therapeutic agent or a vector encoding the therapeutic agent, and a pharmaceutically acceptable excipient, wherein the therapeutic agent promotes exclusion of both a coding exon and a nonsense-mediated RNA decay-inducing exon (NMD exon) from a pre-mRNA, thereby increasing a level of a processed mRNA that is processed from the pre-mRNA and that lacks the coding exon and the NMD exon in a cell, wherein the pre-mRNA is transcribed from an OPA1 gene in the cell and comprises the coding exon and the NMD exon.

Embodiment 159. The pharmaceutical composition of any one of embodiments 147 to 158, wherein the pharmaceutical composition is formulated for intracerebroventricular injection, intraperitoneal injection, intramuscular injection, intrathecal injection, subcutaneous injection, oral administration, synovial injection, intravitreal administration, subretinal injection, topical application, implantation, or intravenous injection.

Embodiment 160. The pharmaceutical composition of any one of embodiments 147 to 158, wherein the pharmaceutical composition is formulated for intravitreal injection.

Embodiment 161. The pharmaceutical composition of any one of embodiments 147 to 160, wherein the pharmaceutical composition further comprises a second therapeutic agent.

Embodiment 162. The pharmaceutical composition of embodiment 161, wherein the second therapeutic agent comprises a small molecule.

Embodiment 163. The pharmaceutical composition of embodiment 161, wherein the second therapeutic agent comprises an antisense oligomer.

Embodiment 164. The pharmaceutical composition of embodiment 161, wherein the second therapeutic agent corrects intron retention.

Embodiment 165. The pharmaceutical composition or composition of any one of embodiments 147 to 160, wherein the antisense oligomer is selected from the group consisting of Compound ID NOs: 1-303.

Embodiment 166. A method of treating or reducing the likelihood of developing a disease or condition in a subject in need thereof by modulating expression of an OPA1 protein in a cell of the subject, comprising contacting to cells of the subject the therapeutic agent of any one of embodiments 147 to 165.

Embodiment 167. The method of embodiment 166, wherein the disease or condition is associated with a loss-of-function mutation in an OPA1 gene.

Embodiment 168. The method of embodiment 166 or 167, wherein the disease or condition is associated with haploinsufficiency of the OPA1 gene, and wherein the subject has a first allele encoding a functional OPA1 protein, and a second allele from which the OPA1 protein is not produced or produced at a reduced level, or a second allele encoding a nonfunctional OPA1 protein or a partially functional OPA1 protein.

Embodiment 169. The method of any one of embodiments 166 to 168, wherein the disease or condition comprises an eye disease or condition.

Embodiment 170. The method of any one of embodiments 166 to 168, wherein the disease or condition comprises ADOA-plus syndrome; a mitochondrial disorder; glaucoma; normal tension glaucoma; Charcot-Marie-Tooth disease; mitochondria dysfunction; diabetic retinopathy; age-related macular degeneration; retinal ganglion cell death; mitochondrial fission-mediated mitochondrial dysfunction; progressive external ophthalmoplegia; deafness; ataxia; motor neuropathy; sensory neuropathy; myopathy; Behr syndrome; brain dysfunction; encephalopathy; peripheral neuropathy; fatal infantile mitochondrial encephalomyopathy; hypertrophic cardiomyopathy; spastic ataxic syndrome; sensory motor peripheral neuropathy; hypotonia; gastrointestinal dysmotility and dysphagia; optic atrophy; optic atrophy plus syndrome; Mitochondrial DNA depletion syndrome 14; late-onset cardiomyopathy; diabetic cardiomyopathy; Alzheimer's Disease; focal segmental glomerulosclerosis; kidney disease; Huntington's Disease; cognitive function decline in healthy aging; Prion diseases; late onset dementia and parkinsonism; mitochondrial myopathy; Leigh syndrome; Friedreich's ataxia; Parkinson's disease; MELAS (Mitochondrial encephalomyopathy, lactic acidosis, and stroke-like episodes); pyruvate dehydrogenase complex deficiency; chronic kidney disease; Leber's hereditary optic neuropathy; obesity; age-related systemic neurodegeneration; skeletal muscle atrophy; heart and brain ischemic damage; massive liver apoptosis; NARP (neuropathy, ataxia, retinitis pigmentosa); MERRF (myoclonic epilepsy and ragged red fibers); Pearsons/Kerns-Sayre syndrome; MIDD (maternally inherited diabetes and deafness); mitochondrial trifunctional protein deficiency; Fuchs corneal endothelial dystrophy; macular telangiectasia; retinitis pigmentosa; Leber congenital amaurosis; inherited maculopathy; Stargardt disease; or Sorsby fundus dystrophy.

Embodiment 171. The method of any one of embodiments 166 to 168, wherein the disease or condition comprises Optic atrophy type 1.

Embodiment 172. The method of any one of embodiments 166 to 168, wherein the disease or condition comprises autosomal dominant optic atrophy (ADOA).

Embodiment 173. The method of embodiment 166 or 167, wherein the disease or condition is associated with an autosomal recessive mutation of OPA1 gene, wherein the subject has a first allele encoding from which:
(i) OPA1 protein is not produced or produced at a reduced level compared to a wild-type allele; or
(ii) the OPA1 protein produced is nonfunctional or partially functional compared to a wild-type allele, and a second allele from which:
(iii) the OPA1 protein is produced at a reduced level compared to a wild-type allele and the OPA1 protein produced is at least partially functional compared to a wild-type allele; or
(iv) the OPA1 protein produced is partially functional compared to a wild-type allele.

Embodiment 174. The method of any one of embodiments 166 to 173, wherein the subject is a human.

Embodiment 175. The method of any one of embodiments 166 to 173, wherein the subject is a non-human animal.

Embodiment 176. The method of any one of embodiments 166 to 173, wherein the subject is a fetus, an embryo, or a child.

Embodiment 177. The method of any one of embodiments 166 to 173, wherein the cells are ex vivo.

Embodiment 178. The method of any one of embodiments 166 to 173, wherein the therapeutic agent is administered by intracerebroventricular injection, intraperitoneal injection, intramuscular injection, intrathecal injection, subcutaneous injection, oral administration, synovial injection, intravitreal administration, subretinal injection, topical application, implantation, or intravenous injection.

Embodiment 179. The method of any one of embodiments 166 to 173, wherein the therapeutic agent is administered by intravitreal injection.

Embodiment 180. The method of any one of embodiments 166 to 179, wherein the method treats the disease or condition.

### EXAMPLES

The present disclosure will be more specifically illustrated by the following Examples. However, it should be understood that the present disclosure is not limited by these examples in any manner.

### Example 1.1: Effects of ASOs on Protein Expression Evaluated by Immunoblotting

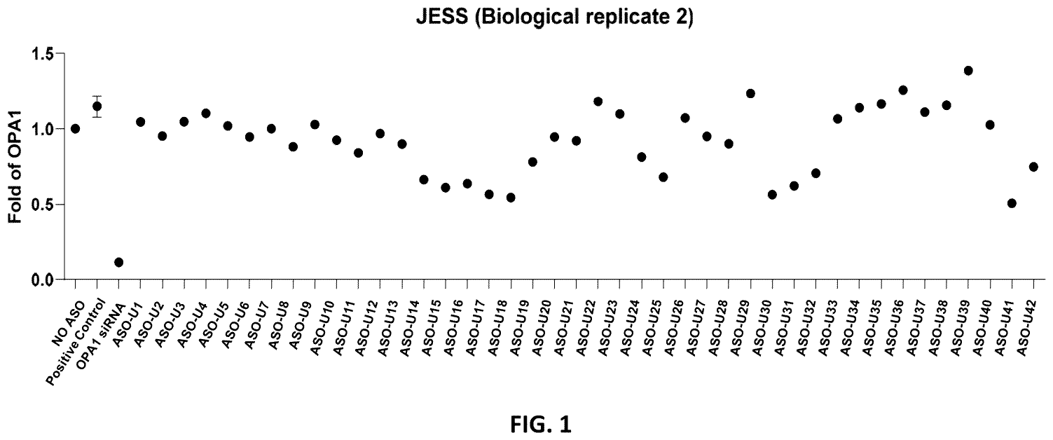

In one experiment, a microwalk was conducted to test exemplary ASOs that have sequences listed in Table 8, which are designed to hybridize to at least a portion of the 5′ UTR of OPA1 processed mRNA transcript. HEK293 cells were transfected with exemplary 2′-MOE ASOs with PS backbone using Lipofectamine RNAiMax (Invitrogen: 13778150). Three days later, total protein lysate was extracted with RIPA buffer from the treated cells and OPA1 protein detection was conducted by Jess chemiluminescent immunoblotting. 0.3 ug of total protein was loaded to the Jess 12-230 kDa cartridge (ProteinSimple: SM-W001) and blotted with anti-OPA1 antibody (Abcam: ab119685) diluted to 1:80. The signal of OPA1 was normalized to total protein in each lane and fold-change was calculated and plotted by normalization to the cells not treated with any of the ASOs (“No-ASO control”). FIG. 1 is a bar graph depicting the fold change of OPA1 protein level in the cells treated with some of the exemplary ASOs: ASO-U1 to ASO-U42, as well as a positive control ASO, a negative control (OPA1 siRNA), and No-ASO control. Among the tested ASOs, at least ASO-U22, ASO-U29, ASO-U33, ASO-U34, ASO-U35, ASO-U36, ASO-U37, ASO-U38, ASO-U39, and ASO-U40 were shown to lead to an increase in OPA protein level in the treated cells as compared to the No-ASO control.

### Example 1.2: Effects of ASOs on Structure of OPA1 Processed mRNA Transcript

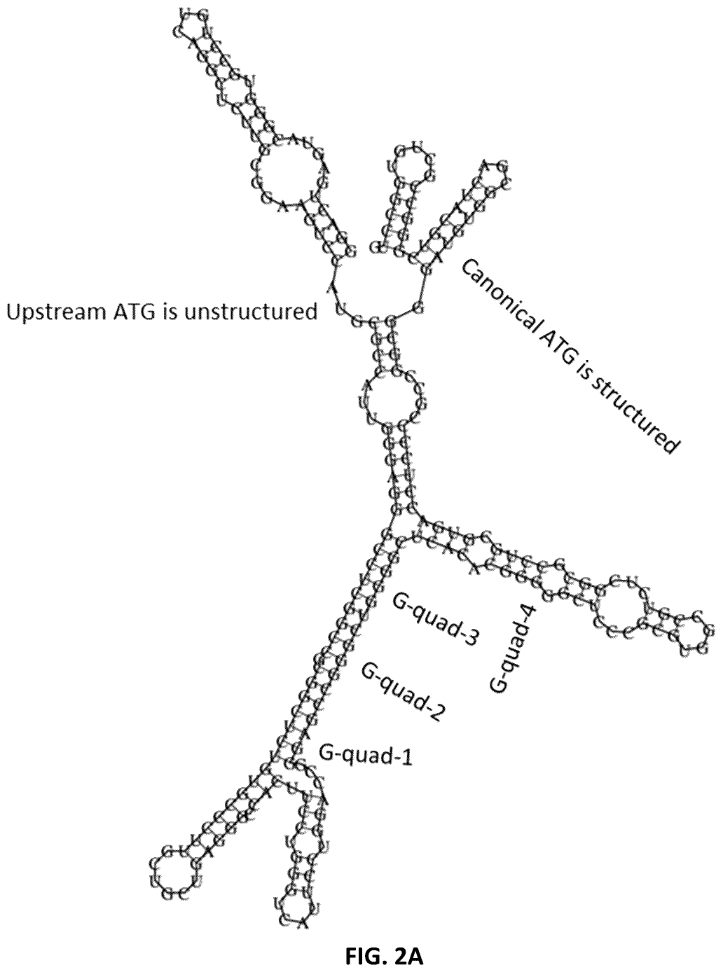

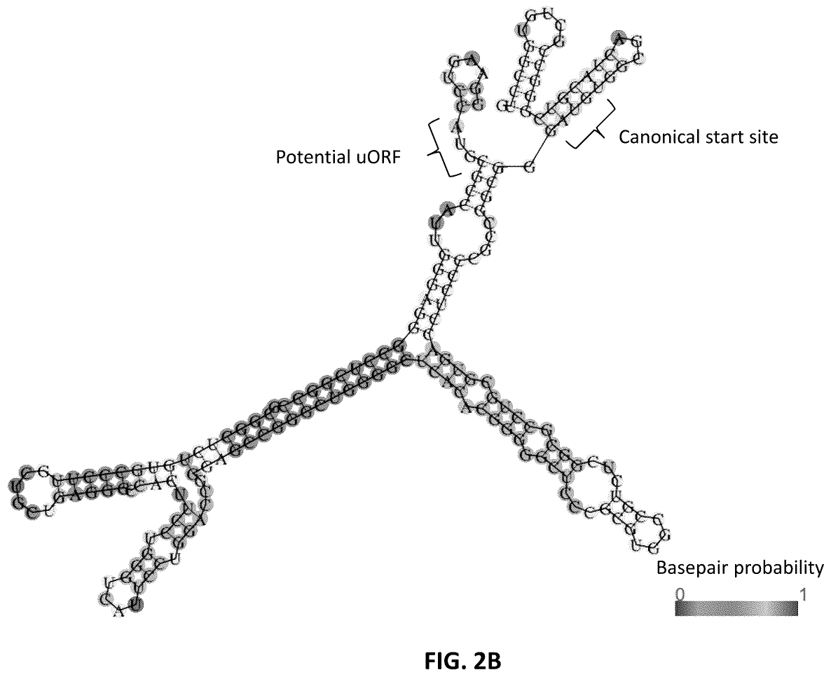

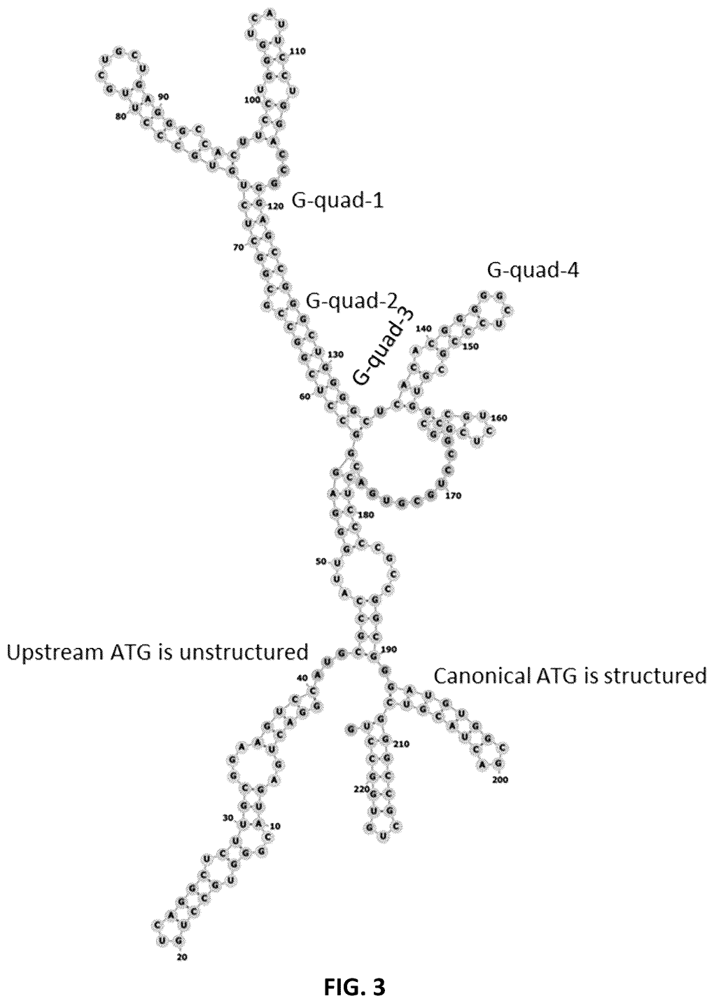

The secondary structure of an exemplary OPA1 processed mRNA transcript can be computationally simulated using available software tools. In one case, the secondary structure of the region covering the 5′ UTR and exon 1 of OPA1 transcript ENST00000361908 is computationally determined as shown in FIG. 2A using RNAFold version 2.4.18 with default parameters. As illustrated in the figure, based on this simulation, the 5′ UTR has a structured canonical ATG (an example of the “main start codon” disclosed herein) and an unstructured upstream ATG (an example of the “upstream start codon” disclosed herein), a G-quad motif, in which all 4 G-repeats are structured. In another case, the secondary structure of the region covering the 5′ UTR and exon 1 of OPA1 transcript ENST00000361908 is computationally determined as shown in FIG. 3 using MXFold2 with default parameters. As illustrated in the figure, based on this simulation, the 5′ UTR also has a structured canonical ATG and an unstructured upstream ATG, a G-quad motif, in which all 4 G-repeats are structured.

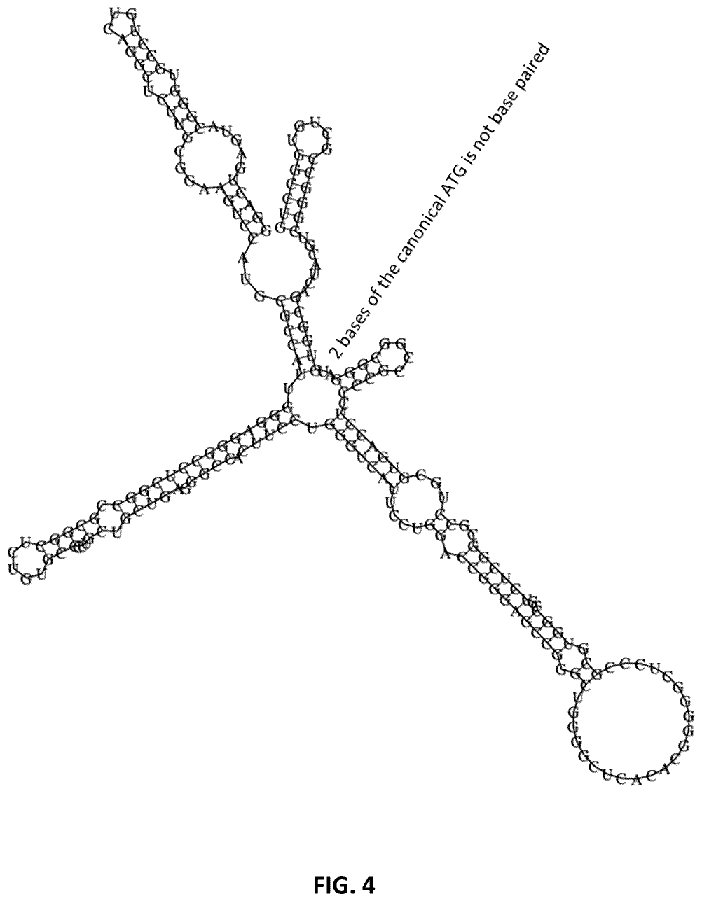

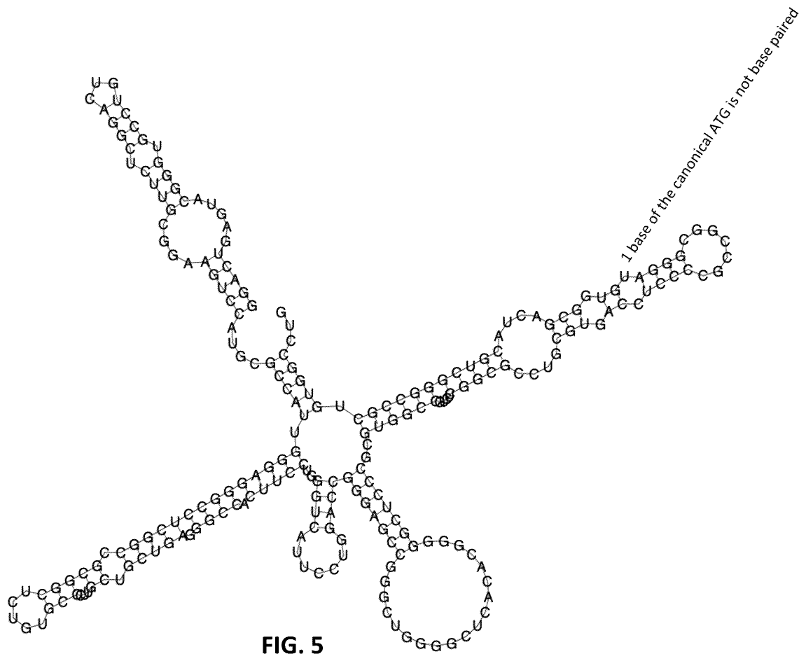

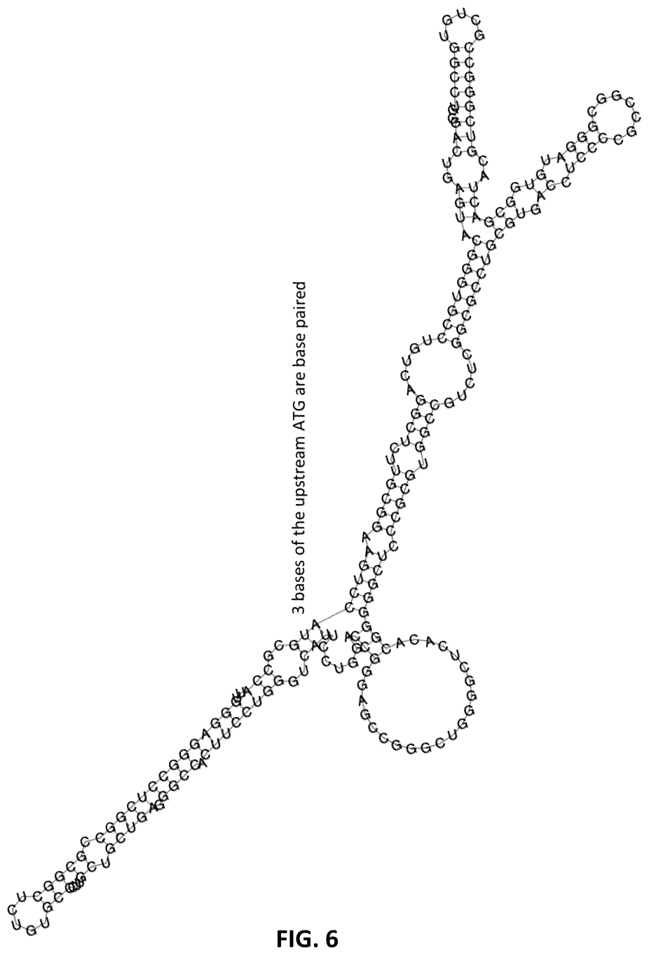

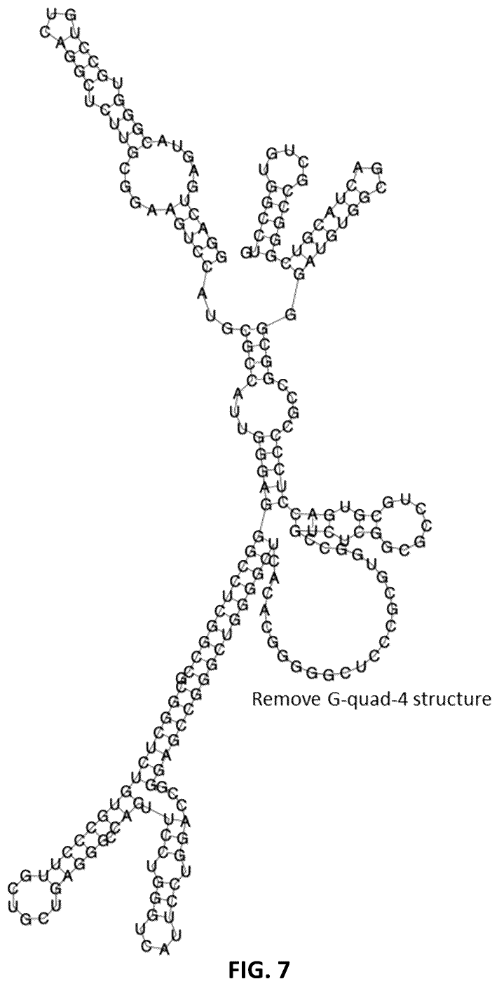

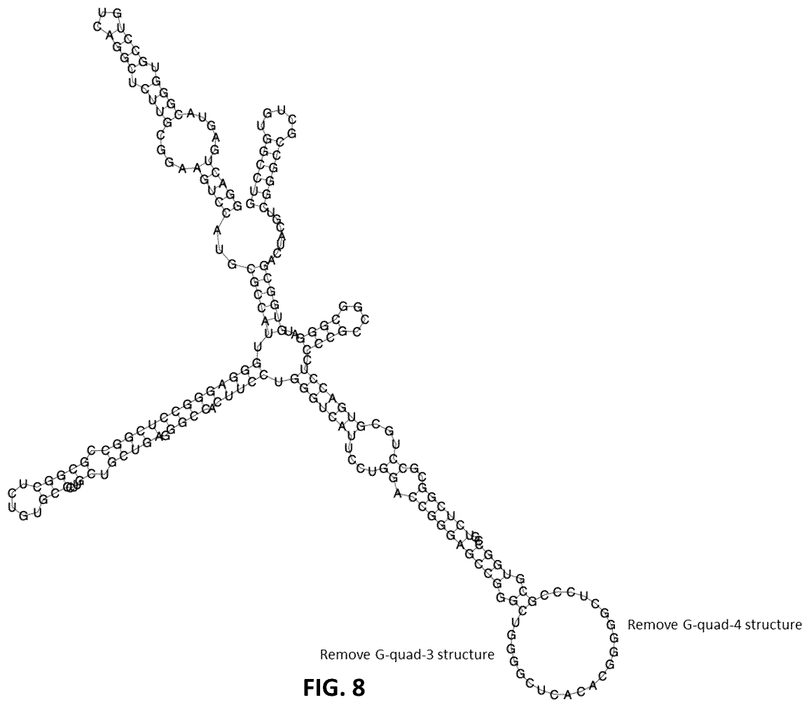

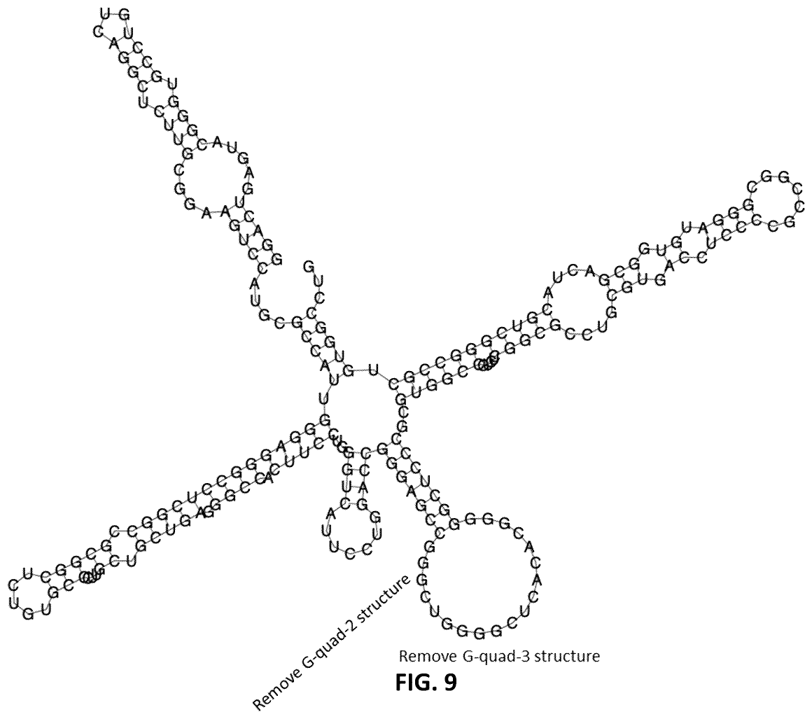

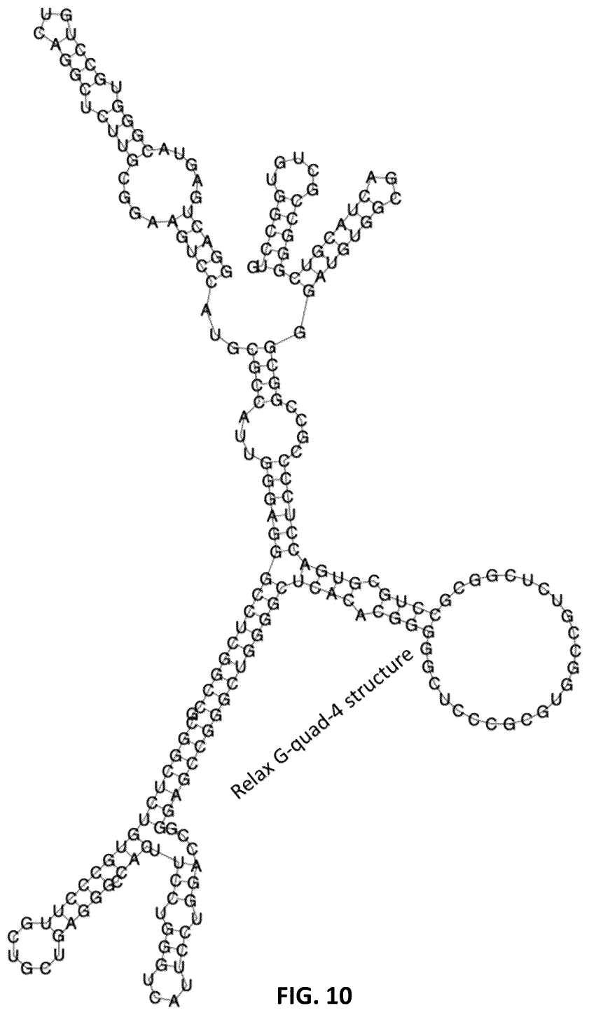

Based on computational structural simulation, various changes in the secondary structure of the mRNA transcript were found to be induced upon binding with some of the exemplary ASOs. The following simulations were conducted using RNAFold version 2.4.18 with default parameters. Based on one computational simulation, ASO-U22 and ASO-U35 were found to relax the structured main start codon (e.g., canonical ATG) from 3 pairing to one pairing (from 3 nucleotides being paired to one nucleotide being paired), while ASO-U33 and ASO-U34 were found to relax the structured main start codon (e.g., canonical ATG) from 3 pairings to 2 pairings. FIGS. 4-5 show the secondary structures of the 5′UTR and main start codon (e.g., canonical ATG) region of the OPA1 processed mRNA upon binding with ASO-U35 and ASO-U34, respectively. Based on another computational simulation, ASO-U33 was found to convert an unstructured upstream start codon (e.g., upstream ATG) to structured in all 3 base pairs, as shown in FIG. 6. Based on another computational simulation, ASO-U36 and ASO-U37 were found to remove the secondary structure one of the structured G-repeats in the G-quad motif (G-quad-4), ASO-U35 was found to remove the secondary structure in two structured G-repeats in the G-quad motif (G-quad-3 and G-quad-4), and ASO-U33 and ASO-U34 were found to remove the secondary structure in two structured G-repeats in the G-quad motif (G-quad-2 and G-quad-3). FIGS. 7-9 show the removal of the secondary structure in the G-repeats of the G-quad motif of the 5′ UTR of the OPA1 processed mRNA upon binding with ASO-U37, ASO-U35, and ASO-U33, respectively. Based on another computational simulation, ASO-U38, ASO-U39, and ASO-U40 were found to relax the secondary structure of one G-repeat in the G-quad motif (G-quad-4) from 4-base pairing to 3-base pairing. FIG. 10 shows the secondary structure of the region covering 5′ UTR and main start codon of the OPA1 processed mRNA upon binding with ASO-U39.

### Example 1.3: OPA1 5′-UTR-Luciferase Assay

This example illustrates the setup of OPA1 5′-UTR-luciferase assay that was used to examine the effect of exemplary agents on the translation of OPA1 mRNA.

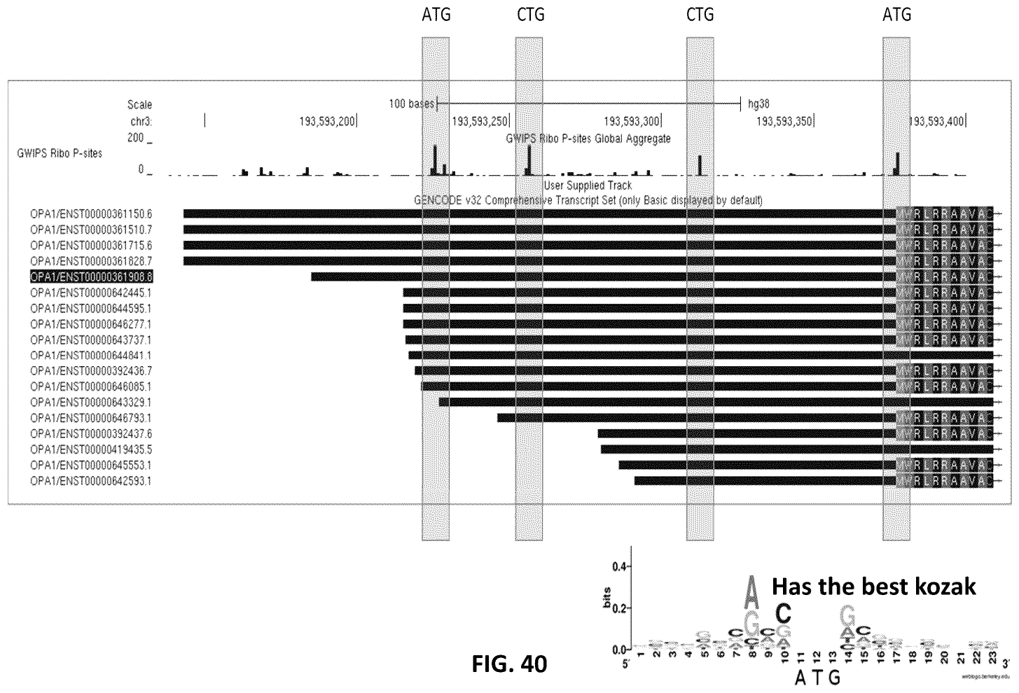

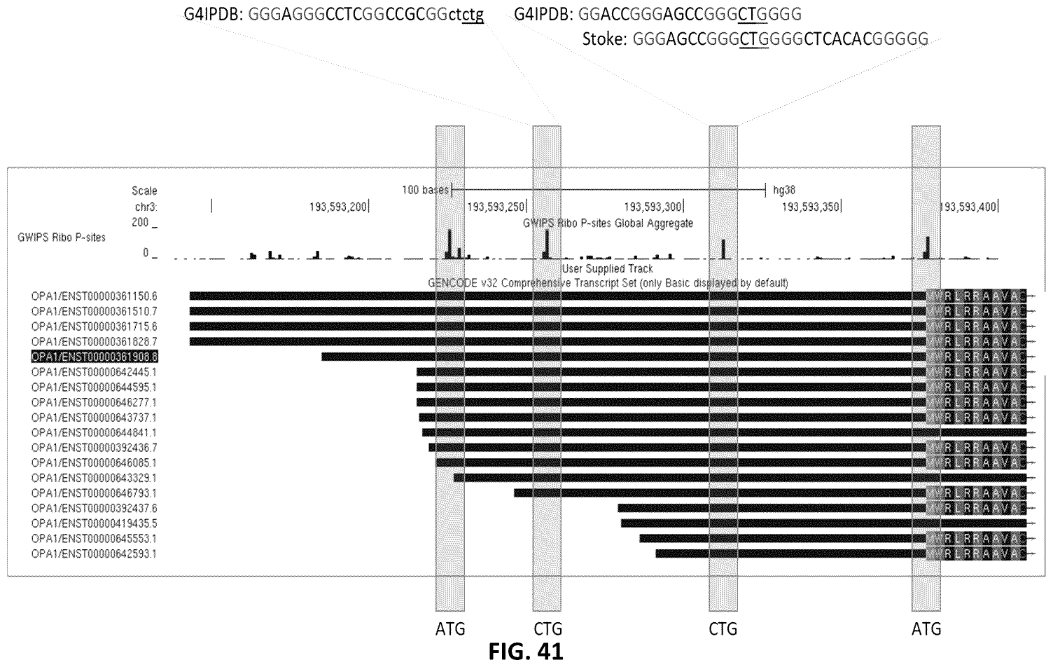

5′-UTR regions of OPA1 processed mRNA transcripts are shown in FIG. 40, with overlaying gray boxes indicating start codons of upstream open reading frames and the main start codon (the rightmost “ATG” shown on the top of the figure).

Two constructs can be used for an OPA1 5′-UTR-luciferase assay: OPA1 5′-UTR-Fluc construct and Rluc construct. The OPA1 5′-UTR-Fluc DNA construct contains, from 5′ end to 3′ end, CMV enhancer, CMV promoter, sequence coding for 5′-UTR of OPA1 mRNA, and coding sequence for Firefly luciferase (Fluc or Luc2P). The Rluc construct contains, from 5′ end to 3′ end, SV40 promoter, chimeric intron, T7 promoter, and coding sequence for Renilla luciferase (Rluc). In this assay, expression of Firefly luciferase can indicate the expression level of Firefly luciferase under the modulation by the OPA1 5′-UTR, while expression of Renilla luciferase can be used as a loading control so that the ratio of Firefly luciferase signal versus Renilla luciferase signal (FL/RL ratio) can indicate the regulation of translation of OPA1 mRNA by its 5′-UTR.

### Example 2.1: Identification of NMD-Inducing Exon Inclusion Events in Transcripts by RNAseq Using Next-Generation Sequencing

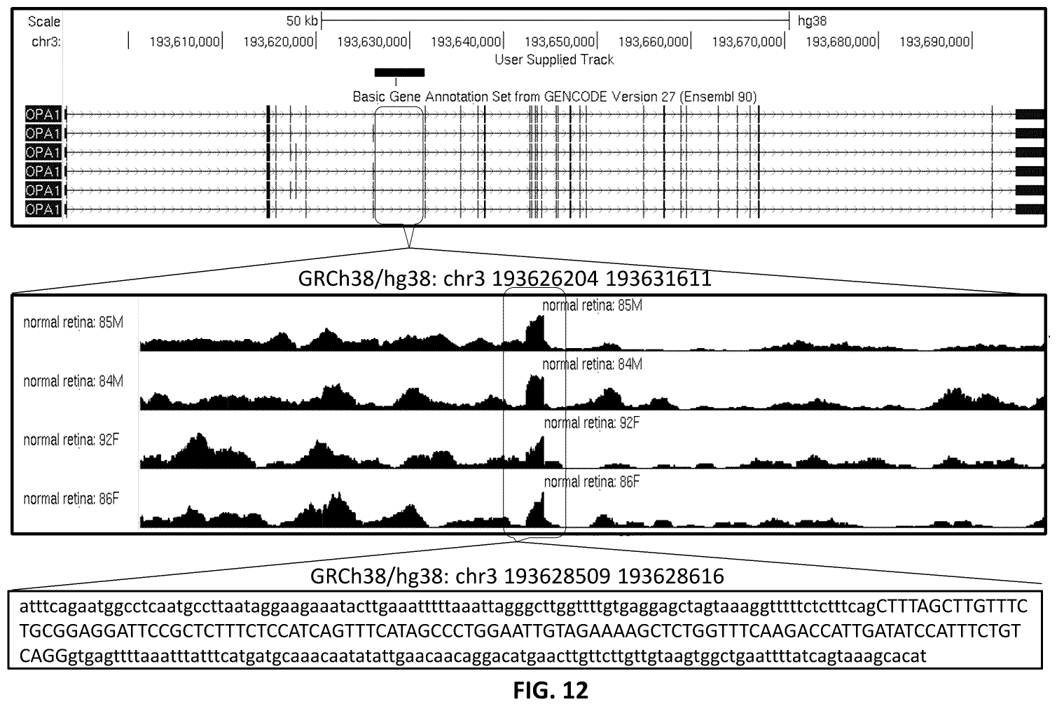

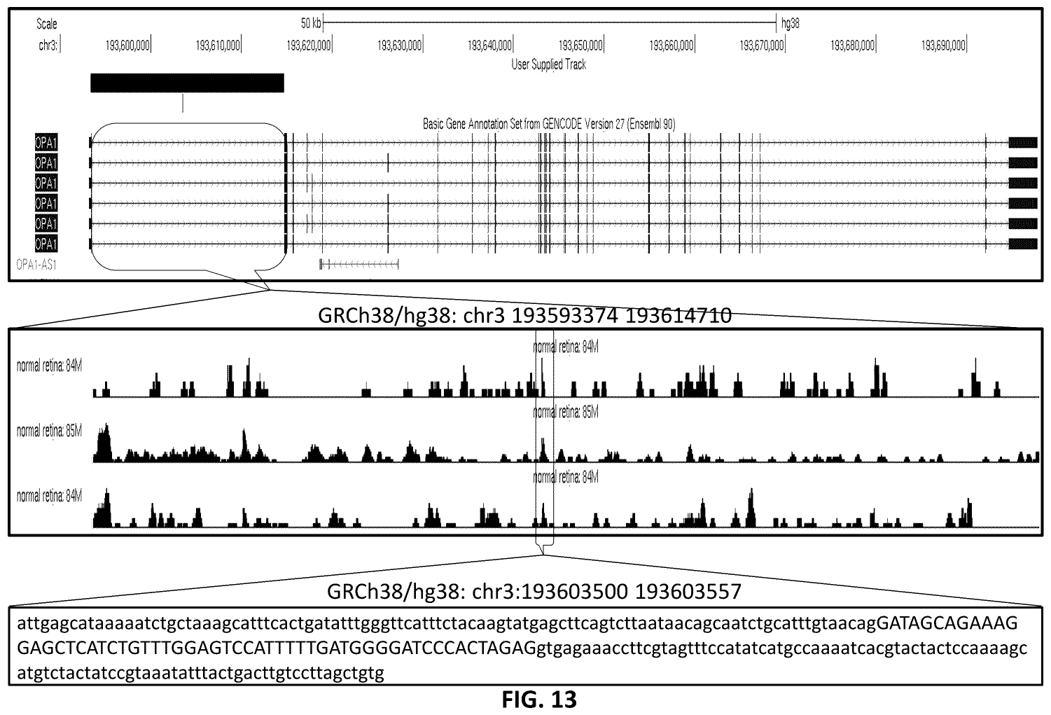

Whole-transcriptome shotgun sequencing is carried out using next-generation sequencing to reveal a snapshot of transcripts produced by the genes described herein to identify NMD exon inclusion events. For this purpose, polyA+RNA from nuclear and cytoplasmic fractions of human cells is isolated and DNA libraries are constructed using Illumina's TruSeq Stranded mRNA library Prep Kit. The libraries are pair-end sequenced resulting in 100-nucleotide reads that are mapped to the human genome (February 2009, GRCh37/hg19 assembly). FIGS. 12 and 13 depict identification of different exemplary nonsense-mediated mRNA decay (NMD)-inducing exons in various genes.

Exemplary genes and intron sequences are summarized in Table 1.1 and Table 1.2 (SEQ ID NOs indicate the corresponding nucleotide sequences represented by the Gene ID Nos). Exemplary mature mRNA sequences and 5′ UTR sequences are summarized in Table 1.3 and Table 1.4. Exemplary target protein sequences are summarized in Table 2. The sequence for each intron is summarized in Table 3 and Table 4. Table 5 lists sequences of OPA1 antisense oligomers of this disclosure.

TABLE 1.1
List of exemplary target gene sequences.

| Gene | Gene | SEQ | | | | |
|---|---|---|---|---|---|---|
| Symbol | ID No. | ID No. | Disease | OMIM | Genetics | Introns |
| OPA1 | 4976 | 1 | Optic atrophy | 165500 | Haploinsufficient | ENST00000361908.7: 6 |
| Organism: | | | type 1; Autosomal | | | ENST00000361908.7: 28 |
| homo | | | dominant optic | | | |
| sapiens | | | atrophy (ADOA) | | | |

TABLE 1.2
List of exemplary target gene sequences.

| Gene | Gene | SEQ | | | | |
|---|---|---|---|---|---|---|
| Symbol | ID No. | ID No. | Disease | OMIM | Genetics | Introns |
| OPA1 | 4976 | 1 | Optic atrophy | 165500 | Haploinsufficient | GRCh38/ |
| Organism: | | | type 1; Autosomal | | | hg38: chr3 |
| homo | | | dominant optic | | | 193626203 |
| sapiens | | | atrophy (ADOA) | | | 193631611 |
| | | | | | | GRCh38/ |
| | | | | | | hg38: chr3 |
| | | | | | | 193593374 |
| | | | | | | 193614710 |

TABLE 1.3
List of Sequences of Exemplary OPA1 Mature mRNA Variants
ENST00000361715.6, SEQ ID NO: 1254, organism: homo sapiens
GTGCTGCCCGCCTAGAAAGGGTGAAGTGGTTGTTTCCGTGACGGACTGAGTACGGGTGCCTGTCAGGCT
CTTGCGGAAGTCCATGCGCCATTGGGAGGGCCTCGGCCGCGGCTCTGTGCCCTTGCTGCTGAGGGCCAC
TTCCTGGGTCATTCCTGGACCGGGAGCCGGGCTGGGGCTCACACGGGGGCTCCCGCGTGGCCGTCTCGG
CGCCTGCGTGACCTCCCCGCCGGCGGGATGTGGCGACTACGTCGGGCCGCTGTGGCCTGTGAGGTCTGC
CAGTCTTTAGTGAAACACAGCTCTGGAATAAAAGGAAGTTTACCACTACAAAAACTACATCTGGTTTCA
CGAAGCATTTATCATTCACATCATCCTACCTTAAAGCTTCAACGACCCCAATTAAGGACATCCTTTCAG
CAGTTCTCTTCTCTGACAAACCTTCCTTTACGTAAACTGAAATTCTCTCCAATTAAATATGGCTACCAG
CCTCGCAGGAATTTTTGGCCAGCAAGATTAGCTACGAGACTCTTAAAACTTCGCTATCTCATACTAGGA
TCGGCTGTTGGGGGTGGCTACACAGCCAAAAAGACTTTTGATCAGTGGAAAGATATGATACCGGACCTT
AGTGAATATAAATGGATTGTGCCTGACATTGTGTGGGAAATTGATGAGTATATCGATTTTGGTCACAAA
TTGGTTAGTGAAGTCATAGGAGCTTCTGACCTACTTCTCTTGTTAGGTTCTCCGGAAGAAACGGCGTTT
AGAGCAACAGATCGTGGATCTGAAAGTGACAAGCATTTTAGAAAGGGTCTGCTTGGTGAGCTCATTCTC
TTACAACAACAAATTCAAGAGCATGAAGAGGAAGCGCGCAGAGCCGCTGGCCAATATAGCACGAGCTAT
GCCCAACAGAAGCGCAAGGTGTCAGACAAAGAGAAAATTGACCAACTTCAGGAAGAACTTCTGCACACT
CAGTTGAAGTATCAGAGAATCTTGGAACGATTAGAAAAGGAGAACAAAGAATTGAGAAAATTAGTATTG
CAGAAAGATGACAAAGGCATTCATCATAGAAAGCTTAAGAAATCTTTGATTGACATGTATTCTGAAGTT
CTTGATGTTCTCTCTGATTATGATGCCAGTTATAATACGCAAGATCATCTGCCACGGGTTGTTGTGGTT
GGAGATCAGAGTGCTGGAAAGACTAGTGTGTTGGAAATGATTGCCCAAGCTCGAATATTCCCAAGAGGA
TCTGGGGAGATGATGACACGTTCTCCAGTTAAGGTGACTCTGAGTGAAGGTCCTCACCATGTGGCCCTA
TTTAAAGATAGTTCTCGGGAGTTTGATCTTACCAAAGAAGAAGATCTTGCAGCATTAAGACATGAAATA
GAACTTCGAATGAGGAAAAATGTGAAAGAAGGCTGTACCGTTAGCCCTGAGACCATATCCTTAAATGTA
AAAGGCCCTGGACTACAGAGGATGGTGCTTGTTGACTTACCAGGTGTGATTAATACTGTGACATCAGGC
ATGGCTCCTGACACAAAGGAAACTATTTTCAGTATCAGCAAAGCTTACATGCAGAATCCTAATGCCATC
ATACTGTGTATTCAAGATGGATCTGTGGATGCTGAACGCAGTATTGTTACAGACTTGGTCAGTCAAATG
GACCCTCATGGAAGGAGAACCATATTCGTTTTGACCAAAGTAGACCTGGCAGAGAAAAATGTAGCCAGT
CCAAGCAGGATTCAGCAGATAATTGAAGGAAAGCTCTTCCCAATGAAAGCTTTAGGTTATTTTGCTGTT
GTAACAGGAAAAGGGAACAGCTCTGAAAGCATTGAAGCTATAAGAGAATATGAAGAAGAGTTTTTTCAG
AATTCAAAGCTCCTAAAGACAAGCATGCTAAAGGCACACCAAGTGACTACAAGAAATTTAAGCCTTGCA
GTATCAGACTGCTTTTGGAAAATGGTACGAGAGTCTGTTGAACAACAGGCTGATAGTTTCAAAGCAACA
CGTTTTAACCTTGAAACTGAATGGAAGAATAACTATCCTCGCCTGCGGGAACTTGACCGGAATGAACTA
TTTGAAAAAGCTAAAAATGAAATCCTTGATGAAGTTATCAGTCTGAGCCAGGTTACACCAAAACATTGG
GAGGAAATCCTTCAACAATCTTTGTGGGAAAGAGTATCAACTCATGTGATTGAAAACATCTACCTTCCA
GCTGCGCAGACCATGAATTCAGGAACTTTTAACACCACAGTGGATATCAAGCTTAAACAGTGGACTGAT
AAACAACTTCCTAATAAAGCAGTAGAGGTTGCTTGGGAGACCCTACAAGAAGAATTTTCCCGCTTTATG
ACAGAACCGAAAGGGAAAGAGCATGATGACATATTTGATAAACTTAAAGAGGCTGTTAAGGAAGAAAGT
ATTAAACGACACAAGTGGAATGACTTTGCGGAGGACAGCTTGAGGGTTATTCAACACAATGCTTTGGAA
GACCGATCCATATCTGATAAACAGCAATGGGATGCAGCTATTTATTTTATGGAAGAGGCTCTGCAGGCT
CGTCTCAAGGATACTGAAAATGCAATTGAAAACATGGTGGGTCCAGACTGGAAAAAGAGGTGGTTATAC
TGGAAGAATCGGACCCAAGAACAGTGTGTTCACAATGAAACCAAGAATGAATTGGAGAAGATGTTGAAA
TGTAATGAGGAGCACCCAGCTTATCTTGCAAGTGATGAAATAACCACAGTCCGGAAGAACCTTGAATCC
CGAGGAGTAGAAGTAGATCCAAGCTTGATTAAGGATACTTGGCATCAAGTTTATAGAAGACATTTTTTA
AAAACAGCTCTAAACCATTGTAACCTTTGTCGAAGAGGTTTTTATTACTACCAAAGGCATTTTGTAGAT
TCTGAGTTGGAATGCAATGATGTGGTCTTGTTTTGGCGTATACAGCGCATGCTTGCTATCACCGCAAAT
ACTTTAAGGCAACAACTTACAAATACTGAAGTTAGGCGATTAGAGAAAAATGTTAAAGAGGTATTGGAA
GATTTTGCTGAAGATGGTGAGAAGAAGATTAAATTGCTTACTGGTAAACGCGTTCAACTGGCGGAAGAC
CTCAAGAAAGTTAGAGAAATTCAAGAAAAACTTGATGCTTTCATTGAAGCTCTTCATCAGGAGAAATAA
ATTAAAATCGTACTCATAATCAGCTCTGCATACATCTGAAGAACAAAAACATCAACGTCTTTTGTCCAG
CCTCTTTTTCTTCTGCTGTTCCACCTTTCTAAACATACAATAAAGTCATGGGATAAAAATAATCGATGT
ATGTTACGGGCGCTTTAACCATCAGCTGCCTCTCGAATGGAAGAACAGTGGTAATGGATTAACATCCTA
TTTTGTTGTACTAAAGTGACAAATCGGAATAATATAATTGGTATGGCCATTAGGTTCAGTCCTTGAAGA
TAAGAAACTTGTTCTCTGTTTGTTGTCTTATTTGTGGTGGCACTCGTTTAATGGATTAACTGAGGTTGC
TCAATGTTCAGTTTCTTTTCCAGAAATACAATGCTAGGTGTTTTGAAATAAAACTTATATAGCAATTGT
TTAAAGTTATCAATTGTATATAAAATCACAGTAGCCTGCTAAATCATTGTATGTGTCTGTAGTATTCTA
TTCCCAGAAACTATTTGACCATGATAATTCAGTTTATATTCACCACATGAAAGAAAAATGGGTAACAGA
AGAACCCTTAAAACAGGTTAATTTGGATTGTAACGTTCAGTGAAAGAAATTTCAACCCTTCATAGCCAG
CGAAGAAATTTGCCTTGGAAGCCAAGTCAGTACCAGCTTACCTATTTGATTCAGTTGCTGTTTTCTCAC
TCTCTATATCCATTTGAAATTGATTTATTTTAGATGTTGTATACTTACGTTAGGCTTTCTGTTAATAGT
GGTTTTTCTCCTGTTGACAGAGCCACCGGATTATGACACAGGATGAGGAAGATTAAGGATAATCAATTG
ACTAATTTCATTTAGAATATTATCAAACATTTCAACTAGGTATCAGAAAAAGGCTTTCTTTCATAAGAC
TATTTTAAATAGAAATTATTTCAACAATTAAAGTAATGTTGACCATCCCCCTCTCAGCTGAATAAAGAA
AAATTTAGTTCAATTTATTGCAATTTAATTACAATACTACCTTCACAACATTTTCATGTGTTTTAAATA
AATATTTTTTAATTGGCTAAAGGACATTCAAGCAAAGAAATGCTTTCTTTACTTAAAATGTCTATCTCA
TTTGCTGCCTTTTCACTAAGCCTTTACTTTGTTAATAAAAGTGTCCATTGTGTGATGTTTTTGATTTTA
CAGTTTGCTAAATCTTATTTTCTTGGAGTTGCTTTTTGGTAACAGCCCCATTGCTACTCCCCATTTTAT
TGTTTTACATCAATGCATGCTTCGTTGTGATCCCTCAAGATGTAACACTTGGTATGCTCGGTTGAGGAT
ATGAAAAAATACTTCCGAAACCAGGAATTCAATGTATGTTTGTTTTATACTGTTTGATAAGAAAAGTAG
GTCCAGCCTTAAGCAGCACAGATGCGCTGGTAGATGCATAGTCAGGAACTTTTTTTATTTCTTTTAGGT
CTAGGGACAGGAGTGAATAGAAAGGGAGGAGAGCTCTATTATGTTCTATACACAGATTAGGAGATGACC
TTACTGGGTACACCCCTCTAACCAGTGCTTACAGGTTAATGCATGTTAATGAATATTTTTGCAGTTGTA
AAGCATAACAATTACAACTACACATCTATTTCTAAAGAATAAAACAGGACCATATTTATTTACTTCTGT
CAACTATAGAAAGAAAGACCTTCAGCTGTATTTCCACAGATTTCTCCCAAGGAAAAGGCTAATATTAGT
CACTACTGTTATCACATCCCTTTGTATAAGTTTTAAAAAGAGATGGAGGGAGATCTTCATTTCTTTGAG
GAGATCAGTATTGTAACGTATGTGAATAGATGATAACAATTAATATTACTAAAAGTCCCACATGAGAGT
CCTGACGCCCTCTCCATGCCCCACAGTAATGTGGCTTCTTTCATGGGTTTTTTTTTCTTCTTTTTAGCT
GATCTCATCCTAAGCATGCTTTATTTTTCCTTGAAAGCTAGGTATTTATCAACTGCAGATGTTATTGAA
AGAAAATAAAATTCAGTCTCAAGAGTAAACCCTGTGTCTTGTGTCTGTAGTTCAAAAGTCAGAAATGAT
TCTAATTTAAACAAAAAGATACTAAATATACAGAAGTTAAATTCGAACTAGCCACAGAATCATTTGTTT
TTATGTCAGAATTTGCAAAGAGTGGAGTGGACAAAGCTCTGTATGGAAGACTGAACAACTGTAAATAGA
TGATATCCAAACTTAATTTGGCTAGGACTTCAATTTTAAAAATCAGTGTACCTAGGCAGTGCACAGCAC
GAAATAAGTGGCCCTTGCAGCTTCCCCGTTTAACCCACTGTGCTATAGTTGCGGGTGGAACAGTCAACC
TTTCTAGTAGTTTATGATATTGCCCTCTTTGTATTCCCATTTTCTACAGTTTTTTCCGCAGACTTCTTT
CTGCAAATTATTCAGCCTCCAAATGCAAATGAATGATATAAAAATAAGTAGGGAACATGGCAGAGAGTG
GTGCTTCCCAGCCTCACAATGTGGGAATTTGACATAGGATGAGAGTCAGAGTATAGGTTTAAAAGATAA
AATCTTTAGTTAATAATTTTGTATTTATTTATTCTAGATGTATGTATCTGAGGAAAGAAATCTGGTATT
TTTGCTTTCCAATAAAGGGGATCAAAGTAATGGTTTTTCTCTCAGTTCTCTAAGCTGGTCTATGTTATA
GCTCTAGCAGTATGGAAATGTGCTTTAAAATATGCTTACCTTTTGAATGATCATGGCTATATGTTGTTG
AGATATTTGAAACTTACCTTGTTTTCACTTGTGCACTGTGAATGAACTTTGTATTATTTTTTTAAAACC
TTCACATTACGTGTAGATATTATTGCAACTTATATTTTGCCTGAGCTTGATCAAAGGTCATTTGTGTAG
ATGAGTAATTAAAAAATATTTAAATCACATTATAATTCTATTATTGGAGAGCATCTTTTAAATTTTTTT
CTGTTTTAACGAGGGAAAGAGAAACCTGTATACCTAGGGTCATTATTTGACCCCATAGTATAACCAGAT
TCATGGTCTAACAAGCTCTCAGTGTGGCTTTTCTCTGAATGCTTGAATTTCACATGCCTTGCATTTCAC
AGTTGTACTCCATGGTCAACCGGTGCTTTTTTTCACATCGTGGTACTTGTCAAAACATTTTGTTATTTT
CCTTGGTAAAATATATAAAAAAGGTTTTCTAATTTC
ENST00000361150.6, SEQ ID NO: 1255, organism: homo sapiens
GTGCTGCCCGCCTAGAAAGGGTGAAGTGGTTGTTTCCGTGACGGACTGAGTACGGGTGCCTGTCAGGCT
CTTGCGGAAGTCCATGCGCCATTGGGAGGGCCTCGGCCGCGGCTCTGTGCCCTTGCTGCTGAGGGCCAC
TTCCTGGGTCATTCCTGGACCGGGAGCCGGGCTGGGGCTCACACGGGGGCTCCCGCGTGGCCGTCTCGG
CGCCTGCGTGACCTCCCCGCCGGCGGGATGTGGCGACTACGTCGGGCCGCTGTGGCCTGTGAGGTCTGC
CAGTCTTTAGTGAAACACAGCTCTGGAATAAAAGGAAGTTTACCACTACAAAAACTACATCTGGTTTCA
CGAAGCATTTATCATTCACATCATCCTACCTTAAAGCTTCAACGACCCCAATTAAGGACATCCTTTCAG
CAGTTCTCTTCTCTGACAAACCTTCCTTTACGTAAACTGAAATTCTCTCCAATTAAATATGGCTACCAG
CCTCGCAGGAATTTTTGGCCAGCAAGATTAGCTACGAGACTCTTAAAACTTCGCTATCTCATACTAGGA
TCGGCTGTTGGGGGTGGCTACACAGCCAAAAAGACTTTTGATCAGTGGAAAGATATGATACCGGACCTT
AGTGAATATAAATGGATTGTGCCTGACATTGTGTGGGAAATTGATGAGTATATCGATTTTGGTTCTCCG
GAAGAAACGGCGTTTAGAGCAACAGATCGTGGATCTGAAAGTGACAAGCATTTTAGAAAGGGTCTGCTT
GGTGAGCTCATTCTCTTACAACAACAAATTCAAGAGCATGAAGAGGAAGCGCGCAGAGCCGCTGGCCAA
TATAGCACGAGCTATGCCCAACAGAAGCGCAAGGTGTCAGACAAAGAGAAAATTGACCAACTTCAGGAA
GAACTTCTGCACACTCAGTTGAAGTATCAGAGAATCTTGGAACGATTAGAAAAGGAGAACAAAGAATTG
AGAAAATTAGTATTGCAGAAAGATGACAAAGGCATTCATCATAGAAAGCTTAAGAAATCTTTGATTGAC
ATGTATTCTGAAGTTCTTGATGTTCTCTCTGATTATGATGCCAGTTATAATACGCAAGATCATCTGCCA
CGGGTTGTTGTGGTTGGAGATCAGAGTGCTGGAAAGACTAGTGTGTTGGAAATGATTGCCCAAGCTCGA
ATATTCCCAAGAGGATCTGGGGAGATGATGACACGTTCTCCAGTTAAGGTGACTCTGAGTGAAGGTCCT
CACCATGTGGCCCTATTTAAAGATAGTTCTCGGGAGTTTGATCTTACCAAAGAAGAAGATCTTGCAGCA
TTAAGACATGAAATAGAACTTCGAATGAGGAAAAATGTGAAAGAAGGCTGTACCGTTAGCCCTGAGACC
ATATCCTTAAATGTAAAAGGCCCTGGACTACAGAGGATGGTGCTTGTTGACTTACCAGGTGTGATTAAT
ACTGTGACATCAGGCATGGCTCCTGACACAAAGGAAACTATTTTCAGTATCAGCAAAGCTTACATGCAG
AATCCTAATGCCATCATACTGTGTATTCAAGATGGATCTGTGGATGCTGAACGCAGTATTGTTACAGAC
TTGGTCAGTCAAATGGACCCTCATGGAAGGAGAACCATATTCGTTTTGACCAAAGTAGACCTGGCAGAG
AAAAATGTAGCCAGTCCAAGCAGGATTCAGCAGATAATTGAAGGAAAGCTCTTCCCAATGAAAGCTTTA
GGTTATTTTGCTGTTGTAACAGGAAAAGGGAACAGCTCTGAAAGCATTGAAGCTATAAGAGAATATGAA
GAAGAGTTTTTTCAGAATTCAAAGCTCCTAAAGACAAGCATGCTAAAGGCACACCAAGTGACTACAAGA
AATTTAAGCCTTGCAGTATCAGACTGCTTTTGGAAAATGGTACGAGAGTCTGTTGAACAACAGGCTGAT
AGTTTCAAAGCAACACGTTTTAACCTTGAAACTGAATGGAAGAATAACTATCCTCGCCTGCGGGAACTT
GACCGGAATGAACTATTTGAAAAAGCTAAAAATGAAATCCTTGATGAAGTTATCAGTCTGAGCCAGGTT
ACACCAAAACATTGGGAGGAAATCCTTCAACAATCTTTGTGGGAAAGAGTATCAACTCATGTGATTGAA
AACATCTACCTTCCAGCTGCGCAGACCATGAATTCAGGAACTTTTAACACCACAGTGGATATCAAGCTT
AAACAGTGGACTGATAAACAACTTCCTAATAAAGCAGTAGAGGTTGCTTGGGAGACCCTACAAGAAGAA
TTTTCCCGCTTTATGACAGAACCGAAAGGGAAAGAGCATGATGACATATTTGATAAACTTAAAGAGGCT
GTTAAGGAAGAAAGTATTAAACGACACAAGTGGAATGACTTTGCGGAGGACAGCTTGAGGGTTATTCAA
CACAATGCTTTGGAAGACCGATCCATATCTGATAAACAGCAATGGGATGCAGCTATTTATTTTATGGAA
GAGGCTCTGCAGGCTCGTCTCAAGGATACTGAAAATGCAATTGAAAACATGGTGGGTCCAGACTGGAAA
AAGAGGTGGTTATACTGGAAGAATCGGACCCAAGAACAGTGTGTTCACAATGAAACCAAGAATGAATTG
GAGAAGATGTTGAAATGTAATGAGGAGCACCCAGCTTATCTTGCAAGTGATGAAATAACCACAGTCCGG
AAGAACCTTGAATCCCGAGGAGTAGAAGTAGATCCAAGCTTGATTAAGGATACTTGGCATCAAGTTTAT
AGAAGACATTTTTTAAAAACAGCTCTAAACCATTGTAACCTTTGTCGAAGAGGTTTTTATTACTACCAA
AGGCATTTTGTAGATTCTGAGTTGGAATGCAATGATGTGGTCTTGTTTTGGCGTATACAGCGCATGCTT
GCTATCACCGCAAATACTTTAAGGCAACAACTTACAAATACTGAAGTTAGGCGATTAGAGAAAAATGTT
AAAGAGGTATTGGAAGATTTTGCTGAAGATGGTGAGAAGAAGATTAAATTGCTTACTGGTAAACGCGTT
CAACTGGCGGAAGACCTCAAGAAAGTTAGAGAAATTCAAGAAAAACTTGATGCTTTCATTGAAGCTCTT
CATCAGGAGAAATAAATTAAAATCGTACTCATAATCAGCTCTGCATACATCTGAAGAACAAAAACATCA
ACGTCTTTTGTCCAGCCTCTTTTTCTTCTGCTGTTCCACCTTTCTAAACATACAATAAAGTCATGGGAT
AAAAATAATCGATGTATGTTACGGGCGCTTTAACCATCAGCTGCCTCTCGAATGGAAGAACAGTGGTAA
TGGATTAACATCCTATTTTGTTGTACTAAAGTGACAAATCGGAATAATATAATTGGTATGGCCATTAGG
TTCAGTCCTTGAAGATAAGAAACTTGTTCTCTGTTTGTTGTCTTATTTGTGGTGGCACTCGTTTAATGG
ATTAACTGAGGTTGCTCAATGTTCAGTTTCTTTTCCAGAAATACAATGCTAGGTGTTTTGAAATAAAAC
TTATATAGCAATTGTTTAAAGTTATCAATTGTATATAAAATCACAGTAGCCTGCTAAATCATTGTATGT
GTCTGTAGTATTCTATTCCCAGAAACTATTTGACCATGATAATTCAGTTTATATTCACCACATGAAAGA
AAAATGGGTAACAGAAGAACCCTTAAAACAGGTTAATTTGGATTGTAACGTTCAGTGAAAGAAATTTCA
ACCCTTCATAGCCAGCGAAGAAATTTGCCTTGGAAGCCAAGTCAGTACCAGCTTACCTATTTGATTCAG
TTGCTGTTTTCTCACTCTCTATATCCATTTGAAATTGATTTATTTTAGATGTTGTATACTTACGTTAGG
CTTTCTGTTAATAGTGGTTTTTCTCCTGTTGACAGAGCCACCGGATTATGACACAGGATGAGGAAGATT
AAGGATAATCAATTGACTAATTTCATTTAGAATATTATCAAACATTTCAACTAGGTATCAGAAAAAGGC
TTTCTTTCATAAGACTATTTTAAATAGAAATTATTTCAACAATTAAAGTAATGTTGACCATCCCCCTCT
CAGCTGAATAAAGAAAAATTTAGTTCAATTTATTGCAATTTAATTACAATACTACCTTCACAACATTTT
CATGTGTTTTAAATAAATATTTTTTAATTGGCTAAAGGACATTCAAGCAAAGAAATGCTTTCTTTACTT
AAAATGTCTATCTCATTTGCTGCCTTTTCACTAAGCCTTTACTTTGTTAATAAAAGTGTCCATTGTGTG
ATGTTTTTGATTTTACAGTTTGCTAAATCTTATTTTCTTGGAGTTGCTTTTTGGTAACAGCCCCATTGC
TACTCCCCATTTTATTGTTTTACATCAATGCATGCTTCGTTGTGATCCCTCAAGATGTAACACTTGGTA
TGCTCGGTTGAGGATATGAAAAAATACTTCCGAAACCAGGAATTCAATGTATGTTTGTTTTATACTGTT
TGATAAGAAAAGTAGGTCCAGCCTTAAGCAGCACAGATGCGCTGGTAGATGCATAGTCAGGAACTTTTT
TTATTTCTTTTAGGTCTAGGGACAGGAGTGAATAGAAAGGGAGGAGAGCTCTATTATGTTCTATACACA
GATTAGGAGATGACCTTACTGGGTACACCCCTCTAACCAGTGCTTACAGGTTAATGCATGTTAATGAAT
ATTTTTGCAGTTGTAAAGCATAACAATTACAACTACACATCTATTTCTAAAGAATAAAACAGGACCATA
TTTATTTACTTCTGTCAACTATAGAAAGAAAGACCTTCAGCTGTATTTCCACAGATTTCTCCCAAGGAA
AAGGCTAATATTAGTCACTACTGTTATCACATCCCTTTGTATAAGTTTTAAAAAGAGATGGAGGGAGAT
CTTCATTTCTTTGAGGAGATCAGTATTGTAACGTATGTGAATAGATGATAACAATTAATATTACTAAAA
GTCCCACATGAGAGTCCTGACGCCCTCTCCATGCCCCACAGTAATGTGGCTTCTTTCATGGGTTTTTTT
TTCTTCTTTTTAGCTGATCTCATCCTAAGCATGCTTTATTTTTCCTTGAAAGCTAGGTATTTATCAACT
GCAGATGTTATTGAAAGAAAATAAAATTCAGTCTCAAGAGTAAACCCTGTGTCTTGTGTCTGTAGTTCA
AAAGTCAGAAATGATTCTAATTTAAACAAAAAGATACTAAATATACAGAAGTTAAATTCGAACTAGCCA
CAGAATCATTTGTTTTTATGTCAGAATTTGCAAAGAGTGGAGTGGACAAAGCTCTGTATGGAAGACTGA
ACAACTGTAAATAGATGATATCCAAACTTAATTTGGCTAGGACTTCAATTTTAAAAATCAGTGTACCTA
GGCAGTGCACAGCACGAAATAAGTGGCCCTTGCAGCTTCCCCGTTTAACCCACTGTGCTATAGTTGCGG
GTGGAACAGTCAACCTTTCTAGTAGTTTATGATATTGCCCTCTTTGTATTCCCATTTTCTACAGTTTTT
TCCGCAGACTTCTTTCTGCAAATTATTCAGCCTCCAAATGCAAATGAATGATATAAAAATAAGTAGGGA
ACATGGCAGAGAGTGGTGCTTCCCAGCCTCACAATGTGGGAATTTGACATAGGATGAGAGTCAGAGTAT
AGGTTTAAAAGATAAAATCTTTAGTTAATAATTTTGTATTTATTTATTCTAGATGTATGTATCTGAGGA
AAGAAATCTGGTATTTTTGCTTTCCAATAAAGGGGATCAAAGTAATGGTTTTTCTCTCAGTTCTCTAAG
CTGGTCTATGTTATAGCTCTAGCAGTATGGAAATGTGCTTTAAAATATGCTTACCTTTTGAATGATCAT
GGCTATATGTTGTTGAGATATTTGAAACTTACCTTGTTTTCACTTGTGCACTGTGAATGAACTTTGTAT
TATTTTTTTAAAACCTTCACATTACGTGTAGATATTATTGCAACTTATATTTTGCCTGAGCTTGATCAA
AGGTCATTTGTGTAGATGAGTAATTAAAAAATATTTAAATCACATTATAATTCTATTATTGGAGAGCAT
CTTTTAAATTTTTTTCTGTTTTAACGAGGGAAAGAGAAACCTGTATACCTAGGGTCATTATTTGACCCC
ATAGTATAACCAGATTCATGGTCTAACAAGCTCTCAGTGTGGCTTTTCTCTGAATGCTTGAATTTCACA
TGCCTTGCATTTCACAGTTGTACTCCATGGTCAACCGGTGCTTTTTTTCACATCGTGGTACTTGTCAAA
ACATTTTGTTATTTTCCTTGGTAAAATATATAAAAAAGGTTTTCTAATTTC
ENST00000361828.7, SEQ ID NO: 1256, organism: homo sapiens
GTGCTGCCCGCCTAGAAAGGGTGAAGTGGTTGTTTCCGTGACGGACTGAGTACGGGTGCCTGTCAGGCT
CTTGCGGAAGTCCATGCGCCATTGGGAGGGCCTCGGCCGCGGCTCTGTGCCCTTGCTGCTGAGGGCCAC
TTCCTGGGTCATTCCTGGACCGGGAGCCGGGCTGGGGCTCACACGGGGGCTCCCGCGTGGCCGTCTCGG
CGCCTGCGTGACCTCCCCGCCGGCGGGATGTGGCGACTACGTCGGGCCGCTGTGGCCTGTGAGGTCTGC
CAGTCTTTAGTGAAACACAGCTCTGGAATAAAAGGAAGTTTACCACTACAAAAACTACATCTGGTTTCA
CGAAGCATTTATCATTCACATCATCCTACCTTAAAGCTTCAACGACCCCAATTAAGGACATCCTTTCAG
CAGTTCTCTTCTCTGACAAACCTTCCTTTACGTAAACTGAAATTCTCTCCAATTAAATATGGCTACCAG
CCTCGCAGGAATTTTTGGCCAGCAAGATTAGCTACGAGACTCTTAAAACTTCGCTATCTCATACTAGGA
TCGGCTGTTGGGGGTGGCTACACAGCCAAAAAGACTTTTGATCAGTGGAAAGATATGATACCGGACCTT
AGTGAATATAAATGGATTGTGCCTGACATTGTGTGGGAAATTGATGAGTATATCGATTTTGAGAAAATT
AGAAAAGCCCTTCCTAGTTCAGAAGACCTTGTAAAGTTAGCACCAGACTTTGACAAGATTGTTGAAAGC
CTTAGCTTATTGAAGGACTTTTTTACCTCAGGTTCTCCGGAAGAAACGGCGTTTAGAGCAACAGATCGT
GGATCTGAAAGTGACAAGCATTTTAGAAAGGTGTCAGACAAAGAGAAAATTGACCAACTTCAGGAAGAA
CTTCTGCACACTCAGTTGAAGTATCAGAGAATCTTGGAACGATTAGAAAAGGAGAACAAAGAATTGAGA
AAATTAGTATTGCAGAAAGATGACAAAGGCATTCATCATAGAAAGCTTAAGAAATCTTTGATTGACATG
TATTCTGAAGTTCTTGATGTTCTCTCTGATTATGATGCCAGTTATAATACGCAAGATCATCTGCCACGG
GTTGTTGTGGTTGGAGATCAGAGTGCTGGAAAGACTAGTGTGTTGGAAATGATTGCCCAAGCTCGAATA
TTCCCAAGAGGATCTGGGGAGATGATGACACGTTCTCCAGTTAAGGTGACTCTGAGTGAAGGTCCTCAC
CATGTGGCCCTATTTAAAGATAGTTCTCGGGAGTTTGATCTTACCAAAGAAGAAGATCTTGCAGCATTA
AGACATGAAATAGAACTTCGAATGAGGAAAAATGTGAAAGAAGGCTGTACCGTTAGCCCTGAGACCATA
TCCTTAAATGTAAAAGGCCCTGGACTACAGAGGATGGTGCTTGTTGACTTACCAGGTGTGATTAATACT
GTGACATCAGGCATGGCTCCTGACACAAAGGAAACTATTTTCAGTATCAGCAAAGCTTACATGCAGAAT
CCTAATGCCATCATACTGTGTATTCAAGATGGATCTGTGGATGCTGAACGCAGTATTGTTACAGACTTG
GTCAGTCAAATGGACCCTCATGGAAGGAGAACCATATTCGTTTTGACCAAAGTAGACCTGGCAGAGAAA
AATGTAGCCAGTCCAAGCAGGATTCAGCAGATAATTGAAGGAAAGCTCTTCCCAATGAAAGCTTTAGGT
TATTTTGCTGTTGTAACAGGAAAAGGGAACAGCTCTGAAAGCATTGAAGCTATAAGAGAATATGAAGAA
GAGTTTTTTCAGAATTCAAAGCTCCTAAAGACAAGCATGCTAAAGGCACACCAAGTGACTACAAGAAAT
TTAAGCCTTGCAGTATCAGACTGCTTTTGGAAAATGGTACGAGAGTCTGTTGAACAACAGGCTGATAGT
TTCAAAGCAACACGTTTTAACCTTGAAACTGAATGGAAGAATAACTATCCTCGCCTGCGGGAACTTGAC
CGGAATGAACTATTTGAAAAAGCTAAAAATGAAATCCTTGATGAAGTTATCAGTCTGAGCCAGGTTACA
CCAAAACATTGGGAGGAAATCCTTCAACAATCTTTGTGGGAAAGAGTATCAACTCATGTGATTGAAAAC
ATCTACCTTCCAGCTGCGCAGACCATGAATTCAGGAACTTTTAACACCACAGTGGATATCAAGCTTAAA
CAGTGGACTGATAAACAACTTCCTAATAAAGCAGTAGAGGTTGCTTGGGAGACCCTACAAGAAGAATTT
TCCCGCTTTATGACAGAACCGAAAGGGAAAGAGCATGATGACATATTTGATAAACTTAAAGAGGCTGTT
AAGGAAGAAAGTATTAAACGACACAAGTGGAATGACTTTGCGGAGGACAGCTTGAGGGTTATTCAACAC
AATGCTTTGGAAGACCGATCCATATCTGATAAACAGCAATGGGATGCAGCTATTTATTTTATGGAAGAG
GCTCTGCAGGCTCGTCTCAAGGATACTGAAAATGCAATTGAAAACATGGTGGGTCCAGACTGGAAAAAG
AGGTGGTTATACTGGAAGAATCGGACCCAAGAACAGTGTGTTCACAATGAAACCAAGAATGAATTGGAG
AAGATGTTGAAATGTAATGAGGAGCACCCAGCTTATCTTGCAAGTGATGAAATAACCACAGTCCGGAAG
AACCTTGAATCCCGAGGAGTAGAAGTAGATCCAAGCTTGATTAAGGATACTTGGCATCAAGTTTATAGA
AGACATTTTTTAAAAACAGCTCTAAACCATTGTAACCTTTGTCGAAGAGGTTTTTATTACTACCAAAGG
CATTTTGTAGATTCTGAGTTGGAATGCAATGATGTGGTCTTGTTTTGGCGTATACAGCGCATGCTTGCT
ATCACCGCAAATACTTTAAGGCAACAACTTACAAATACTGAAGTTAGGCGATTAGAGAAAAATGTTAAA
GAGGTATTGGAAGATTTTGCTGAAGATGGTGAGAAGAAGATTAAATTGCTTACTGGTAAACGCGTTCAA
CTGGCGGAAGACCTCAAGAAAGTTAGAGAAATTCAAGAAAAACTTGATGCTTTCATTGAAGCTCTTCAT
CAGGAGAAATAAATTAAAATCGTACTCATAATCAGCTCTGCATACATCTGAAGAACAAAAACATCAACG
TCTTTTGTCCAGCCTCTTTTTCTTCTGCTGTTCCACCTTTCTAAACATACAATAAAGTCATGGGATAAA
AATAATCGATGTATGTTACGGGCGCTTTAACCATCAGCTGCCTCTCGAATGGAAGAACAGTGGTAATGG
ATTAACATCCTATTTTGTTGTACTAAAGTGACAAATCGGAATAATATAATTGGTATGGCCATTAGGTTC
AGTCCTTGAAGATAAGAAACTTGTTCTCTGTTTGTTGTCTTATTTGTGGTGGCACTCGTTTAATGGATT
AACTGAGGTTGCTCAATGTTCAGTTTCTTTTCCAGAAATACAATGCTAGGTGTTTTGAAATAAAACTTA
TATAGCAATTGTTTAAAGTTATCAATTGTATATAAAATCACAGTAGCCTGCTAAATCATTGTATGTGTC
TGTAGTATTCTATTCCCAGAAACTATTTGACCATGATAATTCAGTTTATATTCACCACATGAAAGAAAA
ATGGGTAACAGAAGAACCCTTAAAACAGGTTAATTTGGATTGTAACGTTCAGTGAAAGAAATTTCAACC
CTTCATAGCCAGCGAAGAAATTTGCCTTGGAAGCCAAGTCAGTACCAGCTTACCTATTTGATTCAGTTG
CTGTTTTCTCACTCTCTATATCCATTTGAAATTGATTTATTTTAGATGTTGTATACTTACGTTAGGCTT
TCTGTTAATAGTGGTTTTTCTCCTGTTGACAGAGCCACCGGATTATGACACAGGATGAGGAAGATTAAG
GATAATCAATTGACTAATTTCATTTAGAATATTATCAAACATTTCAACTAGGTATCAGAAAAAGGCTTT
CTTTCATAAGACTATTTTAAATAGAAATTATTTCAACAATTAAAGTAATGTTGACCATCCCCCTCTCAG
CTGAATAAAGAAAAATTTAGTTCAATTTATTGCAATTTAATTACAATACTACCTTCACAACATTTTCAT
GTGTTTTAAATAAATATTTTTTAATTGGCTAAAGGACATTCAAGCAAAGAAATGCTTTCTTTACTTAAA
ATGTCTATCTCATTTGCTGCCTTTTCACTAAGCCTTTACTTTGTTAATAAAAGTGTCCATTGTGTGATG
TTTTTGATTTTACAGTTTGCTAAATCTTATTTTCTTGGAGTTGCTTTTTGGTAACAGCCCCATTGCTAC
TCCCCATTTTATTGTTTTACATCAATGCATGCTTCGTTGTGATCCCTCAAGATGTAACACTTGGTATGC
TCGGTTGAGGATATGAAAAAATACTTCCGAAACCAGGAATTCAATGTATGTTTGTTTTATACTGTTTGA
TAAGAAAAGTAGGTCCAGCCTTAAGCAGCACAGATGCGCTGGTAGATGCATAGTCAGGAACTTTTTTTA
TTTCTTTTAGGTCTAGGGACAGGAGTGAATAGAAAGGGAGGAGAGCTCTATTATGTTCTATACACAGAT
TAGGAGATGACCTTACTGGGTACACCCCTCTAACCAGTGCTTACAGGTTAATGCATGTTAATGAATATT
TTTGCAGTTGTAAAGCATAACAATTACAACTACACATCTATTTCTAAAGAATAAAACAGGACCATATTT
ATTTACTTCTGTCAACTATAGAAAGAAAGACCTTCAGCTGTATTTCCACAGATTTCTCCCAAGGAAAAG
GCTAATATTAGTCACTACTGTTATCACATCCCTTTGTATAAGTTTTAAAAAGAGATGGAGGGAGATCTT
CATTTCTTTGAGGAGATCAGTATTGTAACGTATGTGAATAGATGATAACAATTAATATTACTAAAAGTC
CCACATGAGAGTCCTGACGCCCTCTCCATGCCCCACAGTAATGTGGCTTCTTTCATGGGTTTTTTTTTC
TTCTTTTTAGCTGATCTCATCCTAAGCATGCTTTATTTTTCCTTGAAAGCTAGGTATTTATCAACTGCA
GATGTTATTGAAAGAAAATAAAATTCAGTCTCAAGAGTAAACCCTGTGTCTTGTGTCTGTAGTTCAAAA
GTCAGAAATGATTCTAATTTAAACAAAAAGATACTAAATATACAGAAGTTAAATTCGAACTAGCCACAG
AATCATTTGTTTTTATGTCAGAATTTGCAAAGAGTGGAGTGGACAAAGCTCTGTATGGAAGACTGAACA
ACTGTAAATAGATGATATCCAAACTTAATTTGGCTAGGACTTCAATTTTAAAAATCAGTGTACCTAGGC
AGTGCACAGCACGAAATAAGTGGCCCTTGCAGCTTCCCCGTTTAACCCACTGTGCTATAGTTGCGGGTG
GAACAGTCAACCTTTCTAGTAGTTTATGATATTGCCCTCTTTGTATTCCCATTTTCTACAGTTTTTTCC
GCAGACTTCTTTCTGCAAATTATTCAGCCTCCAAATGCAAATGAATGATATAAAAATAAGTAGGGAACA
TGGCAGAGAGTGGTGCTTCCCAGCCTCACAATGTGGGAATTTGACATAGGATGAGAGTCAGAGTATAGG
TTTAAAAGATAAAATCTTTAGTTAATAATTTTGTATTTATTTATTCTAGATGTATGTATCTGAGGAAAG
AAATCTGGTATTTTTGCTTTCCAATAAAGGGGATCAAAGTAATGGTTTTTCTCTCAGTTCTCTAAGCTG
GTCTATGTTATAGCTCTAGCAGTATGGAAATGTGCTTTAAAATATGCTTACCTTTTGAATGATCATGGC
TATATGTTGTTGAGATATTTGAAACTTACCTTGTTTTCACTTGTGCACTGTGAATGAACTTTGTATTAT
TTTTTTAAAACCTTCACATTACGTGTAGATATTATTGCAACTTATATTTTGCCTGAGCTTGATCAAAGG
TCATTTGTGTAGATGAGTAATTAAAAAATATTTAAATCACATTATAATTCTATTATTGGAGAGCATCTT
TTAAATTTTTTTCTGTTTTAACGAGGGAAAGAGAAACCTGTATACCTAGGGTCATTATTTGACCCCATA
GTATAACCAGATTCATGGTCTAACAAGCTCTCAGTGTGGCTTTTCTCTGAATGCTTGAATTTCACATGC
CTTGCATTTCACAGTTGTACTCCATGGTCAACCGGTGCTTTTTTTCACATCGTGGTACTTGTCAAAACA
TTTTGTTATTTTCCTTGGTAAAATATATAAAAAAGGTTTTCTAATTTCA
ENST00000361908.8, SEQ ID NO: 1257, organism: homo sapiens
GGACTGAGTACGGGTGCCTGTCAGGCTCTTGCGGAAGTCCATGCGCCATTGGGAGGGCCTCGGCCGCGG
CTCTGTGCCCTTGCTGCTGAGGGCCACTTCCTGGGTCATTCCTGGACCGGGAGCCGGGCTGGGGCTCAC
ACGGGGGCTCCCGCGTGGCCGTCTCGGCGCCTGCGTGACCTCCCCGCCGGCGGGATGTGGCGACTACGT
CGGGCCGCTGTGGCCTGTGAGGTCTGCCAGTCTTTAGTGAAACACAGCTCTGGAATAAAAGGAAGTTTA
CCACTACAAAAACTACATCTGGTTTCACGAAGCATTTATCATTCACATCATCCTACCTTAAAGCTTCAA
CGACCCCAATTAAGGACATCCTTTCAGCAGTTCTCTTCTCTGACAAACCTTCCTTTACGTAAACTGAAA
TTCTCTCCAATTAAATATGGCTACCAGCCTCGCAGGAATTTTTGGCCAGCAAGATTAGCTACGAGACTC
TTAAAACTTCGCTATCTCATACTAGGATCGGCTGTTGGGGGTGGCTACACAGCCAAAAAGACTTTTGAT
CAGTGGAAAGATATGATACCGGACCTTAGTGAATATAAATGGATTGTGCCTGACATTGTGTGGGAAATT
GATGAGTATATCGATTTTGAGAAAATTAGAAAAGCCCTTCCTAGTTCAGAAGACCTTGTAAAGTTAGCA
CCAGACTTTGACAAGATTGTTGAAAGCCTTAGCTTATTGAAGGACTTTTTTACCTCAGGTTCTCCGGAA
GAAACGGCGTTTAGAGCAACAGATCGTGGATCTGAAAGTGACAAGCATTTTAGAAAGGGTCTGCTTGGT
GAGCTCATTCTCTTACAACAACAAATTCAAGAGCATGAAGAGGAAGCGCGCAGAGCCGCTGGCCAATAT
AGCACGAGCTATGCCCAACAGAAGCGCAAGGTGTCAGACAAAGAGAAAATTGACCAACTTCAGGAAGAA
CTTCTGCACACTCAGTTGAAGTATCAGAGAATCTTGGAACGATTAGAAAAGGAGAACAAAGAATTGAGA
AAATTAGTATTGCAGAAAGATGACAAAGGCATTCATCATAGAAAGCTTAAGAAATCTTTGATTGACATG
TATTCTGAAGTTCTTGATGTTCTCTCTGATTATGATGCCAGTTATAATACGCAAGATCATCTGCCACGG
GTTGTTGTGGTTGGAGATCAGAGTGCTGGAAAGACTAGTGTGTTGGAAATGATTGCCCAAGCTCGAATA
TTCCCAAGAGGATCTGGGGAGATGATGACACGTTCTCCAGTTAAGGTGACTCTGAGTGAAGGTCCTCAC
CATGTGGCCCTATTTAAAGATAGTTCTCGGGAGTTTGATCTTACCAAAGAAGAAGATCTTGCAGCATTA
AGACATGAAATAGAACTTCGAATGAGGAAAAATGTGAAAGAAGGCTGTACCGTTAGCCCTGAGACCATA
TCCTTAAATGTAAAAGGCCCTGGACTACAGAGGATGGTGCTTGTTGACTTACCAGGTGTGATTAATACT
GTGACATCAGGCATGGCTCCTGACACAAAGGAAACTATTTTCAGTATCAGCAAAGCTTACATGCAGAAT
CCTAATGCCATCATACTGTGTATTCAAGATGGATCTGTGGATGCTGAACGCAGTATTGTTACAGACTTG
GTCAGTCAAATGGACCCTCATGGAAGGAGAACCATATTCGTTTTGACCAAAGTAGACCTGGCAGAGAAA
AATGTAGCCAGTCCAAGCAGGATTCAGCAGATAATTGAAGGAAAGCTCTTCCCAATGAAAGCTTTAGGT
TATTTTGCTGTTGTAACAGGAAAAGGGAACAGCTCTGAAAGCATTGAAGCTATAAGAGAATATGAAGAA
GAGTTTTTTCAGAATTCAAAGCTCCTAAAGACAAGCATGCTAAAGGCACACCAAGTGACTACAAGAAAT
TTAAGCCTTGCAGTATCAGACTGCTTTTGGAAAATGGTACGAGAGTCTGTTGAACAACAGGCTGATAGT
TTCAAAGCAACACGTTTTAACCTTGAAACTGAATGGAAGAATAACTATCCTCGCCTGCGGGAACTTGAC
CGGAATGAACTATTTGAAAAAGCTAAAAATGAAATCCTTGATGAAGTTATCAGTCTGAGCCAGGTTACA
CCAAAACATTGGGAGGAAATCCTTCAACAATCTTTGTGGGAAAGAGTATCAACTCATGTGATTGAAAAC
ATCTACCTTCCAGCTGCGCAGACCATGAATTCAGGAACTTTTAACACCACAGTGGATATCAAGCTTAAA
CAGTGGACTGATAAACAACTTCCTAATAAAGCAGTAGAGGTTGCTTGGGAGACCCTACAAGAAGAATTT
TCCCGCTTTATGACAGAACCGAAAGGGAAAGAGCATGATGACATATTTGATAAACTTAAAGAGGCTGTT
AAGGAAGAAAGTATTAAACGACACAAGTGGAATGACTTTGCGGAGGACAGCTTGAGGGTTATTCAACAC
AATGCTTTGGAAGACCGATCCATATCTGATAAACAGCAATGGGATGCAGCTATTTATTTTATGGAAGAG
GCTCTGCAGGCTCGTCTCAAGGATACTGAAAATGCAATTGAAAACATGGTGGGTCCAGACTGGAAAAAG
AGGTGGTTATACTGGAAGAATCGGACCCAAGAACAGTGTGTTCACAATGAAACCAAGAATGAATTGGAG
AAGATGTTGAAATGTAATGAGGAGCACCCAGCTTATCTTGCAAGTGATGAAATAACCACAGTCCGGAAG
AACCTTGAATCCCGAGGAGTAGAAGTAGATCCAAGCTTGATTAAGGATACTTGGCATCAAGTTTATAGA
AGACATTTTTTAAAAACAGCTCTAAACCATTGTAACCTTTGTCGAAGAGGTTTTTATTACTACCAAAGG
CATTTTGTAGATTCTGAGTTGGAATGCAATGATGTGGTCTTGTTTTGGCGTATACAGCGCATGCTTGCT
ATCACCGCAAATACTTTAAGGCAACAACTTACAAATACTGAAGTTAGGCGATTAGAGAAAAATGTTAAA
GAGGTATTGGAAGATTTTGCTGAAGATGGTGAGAAGAAGATTAAATTGCTTACTGGTAAACGCGTTCAA
CTGGCGGAAGACCTCAAGAAAGTTAGAGAAATTCAAGAAAAACTTGATGCTTTCATTGAAGCTCTTCAT
CAGGAGAAATAAATTAAAATCGTACTCATAATCAGCTCTGCATACATCTGAAGAACAAAAACATCAACG
TCTTTTGTCCAGCCTCTTTTTCTTCTGCTGTTCCACCTTTCTAAACATACAATAAAGTCATGGGATAAA
AATAATCGATGTATGTTACGGGCGCTTTAACCATCAGCTGCCTCTCGAATGGAAGAACAGTGGTAATGG
ATTAACATCCTATTTTGTTGTACTAAAGTGACAAATCGGAATAATATAATTGGTATGGCCATTAGGTTC
AGTCCTTGAAGATAAGAAACTTGTTCTCTGTTTGTTGTCTTATTTGTGGTGGCACTCGTTTAATGGATT
AACTGAGGTTGCTCAATGTTCAGTTTCTTTTCCAGAAATACAATGCTAGGTGTTTTGAAATAAAACTTA
TATAGCAATTGTTTAAAGTTATCAATTGTATATAAAATCACAGTAGCCTGCTAAATCATTGTATGTGTC
TGTAGTATTCTATTCCCAGAAACTATTTGACCATGATAATTCAGTTTATATTCACCACATGAAAGAAAA
ATGGGTAACAGAAGAACCCTTAAAACAGGTTAATTTGGATTGTAACGTTCAGTGAAAGAAATTTCAACC
CTTCATAGCCAGCGAAGAAATTTGCCTTGGAAGCCAAGTCAGTACCAGCTTACCTATTTGATTCAGTTG
CTGTTTTCTCACTCTCTATATCCATTTGAAATTGATTTATTTTAGATGTTGTATACTTACGTTAGGCTT
TCTGTTAATAGTGGTTTTTCTCCTGTTGACAGAGCCACCGGATTATGACACAGGATGAGGAAGATTAAG
GATAATCAATTGACTAATTTCATTTAGAATATTATCAAACATTTCAACTAGGTATCAGAAAAAGGCTTT
CTTTCATAAGACTATTTTAAATAGAAATTATTTCAACAATTAAAGTAATGTTGACCATCCCCCTCTCAG
CTGAATAAAGAAAAATTTAGTTCAATTTATTGCAATTTAATTACAATACTACCTTCACAACATTTTCAT
GTGTTTTAAATAAATATTTTTTAATTGGCTAAAGGACATTCAAGCAAAGAAATGCTTTCTTTACTTAAA
ATGTCTATCTCATTTGCTGCCTTTTCACTAAGCCTTTACTTTGTTAATAAAAGTGTCCATTGTGTGATG
TTTTTGATTTTACAGTTTGCTAAATCTTATTTTCTTGGAGTTGCTTTTTGGTAACAGCCCCATTGCTAC
TCCCCATTTTATTGTTTTACATCAATGCATGCTTCGTTGTGATCCCTCAAGATGTAACACTTGGTATGC
TCGGTTGAGGATATGAAAAAATACTTCCGAAACCAGGAATTCAATGTATGTTTGTTTTATACTGTTTGA
TAAGAAAAGTAGGTCCAGCCTTAAGCAGCACAGATGCGCTGGTAGATGCATAGTCAGGAACTTTTTTTA
TTTCTTTTAGGTCTAGGGACAGGAGTGAATAGAAAGGGAGGAGAGCTCTATTATGTTCTATACACAGAT
TAGGAGATGACCTTACTGGGTACACCCCTCTAACCAGTGCTTACAGGTTAATGCATGTTAATGAATATT
TTTGCAGTTGTAAAGCATAACAATTACAACTACACATCTATTTCTAAAGAATAAAACAGGACCATATTT
ATTTACTTCTGTCAACTATAGAAAGAAAGACCTTCAGCTGTATTTCCACAGATTTCTCCCAAGGAAAAG
GCTAATATTAGTCACTACTGTTATCACATCCCTTTGTATAAGTTTTAAAAAGAGATGGAGGGAGATCTT
CATTTCTTTGAGGAGATCAGTATTGTAACGTATGTGAATAGATGATAACAATTAATATTACTAAAAGTC
CCACATGAGAGTCCTGACGCCCTCTCCATGCCCCACAGTAATGTGGCTTCTTTCATGGGTTTTTTTTTC
TTCTTTTTAGCTGATCTCATCCTAAGCATGCTTTATTTTTCCTTGAAAGCTAGGTATTTATCAACTGCA
GATGTTATTGAAAGAAAATAAAATTCAGTCTCAAGAGTAAACCCTGTGTCTTGTGTCTGTAGTTCAAAA
GTCAGAAATGATTCTAATTTAAACAAAAAGATACTAAATATACAGAAGTTAAATTCGAACTAGCCACAG
AATCATTTGTTTTTATGTCAGAATTTGCAAAGAGTGGAGTGGACAAAGCTCTGTATGGAAGACTGAACA
ACTGTAAATAGATGATATCCAAACTTAATTTGGCTAGGACTTCAATTTTAAAAATCAGTGTACCTAGGC
AGTGCACAGCACGAAATAAGTGGCCCTTGCAGCTTCCCCGTTTAACCCACTGTGCTATAGTTGCGGGTG
GAACAGTCAACCTTTCTAGTAGTTTATGATATTGCCCTCTTTGTATTCCCATTTTCTACAGTTTTTTCC
GCAGACTTCTTTCTGCAAATTATTCAGCCTCCAAATGCAAATGAATGATATAAAAATAAGTAGGGAACA
TGGCAGAGAGTGGTGCTTCCCAGCCTCACAATGTGGGAATTTGACATAGGATGAGAGTCAGAGTATAGG
TTTAAAAGATAAAATCTTTAGTTAATAATTTTGTATTTATTTATTCTAGATGTATGTATCTGAGGAAAG
AAATCTGGTATTTTTGCTTTCCAATAAAGGGGATCAAAGTAATGGTTTTTCTCTCAGTTCTCTAAGCTG
GTCTATGTTATAGCTCTAGCAGTATGGAAATGTGCTTTAAAATATGCTTACCTTTTGAATGATCATGGC
TATATGTTGTTGAGATATTTGAAACTTACCTTGTTTTCACTTGTGCACTGTGAATGAACTTTGTATTAT
TTTTTTAAAACCTTCACATTACGTGTAGATATTATTGCAACTTATATTTTGCCTGAGCTTGATCAAAGG
TCATTTGTGTAGATGAGTAATTAAAAAATATTTAAATCACATTATAATTCTATTATTGGAGAGCATCTT
TTAAATTTTTTTCTGTTTTAACGAGGGAAAGAGAAACCTGTATACCTAGGGTCATTATTTGACCCCATA
GTATAACCAGATTCATGGTCTAACAAGCTCTCAGTGTGGCTTTTCTCTGAATGCTTGAATTTCACATGC
CTTGCATTTCACAGTTGTACTCCATGGTCAACCGGTGCTTTTTTTCACATCGTGGTACTTGTCAAAACA
TTTTGTTATTTTCCTTGGTAAAATATATAAAAAAGGTTTTCTAATTTCA
ENST00000361510.8, SEQ ID NO: 1258, organism: homo sapiens
AGGCTCTTGCGGAAGTCCATGCGCCATTGGGAGGGCCTCGGCCGCGGCTCTGTGCCCTTGCTGCTGAGG
GCCACTTCCTGGGTCATTCCTGGACCGGGAGCCGGGCTGGGGCTCACACGGGGGCTCCCGCGTGGCCGT
CTCGGCGCCTGCGTGACCTCCCCGCCGGCGGGATGTGGCGACTACGTCGGGCCGCTGTGGCCTGTGAGG
TCTGCCAGTCTTTAGTGAAACACAGCTCTGGAATAAAAGGAAGTTTACCACTACAAAAACTACATCTGG
TTTCACGAAGCATTTATCATTCACATCATCCTACCTTAAAGCTTCAACGACCCCAATTAAGGACATCCT
TTCAGCAGTTCTCTTCTCTGACAAACCTTCCTTTACGTAAACTGAAATTCTCTCCAATTAAATATGGCT
ACCAGCCTCGCAGGAATTTTTGGCCAGCAAGATTAGCTACGAGACTCTTAAAACTTCGCTATCTCATAC
TAGGATCGGCTGTTGGGGGTGGCTACACAGCCAAAAAGACTTTTGATCAGTGGAAAGATATGATACCGG
ACCTTAGTGAATATAAATGGATTGTGCCTGACATTGTGTGGGAAATTGATGAGTATATCGATTTTGAGA
AAATTAGAAAAGCCCTTCCTAGTTCAGAAGACCTTGTAAAGTTAGCACCAGACTTTGACAAGATTGTTG
AAAGCCTTAGCTTATTGAAGGACTTTTTTACCTCAGGTCACAAATTGGTTAGTGAAGTCATAGGAGCTT
CTGACCTACTTCTCTTGTTAGGTTCTCCGGAAGAAACGGCGTTTAGAGCAACAGATCGTGGATCTGAAA
GTGACAAGCATTTTAGAAAGGGTCTGCTTGGTGAGCTCATTCTCTTACAACAACAAATTCAAGAGCATG
AAGAGGAAGCGCGCAGAGCCGCTGGCCAATATAGCACGAGCTATGCCCAACAGAAGCGCAAGGTGTCAG
ACAAAGAGAAAATTGACCAACTTCAGGAAGAACTTCTGCACACTCAGTTGAAGTATCAGAGAATCTTGG
AACGATTAGAAAAGGAGAACAAAGAATTGAGAAAATTAGTATTGCAGAAAGATGACAAAGGCATTCATC
ATAGAAAGCTTAAGAAATCTTTGATTGACATGTATTCTGAAGTTCTTGATGTTCTCTCTGATTATGATG
CCAGTTATAATACGCAAGATCATCTGCCACGGGTTGTTGTGGTTGGAGATCAGAGTGCTGGAAAGACTA
GTGTGTTGGAAATGATTGCCCAAGCTCGAATATTCCCAAGAGGATCTGGGGAGATGATGACACGTTCTC
CAGTTAAGGTGACTCTGAGTGAAGGTCCTCACCATGTGGCCCTATTTAAAGATAGTTCTCGGGAGTTTG
ATCTTACCAAAGAAGAAGATCTTGCAGCATTAAGACATGAAATAGAACTTCGAATGAGGAAAAATGTGA
AAGAAGGCTGTACCGTTAGCCCTGAGACCATATCCTTAAATGTAAAAGGCCCTGGACTACAGAGGATGG
TGCTTGTTGACTTACCAGGTGTGATTAATACTGTGACATCAGGCATGGCTCCTGACACAAAGGAAACTA
TTTTCAGTATCAGCAAAGCTTACATGCAGAATCCTAATGCCATCATACTGTGTATTCAAGATGGATCTG
TGGATGCTGAACGCAGTATTGTTACAGACTTGGTCAGTCAAATGGACCCTCATGGAAGGAGAACCATAT
TCGTTTTGACCAAAGTAGACCTGGCAGAGAAAAATGTAGCCAGTCCAAGCAGGATTCAGCAGATAATTG
AAGGAAAGCTCTTCCCAATGAAAGCTTTAGGTTATTTTGCTGTTGTAACAGGAAAAGGGAACAGCTCTG
AAAGCATTGAAGCTATAAGAGAATATGAAGAAGAGTTTTTTCAGAATTCAAAGCTCCTAAAGACAAGCA
TGCTAAAGGCACACCAAGTGACTACAAGAAATTTAAGCCTTGCAGTATCAGACTGCTTTTGGAAAATGG
TACGAGAGTCTGTTGAACAACAGGCTGATAGTTTCAAAGCAACACGTTTTAACCTTGAAACTGAATGGA
AGAATAACTATCCTCGCCTGCGGGAACTTGACCGGAATGAACTATTTGAAAAAGCTAAAAATGAAATCC
TTGATGAAGTTATCAGTCTGAGCCAGGTTACACCAAAACATTGGGAGGAAATCCTTCAACAATCTTTGT
GGGAAAGAGTATCAACTCATGTGATTGAAAACATCTACCTTCCAGCTGCGCAGACCATGAATTCAGGAA
CTTTTAACACCACAGTGGATATCAAGCTTAAACAGTGGACTGATAAACAACTTCCTAATAAAGCAGTAG
AGGTTGCTTGGGAGACCCTACAAGAAGAATTTTCCCGCTTTATGACAGAACCGAAAGGGAAAGAGCATG
ATGACATATTTGATAAACTTAAAGAGGCTGTTAAGGAAGAAAGTATTAAACGACACAAGTGGAATGACT
TTGCGGAGGACAGCTTGAGGGTTATTCAACACAATGCTTTGGAAGACCGATCCATATCTGATAAACAGC
AATGGGATGCAGCTATTTATTTTATGGAAGAGGCTCTGCAGGCTCGTCTCAAGGATACTGAAAATGCAA
TTGAAAACATGGTGGGTCCAGACTGGAAAAAGAGGTGGTTATACTGGAAGAATCGGACCCAAGAACAGT
GTGTTCACAATGAAACCAAGAATGAATTGGAGAAGATGTTGAAATGTAATGAGGAGCACCCAGCTTATC
TTGCAAGTGATGAAATAACCACAGTCCGGAAGAACCTTGAATCCCGAGGAGTAGAAGTAGATCCAAGCT
TGATTAAGGATACTTGGCATCAAGTTTATAGAAGACATTTTTTAAAAACAGCTCTAAACCATTGTAACC
TTTGTCGAAGAGGTTTTTATTACTACCAAAGGCATTTTGTAGATTCTGAGTTGGAATGCAATGATGTGG
TCTTGTTTTGGCGTATACAGCGCATGCTTGCTATCACCGCAAATACTTTAAGGCAACAACTTACAAATA
CTGAAGTTAGGCGATTAGAGAAAAATGTTAAAGAGGTATTGGAAGATTTTGCTGAAGATGGTGAGAAGA
AGATTAAATTGCTTACTGGTAAACGCGTTCAACTGGCGGAAGACCTCAAGAAAGTTAGAGAAATTCAAG
AAAAACTTGATGCTTTCATTGAAGCTCTTCATCAGGAGAAATAAATTAAAATCGTACTCATAATCAGCT
CTGCATACATCTGAAGAACAAAAACATCAACGTCTTTTGTCCAGCCTCTTTTTCTTCTGCTGTTCCACC
TTTCTAAACATACAATAAAGTCATGGGATAAAAATAATCGATGTATGTTACGGGCGCTTTAACCATCAG
CTGCCTCTCGAATGGAAGAACAGTGGTAATGGATTAACATCCTATTTTGTTGTACTAAAGTGACAAATC
GGAATAATATAATTGGTATGGCCATTAGGTTCAGTCCTTGAAGATAAGAAACTTGTTCTCTGTTTGTTG
TCTTATTTGTGGTGGCACTCGTTTAATGGATTAACTGAGGTTGCTCAATGTTCAGTTTCTTTTCCAGAA
ATACAATGCTAGGTGTTTTGAAATAAAACTTATATAGCAATTGTTTAAAGTTATCAATTGTATATAAAA
TCACAGTAGCCTGCTAAATCATTGTATGTGTCTGTAGTATTCTATTCCCAGAAACTATTTGACCATGAT
AATTCAGTTTATATTCACCACATGAAAGAAAAATGGGTAACAGAAGAACCCTTAAAACAGGTTAATTTG
GATTGTAACGTTCAGTGAAAGAAATTTCAACCCTTCATAGCCAGCGAAGAAATTTGCCTTGGAAGCCAA
GTCAGTACCAGCTTACCTATTTGATTCAGTTGCTGTTTTCTCACTCTCTATATCCATTTGAAATTGATT
TATTTTAGATGTTGTATACTTACGTTAGGCTTTCTGTTAATAGTGGTTTTTCTCCTGTTGACAGAGCCA
CCGGATTATGACACAGGATGAGGAAGATTAAGGATAATCAATTGACTAATTTCATTTAGAATATTATCA
AACATTTCAACTAGGTATCAGAAAAAGGCTTTCTTTCATAAGACTATTTTAAATAGAAATTATTTCAAC
AATTAAAGTAATGTTGACCATCCCCCTCTCAGCTGAATAAAGAAAAATTTAGTTCAATTTATTGCAATT
TAATTACAATACTACCTTCACAACATTTTCATGTGTTTTAAATAAATATTTTTTAATTGGCTAAAGGAC
ATTCAAGCAAAGAAATGCTTTCTTTACTTAAAATGTCTATCTCATTTGCTGCCTTTTCACTAAGCCTTT
ACTTTGTTAATAAAAGTGTCCATTGTGTGATGTTTTTGATTTTACAGTTTGCTAAATCTTATTTTCTTG
GAGTTGCTTTTTGGTAACAGCCCCATTGCTACTCCCCATTTTATTGTTTTACATCAATGCATGCTTCGT
TGTGATCCCTCAAGATGTAACACTTGGTATGCTCGGTTGAGGATATGAAAAAATACTTCCGAAACCAGG
AATTCAATGTATGTTTGTTTTATACTGTTTGATAAGAAAAGTAGGTCCAGCCTTAAGCAGCACAGATGC
GCTGGTAGATGCATAGTCAGGAACTTTTTTTATTTCTTTTAGGTCTAGGGACAGGAGTGAATAGAAAGG
GAGGAGAGCTCTATTATGTTCTATACACAGATTAGGAGATGACCTTACTGGGTACACCCCTCTAACCAG
TGCTTACAGGTTAATGCATGTTAATGAATATTTTTGCAGTTGTAAAGCATAACAATTACAACTACACAT
CTATTTCTAAAGAATAAAACAGGACCATATTTATTTACTTCTGTCAACTATAGAAAGAAAGACCTTCAG
CTGTATTTCCACAGATTTCTCCCAAGGAAAAGGCTAATATTAGTCACTACTGTTATCACATCCCTTTGT
ATAAGTTTTAAAAAGAGATGGAGGGAGATCTTCATTTCTTTGAGGAGATCAGTATTGTAACGTATGTGA
ATAGATGATAACAATTAATATTACTAAAAGTCCCACATGAGAGTCCTGACGCCCTCTCCATGCCCCACA
GTAATGTGGCTTCTTTCATGGGTTTTTTTTTCTTCTTTTTAGCTGATCTCATCCTAAGCATGCTTTATT
TTTCCTTGAAAGCTAGGTATTTATCAACTGCAGATGTTATTGAAAGAAAATAAAATTCAGTCTCAAGAG
TAAACCCTGTGTCTTGTGTCTGTAGTTCAAAAGTCAGAAATGATTCTAATTTAAACAAAAAGATACTAA
ATATACAGAAGTTAAATTCGAACTAGCCACAGAATCATTTGTTTTTATGTCAGAATTTGCAAAGAGTGG
AGTGGACAAAGCTCTGTATGGAAGACTGAACAACTGTAAATAGATGATATCCAAACTTAATTTGGCTAG
GACTTCAATTTTAAAAATCAGTGTACCTAGGCAGTGCACAGCACGAAATAAGTGGCCCTTGCAGCTTCC
CCGTTTAACCCACTGTGCTATAGTTGCGGGTGGAACAGTCAACCTTTCTAGTAGTTTATGATATTGCCC
TCTTTGTATTCCCATTTTCTACAGTTTTTTCCGCAGACTTCTTTCTGCAAATTATTCAGCCTCCAAATG
CAAATGAATGATATAAAAATAAGTAGGGAACATGGCAGAGAGTGGTGCTTCCCAGCCTCACAATGTGGG
AATTTGACATAGGATGAGAGTCAGAGTATAGGTTTAAAAGATAAAATCTTTAGTTAATAATTTTGTATT
TATTTATTCTAGATGTATGTATCTGAGGAAAGAAATCTGGTATTTTTGCTTTCCAATAAAGGGGATCAA
AGTAATGGTTTTTCTCTCAGTTCTCTAAGCTGGTCTATGTTATAGCTCTAGCAGTATGGAAATGTGCTT
TAAAATATGCTTACCTTTTGAATGATCATGGCTATATGTTGTTGAGATATTTGAAACTTACCTTGTTTT
CACTTGTGCACTGTGAATGAACTTTGTATTATTTTTTTAAAACCTTCACATTACGTGTAGATATTATTG
CAACTTATATTTTGCCTGAGCTTGATCAAAGGTCATTTGTGTAGATGAGTAATTAAAAAATATTTAAAT
CACATTATAATTCTATTATTGGAGAGCATCTTTTAAATTTTTTTCTGTTTTAACGAGGGAAAGAGAAAC
CTGTATACCTAGGGTCATTATTTGACCCCATAGTATAACCAGATTCATGGTCTAACAAGCTCTCAGTGT
GGCTTTTCTCTGAATGCTTGAATTTCACATGCCTTGCATTTCACAGTTGTACTCCATGGTCAACCGGTG
CTTTTTTTCACATCGTGGTACTTGTCAAAACATTTTGTTATTTTCCTTGGTAAAATATATAAAAAAGGT
TTTCTAATTTCA
ENST00000392436.7, SEQ ID NO: 1259, organism: homo sapiens
AAGTCCATGCGCCATTGGGAGGGCCTCGGCCGCGGCTCTGTGCCCTTGCTGCTGAGGGCCACTTCCTGG
GTCATTCCTGGACCGGGAGCCGGGCTGGGGCTCACACGGGGGCTCCCGCGTGGCCGTCTCGGCGCCTGC
GTGACCTCCCCGCCGGCGGGATGTGGCGACTACGTCGGGCCGCTGTGGCCTGTGAGGTCTGCCAGTCTT
TAGTGAAACACAGCTCTGGAATAAAAGGAAGTTTACCACTACAAAAACTACATCTGGTTTCACGAAGCA
TTTATCATTCACATCATCCTACCTTAAAGCTTCAACGACCCCAATTAAGGACATCCTTTCAGCAGTTCT
CTTCTCTGACAAACCTTCCTTTACGTAAACTGAAATTCTCTCCAATTAAATATGGCTACCAGCCTCGCA
GGAATTTTTGGCCAGCAAGATTAGCTACGAGACTCTTAAAACTTCGCTATCTCATACTAGGATCGGCTG
TTGGGGGTGGCTACACAGCCAAAAAGACTTTTGATCAGTGGAAAGATATGATACCGGACCTTAGTGAAT
ATAAATGGATTGTGCCTGACATTGTGTGGGAAATTGATGAGTATATCGATTTTGAGAAAATTAGAAAAG
CCCTTCCTAGTTCAGAAGACCTTGTAAAGTTAGCACCAGACTTTGACAAGATTGTTGAAAGCCTTAGCT
TATTGAAGGACTTTTTTACCTCAGGTTCTCCGGAAGAAACGGCGTTTAGAGCAACAGATCGTGGATCTG
AAAGTGACAAGCATTTTAGAAAGGTGTCAGACAAAGAGAAAATTGACCAACTTCAGGAAGAACTTCTGC
ACACTCAGTTGAAGTATCAGAGAATCTTGGAACGATTAGAAAAGGAGAACAAAGAATTGAGAAAATTAG
TATTGCAGAAAGATGACAAAGGCATTCATCATAGAAAGCTTAAGAAATCTTTGATTGACATGTATTCTG
AAGTTCTTGATGTTCTCTCTGATTATGATGCCAGTTATAATACGCAAGATCATCTGCCACGGGTTGTTG
TGGTTGGAGATCAGAGTGCTGGAAAGACTAGTGTGTTGGAAATGATTGCCCAAGCTCGAATATTCCCAA
GAGGATCTGGGGAGATGATGACACGTTCTCCAGTTAAGGTGACTCTGAGTGAAGGTCCTCACCATGTGG
CCCTATTTAAAGATAGTTCTCGGGAGTTTGATCTTACCAAAGAAGAAGATCTTGCAGCATTAAGACATG
AAATAGAACTTCGAATGAGGAAAAATGTGAAAGAAGGCTGTACCGTTAGCCCTGAGACCATATCCTTAA
ATGTAAAAGGCCCTGGACTACAGAGGATGGTGCTTGTTGACTTACCAGGTGTGATTAATACTGTGACAT
CAGGCATGGCTCCTGACACAAAGGAAACTATTTTCAGTATCAGCAAAGCTTACATGCAGAATCCTAATG
CCATCATACTGTGTATTCAAGATGGATCTGTGGATGCTGAACGCAGTATTGTTACAGACTTGGTCAGTC
AAATGGACCCTCATGGAAGGAGAACCATATTCGTTTTGACCAAAGTAGACCTGGCAGAGAAAAATGTAG
CCAGTCCAAGCAGGATTCAGCAGATAATTGAAGGAAAGCTCTTCCCAATGAAAGCTTTAGGTTATTTTG
CTGTTGTAACAGGAAAAGGGAACAGCTCTGAAAGCATTGAAGCTATAAGAGAATATGAAGAAGAGTTTT
TTCAGAATTCAAAGCTCCTAAAGACAAGCATGCTAAAGGCACACCAAGTGACTACAAGAAATTTAAGCC
TTGCAGTATCAGACTGCTTTTGGAAAATGGTACGAGAGTCTGTTGAACAACAGGCTGATAGTTTCAAAG
CAACACGTTTTAACCTTGAAACTGAATGGAAGAATAACTATCCTCGCCTGCGGGAACTTGACCGGAATG
AACTATTTGAAAAAGCTAAAAATGAAATCCTTGATGAAGTTATCAGTCTGAGCCAGGTTACACCAAAAC
ATTGGGAGGAAATCCTTCAACAATCTTTGTGGGAAAGAGTATCAACTCATGTGATTGAAAACATCTACC
TTCCAGCTGCGCAGACCATGAATTCAGGAACTTTTAACACCACAGTGGATATCAAGCTTAAACAGTGGA
CTGATAAACAACTTCCTAATAAAGCAGTAGAGGTTGCTTGGGAGACCCTACAAGAAGAATTTTCCCGCT
TTATGACAGAACCGAAAGGGAAAGAGCATGATGACATATTTGATAAACTTAAAGAGGCTGTTAAGGAAG
AAAGTATTAAACGACACAAGTGGAATGACTTTGCGGAGGACAGCTTGAGGGTTATTCAACACAATGCTT
TGGAAGACCGATCCATATCTGATAAACAGCAATGGGATGCAGCTATTTATTTTATGGAAGAGGCTCTGC
AGGCTCGTCTCAAGGATACTGAAAATGCAATTGAAAACATGGTGGGTCCAGACTGGAAAAAGAGGTGGT
TATACTGGAAGAATCGGACCCAAGAACAGTGTGTTCACAATGAAACCAAGAATGAATTGGAGAAGATGT
TGAAATGTAATGAGGAGCACCCAGCTTATCTTGCAAGTGATGAAATAACCACAGTCCGGAAGAACCTTG
AATCCCGAGGAGTAGAAGTAGATCCAAGCTTGATTAAGGATACTTGGCATCAAGTTTATAGAAGACATT
TTTTAAAAACAGCTCTAAACCATTGTAACCTTTGTCGAAGAGGTTTTTATTACTACCAAAGGCATTTTG
TAGATTCTGAGTTGGAATGCAATGATGTGGTCTTGTTTTGGCGTATACAGCGCATGCTTGCTATCACCG
CAAATACTTTAAGGCAACAACTTACAAATACTGAAGTTAGGCGATTAGAGAAAAATGTTAAAGAGGTAT
TGGAAGATTTTGCTGAAGATGGTGAGAAGAAGATTAAATTGCTTACTGGTAAACGCGTTCAACTGGCGG
AAGACCTCAAAATGTTCTTTTTCCCCAGCTCGGACTCAACACTACACACCAGGCCACCACATGGCGCCG
CACCCAGCATTGACACTTCTTTTACCTTGTCTGGGCTCTGACATCCGTGCCAGGTTGCTCTTCGTCATG
GAGTCCCTTTTACTGAGCTCTGCTCTGACGCTTTGTGCCAGGTGCCTCTCCATCTCATCCTTCCCACCC
GCTAGCCTCTGCCCGACCCCAGACAGATTCCTTCCTCACCTGAAGCCAGACCATGCCTTTGTGGAGATA
CCCTCTTTACCCTGCCTGTGCTTCGCCAGCCTGCACCAGGCCACCCTCCTGCACAGATACTCTCCTCAG
TACTGGACCAGGCTACCAACAGCCCCATGTGAACCCATTGTAACCCAGGTCAGGCATTAACACCTGCAG
TAGGCTACCATGGCTTCCCCTTCCCACCCCCCTAGCTTGGCCCTACTAATAATCACTTTGTCACTGTTT
GGGGTTGATATTTGGTTGTTTCTTGTAGGTTCCTAGCTTTAAGATAGGATTGCATACTAAAATTTACTT
AGATCTTTGAGAACTCAAGGAAATCAGTGAAACATTATTGTTATTAAATAAAAATAAAAT
ENST00000646793.1, SEQ ID NO: 1260, organism: homo sapiens
GGCCGCGGCTCTGTGCCCTTGCTGCTGAGGGCCACTTCCTGGGTCATTCCTGGACCGGGAGCCGGGCTG
GGGCTCACACGGGGGCTCCCGCGTGGCCGTCTCGGCGCCTGCGTGACCTCCCCGCCGGCGGGATGTGGC
GACTACGTCGGGCCGCTGTGGCCTGTGAGGTCTGCCAGTCTTTAGTGAAACACAGCTCTGGAATAAAAG
GAAGTTTACCACTACAAAAACTACATCTGGTTTCACGAAGCATTTATCATTCACATCATCCTACCTTAA
AGCTTCAACGACCCCAATTAAGGACATCCTTTCAGCAGTTCTCTTCTCTGACAAACCTTCCTTTACGTA
AACTGAAATTCTCTCCAATTAAATATGGCTACCAGCCTCGCAGGAATTTTTGGCCAGCAAGATTAGCTA
CGAGACTCTTAAAACTTCGCTATCTCATACTAGGATCGGCTGTTGGGGGTGGCTACACAGCCAAAAAGA
CTTTTGATCAGTGGAAAGATATGATACCGGACCTTAGTGAATATAAATGGATTGTGCCTGACATTGTGT
GGGAAATTGATGAGTATATCGATTTTGGTTCTCCGGAAGAAACGGCGTTTAGAGCAACAGATCGTGGAT
CTGAAAGTGACAAGCATTTTAGAAAGGTGTCAGACAAAGAGAAAATTGACCAACTTCAGGAAGAACTTC
TGCACACTCAGTTGAAGTATCAGAGAATCTTGGAACGATTAGAAAAGGAGAACAAAGAATTGAGAAAAT
TAGTATTGCAGAAAGATGACAAAGGCATTCATCATAGAAAGCTTAAGAAATCTTTGATTGACATGTATT
CTGAAGTTCTTGATGTTCTCTCTGATTATGATGCCAGTTATAATACGCAAGATCATCTGCCACGGGTTG
TTGTGGTTGGAGATCAGAGTGCTGGAAAGACTAGTGTGTTGGAAATGATTGCCCAAGCTCGAATATTCC
CAAGAGGATCTGGGGAGATGATGACACGTTCTCCAGTTAAGGTGACTCTGAGTGAAGGTCCTCACCATG
TGGCCCTATTTAAAGATAGTTCTCGGGAGTTTGATCTTACCAAAGAAGAAGATCTTGCAGCATTAAGAC
ATGAAATAGAACTTCGAATGAGGAAAAATGTGAAAGAAGGCTGTACCGTTAGCCCTGAGACCATATCCT
TAAATGTAAAAGGCCCTGGACTACAGAGGATGGTGCTTGTTGACTTACCAGGTGTGATTAATACTGTGA
CATCAGGCATGGCTCCTGACACAAAGGAAACTATTTTCAGTATCAGCAAAGCTTACATGCAGAATCCTA
ATGCCATCATACTGTGTATTCAAGATGGATCTGTGGATGCTGAACGCAGTATTGTTACAGACTTGGTCA
GTCAAATGGACCCTCATGGAAGGAGAACCATATTCGTTTTGACCAAAGTAGACCTGGCAGAGAAAAATG
TAGCCAGTCCAAGCAGGATTCAGCAGATAATTGAAGGAAAGCTCTTCCCAATGAAAGCTTTAGGTTATT
TTGCTGTTGTAACAGGAAAAGGGAACAGCTCTGAAAGCATTGAAGCTATAAGAGAATATGAAGAAGAGT
TTTTTCAGAATTCAAAGCTCCTAAAGACAAGCATGCTAAAGGCACACCAAGTGACTACAAGAAATTTAA
GCCTTGCAGTATCAGACTGCTTTTGGAAAATGGTACGAGAGTCTGTTGAACAACAGGCTGATAGTTTCA
AAGCAACACGTTTTAACCTTGAAACTGAATGGAAGAATAACTATCCTCGCCTGCGGGAACTTGACCGGA
ATGAACTATTTGAAAAAGCTAAAAATGAAATCCTTGATGAAGTTATCAGTCTGAGCCAGGTTACACCAA
AACATTGGGAGGAAATCCTTCAACAATCTTTGTGGGAAAGAGTATCAACTCATGTGATTGAAAACATCT
ACCTTCCAGCTGCGCAGACCATGAATTCAGGAACTTTTAACACCACAGTGGATATCAAGCTTAAACAGT
GGACTGATAAACAACTTCCTAATAAAGCAGTAGAGGTTGCTTGGGAGACCCTACAAGAAGAATTTTCCC
GCTTTATGACAGAACCGAAAGGGAAAGAGCATGATGACATATTTGATAAACTTAAAGAGGCTGTTAAGG
AAGAAAGTATTAAACGACACAAGTGGAATGACTTTGCGGAGGACAGCTTGAGGGTTATTCAACACAATG
CTTTGGAAGACCGATCCATATCTGATAAACAGCAATGGGATGCAGCTATTTATTTTATGGAAGAGGCTC
TGCAGGCTCGTCTCAAGGATACTGAAAATGCAATTGAAAACATGGTGGGTCCAGACTGGAAAAAGAGGT
GGTTATACTGGAAGAATCGGACCCAAGAACAGTGTGTTCACAATGAAACCAAGAATGAATTGGAGAAGA
TGTTGAAATGTAATGAGGAGCACCCAGCTTATCTTGCAAGTGATGAAATAACCACAGTCCGGAAGAACC
TTGAATCCCGAGGAGTAGAAGTAGATCCAAGCTTGATTAAGGATACTTGGCATCAAGTTTATAGAAGAC
ATTTTTTAAAAACAGCTCTAAACCATTGTAACCTTTGTCGAAGAGGTTTTTATTACTACCAAAGGCATT
TTGTAGATTCTGAGTTGGAATGCAATGATGTGGTCTTGTTTTGGCGTATACAGCGCATGCTTGCTATCA
CCGCAAATACTTTAAGGCAACAACTTACAAATACTGAAGTTAGGCGATTAGAGAAAAATGTTAAAGAGG
TATTGGAAGATTTTGCTGAAGATGGTGAGAAGAAGATTAAATTGCTTACTGGTAAACGCGTTCAACTGG
CGGAAGACCTCAAGAAAGTTAGAGAAATTCAAGAAAAACTTGATGCTTTCATTGAAGCTCTTCATCAGG
AGAAATAAATTAAAATCGTACTCATAATCAGCTCTGCATACATCTGAAGAACAAAAACATCAACGTCTT
TTGTCCAGCCTCTTTTTCTTCTGCTGTTCCACCTTTCTAAACATACAATAAAGTCATGGGATAAAAATA
ATCGATGTATGTTACGGGCGCTTTAACCATCAGCTGCCTCTCGAATGGAAGAACAGTGGTAATGGATTA
ACATCCTATTTTGTTGTACTAAAGTGACAAATCGGAATAATATAATTGGTATGGCCATTAGGTTCAGTC
CTTGAAGATAAGAAACTTGTTCTCTGTTTGTTGTCTTATTTGTGGTGGCACTCGTTTAATGGATTAACT
GAGGTTGCTCAATGTTCAGTTTCTTTTCCAGAAATACAATGCTAGGTGTTTTGAAATAAAACTTATATA
GCAATTGTTTAAAGTTATCAATTGTATATAAAATCACAGTAGCCTGCTAAATCATTGTATGTGTCTGTA
GTATTCTATTCCCAGAAACTATTTGACCATGATAATTCAGTTTATATTCACCACATGAAAGAAAAATGG
GTAACAGAAGAACCCTTAAAACAGGTTAATTTGGATTGTAACGTTCAGTGAAAGAAATTTCAACCCTTC
ATAGCCAGCGAAGAAATTTGCCTTGGAAGCCAAGTCAGTACCAGCTTACCTATTTGATTCAGTTGCTGT
TTTCTCACTCTCTATATCCATTTGAAATTGATTTATTTTAGATGTTGTATACTTACGTTAGGCTTTCTG
TTAATAGTGGTTTTTCTCCTGTTGACAGAGCCACCGGATTATGACACAGGATGAGGAAGATTAAGGATA
ATCAATTGACTAATTTCATTTAGAATATTATCAAACATTTCAACTAGGTATCAGAAAAAGGCTTTCTTT
CATAAGACTATTTTAAATAGAAATTATTTCAACAATTAAAGTAATGTTGACCATCCCCCTCTCAGCTGA
ATAAAGAAAAATTTAGTTCAATTTATTGCAATTTAATTACAATACTACCTTCACAACATTTTCATGTGT
TTTAAATAAATATTTTTTAATTGGCTAAAGGACATTCAAGCAAAGAAATGCTTTCTTTACTTAAAATGT
CTATCTCATTTGCTGCCTTTTCACTAAGCCTTTACTTTGTTAATAAAAGTGTCCATTGTGTGATGTTTT
TGATTTTACAGTTTGCTAAATCTTATTTTCTTGGAGTTGCTTTTTGGTAACAGCCCCATTGCTACTCCC
CATTTTATTGTTTTACATCAATGCATGCTTCGTTGTGATCCCTCAAGATGTAACACTTGGTATGCTCGG
TTGAGGATATGAAAAAATACTTCCGAAACCAGGAATTCAATGTATGTTTGTTTTATACTGTTTGATAAG
AAAAGTAGGTCCAGCCTTAAGCAGCACAGATGCGCTGGTAGATGCATAGTCAGGAACTTTTTTTATTTC
TTTTAGGTCTAGGGACAGGAGTGAATAGAAAGGGAGGAGAGCTCTATTATGTTCTATACACAGATTAGG
AGATGACCTTACTGGGTACACCCCTCTAACCAGTGCTTACAGGTTAATGCATGTTAATGAATATTTTTG
CAGTTGTAAAGCATAACAATTACAACTACACATCTATTTCTAAAGAATAAAACAGGACCATATTTATTT
ACTTCTGTCAACTATAGAAAGAAAGACCTTCAGCTGTATTTCCACAGATTTCTCCCAAGGAAAAGGCTA
ATATTAGTCACTACTGTTATCACATCCCTTTGTATAAGTTTTAAAAAGAGATGGAGGGAGATCTTCATT
TCTTTGAGGAGATCAGTATTGTAACGTATGTGAATAGATGATAACAATTAATATTACTAAAAGTCCCAC
ATGAGAGTCCTGACGCCCTCTCCATGCCCCACAGTAATGTGGCTTCTTTCATGGGTTTTTTTTTCTTCT
TTTTAGCTGATCTCATCCTAAGCATGCTTTATTTTTCCTTGAAAGCTAGGTATTTATCAACTGCAGATG
TTATTGAAAGAAAATAAAATTCAGTCTCAAGAGTAAACCCTGTGTCTTGTGTCTGTAGTTCAAAAGTCA
GAAATGATTCTAATTTAAACAAAAAGATACTAAATATACAGAAGTTAAATTCGAACTAGCCACAGAATC
ATTTGTTTTTATGTCAGAATTTGCAAAGAGTGGAGTGGACAAAGCTCTGTATGGAAGACTGAACAACTG
TAAATAGATGATATCCAAACTTAATTTGGCTAGGACTTCAATTTTAAAAATCAGTGTACCTAGGCAGTG
CACAGCACGAAATAAGTGGCCCTTGCAGCTTCCCCGTTTAACCCACTGTGCTATAGTTGCGGGTGGAAC
AGTCAACCTTTCTAGTAGTTTATGATATTGCCCTCTTTGTATTCCCATTTTCTACAGTTTTTTCCGCAG
ACTTCTTTCTGCAAATTATTCAGCCTCCAAATGCAAATGAATGATATAAAAATAAGTAGGGAACATGGC
AGAGAGTGGTGCTTCCCAGCCTCACAATGTGGGAATTTGACATAGGATGAGAGTCAGAGTATAGGTTTA
AAAGATAAAATCTTTAGTTAATAATTTTGTATTTATTTATTCTAGATGTATGTATCTGAGGAAAGAAAT
CTGGTATTTTTGCTTTCCAATAAAGGGGATCAAAGTAATGGTTTTTCTCTCAGTTCTCTAAGCTGGTCT
ATGTTATAGCTCTAGCAGTATGGAAATGTGCTTTAAAATATGCTTACCTTTTGAATGATCATGGCTATA
TGTTGTTGAGATATTTGAAACTTACCTTGTTTTCACTTGTGCACTGTGAATGAACTTTGTATTATTTTT
TTAAAACCTTCACATTACGTGTAGATATTATTGCAACTTATATTTTGCCTGAGCTTGATCAAAGGTCAT
TTGTGTAGATGAGTAATTAAAAAATATTTAAATC
ENST00000392437.6, SEQ ID NO: 1261, organism: homo sapiens
ACTTCCTGGGTCATTCCTGGACCGGGAGCCGGGCTGGGGCTCACACGGGGGCTCCCGCGTGGCCGTCTC
GGCGCCTGCGTGACCTCCCCGCCGGCGGGATGTGGCGACTACGTCGGGCCGCTGTGGCCTGTGAGGTCT
GCCAGTCTTTAGTGAAACACAGCTCTGGAATAAAAGGAAGTTTACCACTACAAAAACTACATCTGGTTT
CACGAAGCATTTATCATTCACATCATCCTACCTTAAAGCTTCAACGACCCCAATTAAGGACATCCTTTC
AGCAGTTCTCTTCTCTGACAAACCTTCCTTTACGTAAACTGAAATTCTCTCCAATTAAATATGGCTACC
AGCCTCGCAGGAATTTTTGGCCAGCAAGATTAGCTACGAGACTCTTAAAACTTCGCTATCTCATACTAG
GATCGGCTGTTGGGGGTGGCTACACAGCCAAAAAGACTTTTGATCAGTGGAAAGATATGATACCGGACC
TTAGTGAATATAAATGGATTGTGCCTGACATTGTGTGGGAAATTGATGAGTATATCGATTTTGAGAAAA
TTAGAAAAGCCCTTCCTAGTTCAGAAGACCTTGTAAAGTTAGCACCAGACTTTGACAAGATTGTTGAAA
GCCTTAGCTTATTGAAGGACTTTTTTACCTCAGGTCACAAATTGGTTAGTGAAGTCATAGGAGCTTCTG
ACCTACTTCTCTTGTTAGGTTCTCCGGAAGAAACGGCGTTTAGAGCAACAGATCGTGGATCTGAAAGTG
ACAAGCATTTTAGAAAGGTGTCAGACAAAGAGAAAATTGACCAACTTCAGGAAGAACTTCTGCACACTC
AGTTGAAGTATCAGAGAATCTTGGAACGATTAGAAAAGGAGAACAAAGAATTGAGAAAATTAGTATTGC
AGAAAGATGACAAAGGCATTCATCATAGAAAGCTTAAGAAATCTTTGATTGACATGTATTCTGAAGTTC
TTGATGTTCTCTCTGATTATGATGCCAGTTATAATACGCAAGATCATCTGCCACGGGTTGTTGTGGTTG
GAGATCAGAGTGCTGGAAAGACTAGTGTGTTGGAAATGATTGCCCAAGCTCGAATATTCCCAAGAGGAT
CTGGGGAGATGATGACACGTTCTCCAGTTAAGGTGACTCTGAGTGAAGGTCCTCACCATGTGGCCCTAT
TTAAAGATAGTTCTCGGGAGTTTGATCTTACCAAAGAAGAAGATCTTGCAGCATTAAGACATGAAATAG
AACTTCGAATGAGGAAAAATGTGAAAGAAGGCTGTACCGTTAGCCCTGAGACCATATCCTTAAATGTAA
AAGGCCCTGGACTACAGAGGATGGTGCTTGTTGACTTACCAGGTGTGATTAATACTGTGACATCAGGCA
TGGCTCCTGACACAAAGGAAACTATTTTCAGTATCAGCAAAGCTTACATGCAGAATCCTAATGCCATCA
TACTGTGTATTCAAGATGGATCTGTGGATGCTGAACGCAGTATTGTTACAGACTTGGTCAGTCAAATGG
ACCCTCATGGAAGGAGAACCATATTCGTTTTGACCAAAGTAGACCTGGCAGAGAAAAATGTAGCCAGTC
CAAGCAGGATTCAGCAGATAATTGAAGGAAAGCTCTTCCCAATGAAAGCTTTAGGTTATTTTGCTGTTG
TAACAGGAAAAGGGAACAGCTCTGAAAGCATTGAAGCTATAAGAGAATATGAAGAAGAGTTTTTTCAGA
ATTCAAAGCTCCTAAAGACAAGCATGCTAAAGGCACACCAAGTGACTACAAGAAATTTAAGCCTTGCAG
TATCAGACTGCTTTTGGAAAATGGTACGAGAGTCTGTTGAACAACAGGCTGATAGTTTCAAAGCAACAC
GTTTTAACCTTGAAACTGAATGGAAGAATAACTATCCTCGCCTGCGGGAACTTGACCGGAATGAACTAT
TTGAAAAAGCTAAAAATGAAATCCTTGATGAAGTTATCAGTCTGAGCCAGGTTACACCAAAACATTGGG
AGGAAATCCTTCAACAATCTTTGTGGGAAAGAGTATCAACTCATGTGATTGAAAACATCTACCTTCCAG
CTGCGCAGACCATGAATTCAGGAACTTTTAACACCACAGTGGATATCAAGCTTAAACAGTGGACTGATA
AACAACTTCCTAATAAAGCAGTAGAGGTTGCTTGGGAGACCCTACAAGAAGAATTTTCCCGCTTTATGA
CAGAACCGAAAGGGAAAGAGCATGATGACATATTTGATAAACTTAAAGAGGCTGTTAAGGAAGAAAGTA
TTAAACGACACAAGTGGAATGACTTTGCGGAGGACAGCTTGAGGGTTATTCAACACAATGCTTTGGAAG
ACCGATCCATATCTGATAAACAGCAATGGGATGCAGCTATTTATTTTATGGAAGAGGCTCTGCAGGCTC
GTCTCAAGGATACTGAAAATGCAATTGAAAACATGGTGGGTCCAGACTGGAAAAAGAGGTGGTTATACT
GGAAGAATCGGACCCAAGAACAGTGTGTTCACAATGAAACCAAGAATGAATTGGAGAAGATGTTGAAAT
GTAATGAGGAGCACCCAGCTTATCTTGCAAGTGATGAAATAACCACAGTCCGGAAGAACCTTGAATCCC
GAGGAGTAGAAGTAGATCCAAGCTTGATTAAGGATACTTGGCATCAAGTTTATAGAAGACATTTTTTAA
AAACAGCTCTAAACCATTGTAACCTTTGTCGAAGAGGTTTTTATTACTACCAAAGGCATTTTGTAGATT
CTGAGTTGGAATGCAATGATGTGGTCTTGTTTTGGCGTATACAGCGCATGCTTGCTATCACCGCAAATA
CTTTAAGGCAACAACTTACAAATACTGAAGTTAGGCGATTAGAGAAAAATGTTAAAGAGGTATTGGAAG
ATTTTGCTGAAGATGGTGAGAAGAAGATTAAATTGCTTACTGGTAAACGCGTTCAACTGGCGGAAGACC
TCAAGAAAGTTAGAGAAATTCAAGAAAAACTTGATGCTTTCATTGAAGCTCTTCATCAGGAGAAATAAA
TTAAAATCGTACTCATAATCAGCTCTGCATACATCTGAAGAACAAAAACATCAACGTCTTTTGTCCAGC
CTCTTTTTCTTCTGCTGTTCCACCTTTCTAAACATACAATAAAGTCATGGGATAAAAATAATCGATGTA
TGTTACGGGCGCTTTAACCATCAGCTGCCTCTCGAATGGAAGAACAGTGGTAATGGATTAACATCCTAT
TTTGTTGTACTAAAGTGACAAATCGGAATAATATAATTGGTATGGCCATTAGGTTCAGTCCTTGAAGAT
AAGAAACTTGTTCTCTGTTTGTTGTCTTATTTGTGGTGGCACTCGTTTAATGGATTAACTGAGGTTGCT
CAATGTTCAGTTTCTTTTCCAGAAATACAATGCTAGGTGTTTTGAAATAAAACTTATATAGCAATTGTT
TAAAGTTATCAATTGTATATAAAATCACAGTAGCCTGCTAAATCATTGTATGTGTCTGTAGTATTCTAT
TCCCAGAAACTATTTGACCATGATAATTCAGTTTATATTCACCACATGAAAGAAAAATGGGTAACAGAA
GAACCCTTAAAACAGGTTAATTTGGATTGTAACGTTCAGTGAAAGAAATTTCAACCCTTCATAGCCAGC
GAAGAAATTTGCCTTGGAAGCCAAGTCAGTACCAGCTTACCTATTTGATTCAGTTGCTGTTTTCTCACT
CTCTATATCCATTTGAAATTGATTTATTTTAGATGTTGTATACTTACGTTAGGCTTTCTGTTAATAGTG
GTTTTTCTCCTGTTGACAGAGCCACCGGATTATGACACAGGATGAGGAAGATTAAGGATAATCAATTGA
CTAATTTCATTTAGAATATTATCAAACATTTCAACTAGGTATCAGAAAAAGGCTTTCTTTCATAAGACT
ATTTTAAATAGAAATTATTTCAACAATTAAAGTAATGTTGACCATCCCCCTCTCAGCTGAATAAAGAAA
AATTTAGTTCAATTTATTGCAATTTAATTACAATACTACCTTCACAACATTTTCATGTGTTTTAAATAA
ATATTTTTTAATTGGC
ENST00000645553.1, SEQ ID NO: 1262, organism: homo sapiens
GGGTCATTCCTGGACCGGGAGCCGGGCTGGGGCTCACACGGGGGCTCCCGCGTGGCCGTCTCGGCGCCT
GCGTGACCTCCCCGCCGGCGGGATGTGGCGACTACGTCGGGCCGCTGTGGCCTGTGAGGTCTGCCAGTC
TTTAGTGAAACACAGCTCTGGAATAAAAGGAAGTTTACCACTACAAAAACTACATCTGGTTTCACGAAG
CATTTATCATTCACATCATCCTACCTTAAAGCTTCAACGACCCCAATTAAGGACATCCTTTCAGCAGTT
CTCTTCTCTGACAAACCTTCCTTTACGTAAACTGAAATTCTCTCCAATTAAATATGGCTACCAGCCTCG
CAGGAATTTTTGGCCAGCAAGATTAGCTACGAGACTCTTAAAACTTCGCTATCTCATACTAGGATCGGC
TGTTGGGGGTGGCTACACAGCCAAAAAGACTTTTGATCAGTGGAAAGATATGATACCGGACCTTAGTGA
ATATAAATGGATTGTGCCTGACATTGTGTGGGAAATTGATGAGTATATCGATTTTGAGAAAATTAGAAA
AGCCCTTCCTAGTTCAGAAGACCTTGTAAAGTTAGCACCAGACTTTGACAAGATTGTTGAAAGCCTTAG
CTTATTGAAGGACTTTTTTACCTCAGGTTCTCCGGAAGAAACGGCGTTTAGAGCAACAGATCGTGGATC
TGAAAGTGACAAGCATTTTAGAAAGGTGTCAGACAAAGAGAAAATTGACCAACTTCAGGAAGAACTTCT
GCACACTCAGAGCAAAATTTGGAAGTTGAAGTATCAGAGAATCTTGGAACGATTAGAAAAGGAGAACAA
AGAATTGAGAAAATTAGTATTGCAGAAAGATGACAAAGGCATTCATCATAGAAAGCTTAAGAAATCTTT
GATTGACATGTATTCTGAAGTTCTTGATGTTCTCTCTGATTATGATGCCAGTTATAATACGCAAGATCA
TCTGCCACGGGTTGTTGTGGTTGGAGATCAGAGTGCTGGAAAGACTAGTGTGTTGGAAATGATTGCCCA
AGCTCGAATATTCCCAAGAGGATCTGGGGAGATGATGACACGTTCTCCAGTTAAGGTGACTCTGAGTGA
AGGTCCTCACCATGTGGCCCTATTTAAAGATAGTTCTCGGGAGTTTGATCTTACCAAAGAAGAAGATCT
TGCAGCATTAAGACATGAAATAGAACTTCGAATGAGGAAAAATGTGAAAGAAGGCTGTACCGTTAGCCC
TGAGACCATATCCTTAAATGTAAAAGGCCCTGGACTACAGAGGATGGTGCTTGTTGACTTACCAGGTGT
GATTAATACTGTGACATCAGGCATGGCTCCTGACACAAAGGAAACTATTTTCAGTATCAGCAAAGCTTA
CATGCAGAATCCTAATGCCATCATACTGTGTATTCAAGATGGATCTGTGGATGCTGAACGCAGTATTGT
TACAGACTTGGTCAGTCAAATGGACCCTCATGGAAGGAGAACCATATTCGTTTTGACCAAAGTAGACCT
GGCAGAGAAAAATGTAGCCAGTCCAAGCAGGATTCAGCAGATAATTGAAGGAAAGCTCTTCCCAATGAA
AGCTTTAGGTTATTTTGCTGTTGTAACAGGAAAAGGGAACAGCTCTGAAAGCATTGAAGCTATAAGAGA
ATATGAAGAAGAGTTTTTTCAGAATTCAAAGCTCCTAAAGACAAGCATGCTAAAGGCACACCAAGTGAC
TACAAGAAATTTAAGCCTTGCAGTATCAGACTGCTTTTGGAAAATGGTACGAGAGTCTGTTGAACAACA
GGCTGATAGTTTCAAAGCAACACGTTTTAACCTTGAAACTGAATGGAAGAATAACTATCCTCGCCTGCG
GGAACTTGACCGGAATGAACTATTTGAAAAAGCTAAAAATGAAATCCTTGATGAAGTTATCAGTCTGAG
CCAGGTTACACCAAAACATTGGGAGGAAATCCTTCAACAATCTTTGTGGGAAAGAGTATCAACTCATGT
GATTGAAAACATCTACCTTCCAGCTGCGCAGACCATGAATTCAGGAACTTTTAACACCACAGTGGATAT
CAAGCTTAAACAGTGGACTGATAAACAACTTCCTAATAAAGCAGTAGAGGTTGCTTGGGAGACCCTACA
AGAAGAATTTTCCCGCTTTATGACAGAACCGAAAGGGAAAGAGCATGATGACATATTTGATAAACTTAA
AGAGGCTGTTAAGGAAGAAAGTATTAAACGACACAAGTGGAATGACTTTGCGGAGGACAGCTTGAGGGT
TATTCAACACAATGCTTTGGAAGACCGATCCATATCTGATAAACAGCAATGGGATGCAGCTATTTATTT
TATGGAAGAGGCTCTGCAGGCTCGTCTCAAGGATACTGAAAATGCAATTGAAAACATGGTGGGTCCAGA
CTGGAAAAAGAGGTGGTTATACTGGAAGAATCGGACCCAAGAACAGTGTGTTCACAATGAAACCAAGAA
TGAATTGGAGAAGATGTTGAAATGTAATGAGGAGCACCCAGCTTATCTTGCAAGTGATGAAATAACCAC
AGTCCGGAAGAACCTTGAATCCCGAGGAGTAGAAGTAGATCCAAGCTTGATTAAGGATACTTGGCATCA
AGTTTATAGAAGACATTTTTTAAAAACAGCTCTAAACCATTGTAACCTTTGTCGAAGAGGTTTTTATTA
CTACCAAAGGCATTTTGTAGATTCTGAGTTGGAATGCAATGATGTGGTCTTGTTTTGGCGTATACAGCG
CATGCTTGCTATCACCGCAAATACTTTAAGGCAACAACTTACAAATACTGAAGTTAGGCGATTAGAGAA
AAATGTTAAAGAGGTATTGGAAGATTTTGCTGAAGATGGTGAGAAGAAGATTAAATTGCTTACTGGTAA
ACGCGTTCAACTGGCGGAAGACCTCAAGAAAGTTAGAGAAATTCAAGAAAAACTTGATGCTTTCATTGA
AGCTCTTCATCAGGAGAAATAAATTAAAATCGTACTCATAATCAGCTCTGCATACATCTGAAGAACAAA
AACATCAACGTCTTTTGTCCAGCCTCTTTTTCTTCTGCTGTTCCACCTTTCTAAACATACAATAAAGTC
ATGGGATAAAAATAATCGATGTATGTTACGGGCGCTTTAACCATCAGCTGCCTCTCGAATGGAAGAACA
GTGGTAATGGATTAACATCCTATTTTGTTGTACTAAAGTGACAAATCGGAATAATATAATTGGTATGGC
CATTAGGTTCAGTCCTTGAAGATAAGAAACTTGTTCTCTGTTTGTTGTCTTATTTGTGGTGGCACTCGT
TTAATGGATTAACTGAGGTTGCTCAATGTTCAGTTTCTTTTCCAGAAATACAATGCTAGGTGTTTTGAA
ATAAAACTTATATAGCAATTGTTTAAAGTTATCAATTGTATATAAAATCACAGTAGCCTGCTAAATCAT
TGTATGTGTCTGTAGTATTCTATTCCCAGAAACTATTTGACCATGATAATTCAGTTTATATTCACCACA
TGAAAGAAAAATGGGTAACAGAAGAACCCTTAAAACAGGTTAATTTGGATTGTAACGTTCAGTGAAAGA
AATTTCAACCCTTCATAGCCAGCGAAGAAATTTGCCTTGGAAGCCAAGTCAGTACCAGCTTACCTATTT
GATTCAGTTGCTGTTTTCTCACTCTCTATATCCATTTGAAATTGATTTATTTTAGATGTTGTATACTTA
CGTTAGGCTTTCTGTTAATAGTGGTTTTTCTCCTGTTGACAGAGCCACCGGATTATGACACAGGATGAG
GAAGATTAAGGATAATCAATTGACTAATTTCATTTAGAATATTATCAAACATTTCAACTAGGTATCAGA
AAAAGGCTTTCTTTCATAAGACTATTTTAAATAGAAATTATTTCAACAATTAAAGTAATGTTGACCATC
CCCCTCTCAGCTGAATAAAGAAAAATTTAGTTCAATTTATTGCAATTTAATTACAATACTACCTTCACA
ACATTTTCATGTGTTTTAAATAAATATTTTTTAATTGGCTAAAGGACATTCAAGCAAAGAAATGCTTTC
TTTACTTAAAATGTCTATCTCATTTGCTGCCTTTTCACTAAGCCTTTACTTTGTTAATAAAAGTGTCCA
TTGTGTGATGTTTTTGATTTTACAGTTTGCTAAATCTTATTTTCTTGGAGTTGCTTTTTGGTAACAGCC
CCATTGCTACTCCCCATTTTATTGTTTTACATCAATGCATGCTTCGTTGTGATCCCTCAAGATGTAACA
CTTGGTATGCTCGGTTGAGGATATGAAAAAATACTTCCGAAACCAGGAATTCAATGTATGTTTGTTTTA
TACTGTTTGATAAGAAAAGTAGGTCCAGCCTTAAGCAGCACAGATGCGCTGGTAGATGCATAGTCAGGA
ACTTTTTTTATTTCTTTTAGGTCTAGGGACAGGAGTGAATAGAAAGGGAGGAGAGCTCTATTATGTTCT
ATACACAGATTAGGAGATGACCTTACTGGGTACACCCCTCTAACCAGTGCTTACAGGTTAATGCATGTT
AATGAATATTTTTGCAGTTGTAAAGCATAACAATTACAACTACACATCTATTTCTAAAGAATAAAACAG
GACCATATTTATTTACTTCTGTCAACTATAGAAAGAAAGACCTTCAGCTGTATTTCCACAGATTTCTCC
CAAGGAAAAGGCTAATATTAGTCACTACTGTTATCACATCCCTTTGTATAAGTTTTAAAAAGAGATGGA
GGGAGATCTTCATTTCTTTGAGGAGATCAGTATTGTAACGTATGTGAATAGATGATAACAATTAATATT
ACTAAAAGTCCCACATGAGAGTCCTGACGCCCTCTCCATGCCCCACAGTAATGTGGCTTCTTTCATGGG
TTTTTTTTTCTTCTTTTTAGCTGATCTCATCCTAAGCATGCTTTATTTTTCCTTGAAAGCTAGGTATTT
ATCAACTGCAGATGTTATTGAAAGAAAATAAAATTCAGTC

TABLE 1.4
List of Exemplary OPA1 mRNA 5′ UTR Sequences

ENST00000361715.6 chr3:193593143-193593377, SEQ ID
NO: 1263, organism: homo sapiens
GTGCTGCCCGCCTAGAAAGGGTGAAGTGGTTGTTTCCGTGACGGACTGAG
TACGGGTGCCTGTCAGGCTCTTGCGGAAGTCCATGCGCCATTGGGAGGGC
CTCGGCCGCGGCTCTGTGCCCTTGCTGCTGAGGGCCACTTCCTGGGTCAT
TCCTGGACCGGGAGCCGGGCTGGGGCTCACACGGGGGCTCCCGCGTGGCC
GTCTCGGCGCCTGCGTGACCTCCCCGCCGGCGGG
ENST00000361150.6 chr3:193593143-193593377, SEQ ID
NO: 1264, organism: homo sapiens
GTGCTGCCCGCCTAGAAAGGGTGAAGTGGTTGTTTCCGTGACGGACTGAG
TACGGGTGCCTGTCAGGCTCTTGCGGAAGTCCATGCGCCATTGGGAGGGC
CTCGGCCGCGGCTCTGTGCCCTTGCTGCTGAGGGCCACTTCCTGGGTCAT
TCCTGGACCGGGAGCCGGGCTGGGGCTCACACGGGGGCTCCCGCGTGGCC
GTCTCGGCGCCTGCGTGACCTCCCCGCCGGCGGG
ENST00000361828.7 chr3:193593143-193593377, SEQ ID
NO: 1265, organism: homo sapiens
GTGCTGCCCGCCTAGAAAGGGTGAAGTGGTTGTTTCCGTGACGGACTGAG
TACGGGTGCCTGTCAGGCTCTTGCGGAAGTCCATGCGCCATTGGGAGGGC
CTCGGCCGCGGCTCTGTGCCCTTGCTGCTGAGGGCCACTTCCTGGGTCAT
TCCTGGACCGGGAGCCGGGCTGGGGCTCACACGGGGGCTCCCGCGTGGCC
GTCTCGGCGCCTGCGTGACCTCCCCGCCGGCGGG
ENST00000361908.8 chr3:193593185-193593377, SEQ ID
NO: 1266, organism: homo sapiens
GGACTGAGTACGGGTGCCTGTCAGGCTCTTGCGGAAGTCCATGCGCCATT
GGGAGGGCCTCGGCCGCGGCTCTGTGCCCTTGCTGCTGAGGGCCACTTCC
TGGGTCATTCCTGGACCGGGAGCCGGGCTGGGGCTCACACGGGGGCTCCC
GCGTGGCCGTCTCGGCGCCTGCGTGACCTCCCCGCCGGCGGG
ENST00000361510.8 chr3:193593207-193593377, SEQ ID
NO: 1267, organism: homo sapiens
AGGCTCTTGCGGAAGTCCATGCGCCATTGGGAGGGCCTCGGCCGCGGCTC
TGTGCCCTTGCTGCTGAGGGCCACTTCCTGGGTCATTCCTGGACCGGGAG
CCGGGCTGGGGCTCACACGGGGGCTCCCGCGTGGCCGTCTCGGCGCCTGC
GTGACCTCCCCGCCGGCGGG
ENST00000392436.7 chr3:193593219-193593377, SEQ ID
NO: 1268, organism: homo sapiens
AAGTCCATGCGCCATTGGGAGGGCCTCGGCCGCGGCTCTGTGCCCTTGCT
GCTGAGGGCCACTTCCTGGGTCATTCCTGGACCGGGAGCCGGGCTGGGGC
TCACACGGGGGCTCCCGCGTGGCCGTCTCGGCGCCTGCGTGACCTCCCCG
CCGGCGGG
ENST00000646793.1 chr3:193593246-193593377, SEQ ID
NO: 1269, organism: homo sapiens
GGCCGCGGCTCTGTGCCCTTGCTGCTGAGGGCCACTTCCTGGGTCATTCC
TGGACCGGGAGCCGGGCTGGGGCTCACACGGGGGCTCCCGCGTGGCCGTC
TCGGCGCCTGCGTGACCTCCCCGCCGGCGGG
ENST00000392437.6 chr3:193593279-193593377, SEQ ID
NO: 1270, organism: homo sapiens
ACTTCCTGGGTCATTCCTGGACCGGGAGCCGGGCTGGGGCTCACACGGGG
GCTCCCGCGTGGCCGTCTCGGCGCCTGCGTGACCTCCCCGCCGGCGGG
ENST00000645553.1 chr3:193593286-193593377, SEQ ID
NO: 1271, organism: homo sapiens
GGGTCATTCCTGGACCGGGAGCCGGGCTGGGGCTCACACGGGGGCTCCCG
CGTGGCCGTCTCGGCGCCTGCGTGACCTCCCCGCCGGCGGG

TABLE 2
List of Exemplary OPA1 Protein Sequences

ENST00000361715.6, SEQ ID NO: 1272, organism: homo sapiens
MWRLRRAAVACEVCQSLVKHSSGIKGSLPLQKLHLVSRSIYHSHHPTLKLQRPQLRTSFQQFSSLTNLP
LRKLKFSPIKYGYQPRRNFWPARLATRLLKLRYLILGSAVGGGYTAKKTFDQWKDMIPDLSEYKWIVPD
IVWEIDEYIDFGHKLVSEVIGASDLLLLLGSPEETAFRATDRGSESDKHFRKGLLGELILLQQQIQEHE
EEARRAAGQYSTSYAQQKRKVSDKEKIDQLQEELLHTQLKYQRILERLEKENKELRKLVLQKDDKGIHH
RKLKKSLIDMYSEVLDVLSDYDASYNTQDHLPRVVVVGDQSAGKTSVLEMIAQARIFPRGSGEMMTRSP
VKVTLSEGPHHVALFKDSSREFDLTKEEDLAALRHEIELRMRKNVKEGCTVSPETISLNVKGPGLQRMV
LVDLPGVINTVTSGMAPDTKETIFSISKAYMQNPNAIILCIQDGSVDAERSIVTDLVSQMDPHGRRTIF
VLTKVDLAEKNVASPSRIQQIIEGKLFPMKALGYFAVVTGKGNSSESIEAIREYEEEFFQNSKLLKTSM
LKAHQVTTRNLSLAVSDCFWKMVRESVEQQADSFKATRFNLETEWKNNYPRLRELDRNELFEKAKNEIL
DEVISLSQVTPKHWEEILQQSLWERVSTHVIENIYLPAAQTMNSGTFNTTVDIKLKQWTDKQLPNKAVE
VAWETLQEEFSRFMTEPKGKEHDDIFDKLKEAVKEESIKRHKWNDFAEDSLRVIQHNALEDRSISDKQQ
WDAAIYFMEEALQARLKDTENAIENMVGPDWKKRWLYWKNRTQEQCVHNETKNELEKMLKCNEEHPAYL
ASDEITTVRKNLESRGVEVDPSLIKDTWHQVYRRHFLKTALNHCNLCRRGFYYYQRHFVDSELECNDVV
LFWRIQRMLAITANTLRQQLTNTEVRRLEKNVKEVLEDFAEDGEKKIKLLTGKRVQLAEDLKKVREIQE
KLDAFIEALHQEK
ENST00000361150.6, SEQ ID NO: 1273, organism: homo sapiens
MWRLRRAAVACEVCQSLVKHSSGIKGSLPLQKLHLVSRSIYHSHHPTLKLQRPQLRTSFQQFSSLTNLP
LRKLKFSPIKYGYQPRRNFWPARLATRLLKLRYLILGSAVGGGYTAKKTFDQWKDMIPDLSEYKWIVPD
IVWEIDEYIDFGSPEETAFRATDRGSESDKHFRKGLLGELILLQQQIQEHEEEARRAAGQYSTSYAQQK
RKVSDKEKIDQLQEELLHTQLKYQRILERLEKENKELRKLVLQKDDKGIHHRKLKKSLIDMYSEVLDVL
SDYDASYNTQDHLPRVVVVGDQSAGKTSVLEMIAQARIFPRGSGEMMTRSPVKVTLSEGPHHVALFKDS
SREFDLTKEEDLAALRHEIELRMRKNVKEGCTVSPETISLNVKGPGLQRMVLVDLPGVINTVTSGMAPD
TKETIFSISKAYMQNPNAIILCIQDGSVDAERSIVTDLVSQMDPHGRRTIFVLTKVDLAEKNVASPSRI
QQIIEGKLFPMKALGYFAVVTGKGNSSESIEAIREYEEEFFQNSKLLKTSMLKAHQVTTRNLSLAVSDC
FWKMVRESVEQQADSFKATRFNLETEWKNNYPRLRELDRNELFEKAKNEILDEVISLSQVTPKHWEEIL
QQSLWERVSTHVIENIYLPAAQTMNSGTENTTVDIKLKQWTDKQLPNKAVEVAWETLQEEFSRFMTEPK
GKEHDDIFDKLKEAVKEESIKRHKWNDFAEDSLRVIQHNALEDRSISDKQQWDAAIYFMEEALQARLKD
TENAIENMVGPDWKKRWLYWKNRTQEQCVHNETKNELEKMLKCNEEHPAYLASDEITTVRKNLESRGVE
VDPSLIKDTWHQVYRRHFLKTALNHCNLCRRGFYYYQRHFVDSELECNDVVLFWRIQRMLAITANTLRQ
QLTNTEVRRLEKNVKEVLEDFAEDGEKKIKLLTGKRVQLAEDLKKVREIQEKLDAFIEALHQEK
ENST00000361828.7, SEQ ID NO: 1274, organism: homo sapiens
MWRLRRAAVACEVCQSLVKHSSGIKGSLPLQKLHLVSRSIYHSHHPTLKLQRPQLRTSFQQFSSLTNLP
LRKLKFSPIKYGYQPRRNFWPARLATRLLKLRYLILGSAVGGGYTAKKTFDQWKDMIPDLSEYKWIVPD
IVWEIDEYIDFEKIRKALPSSEDLVKLAPDFDKIVESLSLLKDFFTSGSPEETAFRATDRGSESDKHFR
KVSDKEKIDQLQEELLHTQLKYQRILERLEKENKELRKLVLQKDDKGIHHRKLKKSLIDMYSEVLDVLS
DYDASYNTQDHLPRVVVVGDQSAGKTSVLEMIAQARIFPRGSGEMMTRSPVKVTLSEGPHHVALFKDSS
REFDLTKEEDLAALRHEIELRMRKNVKEGCTVSPETISLNVKGPGLQRMVLVDLPGVINTVTSGMAPDT
KETIFSISKAYMQNPNAIILCIQDGSVDAERSIVTDLVSQMDPHGRRTIFVLTKVDLAEKNVASPSRIQ
QIIEGKLFPMKALGYFAVVTGKGNSSESIEAIREYEEEFFQNSKLLKTSMLKAHQVTTRNLSLAVSDCF
WKMVRESVEQQADSFKATRFNLETEWKNNYPRLRELDRNELFEKAKNEILDEVISLSQVTPKHWEEILQ
QSLWERVSTHVIENIYLPAAQTMNSGTFNTTVDIKLKQWTDKQLPNKAVEVAWETLQEEFSRFMTEPKG
KEHDDIFDKLKEAVKEESIKRHKWNDFAEDSLRVIQHNALEDRSISDKQQWDAAIYFMEEALQARLKDT
ENAIENMVGPDWKKRWLYWKNRTQEQCVHNETKNELEKMLKCNEEHPAYLASDEITTVRKNLESRGVEV
DPSLIKDTWHQVYRRHFLKTALNHCNLCRRGFYYYQRHFVDSELECNDVVLFWRIQRMLAITANTLRQQ
LTNTEVRRLEKNVKEVLEDFAEDGEKKIKLLTGKRVQLAEDLKKVREIQEKLDAFIEALHQEK
ENST00000361908.8, SEQ ID NO: 1275, organism: homo sapiens
MWRLRRAAVACEVCQSLVKHSSGIKGSLPLQKLHLVSRSIYHSHHPTLKLQRPQLRTSFQQFSSLTNLP
LRKLKFSPIKYGYQPRRNFWPARLATRLLKLRYLILGSAVGGGYTAKKTFDQWKDMIPDLSEYKWIVPD
IVWEIDEYIDFEKIRKALPSSEDLVKLAPDFDKIVESLSLLKDFFTSGSPEETAFRATDRGSESDKHFR
KGLLGELILLQQQIQEHEEEARRAAGQYSTSYAQQKRKVSDKEKIDQLQEELLHTQLKYQRILERLEKE
NKELRKLVLQKDDKGIHHRKLKKSLIDMYSEVLDVLSDYDASYNTQDHLPRVVVVGDQSAGKTSVLEMI
AQARIFPRGSGEMMTRSPVKVTLSEGPHHVALFKDSSREFDLTKEEDLAALRHEIELRMRKNVKEGCTV
SPETISLNVKGPGLQRMVLVDLPGVINTVTSGMAPDTKETIFSISKAYMQNPNAIILCIQDGSVDAERS
IVTDLVSQMDPHGRRTIFVLTKVDLAEKNVASPSRIQQIIEGKLFPMKALGYFAVVTGKGNSSESIEAI
REYEEEFFQNSKLLKTSMLKAHQVTTRNLSLAVSDCFWKMVRESVEQQADSFKATRFNLETEWKNNYPR
LRELDRNELFEKAKNEILDEVISLSQVTPKHWEEILQQSLWERVSTHVIENIYLPAAQTMNSGTFNTTV
DIKLKQWTDKQLPNKAVEVAWETLQEEFSRFMTEPKGKEHDDIFDKLKEAVKEESIKRHKWNDFAEDSL
RVIQHNALEDRSISDKQQWDAAIYFMEEALQARLKDTENAIENMVGPDWKKRWLYWKNRTQEQCVHNET
KNELEKMLKCNEEHPAYLASDEITTVRKNLESRGVEVDPSLIKDTWHQVYRRHFLKTALNHCNLCRRGF
YYYQRHFVDSELECNDVVLFWRIQRMLAITANTLRQQLTNTEVRRLEKNVKEVLEDFAEDGEKKIKLLT
GKRVQLAEDLKKVREIQEKLDAFIEALHQEK
ENST00000361510.8, SEQ ID NO: 1276, organism: homo sapiens
MWRLRRAAVACEVCQSLVKHSSGIKGSLPLQKLHLVSRSIYHSHHPTLKLQRPQLRTSFQQFSSLTNLP
LRKLKFSPIKYGYQPRRNFWPARLATRLLKLRYLILGSAVGGGYTAKKTFDQWKDMIPDLSEYKWIVPD
IVWEIDEYIDFEKIRKALPSSEDLVKLAPDFDKIVESLSLLKDFFTSGHKLVSEVIGASDLLLLLGSPE
ETAFRATDRGSESDKHFRKGLLGELILLQQQIQEHEEEARRAAGQYSTSYAQQKRKVSDKEKIDQLQEE
LLHTQLKYQRILERLEKENKELRKLVLQKDDKGIHHRKLKKSLIDMYSEVLDVLSDYDASYNTQDHLPR
VVVVGDQSAGKTSVLEMIAQARIFPRGSGEMMTRSPVKVTLSEGPHHVALFKDSSREFDLTKEEDLAAL
RHEIELRMRKNVKEGCTVSPETISLNVKGPGLQRMVLVDLPGVINTVTSGMAPDTKETIFSISKAYMQN
PNAIILCIQDGSVDAERSIVTDLVSQMDPHGRRTIFVLTKVDLAEKNVASPSRIQQIIEGKLFPMKALG
YFAVVTGKGNSSESIEAIREYEEEFFQNSKLLKTSMLKAHQVTTRNLSLAVSDCFWKMVRESVEQQADS
FKATRFNLETEWKNNYPRLRELDRNELFEKAKNEILDEVISLSQVTPKHWEEILQQSLWERVSTHVIEN
IYLPAAQTMNSGTFNTTVDIKLKQWTDKQLPNKAVEVAWETLQEEFSRFMTEPKGKEHDDIFDKLKEAV
KEESIKRHKWNDFAEDSLRVIQHNALEDRSISDKQQWDAAIYFMEEALQARLKDTENAIENMVGPDWKK
RWLYWKNRTQEQCVHNETKNELEKMLKCNEEHPAYLASDEITTVRKNLESRGVEVDPSLIKDTWHQVYR
RHFLKTALNHCNLCRRGFYYYQRHFVDSELECNDVVLFWRIQRMLAITANTLRQQLTNTEVRRLEKNVK
EVLEDFAEDGEKKIKLLTGKRVQLAEDLKKVREIQEKLDAFIEALHQEK
ENST00000392436.7, SEQ ID NO: 1277, organism: homo sapiens
MWRLRRAAVACEVCQSLVKHSSGIKGSLPLQKLHLVSRSIYHSHHPTLKLQRPQLRTSFQQFSSLTNLP
LRKLKFSPIKYGYQPRRNFWPARLATRLLKLRYLILGSAVGGGYTAKKTFDQWKDMIPDLSEYKWIVPD
IVWEIDEYIDFEKIRKALPSSEDLVKLAPDFDKIVESLSLLKDFFTSGSPEETAFRATDRGSESDKHFR
KVSDKEKIDQLQEELLHTQLKYQRILERLEKENKELRKLVLQKDDKGIHHRKLKKSLIDMYSEVLDVLS
DYDASYNTQDHLPRVVVVGDQSAGKTSVLEMIAQARIFPRGSGEMMTRSPVKVTLSEGPHHVALFKDSS
REFDLTKEEDLAALRHEIELRMRKNVKEGCTVSPETISLNVKGPGLQRMVLVDLPGVINTVTSGMAPDT
KETIFSISKAYMQNPNAIILCIQDGSVDAERSIVTDLVSQMDPHGRRTIFVLTKVDLAEKNVASPSRIQ
QIIEGKLFPMKALGYFAVVTGKGNSSESIEAIREYEEEFFQNSKLLKTSMLKAHQVTTRNLSLAVSDCF
WKMVRESVEQQADSFKATRFNLETEWKNNYPRLRELDRNELFEKAKNEILDEVISLSQVTPKHWEEILQ
QSLWERVSTHVIENIYLPAAQTMNSGTFNTTVDIKLKQWTDKQLPNKAVEVAWETLQEEFSRFMTEPKG
KEHDDIFDKLKEAVKEESIKRHKWNDFAEDSLRVIQHNALEDRSISDKQQWDAAIYFMEEALQARLKDT
ENAIENMVGPDWKKRWLYWKNRTQEQCVHNETKNELEKMLKCNEEHPAYLASDEITTVRKNLESRGVEV
DPSLIKDTWHQVYRRHFLKTALNHCNLCRRGFYYYQRHFVDSELECNDVVLFWRIQRMLAITANTLRQQ
LTNTEVRRLEKNVKEVLEDFAEDGEKKIKLLTGKRVQLAEDLKMFFFPSSDSTLHTRPPHGAAPSIDTS
FTLSGL
ENST00000646793.1, SEQ ID NO: 1278, organism: homo sapiens
MWRLRRAAVACEVCQSLVKHSSGIKGSLPLQKLHLVSRSIYHSHHPTLKLQRPQLRTSFQQFSSLTNLP
LRKLKFSPIKYGYQPRRNFWPARLATRLLKLRYLILGSAVGGGYTAKKTFDQWKDMIPDLSEYKWIVPD
IVWEIDEYIDFGSPEETAFRATDRGSESDKHFRKVSDKEKIDQLQEELLHTQLKYQRILERLEKENKEL
RKLVLQKDDKGIHHRKLKKSLIDMYSEVLDVLSDYDASYNTQDHLPRVVVVGDQSAGKTSVLEMIAQAR
IFPRGSGEMMTRSPVKVTLSEGPHHVALFKDSSREFDLTKEEDLAALRHEIELRMRKNVKEGCTVSPET
ISLNVKGPGLQRMVLVDLPGVINTVTSGMAPDTKETIFSISKAYMQNPNAIILCIQDGSVDAERSIVTD
LVSQMDPHGRRTIFVLTKVDLAEKNVASPSRIQQIIEGKLFPMKALGYFAVVTGKGNSSESIEAIREYE
EEFFQNSKLLKTSMLKAHQVTTRNLSLAVSDCFWKMVRESVEQQADSFKATRFNLETEWKNNYPRLREL
DRNELFEKAKNEILDEVISLSQVTPKHWEEILQQSLWERVSTHVIENIYLPAAQTMNSGTFNTTVDIKL
KQWTDKQLPNKAVEVAWETLQEEFSRFMTEPKGKEHDDIFDKLKEAVKEESIKRHKWNDFAEDSLRVIQ
HNALEDRSISDKQQWDAAIYFMEEALQARLKDTENAIENMVGPDWKKRWLYWKNRTQEQCVHNETKNEL
EKMLKCNEEHPAYLASDEITTVRKNLESRGVEVDPSLIKDTWHQVYRRHFLKTALNHCNLCRRGFYYYQ
RHFVDSELECNDVVLFWRIQRMLAITANTLRQQLTNTEVRRLEKNVKEVLEDFAEDGEKKIKLLTGKRV
QLAEDLKKVREIQEKLDAFIEALHQEK
ENST00000392437.6, SEQ ID NO: 1279, organism: homo sapiens
MWRLRRAAVACEVCQSLVKHSSGIKGSLPLQKLHLVSRSIYHSHHPTLKLQRPQLRTSFQQFSSLTNLP
LRKLKFSPIKYGYQPRRNFWPARLATRLLKLRYLILGSAVGGGYTAKKTFDQWKDMIPDLSEYKWIVPD
IVWEIDEYIDFEKIRKALPSSEDLVKLAPDFDKIVESLSLLKDFFTSGHKLVSEVIGASDLLLLLGSPE
ETAFRATDRGSESDKHFRKVSDKEKIDQLQEELLHTQLKYQRILERLEKENKELRKLVLQKDDKGIHHR
KLKKSLIDMYSEVLDVLSDYDASYNTQDHLPRVVVVGDQSAGKTSVLEMIAQARIFPRGSGEMMTRSPV
KVTLSEGPHHVALFKDSSREFDLTKEEDLAALRHEIELRMRKNVKEGCTVSPETISLNVKGPGLQRMVL
VDLPGVINTVTSGMAPDTKETIFSISKAYMQNPNAIILCIQDGSVDAERSIVTDLVSQMDPHGRRTIFV
LTKVDLAEKNVASPSRIQQIIEGKLFPMKALGYFAVVTGKGNSSESIEAIREYEEEFFQNSKLLKTSML
KAHQVTTRNLSLAVSDCFWKMVRESVEQQADSFKATRFNLETEWKNNYPRLRELDRNELFEKAKNEILD
EVISLSQVTPKHWEEILQQSLWERVSTHVIENIYLPAAQTMNSGTFNTTVDIKLKQWTDKQLPNKAVEV
AWETLQEEFSRFMTEPKGKEHDDIFDKLKEAVKEESIKRHKWNDFAEDSLRVIQHNALEDRSISDKQQW
DAAIYFMEEALQARLKDTENAIENMVGPDWKKRWLYWKNRTQEQCVHNETKNELEKMLKCNEEHPAYLA
SDEITTVRKNLESRGVEVDPSLIKDTWHQVYRRHFLKTALNHCNLCRRGFYYYQRHFVDSELECNDVVL
FWRIQRMLAITANTLRQQLTNTEVRRLEKNVKEVLEDFAEDGEKKIKLLTGKRVQLAEDLKKVREIQEK
LDAFIEALHQEK
ENST00000645553.1, SEQ ID NO: 1280, organism: homo sapiens
MWRLRRAAVACEVCQSLVKHSSGIKGSLPLQKLHLVSRSIYHSHHPTLKLQRPQLRTSFQQFSSLTNLP
LRKLKFSPIKYGYQPRRNFWPARLATRLLKLRYLILGSAVGGGYTAKKTFDQWKDMIPDLSEYKWIVPD
IVWEIDEYIDFEKIRKALPSSEDLVKLAPDFDKIVESLSLLKDFFTSGSPEETAFRATDRGSESDKHFR
KVSDKEKIDQLQEELLHTQSKIWKLKYQRILERLEKENKELRKLVLQKDDKGIHHRKLKKSLIDMYSEV
LDVLSDYDASYNTQDHLPRVVVVGDQSAGKTSVLEMIAQARIFPRGSGEMMTRSPVKVTLSEGPHHVAL
FKDSSREFDLTKEEDLAALRHEIELRMRKNVKEGCTVSPETISLNVKGPGLQRMVLVDLPGVINTVTSG
MAPDTKETIFSISKAYMQNPNAIILCIQDGSVDAERSIVTDLVSQMDPHGRRTIFVLTKVDLAEKNVAS
PSRIQQIIEGKLFPMKALGYFAVVTGKGNSSESIEAIREYEEEFFQNSKLLKTSMLKAHQVTTRNLSLA
VSDCFWKMVRESVEQQADSFKATRFNLETEWKNNYPRLRELDRNELFEKAKNEILDEVISLSQVTPKHW
EEILQQSLWERVSTHVIENIYLPAAQTMNSGTFNTTVDIKLKQWTDKQLPNKAVEVAWETLQEEFSRFM
TEPKGKEHDDIFDKLKEAVKEESIKRHKWNDFAEDSLRVIQHNALEDRSISDKQQWDAAIYFMEEALQA
RLKDTENAIENMVGPDWKKRWLYWKNRTQEQCVHNETKNELEKMLKCNEEHPAYLASDEITTVRKNLES
RGVEVDPSLIKDTWHQVYRRHFLKTALNHCNLCRRGFYYYQRHFVDSELECNDVVLFWRIQRMLAITAN
TLRQQLTNTEVRRLEKNVKEVLEDFAEDGEKKIKLLTGKRVQLAEDLKKVREIQEKLDAFIEALHQEK

TABLE 3
Sequences of exemplary target introns in pre-mRNA transcripts.

| | SEQ ID | |
|---|---|---|
| Gene | NO. | Intron |
| OPA1 | 2 | Intron 6: |
| organism: | | gtgatggatggtttaagggggctaccgatacattcacactaatcagccatttc |
| homo | | tgccaagatcatgtcacctcaatctgttcatggactccaaatacaagaaatta |
| sapiens | | atttgacaaagtgaaaatataaaagatgcatcatataaatatgtaacttttct |
| | | ggagtgggtagtataggtaaagccaaaagaaacaaattcaagcagaggaattt |
| | | tggtttctgaaaattaggttgtctgtagggtccctgtatttatacttagaaca |
| | | aaattaggaatttctgtttatgtggtccagttattgagtcaccctaagtttgt |
| | | aggcatcttacctacctacttgctccccaagtttttatttctaaaatgaaaag |
| | | cattgctgtagatgaccagtttacactaaagaataacatttatttatttgttt |
| | | tagctaaagtatatggacagggaacattcatattcttgtagaagaaaattatt |
| | | ttgacttttgggcaaaagcatgtagttcttatacactttgacaaactcattgc |
| | | gtacatttttcacattaatcaaagtcagcacaaataaattttcaccttggacc |
| | | acggagggtttgaacactggaaatttgatataattctggttgctaaagaacaa |
| | | gttctaataaaagcttaagtgtataccaatatgtggctgttggtgcaatcagc |
| | | aggtccgtaaaaatatgattttaatggttaggtaatcccacaacggagatccc |
| | | aaagttcatgtttggaagagacttttgggtcaaagtgaaatcagtgtaatgaa |
| | | tttaaaattatactctgagatcttgaaatcagctaattatgttacatcttatt |
| | | agctcagaaaagttttgaagttatatacaaatgctagtcaggaaaaaagattc |
| | | agtcatgtaattcttgtacattctactatttaaatcaaccaatattatagatt |
| | | atgatttagtgcagtaattctgctggctaaccttatctcatttggtggtggtt |
| | | agtacttcagagtactcaccatagtttcatttatgttttcagcatcacttcct |
| | | ggtttttctcaattccatggctgtggaatcaattcatatgtatatttagcttc |
| | | ggtgagcaaaaacatagctagaaaaagaaaagaagtgagtttcctacctggtt |
| | | aaattaaagtcgatgtgttaagccaaggaggacttcttttgaatggtacttta |
| | | acaatccctgttctgtatactgtgaatatatcatttaaatagcctaataaatt |
| | | ggatgcttaggctgagccacctatactttagttttgttatggaaagaagggag |
| | | aggagcaagtatgttcttatatgttacttagaaataagaatgtagctgtagtt |
| | | acacattgttcttaagtttttttcgtaagacaacttgaaatgagtcccatagg |
| | | cctgctatttaacattctaagatatgacttaaggttaatgatgagcttttgaa |
| | | tctgacaattcaagagatatccataatgaatactgattcattttctacattgc |
| | | tgaaagctaatgttcattttaagcctactttagtagcctttatttgggcttag |
| | | agatgttattcctctttctgatatttattgggttatctgtttaacccttttat |
| | | atctccctttcccgatttgtaaattagagactggcaagactttttaccctgag |
| | | tagagcaccaaacatggcttgtttctgcccacactgtagttaccttgagggga |
| | | agtaaatgggactttaaaagcaatttatgctcttttatagtgaaattatccct |
| | | cttactatcccgaaagactgttaccttacaatatcctccactcctttccccct |
| | | gtagttactatagagatgacttttcggttcttcactgccataatgatcaaaat |
| | | cctaattcatgagatttttatcattccaggcatgtgaggtttacttgatgcat |
| | | aaaaccgcaagtactttttgttgttttttaattgttttttctctcttatcttc |
| | | ttgaaagtctaagtagatcatcatttttgatgtcttattagtagcaactaata |
| | | aattttccctgtatcttctcagcaaaagaactcaagcagagacagaagattag |
| | | aactaccattggtagttttgcttcctatggatatgttcacatacatagaaatt |
| | | tttacaatgacctttttatatatgtatttcagaatttcagaatggcctcaatg |
| | | ccttaataggaagaaatacttgaaatttttaaattagggcttggttttgtgag |
| | | gagctagtaaaggtttttctctttcagctttagcttgtttctgcggaggattc |
| | | cgctctttctccatcagtttcatagccctggaattgtagaaaagctctggttt |
| | | caagaccattgatatccatttctgtcagggtgagttttaaatttatttcatga |
| | | tgcaaacaatatattgaacaacaggacatgaacttgttcttgttgtaagtggc |
| | | tgaattttatcagtaaagcacatcaaaataaaatataccccaattgctagtta |
| | | agacctagagtgacagattgaaaatagcttgtgttattctcttaagaaaatat |
| | | ataaaaattatcatctcatcaatctttaatgtttgttttataaatctaaatgt |
| | | ttttatattgtttcctaggaaatattaggtctaattttttactttaccaccag |
| | | ctgtcttttattttactctttttttgagacggagtttcgctcttgttgcttag |
| | | gctagagtgcagtggcactatctcagctcactgcgacctctgcctcccgggtt |
| | | caagcgattctcctgcctcagtctcccgagtagctgggattacaggcacatgc |
| | | cactacaccaggctaattttgtatttttagtagagacggggtttcttcatgtt |
| | | ggtcaggctggtctcgaactcccgacctcaggtgatccgcctgcctcggcctc |
| | | ccagagtgctgggattacaggcatgagccaccgcacctggccagctgtctttt |
| | | aatataacattatgattaattgtgatgttccattaaactaagcggagaggaaa |
| | | catgctggtaaaccatgtgtgagttattcattgtaccagaaaggcaaatgata |
| | | cattttatcctaaaattcaaatttataaacatcttaacacttgtgatcattaa |
| | | atactactaatctagcatataaattatatttgtaggcggggcacggtggctca |
| | | cgcctgtaatcccagcactttgggaggctgaggtgggcagatcacgaggtcag |
| | | gagatcgagaccatcctggctaacatggtgaaaccccatctctactaaaaata |
| | | caaaaaaaattagctgggtgtgctggcgggcacctgtagtcccagctacttgg |
| | | gaggctgaggcaggagaatggcgtgaccccaggaggcagagcttccagcctgg |
| | | gcgactccgtctcaaaaaaaaagaaaaaagaaattatatttgtaatattctac |
| | | taaccttatatcattttaactttttatataacttttttattttaccaaattaa |
| | | gttaaccttttatagcccttggcttatactaaacatcctaacttttttgttta |
| | | attgtattagtttttaagttattgccccagatgtcaagtaatgttggattttc |
| | | tataataatttaggatatattgcatgaagtcagttagtatttacatttaaaac |
| | | taaaacaatttatactaatacagtttatacatttcatactaatttagctacag |
| | | ttggataaatatttaatggaacaaagtaaatcaaagtaccttttcaaatgaat |
| | | tggaaattaaatccacataacaattttttatgaccacactattacagtgtgat |
| | | ggcatgccaaatgatcataatgtggaattatgtatttcttcattggctttcaa |
| | | gattctgttctttagtttgtgggctcctctccaacttgcttgtctcctcacag |
| | | tttaggcgactgtttataattcttgtccatcctgcataaacacacacagtcaa |
| | | aatgaaaaaaagcttctatcagcagatctgtgcttgctgtacagaaatgggaa |
| | | aacaattgaagtttgcattatcttttttctaattaccagatcgtttttggagc |
| | | tatttaggcatacgcttttaaggaaaaaagaaaaaaagagtgtaccttttgtt |
| | | tctaacaaaggttgttatctatattattgaaataaaaaattggggatagttat |
| | | gacaaagtatttagaaataggaattaaaatcttaaaataacttttcatagcat |
| | | ggacaagacttattaatgtctacctcaataagcaaatcatttaaaaatttttc |
| | | atgtatatttgctgccatgatgtgttgtgattgcttaaataaccaatgaatga |
| | | agatcaacaaggatttaaatgaagaagaatatggatttaactattttctcctg |
| | | tgaaataagttcatatttacaagttttgattttcagaaattagacaattattt |
| | | ttaaaggctgggatgacaacttctgcctcttaccaagaagtcaaagcacagtt |
| | | atgtgaattcatcataaatcacatcatttttattatattttgtatttataatt |
| | | gtattgtgactactttaaaacctgttataaaataaaattgttttttaatattt |
| | | tattttagaattattagcattaataacaatttgaagtagtttacacaatacct |
| | | gtgagttttatttttgttttatattgaaattaattttagttgctttacttggc |
| | | ttcattgctatggatgcattctctgtgttacgagttagcagatctttccttgg |
| | | aactgaatttaaaagcaagcatttggctccacttaaatctctgaaaatgcaac |
| | | ttgttctttgcatttattacataattcgctacttatggtacagaaatggatac |
| | | aatacaaaaatatttccttataagatacactgtgaccaatgagctttttaaat |
| | | agctgtaatcagtaacatgtatttgacttttcaaaacacatttctggagggat |
| | | atcagtgctttatttccccaaatatctgaatccctatgctttagtacaaaaca |
| | | acttctgaagaatttagtaaccatatgtgttgatctcttgtttttctaactag |
| | | tctttcataagaaatgactagaatagcaacagggaaatgattgccttttaagg |
| | | tttttgtttctcaatataaaattttggtgaaccatttttattgataaatacag |
| | | gtatttttactttcttaaatcacttgatttaaaattactttgattaaatatgc |
| | | atataaagtcagttgtttttaactctcaatacttatcaaaaaaatttaacttg |
| | | ctgtacattctgtataaacctaattctattcaactaaaattattttaaacatt |
| | | tag |
| | 3 | Intron 28: |
| | | gtgagtagttcttactgccctctaccttactacctttccacctttcccatttc |
| | | catttgtttgttgatccatttaatctcaaacttacagaaaagttacaaggaac |
| | | tgggctgagcacggtggctcacgcttgtaatcccagcactttgggaggccaag |
| | | atgggtggaaaacaaggtcagaagatcaagaccatcctggctaacacagtgaa |
| | | accccgtctctactaaaaatacaaaaaacttagccaggtgtggtggtgggtgc |
| | | ctatagtcccagctacttgggaggctgaggcaggagaatggtgtgaacccggg |
| | | aggcggagcttgcggtgagccaagatcctgccactgcactccagcctgagcga |
| | | cagggcgagattctgtctcaaaaaaaaaaaaaaaaaaaaagttacaaggaatt |
| | | tttttcttctctgaagtatttgagagtaagttgctgaccttaagtcctatcac |
| | | ttccaagtaggttcatgtatagttcttagaaacagattttctcatagcaaccg |
| | | aacattgataaattacaatatctaattctcagacccctttcaagtttcacccg |
| | | ttgtcccagtattatccctccatataacaagatgttccaggctcaatacctga |
| | | cccagcttcctttttttgaagaatggtgtttagaaatggagacctagaaatta |
| | | tatatgctgttattggaatatcactgttccctggtttctcagtggaaagagct |
| | | aggaactaagtgttgtgaatgtttgtgtgtgtgcaggtgaatatacacacact |
| | | gacatctgtattcctaaatcatgtgtatatttatttattaaaaactgtgagtt |
| | | gatgctgatacttcccattttaatccagcattacaaggtttgttctagtgttc |
| | | tccctttcgatatttgtcacttgctttcctgatagaaaacgggcttctagtat |
| | | ccttaatatattttcatattttggtcagtcctcctatacgtaacccaacttga |
| | | atgaagatatgtccttttccattgcagaaatgttctttttccccagctcggac |
| | | tcaacactacacaccaggccaccacatggcgccgcacccagcattgacacttc |
| | | ttttaccttgtctgggctctgacatccgtgccaggttgctcttcgtcatggag |
| | | tcccttttactgagctctgctctgacgctttgtgccaggtgcctctccatctc |
| | | atccttcccacccgctagcctctgcccgaccccagacagattccttcctcacc |
| | | tgaagccagaccatgcctttgtggagataccctctttaccctgcctgtgcttc |
| | | gccagcctgcaccaggccaccctcctgcacagatactctcctcagtactggac |
| | | caggctaccaacagccccatgtgaacccattgtaacccaggtcaggcattaac |
| | | acctgcagtaggctaccatggcttccccttcccacccccctagcttggcccta |
| | | ctaataatcactttgtcactgtttggggttgatatttggttgtttcttgtagg |
| | | ttcctagctttaagataggattgcatactaaaatttacttagatctttgagaa |
| | | ctcaaggaaatcagtgaaacattattgttattaaataaaaataaaatacctgt |
| | | agttggtacctctgtttgagcctgccttgttacaagtttcactgacttcagct |
| | | tcgtgtaacaaagtatctttttctttcaacgtgtacttaaatttcctgtctta |
| | | ttagttttctgatatctaaaaggaaaaaaagcagatatcgttaataaattaga |
| | | aagaagttctgcaaatttaaaagtgccttctaagctgagttgtaggattacag |
| | | tacaatccatagggttatcctgaagaagccaggcagggctcttctgtgttaca |
| | | ccctgtgcctgcgcagcatgctcaccccttgccatcagcgcttgcggccccat |
| | | tctctccctctagtaataatctaagttctgcattgctttctcctttccttttc |
| | | tttcttcctttaaatattcttctttcgagacatatctcattttaacttttatt |
| | | ttcattttctgtcacttttggtttttctcatgccaccttggcaatgtagttaa |
| | | gtttgtgctaacgtagaagattagtgctcaaatctgaattgccatttactact |
| | | agctgtgtcatcttcggcagggaatctcccagagccttagcttctttatttgt |
| | | aaaatgactattatagtggttatttctcaggattgttagaattacttccgcaa |
| | | acatttgcaagtccctggttcataatttcatgctaaattagtaccgttacagg |
| | | aagtggtatatcattgtcacagtgtatacaaatatatttcttttatatccctc |
| | | gtgatataattatcaagacagtgaaacaattcaatgaattttaccagcataac |
| | | acatttttaagtgattggaaaatcataagtatcttttcttatgtttttagtag |
| | | aggctttgcaaccccattactctccgctcccaatttgattatttaaaggaagt |
| | | ggattactaactcagatatgtacactgtcaagccaagttctatgttctactgc |
| | | tggttttcctgagaaagcagtcatataactcccttgaaatgatttactacttt |
| | | tgtacatataaaattataatggtgttaatgtaccaaataatgtccttggaagc |
| | | aagggttttgccagtaactcagctgcatcagtcaccctcaaggagatgagcca |
| | | tgactttgttcattagttggaaaagagtctggagagtgccttttcgttactgt |
| | | ttatctttggtctgacacttgggaatagggtcatggatacttcagccagaaaa |
| | | ctttccaaatttaagttattaatgtattataaggatcaaagtttctagtatag |
| | | cctgttcaattagaacatagtgtgttggttgattggatttggagaaagggagg |
| | | caatcaaatttttactacagtttcagcctgttacagaatattgtatagagtgt |
| | | taaaatgttgatgcattcatatttttgccagttttaagcttgtacgattttaa |
| | | atcatttccttaccttggagacttcccccccacctttttttttttttttgaga |
| | | tggagtctcgctgtgtcgcccaggctagagtgcagtggcacgatctcggctca |
| | | ctgcaaggtggttctcccacctctgcctcccgagtagctggggctacaggcgc |
| | | ccgccaccatgcctggcttattttttgtatttttagtagagacggggtttccc |
| | | catgttagccaggatggtctcgatctcctgacctcatgatccgcccgcctcgg |
| | | cctcccaaagtgctggaattataggcatgagccaccatgcccagccctgactg |
| | | ccctttaagatgagtacataagtagtagtagtacatttttctttcacatcctg |
| | | gagaagatatactgtgttcactattgaaatgaaaccataaagctagagttagg |
| | | aagattgaagaaatgaaaaaggagctcacatgattttgtctcaggagaggctc |
| | | ttccaggattctttggagatatggtagattccatagctggagcagggaaagga |
| | | caggatgagcctgtgggtgtagaaaggaagggagtgcttgaaagatgatgagg |
| | | agatgtcagcaggtcacagaaaccctctgaaggaggctccaactggccaggct |
| | | ggggacaatttgggccccaaaataatgacagtaacaaattgtaactcattgaa |
| | | tgaaataggaatccatacattggtaattatataaataagggaataaaaccatg |
| | | atgcaaaaagggatgtttatgtcatcacgcaaaatatgttcacagaaaatatg |
| | | tactaattaaaagagggaaaagagtaactttacagtggatgaagcctggcaat |
| | | catcactttaagcaagtggtcagagttaatattatcagtaatggtcaaatcaa |
| | | aaccatatgcaagaagactctaaaatgcaagaagactcctgaagtacttctta |
| | | ccaaagatgtagaacttaaattcagtcataacaatacatgagacaaacccaag |
| | | ttagagcacagtctgcaaaataactggcctgtaatcttcaaatgcatcaagat |
| | | catgaaagacaaggaaagagtgaagagctgctccagttggaagagacttaaaa |
| | | ctaaatgcaatgtatgatcctagattggatctttttgctctaaggacattaat |
| | | gggccagttagtgatatttgaaggggatccgagggttccattgtagtaatata |
| | | tcagtgttaatttttaaatttttattaggttgggattattttggaaaatacca |
| | | ttattcatagcgaatacaaagtagaatatttggggatgataatgcatgattac |
| | | aacaaatgtttcaggagaaatatgatctttgtagtggtcttgcaacttttctg |
| | | taagtctgaaattgtttatgcataaaaggttaaaaaaaggttaaattttgttt |
| | | ttataactaataatggattagggtcatgtgaaagtactttagaggaaatgaga |
| | | cttttgagaacatcatccctgaagacgttgaaacactgagttacctcatggat |
| | | aatttaataggatatgcagctgatttttctaccttaatttcttgtttgcagta |
| | | tctacccatacttagaattgtctggtgttaaaatatgcccactgggactttca |
| | | tgaaattcttttgattttctagaaaattcagtttcaaaggattttttaaatag |
| | | atattttaagtttggtgtcaacttagataaaatctgtttggagtcccagtgta |
| | | agttttagtaatgtgtccaatctgtttattgaaatagtataactttagaatac |
| | | tttctttggagagatgaagattggtatgttatagttcaattcaaagttgttct |
| | | ttctattatgatctattttataattcataaaatctatcttatgattgtcatca |
| | | taagtgcaatttgttttttgccccattctacctcagaaactaagtatctgggc |
| | | atcaataacaattggtagtagtgtttgctgctaagccaagtttcaccagtaca |
| | | gtgtggaattattttattgttttttctgtgaacattgtatctgctgttactag |
| | | gttattgtgaggtattgggccttcatagaaattgcctggaacccttgttcact |
| | | aaagcctgttacactttttattctctgtgcgtgtaatcagagacttattgata |
| | | ctgacacattcaaggggcattattgatcatttagattgctctaagacctaagg |
| | | agtcttggccggatgcggtgcctcacgcctgtaatcccagcactatgggaggc |
| | | cgaggcgggtggatcacctgaggtcaggagttcgagatcagcctggccaacat |
| | | ggtgaaaccccgtctctactaaaaatgcgaaaattagctgggcatggtggcag |
| | | gcgcctgtaattccagctactcgggaggctgagacaggagaatcgcctgaacc |
| | | cgggaggcagaggttgcagtgagccaagactgtgccattgcattccagcctgg |
| | | gtaacagagcgagactccatctcaaaaaaaaaaaaaaaacgaaaaacaaaaac |
| | | ctaaggagtcttttctccttattttacaataaattccttttgattttgtgtaa |
| | | aaacttgaaactgtttatgaatgtaaaataacatttgaatacttttcttgtgc |
| | | cagatattaggttaaatgctttatgtgaattttcatttgattctcacaacttt |
| | | tgagttaggtagttatttttctcattttacagatgaaatggagggttaggaac |
| | | tcgtaggtagtagatgctgaagctgagatttgggcctgggtcttttcactact |
| | | gtgccagaatcatttgggagggagtaaaaactcaagcctttggaaaatatgat |
| | | gacataaaattgtcctttatattgagaagcttccatagttaccagtgtccttc |
| | | acagggttgatcggaaagacatacatgttagtgatgatgataatgatgaagat |
| | | aatcattattaccacaggtacttcctataatataagcatctttcaaattgtat |
| | | gagaactttcatagaacatctgagtaaatgaacagtacagtgtgcatgaaacc |
| | | actaagcaaaccaagggaagttaattttctttatatgaattgtaaacatgtct |
| | | ctagatatcctttatcagattccaccatgcgtaagtagtgtctaagttgcccc |
| | | atatttagagtttttcaatgaggttgtgttcctacttagaatcctaaagttca |
| | | gctataacagatatattaataaaatctgtggaatctttaattgagcataatgg |
| | | tggctgttattttaacttgaggctttttgttgagctggattggaagtgcaact |
| | | tattagaaattacagtgtatttattcctatttcttgttctttatgtgagagaa |
| | | gatatactttagtagactgaatacttcagagctgtatctcatttaccaataaa |
| | | atgtgaaaacagtggtaaattccttcacttgggctaccattgtacaggcctat |
| | | tttaatggtatagtttgatatccttaatgttaaaagcaatatagcttaaagag |
| | | gctggtaaattagaattttccaatatcctcagctttttttcctctcacagtta |
| | | atttgctctgctgactccctacgcgaggtggcaacagctggcccttttactgg |
| | | agcttgtggggattagagagtcgggctcgcagcagcgtgctcggcctcttgcc |
| | | tctgttgactgttctttattgtttgatgcctgagcatctcccagacagcgagc |
| | | aattgtttctggaaacttaaagtttgtttctcttgggagtagacaatgctttt |
| | | ggggcttgtctttgtgtttcttcactttcccagtctcctcttatccttcatcc |
| | | tgtgctttctcttgataattagaaaggagcaaagataccacctttttatttag |
| | | gtctgcatgagattctaaaacttagaagtataggctatagatgaaagtttctt |
| | | ttttcagtaagccacctcagtaacaaatcatgttttaaatgaaaactttgttc |
| | | ttcataatatcatttagtgagagaaaacaaatgcatgagtgcatttttgaaat |
| | | tatggtactaaaagggagcagcagcaaggtgacctaatactgccattttaaaa |
| | | gctaggattagaaatgtatcataactgcttaaatctaaaaagattctttcact |
| | | gaatccaaaatatagttctaatttataggatagttataagaaatctctatgcc |
| | | atgtggaaacatgaataaaaagtagtcagaacatagctaaatagaaccctgag |
| | | gtaggcagaatgattttattcttcacatttagaaaagaaaacatcaaggtacc |
| | | ctggaacttaatttctacagtgacttcacattccgacacttctcccatacctg |
| | | ccatacccttgagtgttgttacggatgagaatatcgtctgtgaagtagtatga |
| | | gatggaaattttcctagaaagattattgtactcggaatttggaactgaaaagt |
| | | gtagaaaggggaagtgatgtgtttaaaactgtttgcggaggtggggctctgcc |
| | | atgtgtattttgacaaagctacacaggtgattcttgccatccccgattaccgt |
| | | gtacccgcctgcccctgagctggcactccaaagagttctttcagtgcatagca |
| | | agacaatttttcatgctattaattgggataaaattgacatacattcatttgta |
| | | gagtctgagacacaacgtcactttggaaaatttggtgagcaatttgaactgca |
| | | tctgcactggtgtgttctttttgtttctgtagacttaaccaaagaaaatgaac |
| | | tttaaagggactttaaaggcatctgcactggtgtgttctttttgtttctgtag |
| | | acttaaccaaagaaaatgaattttaaaggaagagagggtgataccaagttgta |
| | | gaattctaggtatgtaggttcagaggagattttttttttttaagaaaaaaaaa |
| | | aaaaaaaaaaaaaacacccaatcaagaagaatagagcagggtgtcccgaagag |
| | | aacgtgtgagctcgaagcatcccggcagcatctttcatatctcagtactgttg |
| | | ctctgtttcttgggctcacaacaccatttcctctctcctggcttttaacacat |
| | | ctcgaggcaaccttttccctttctttttatgcacttctctcactgcgtctctt |
| | | ctatatcatcatcacttcaacctaacccagtatttttatcccacctgcttatt |
| | | taccttccttcagtgactaaaaaccttactcagatactgccagtgttgtttaa |
| | | ttgagcagaatagaggcttctcactataggcaactgtaaatcaatgaaaataa |
| | | ccatttaaagaagaaaaacattttcatgtctatcacggtcgatcccttctgcc |
| | | aaagtgatttggttcattcataaattccccatacctcgtgtgttacatattgt |
| | | actgtacacatttactgaatgttcgattgtgatcttgtaatacagactgttca |
| | | ttagcccccttctcttgacttaaaaagttggggggaactaactcttttcatcc |
| | | caaggaaactttcttctactctgtcttgccagaaagttactgctcatttctct |
| | | tgtagagcagcttgcctgtgtggcattcactcctgttctgcccactcccttcc |
| | | taatatcgtgcagtctggctttcatctatatcaaaaccacttattgatagatc |
| | | accaatgatttcctaatgccagtctacccagttcaccaggaaactttaataac |
| | | tttttatgtttattaggaatttttaagttcattggaatacattcaagtacttt |
| | | ttggaatgattatatgatgtagaaatgtgtatgtttgagagacagaaaaattg |
| | | atttttttttcctcttcactacagaataaataatgtatttgttttatggtagc |
| | | aatacttgaactctttaaggcatcttttcatggtaaatctggcaattttaaaa |
| | | atctgggctttgtaaaataattttttttatagtaaggcagttaacacattaaa |
| | | gcaactaggaaagatagtgaagaattatttttaccttgagtctgtatagatga |
| | | agtaggctctgctttgtgttggaacagaacaaacaaacaaaaaaacctgagtt |
| | | gatacaaagataaagtaatcctcaaggaaagtcctctctgttagagaagtggt |
| | | tatttacacacagaattccacatgacaacgcctgagtggtgtggtttccaggt |
| | | tattgatgagaaaatcgagactcaaaatgggtcttttagaatgaagtacattt |
| | | ttcatggcctaagtctgtctttaaaagtcaccgttgtggccgggtgtggtggc |
| | | tcacgcctgtaatcccagcactttaggaggccaaggtgggcggatcacaaggt |
| | | caggagatccagaccatcctggctaacacagtgaaaccccgtctctactaaaa |
| | | atacaaaaaatttagccaggcgtggtggcgggcgcctgtagtcccagctgctg |
| | | gggaggctgaggcaggagaatggcgtgaacctgggaggcggagcttgcggtga |
| | | gccgagatcgcgccactgcactccagcctgggtgacagagcaagactcgtctc |
| | | aaaaaaaaaaaaaaaaaaaaaaaaagtcactgttgaagaatatcaataaatta |
| | | gtacaagcgtaaaagaacattttcttttctataatattatacatgctgctggt |
| | | aatcaacactttactagcaagtatattcttttgctttaaactcaagttttaac |
| | | tgattaagaataaagacaagaatgttctctacaataatgtatggattgaattt |
| | | gccatttatcattttaatgtaggttttacttatatactattgtgaaaatactc |
| | | ttaatgtattcaaaaggccagtgcacaatttttttttcttttacttctttttt |
| | | ttttttttttttctttagaaagagtgtcacttgctgcccaggctagagtgcag |
| | | tggtgtgatcatggctcactgcagccttgaactcctgggctcaagtgatctaa |
| | | tacctttaaagttgggaataaactttatcttaagcgtttttatttttaaatta |
| | | tgtttttgcatatttgatagaaaaagtagaatgtagtaattgaaaacctaatc |
| | | acaaaacaattcattggactctgcaacagtatataaaaaataaaattaaacga |
| | | gataggaaatcttaagggattggtggattgatgcacatgaaactggtaacctc |
| | | tgttaagtacagttctccaggtagttggagaaattagttaaatgtgaagagaa |
| | | ttttaattttgcactattttgtacatttctaaactgtgtctcccacagccctt |
| | | ctcccccagtgagcacgattcagaattactttgaaatgttgtagtcttaatta |
| | | tcctattcatggaaatgacgaagctaatacacgatgtgctctatcttaaaagt |
| | | aacagatattttcccaagtaacctactgctggttgtgatgctgagggacattt |
| | | catgggactgcatggtcgttgctcatcgtgataccatcctcagtggttggggg |
| | | attcacagtgaattctcatatcctgtaactatgcatcatggatctatcatctg |
| | | aaaataaatcaaaatctttgttgaactcacagtttccacacttgtatcaccca |
| | | tttaagattgtttcattgttacctcctgtgtacagaatatttcatttcaattt |
| | | ctcttagaacagctcattcatctattctctagtttcaatattctgagcagtag |
| | | aagtttgctgttttgattaacttcagttagatctcttttctgggccaagaatt |
| | | aaagccattttatctttagtctctccttttgttggcactgcttcatagactgt |
| | | gtcatatatacagatctgtctttagactgatctttaccaaagtacactactgg |
| | | aatttgagggtttttttttttaacatccttttcattatgagagagctagtgta |
| | | tatgcattgtgggaaattagaaactatagatggcaaaattttaaaaaataatt |
| | | gccaccacccagagattgcactgtagtttaagacacttttgaatgtggtccta |
| | | gggacataatttctggaacacatttttcgtgaagaggtctcaggttggcttct |
| | | tatacccacagctcgttgtcattgcccctagttttaatttcccatcgctcagt |
| | | gggctagattttttttcattttcttcatataaacttatttcagaaatgttcat |
| | | taagaggaataagcagcattagtaaaaatgaaacctatggtacccattacttt |
| | | atatagttcaagtattctggaagccatattgtagcatagcatgtactgaaaat |
| | | cactctcctttgaacagtaatcccatacctgtatttgggacctggccttcctt |
| | | tgtgtgcttgtgtattcattatatcccctttctctcttcaaagatgctcaagt |
| | | cattctcatcttaaaactaatgggttgaaccttccatgcagtctagtagctac |
| | | tgtgaactctaatctctattacaaaggttagctctttgagtctcacttctact |
| | | gaagttgtttttttttcccaagattactgaaaatttaagagaaaataatggcc |
| | | caggcatgcattcaggactagaaaatacttccatgtacagaaaaccaaacacc |
| | | acatgttctcactcataagtgggaattgaacattgagaacacatggacacagg |
| | | gaggggaacagcatacgccagggcctgttggggcgtggggggcgaggggaggg |
| | | aacttagaggacttaagtgcagcagaccaccatggcacacgtatacctgtgta |
| | | gcctgcacattctgcacatagagcccgcttttgtttttgtttttgtttttaag |
| | | aagaaataacggggaaaaaaaaggtttcaaaactcataaagaaagagaaagag |
| | | agggagggagggagggaagaaaatgcttccatgtaactgcatcatttggtact |
| | | ttggagtccatatcctacttgaaactctaggatctggccctcacatttatgta |
| | | gtgctttattttacagtttacaaaacttctgcttgtccatgtgtgtctgtaaa |
| | | gtcatatgaggcattatgcccattgttcagatagagaaattaacgttcattga |
| | | cataaatggttaagcccattatgtaaatatttatggcaaagctggggctaatc |
| | | atatgtgttacagataggactttttttaaagaattgtttaggtattctgttca |
| | | tcattagtctctgggtttgtgtttgtggtaaccatagacaaccaagttcatat |
| | | aatttggcttcttttttatgtgatttttgatacgtgttaaggatctataacaa |
| | | tgaatttgcctcctaaagaggtacataatgttttcattcctccaaaaagataa |
| | | ttctaggtttataaatctatgtatgctcagtgccagttgaattttgtgattgt |
| | | tcaatagaaaagaaattgtgacttaaaggtgattttccagtttaatggaataa |
| | | atgaaattagtttagaagttatttttatttttctgagcctgattctcactcag |
| | | ttgtgataaacagcacctctgtaagataaactcggtgataaaccgagaacttc |
| | | tgaaatcagcctaacatgaatacctgttcttcttgtgctaagtttcataatgc |
| | | tttatcctaatacaccatttttttaagaaatggaacttgtatttcatttttgc |
| | | tttcatctcacctaattcataattttattaaaacctacgatttttaatttctt |
| | | tttttatgaatttttagtttggtgtataaatcagaattacattctctgatctt |
| | | ttacttttaaaattacagtgatgaactgactgtttaagaatcattctcatgat |
| | | tcattcgtctgttatgcctcctttttaaagcttcagcactgaaggtcttttga |
| | | caaaccaatatttataacagtttgacagcaggatgaggaacagcgtttgtctt |
| | | tgtaacagcttgaagaaagaccctttccaggacccagtcatgcagttacaatc |
| | | ttgacctctttcttatgctgggaacatgcatacagcagcacctcccatgtgtt |
| | | ttcttgtcccattgactgtccattcacttcccatctgttttgcagtcttaaag |
| | | gaacagaaggggccttcttataaatctgtctttgcaggtgataaatgatgcct |
| | | acctctttaagagctgcctgggtggttttccttttcttagaacatttctgctt |
| | | tcctcctaactaaatcagggaaaaatacaattttaggaataagagaaaaagaa |
| | | gaaaagatgaatttttaaagcatttaattgactaagaatattttactgatctt |
| | | ttttaatcttcccaattaattgcctaaatcatattttttaaaatgtattatcg |
| | | atatttagatttttgtcagggagtaaaatgaatgtattcattttgaaataatg |
| | | taactcttttttgagaaaacaaagccatgtatcattaatgagttaacatataa |
| | | aataactttttaagttttttgtgataatttaagtgtggagcatcttatgtatt |
| | | ggatacaaaagtaaaatatttcagagtaaatcattgtaatcttatggtaaaat |
| | | ctattcattttttacatttaaaaagatgatcataaatcccataaacatttatg |
| | | cttttacttctgttgctgaaaataagtattgtaggaatagatattgatatcat |
| | | tgggttttctaagaattcagcagaaataaaaataatttactttttctcccatg |
| | | cagaaattatttatgcaaggttttatgtaacaaatattgtccctctatggccc |
| | | tgcagaatattcttaaattactgatttaaaaactattaccagtataaaatgac |
| | | cacttttagaatattgtggtgtattatgtgaatcagctggctaataatatatc |
| | | ttctgtggactagcttgttagtttgtttattaattccctggcatattccaaaa |
| | | ggaatttgaggcagcttacatatatcctacgcaaaagataaaactacttaagt |
| | | gaaaaatttgggttgaaagaaaaggaaaatccaggcaagtgaaataaagtaaa |
| | | ctttcagataaaattggtgcccctcaaagtgcatgctcaagggttctacgtac |
| | | aggcagacctcattgtattgcatgtcactttattgcacttcacagttattgca |
| | | tttttaacaatagaagttttgtggcaaccctgcattgaacaagcctgttggca |
| | | ctattttcccaacagccatgtgctcacctcatgtcactgtcacattttggtaa |
| | | ttcttgcaatatttcaaatttttccattattattctgtctgtcatggtgatct |
| | | ttgatgtttgtattgtagctattttgggtaccactaactgtgcccatattagt |
| | | cagtgaccttaatcagtaaacgtgtgtattctggctgttccaccaactagaca |
| | | ttccctgtctctctcctcctcttcaggcctccctattccataggacacaacaa |
| | | tattgaaatttggccagctaataaccctacaatggcctctacatgttcaagtg |
| | | aaagaaagagtgccatatttcactttaaatcaacaactagaaatgattaagct |
| | | tagtaaaggaggtttgttgaaagccaaaatgggctattagccaaattgtgaat |
| | | gcaaaagaaaagttcttgaaggaaattaaaagtgttattccagtgaacacacg |
| | | aatgataaagcagaacagccttattgcctgagacgcaggaagtttcactggtc |
| | | tggatagaagatcaaaccagccataacattcccttaagctaaaacctaatcca |
| | | gagcaagttcctaactctattcaattctccgaaagctgagaggtgaggaagct |
| | | gcagaataaaatttgaagctagcaaagtttggttcataaggtttaagaggaaa |
| | | aaagccattctgcaacatgaaagtgcaaggtgctgatgtagcagctgcagcaa |
| | | gttatcaagaatatctaactaagataattgatgaaggtgattatactaaacaa |
| | | cagattcttgatgcagatgaagtagctgtctattggaagacgatgccatctag |
| | | taatttaatagctagagagaagtcaatgcccagcttcgaggcttcgaaagaga |
| | | ggctatcccctcattttgggtgccaatgcagcaggtgcctttaagttgaagcc |
| | | aacctaaagaatttaccattctgaaaatcctagggcccttaaggattatgcta |
| | | agtctatcctgcttgttttctaaaagtggaacaaaaagcctggatgacagcac |
| | | atctgtttacagcatggtttactgaatattataactctcgagacctgctcaga |
| | | aaagttttctttcaaaatattactgctcattgacaatgcatctggtcaagcaa |
| | | gagttctgagggagatgtacaaggagatttatgttgtttttgtgcctgctagc |
| | | acaacatccattctgcagcccatggatcaaggaatactttcaaccttgaagtc |
| | | ttattattttaaaaatacgtgtcttaaggccctagctgccatagatagtgatt |
| | | cctctgatggatttaggagaaaaaaaaaggaaaagcttctggaaaggactcac |
| | | cattttagatgctgttaagaccattcaggattcatgggaggaggtcagaatgt |
| | | caccattaacagtttggaagaagttgattccaaccctcatggatgactttgaa |
| | | gagtttgggacttaagaggaggaagtaactgcagatatggtagagacagcaat |
| | | agaactagaattagttctgttgtaatatgataaaacttgaacagatgaaacat |
| | | tgctttttatggacaagcaaagaaagtggtttcttttttcttttttttttttt |
| | | tggcagtctcagtttgaagaaagtggtttcttgagatggaatctgttcctggt |
| | | gaagatgctgtgaacattgttgaaatggcagtaaaggatttagaatattacat |
| | | aaacttagtagataaagcagctgcagggtttgagaagatagtgtcccaatttt |
| | | taaagaagaaaaatttgagtaaatttgggtaaaatttacccaaaattacctat |
| | | tgtgggtaaaatgctatcagacagcatcacatcctactgtgaaatctttcatg |
| | | aaaggaagaatcaatcagtgcagcaaactacaattgttgtcttattttaagaa |
| | | attgccatagccaccgtaacctgcaacagccaccaccctgatcagtcagcagc |
| | | catcaacgtcagggccagaccctccaccagcaaaaagattatgacttgctgaa |
| | | ggctcaggtgatccttagcatttgttagcaataaagtacttttaaataagtta |
| | | tgtacattgtctttttagacataatgctattacacacttaatatattacagta |
| | | tactgtaaacgtaacttaaacgcaccggaaaaccaaaaaaccttatgtgactc |
| | | actttattgtgatatacgctttattgtggcagtctagaaccaaacttgcatat |
| | | ctcccaagtatgctgggactttgctagaggtaagctgcaaatttagccctcag |
| | | tttcctggtggctggcagttacaaaatggaaagcagaggtcattccatcattc |
| | | atggtggccatcagacaacaacacagcagttgcttaggagaagcatgggtctt |
| | | cttcgtacgcacaactgagagaaatttcccttaaagtggacactgagttagat |
| | | gatacaatgaatctaatggctacacataatcatgaaaatcatggggcccttta |
| | | ttgtaatgtttctcatgcgggctaacatgcgtagttctagggaaaatatgatg |
| | | ctgtccaaacatacagctatttggtttggcttatctaaagataaaatacatag |
| | | tatccagagaaatagatgaactgtatgtcctccatacagtctcccataaatat |
| | | tatttctttttgcagctgatccttttagtaaatatcaggtagccagaagttca |
| | | agattttacactcattgacattgacaagcacctggaatggtactacctttttt |
| | | ttttttttttttttttgagacagagtcttgctctgtcacccaggctggagtgc |
| | | agtggcatgatcttggctcactacaacctccgcctcctggattcaagtgattc |
| | | tcctgcctcagcctcccaggtagctgggattacaggcgcccgccactacgccc |
| | | ggctaatttttgtatttttagtagagatgggttttcgccatgttggccagggt |
| | | gatcttgaactcctgacctcatgtgatccacccgcctcggcctcccaaagtgc |
| | | tgggattacaggcgtgagccactgcgcccagccaagtactatttttattagtt |
| | | aagtcagagccataatcattataactgagctgaaattagaattgccatccact |
| | | taagaaagttgagtggtctaacaagtataaaagcctaaatataaggctaattc |
| | | atgttcatactgaagccttttggggaataggccttaaaatatgtagaaagtat |
| | | ttgaagcggttttaattgtactagccaaaaggagcctagtagaaatgcttgtg |
| | | ttataagagtttattttttaaaaagctgaatttatctgaccaggcgcggtggt |
| | | tcacgcctgtaatcccagcactttgggaggccaaggcaggtggatcacgaggt |
| | | caggagtttgagaccagcctagccaatatggtgaaaccccatcactactaaaa |
| | | atacaaaaaaattggccaggcatggtgatgcctgcctgtagtcccagctactc |
| | | cggaggctgaggcagaagaatcatttgaaaccgggaggcggaggttgcagtga |
| | | gccgagattgcgccactgcactccagcctggacgacagagcgagactccatct |
| | | caaaaaaaaaaaaagctgaatttatcaacaaattgctgtggagttttttatat |
| | | attcagcaggcatcagttgtaatttacctcacagactttcttaaggttgcttt |
| | | ctttctaaattatactttatgggggtcacaaaatagcaatttttaaataatca |
| | | cctttaatgattaagtattgtttaagtcagatcactcaactatgaatgcatga |
| | | atattcatggacatctattacatagcaagcagtgctatgctgggccgagtgat |
| | | tttaaatgacagactttttggtaagtagagaatttacccaagcagtccttgct |
| | | gttctccacattaatgctcagaaaaaatacattataaaaatgatctttccaaa |
| | | atgaattatgaagccccatgagaatgatatggcaatttgtggttacatatttt |
| | | actagaggattaatatccaataaataaaaagatactaaggaataaacaaaaaa |
| | | aatttaaaagatgaagtatataatgaattagaacaatacattttaatcataag |
| | | ttttaaattagtgtggactttgaattctcctggacagattccttcattttata |
| | | gataaagctaggactgtgacttatccagttatgaggttaacggcgaatacaac |
| | | attgtcatatattttaaatgacacacattacaacatgttctctgctttataaa |
| | | aatcatatcaaataattgccccatagattattaaaggtgttagactagggatt |
| | | cttaaaaaaaattttcatcaaatgtttctttcattattaatcccatgaagtcc |
| | | atgttacagaagattttgtctacaacagtgcagttacattcttctcgttagaa |
| | | atacaaccaccagttagagttcctaatcagtataaggaagtagttgttaggag |
| | | aggggatgggtttcttgtccaaatgaagttttccatttgagtttttgaagtag |
| | | tgaaactaacccagcgtttacaggccccagaaatctgggaacctcagctttca |
| | | aagtactgtaccagtctttaacagttttcctggacgtgtgaattgatgcctcc |
| | | ttctgtaacatgcaggagtgttctgtctgtcttcattgagtgttaaaaaataa |
| | | tcatgcctatttcaaggaaaaaatctacagaactaagatgcagaagataagtg |
| | | ctagatttaatcatattccttcatctatctgtttggttcaacctttcatcaac |
| | | taaaagatgcaccttttttcttgtgctaactctaagattttagctacagtttt |
| | | gagaatcttgagtgtagtctcttgtttaccttttttcctttttttgtttcccc |
| | | cacaccctagattcatttaaatactgaacttctaaagggcaagtatatagtgt |
| | | agtttaataaaaagcaaaccttttcatgaacaatatatattacataataagaa |
| | | gcgttcctttacttttcagtactctagtgaatagctttctacagtagaatctc |
| | | acttagagggtgtcttaaagcttaacaccaagtgctcaggcagcatgttatac |
| | | aacagttccattaaggtacatttggatctttggatgtgtggtttgcttaaagt |
| | | acactgcattagtaagttggcagcttgctttctttaaaaacatcaaaagtttt |
| | | aaaaggtttatttcagggcatgtgttagtgttttgtgtgtggttctttgttcc |
| | | tgttctaaactgttattaaccactgaagtgaaccttctcccgggtttggcctt |
| | | ttggtattcacagtgtattcaaaacctaattacagattagtctatatttgaga |
| | | cttttagagcaagtatcagaagacccaaaaagaaaatgagagtagcagtatca |
| | | tttcatgtagagataaagagacccaaaacatgaatgggtgtcaagtcagctga |
| | | agaaaagaaaaaagagaaggaacttcattcactgagacggtttatgagttggg |
| | | gattatgggaatattcatgactcaatcaagaagcacagtgaattgatgtttga |
| | | aatagctcatcttttaagtaaacattggataaatggaaagtagactcagtatt |
| | | cactacacgtagaaatagctatttctgtatagcagaaatagcagtttgttaat |
| | | cccttcctgagttggtttaatttaccaagtaaatcacaaattttattctttat |
| | | ttgtgaatatttaattcaaatatttaatggaaatatgagtttgctttataatt |
| | | agtcatgctgatccatacacgtatttctgagagaaagcaatttctaatggtga |
| | | aatagttacaataatatttttgaaatttgaaagcaccgtgatactgaagcatt |
| | | aatctgaaggatcggaaagtagggagtttttgttgccaacatttaacttcatt |
| | | gtttatggataacttggttttctgggcagccagatggcacagttagtatacag |
| | | acattcttggaaacttgtatcaaaatttaaaatgaatgaatttatgagaaata |
| | | attctgcttattatttgtaatgtagctttcttgaaaagcaagaaatcggaatg |
| | | tagtttctaaagctgcaagtgaatatgtatacatagccagctctttcagcctt |
| | | gataataaggtgcaaccattaagatgaagggatttttttttcccacttgtgtt |
| | | tttgggcccgagtatcctgatctgtgttgcttgtctggttcaggtgtgagcca |
| | | ccagctttctttgactttcattatctatgtgtatcttgcctcctgttcccagg |
| | | cttgctctagctcttctgatcctgtcttcctccctcttgatcactagtgtagt |
| | | attcatgaagccagctaagttagtttttccctttgaaaaccacagcccttatc |
| | | ttctgtgccatattttgggcaacttcgtttatcattgattgaccgtacgcagt |
| | | gatcaggccttgttctagacactgaagactctgagcatttttgggcccatttt |
| | | gtactcctgtattgttctccaggggcttctccaagtgtgcgtcaatttagtct |
| | | tctcaagagggcatcattttcatcagaatatgatagcatattatggagtgtcc |
| | | ggtcatccttaggcatagactacttaggaggtgtaactgttttgttccctgat |
| | | ttttactgaaatgggtcttttcttttttttttttttttttttttttttttttt |
| | | ttttttttttttttttgagacagagtctcgctatgtcaccaggctggagtgca |
| | | gtggcatgatctcagctcactgcaacctccgcctcccgattctccctgaaatg |
| | | cgtcttattttaagtcaaaggtaatacttaaaaaagaccaaagagacttaaaa |
| | | taacagcatttgcttcgtcactatgagctttgttattatgagttaacatacag |
| | | tagcagactgggtgtagtagctcacgccctgtaattccagcagtttgtgaagc |
| | | cgagtggggaggattgcttgaggccaagacttcgagaccagcctgggcaacat |
| | | agtgagacccccatcttgacaaaaaaaattgttttaaattagccaggtgtggt |
| | | gctgcatgcctgtggtcccagctacttggaaggctaaggtaggagaatcgctt |
| | | gagcctgggaggtcgaggctgtagtgagccgtgtttgcatcactgcactcctg |
| | | ggtgacagtgcaagactctgcgtcagacaaacaaacatcgtagcagatgtgtt |
| | | tcttaatcagagaagtgtagacaaggctaactccaggctttaatgtcctcata |
| | | tttagcaatgatacctgcaaggttgtatgagaaccaaatgaaacgccaaattt |
| | | ggaaatacatagtagatacatcatagcagagtaagccaggaatgcttctcaaa |
| | | ggtaggatatcatctgtgtcctcatatcactttatgaagtacattgtgaaagt |
| | | gaaagaacaaagaaataaatgttttttagttaatgtttaaaggatacatttat |
| | | cataattgctcttttaacactcacctccagtctcccctccgttcacacctcct |
| | | acccccattacttcctggtaacttagttaagtgtcctttgtcattcctgaggt |
| | | ttcaaggcatggtagtactgtgtcctgatattctaatcgtaaatatttaaggg |
| | | aaattcggcattttttcattttgtggttttcatattaaagtacattaaatagt |
| | | ctttttgcttttatttaggaaaaaaactgcttacctgttaattttagaaaaat |
| | | ctgattttcatttagaccttacagggtgagacacctgcatcagggtggctctt |
| | | ggtatctttcaattcaattggatcttctctgaatagtctcttgtagggagtga |
| | | ggctgctgtaccacctccctgcagtagtccatccagcttaagatgggggtcac |
| | | cagtaggccaaaagaatgggtagacctggccatgcactgccctattgtactca |
| | | aatcgtgtatcaaatggagttggatttcttctcttcatacagtacagcatttc |
| | | caagtagaaatatttctcaatgaaatgtggagagaagcacccgtttgagattc |
| | | ccgtgtgttgtgtgatttaagttagatggttttttaagaccacattcatttcc |
| | | agcattctaggtaacaatttagaaaatgtctttctcctaacctccccactttt |
| | | taaaaatcctccaactgatgaactgatgtgaaactttcttacattcactgaaa |
| | | aaaaaaaaaaataggttaagctgtttctaagcaactagatgaattaattttta |
| | | aactaagaatgtggccttattttgggaaaacaagaatatttacttgtttgtct |
| | | gctgtttaaaaaatggaagtcagcctaccaaaaaattgagactcaacttctag |
| | | gagatgggttaggatttttttttttaagtttctctagtttaattttatatata |
| | | aggggttaatgctaccttcataataactattatcatattttctcaatacatag |
| | | cttgattaaaacaactggactccccccccaccccaccccacacacacacagat |
| | | tttatatcagtctgaatctaatgcctagaataagaagtgcttcagccaggcat |
| | | agtggcactcacctgtagtctcagctactcaggaggctgaggcagcaggatca |
| | | attgagcccaggagtctgagtctagcctgggcaacatagtgagacctagaagt |
| | | tttaaattactggaaaaataatatgaaaagaataaattactggaaaaagaata |
| | | tgaaaatgttacgttctttatatccaaccgtggtaggcttttttgagttcctg |
| | | caatgctaataagaattcataaaaaggacaattcttcattttcttgggtactc |
| | | atcactaatagctgcctcgctggtaaaaaggaatacatgtatcttcaattgca |
| | | gattatttacttttaaatataaaagatataaatgtcaaatattaaatgcatct |
| | | tacatggttttcctacatagtgaaagtagaatgcttgccagttttgcctctag |
| | | gtcactcactttgaaccagccaacccaccttaattgatcatttccactaatat |
| | | gttaaattaccttaaaagaacaaaaatatttatcatgcttactataacctgtg |
| | | ttttaaaataggaggccaggcacagtggctcacacctgtaatcccaggacttt |
| | | gggaggccaaggcaggaggatcacttgagcccaggtgttcaggaccagcctgg |
| | | gcaacaaagtgagatcctatctccacaaaaaaattaaaaataaaaacttagcc |
| | | aggcgtggtggcacgtgcctgtggtcttagctacgtgggaggccaaggcggga |
| | | ggatcacctgagctcaggaggttgaggctgcagtaagccctgccaacaccact |
| | | gcacgccaacctgggcgacagagtaggacccccatctcagaatataaaataaa |
| | | gtaggaggtgcatgtgaagtagtatagatcatgacttttccaattttaagagg |
| | | ggattggcatgtactatgagcagttcacatttgtggaggaaatctacatttca |
| | | gagagtatatatttcatttggaagtctataaacatgaaaacctaaaataaata |
| | | atgtaaatctacctctagtggctctggtatttttaaacttatttatagctggc |
| | | aaagtacttttttgtatgtatttttatagcaccattgcacttctcatgtttgt |
| | | tgcaagcatctcccacagcttcctttgtcttttaattttatgacatataaata |
| | | aaagtatacatttcaatatggccatattgattgatcttttcctttgtaactct |
| | | tactactttatatttaaaaagtcatttcccagtctaaggccacctctattttc |
| | | ttttagttttttaaaatggtttcattgttttatatttgcctatgatccagaca |
| | | ttagtaactgtgggttcttaattgggcttcagagaatctgagaattccttaaa |
| | | attctctacataattgtacatgtacttaatacatgcttttttccatgttaaga |
| | | gtccagagtttttgttagatcctcaaaggggtcagtcagtctctcctcccact |
| | | tccaaaaaatgtctgagacctactactataatccatctggactttatttgggt |
| | | aaaaggtggtatggtgagactcatatttttctttttcccgcaaatagttaagt |
| | | ataccaaccatttagtaaataattacctcctgatttgtgatacctttgaaaaa |
| | | taaatgtttttctttatttttatctccacag |

TABLE 4
Exemplary target gene intron sequences

SEQ ID NO: 4
Organism: homo sapiens
Gene: OPA1
Intron: GRCh38/hg38: chr3 193626203 to 193631611
Intron Sequence:
gtgatggatggtttaagggggctaccgatacattcacactaatcagccatttctgccaagat
catgtcacctcaatctgttcatggactccaaatacaagaaattaatttgacaaagtgaaaat
ataaaagatgcatcatataaatatgtaacttttctggagtgggtagtataggtaaagccaaa
agaaacaaattcaagcagaggaattttggtttctgaaaattaggttgtctgtagggtccctg
tatttatacttagaacaaaattaggaatttctgtttatgtggtccagttattgagtcaccct
aagtttgtaggcatcttacctacctacttgctccccaagtttttatttctaaaatgaaaagc
attgctgtagatgaccagtttacactaaagaataacatttatttatttgttttagctaaagt
atatggacagggaacattcatattcttgtagaagaaaattattttgacttttgggcaaaagc
atgtagttcttatacactttgacaaactcattgcgtacatttttcacattaatcaaagtcag
cacaaataaattttcaccttggaccacggagggtttgaacactggaaatttgatataattct
ggttgctaaagaacaagttctaataaaagcttaagtgtataccaatatgtggctgttggtgc
aatcagcaggtccgtaaaaatatgattttaatggttaggtaatcccacaacggagatcccaa
agttcatgtttggaagagacttttgggtcaaagtgaaatcagtgtaatgaatttaaaattat
actctgagatcttgaaatcagctaattatgttacatcttattagctcagaaaagttttgaag
ttatatacaaatgctagtcaggaaaaaagattcagtcatgtaattcttgtacattctactat
ttaaatcaaccaatattatagattatgatttagtgcagtaattctgctggctaaccttatct
catttggtggtggttagtacttcagagtactcaccatagtttcatttatgttttcagcatca
cttcctggtttttctcaattccatggctgtggaatcaattcatatgtatatttagcttcggt
gagcaaaaacatagctagaaaaagaaaagaagtgagtttcctacctggttaaattaaagtcg
atgtgttaagccaaggaggacttcttttgaatggtactttaacaatccctgttctgtatact
gtgaatatatcatttaaatagcctaataaattggatgcttaggctgagccacctatacttta
gttttgttatggaaagaagggagaggagcaagtatgttcttatatgttacttagaaataaga
atgtagctgtagttacacattgttcttaagtttttttcgtaagacaacttgaaatgagtccc
ataggcctgctatttaacattctaagatatgacttaaggttaatgatgagcttttgaatctg
acaattcaagagatatccataatgaatactgattcattttctacattgctgaaagctaatgt
tcattttaagcctactttagtagcctttatttgggcttagagatgttattcctctttctgat
atttattgggttatctgtttaacccttttatatctccctttcccgatttgtaaattagagac
tggcaagactttttaccctgagtagagcaccaaacatggcttgtttctgcccacactgtagt
taccttgaggggaagtaaatgggactttaaaagcaatttatgctcttttatagtgaaattat
ccctcttactatcccgaaagactgttaccttacaatatcctccactcctttccccctgtagt
tactatagagatgacttttcggttcttcactgccataatgatcaaaatcctaattcatgaga
tttttatcattccaggcatgtgaggtttacttgatgcataaaaccgcaagtactttttgttg
ttttttaattgttttttctctcttatcttcttgaaagtctaagtagatcatcatttttgatg
tcttattagtagcaactaataaattttccctgtatcttctcagcaaaagaactcaagcagag
acagaagattagaactaccattggtagttttgcttcctatggatatgttcacatacatagaa
atttttacaatgacctttttatatatgtatttcagaatttcagaatggcctcaatgccttaa
taggaagaaatacttgaaatttttaaattagggcttggttttgtgaggagctagtaaaggtt
tttctctttcagctttagcttgtttctgcggaggattccgctctttctccatcagtttcata
gccctggaattgtagaaaagctctggtttcaagaccattgatatccatttctgtcagggtga
gttttaaatttatttcatgatgcaaacaatatattgaacaacaggacatgaacttgttcttg
ttgtaagtggctgaattttatcagtaaagcacatcaaaataaaatataccccaattgctagt
taagacctagagtgacagattgaaaatagcttgtgttattctcttaagaaaatatataaaaa
ttatcatctcatcaatctttaatgtttgttttataaatctaaatgtttttatattgtttcct
aggaaatattaggtctaattttttactttaccaccagctgtcttttattttactcttttttt
gagacggagtttcgctcttgttgcttaggctagagtgcagtggcactatctcagctcactgc
gacctctgcctcccgggttcaagcgattctcctgcctcagtctcccgagtagctgggattac
aggcacatgccactacaccaggctaattttgtatttttagtagagacggggtttcttcatgt
tggtcaggctggtctcgaactcccgacctcaggtgatccgcctgcctcggcctcccagagtg
ctgggattacaggcatgagccaccgcacctggccagctgtcttttaatataacattatgatt
aattgtgatgttccattaaactaagcggagaggaaacatgctggtaaaccatgtgtgagtta
ttcattgtaccagaaaggcaaatgatacattttatcctaaaattcaaatttataaacatctt
aacacttgtgatcattaaatactactaatctagcatataaattatatttgtaggcggggcac
ggtggctcacgcctgtaatcccagcactttgggaggctgaggtgggcagatcacgaggtcag
gagatcgagaccatcctggctaacatggtgaaaccccatctctactaaaaatacaaaaaaaa
ttagctgggtgtgctggcgggcacctgtagtcccagctacttgggaggctgaggcaggagaa
tggcgtgaccccaggaggcagagcttccagcctgggcgactccgtctcaaaaaaaaagaaaa
aagaaattatatttgtaatattctactaaccttatatcattttaactttttatataactttt
ttattttaccaaattaagttaaccttttatagcccttggcttatactaaacatcctaacttt
tttgtttaattgtattagtttttaagttattgccccagatgtcaagtaatgttggattttct
ataataatttaggatatattgcatgaagtcagttagtatttacatttaaaactaaaacaatt
tatactaatacagtttatacatttcatactaatttagctacagttggataaatatttaatgg
aacaaagtaaatcaaagtaccttttcaaatgaattggaaattaaatccacataacaattttt
tatgaccacactattacagtgtgatggcatgccaaatgatcataatgtggaattatgtattt
cttcattggctttcaagattctgttctttagtttgtgggctcctctccaacttgcttgtctc
ctcacagtttaggcgactgtttataattcttgtccatcctgcataaacacacacagtcaaaa
tgaaaaaaagcttctatcagcagatctgtgcttgctgtacagaaatgggaaaacaattgaag
tttgcattatcttttttctaattaccagatcgtttttggagctatttaggcatacgctttta
aggaaaaaagaaaaaaagagtgtaccttttgtttctaacaaaggttgttatctatattattg
aaataaaaaattggggatagttatgacaaagtatttagaaataggaattaaaatcttaaaat
aacttttcatagcatggacaagacttattaatgtctacctcaataagcaaatcatttaaaaa
tttttcatgtatatttgctgccatgatgtgttgtgattgcttaaataaccaatgaatgaaga
tcaacaaggatttaaatgaagaagaatatggatttaactattttctcctgtgaaataagttc
atatttacaagttttgattttcagaaattagacaattatttttaaaggctgggatgacaact
tctgcctcttaccaagaagtcaaagcacagttatgtgaattcatcataaatcacatcatttt
tattatattttgtatttataattgtattgtgactactttaaaacctgttataaaataaaatt
gttttttaatattttattttagaattattagcattaataacaatttgaagtagtttacacaa
tacctgtgagttttatttttgttttatattgaaattaattttagttgctttacttggcttca
ttgctatggatgcattctctgtgttacgagttagcagatctttccttggaactgaatttaaa
agcaagcatttggctccacttaaatctctgaaaatgcaacttgttctttgcatttattacat
aattcgctacttatggtacagaaatggatacaatacaaaaatatttccttataagatacact
gtgaccaatgagctttttaaatagctgtaatcagtaacatgtatttgacttttcaaaacaca
tttctggagggatatcagtgctttatttccccaaatatctgaatccctatgctttagtacaa
aacaacttctgaagaatttagtaaccatatgtgttgatctcttgtttttctaactagtcttt
cataagaaatgactagaatagcaacagggaaatgattgccttttaaggtttttgtttctcaa
tataaaattttggtgaaccatttttattgataaatacaggtatttttactttcttaaatcac
ttgatttaaaattactttgattaaatatgcatataaagtcagttgtttttaactctcaatac
ttatcaaaaaaatttaacttgctgtacattctgtataaacctaattctattcaactaaaatt
attttaaacatttag
SEQ ID NO: 5
Organism: homo sapiens
Gene: OPA1
Intron: GRCh38/hg38: chr3 193593374 to 193614710
Intron Sequence:
gtaagtgcaggctctaatctggccccgttaattctggggcctcttgagagtggggctgtctt
atctctatctccaaaaatgtgcaggtgactctcaggccaggccgacggcagttggagaattc
ccagatgttcttgaggacccagaatgacaggagccctggctgggcttacgttcggagccggc
ttcaatactggcccttttctctggccctacccaacccgaaaattctggacgcctctcaatct
tggcccgtctctattgtccttttgtctctgccctttacacccttgtgtcttcagtgttctgt
ctgtctctggttgcctcttttgccttttttctgtcctctccctgccaggtttggctctgtcc
atgagtcacctctctccacatttctcctaactctcggtgtcttctttttcttccatttccac
gccatgtgtacattgcatcttcaggtacctgggctcttctatcggggaaaggggcgtccgtc
tctttccctagcccgctgatagaagtcagaactagagcaatgacgcacacggtgtcagagac
ggtgattcgagatgccctttcaatagcagcttttttctgtgtttcgggagggagacttactt
tttgatgcaaggtcgtgaacgtggcaccacctttctaatctcaatcattgttgccctggggt
ggtttaattctaaatagaaaatcatagaaatcttttcatttctgtgcgttactatatgcatt
gtaatgagattaaattggattttataggaaattttgttctagtatcattagataccttcaag
cttagctcattgttgcaggcatttgataggaagtaagatgcatcaagcaaaattggaaaaac
gtggttttcctgaattaacttctaagcagttgttttgaattttttccagacctttttaagtg
gtatagataatttatcgtgtttataaggaatggaatgcattcgttagtttgtttttgttttg
ttttgagacggagtcttgctctgtcgtccaggctggagtgcagtagcgctatctcggctcac
tgcaacctccgcctcccaggttcaagcaattctcctgtctccgcctccggagtagctggaat
tacaggcacgcgccagcacgcctagctaatttttgtatttttagtagagagggggtttcacc
attttggccaggctggtctcgaactcctgacctcatgtgatccaccctcctcgacttcccaa
agtgctgggattacaaccgtgagccaccgcgcccggcccaatttgttttatataggttaact
ggagtccaaaatacagaactagatgagataacaatagttaacagtgttagtcagttagaatt
attgcataggtatttttaatctcatggaattttagtctttgagtaagttcacagcccttggt
attaaagtaagttatttacaacccttgcatttctacttctcaatatttagtgaggaaacata
tctgattttctttaaataaaaagagaaaagactgcagaagatagcattctctgttggagcaa
ttaagatgtataagaagaactacaaagacggagttttaaaacaaactgatttataagtggta
tttatttaattggctgtcattgggctaaattatttctaaagttaccatggatgccattgagt
catggcttaaaaatgtctcctggtgatggcacagtttagctacctaaagaagtagagatgtg
ggaagccagaagccccaagctctgcagtttttcttttgctatagttcctttgcatgttgtga
aagaatacagttaaattcctgctccctaacagatgagagcataagcatttctttgggcatac
atatgtaaatacatgctcatggacatgtgaaaagatcaatactaacatttgggtgcaataaa
taattgtgtaaaattatttttaaaagaattacatattaggaaatgatatattgattaaaagt
gatagtcaatgaacaagagagtagatttctgggggaaacctattttgcatcatacttgattt
ttagttttgactgaatattgaagtctatattcaaaattcttttcctttagaactgtaaaggc
attgctgcattttcttctaatgtaattgtttattgctgctgagaattcttatgacaatctga
ttttttcatcttcatgattatcttgtttttcccttcatggaatctgttagggtcttgacttt
atcctttatcctaaatttctcaaggcttggaccaggtgtgggtttggttttgttttcttttg
ctactcatttgacttggcacactcagtgggcctttccctttatctttcttcatttctgagac
gtttttctctcttattttttattatcttcctttcatttttcctgtcctttttctttctagac
atctcttaggaggatagtggtcctcttagattgatatgttatgtccgtgatttccaaagtaa
gatttgtactcgtcgtctgttaaaaggaaaagcatacatataccctatgtatatatgcacac
ttttttatttttaaattatatatgtatctgtactaattatttacattgtaagtcaaccctaa
cataatcttaaaggataagatacaaaacatactgcatctagaagcttcagtactttcttcct
gaatcccagtagatccttttgttcatcccacgggatgcattccgcccccatcctcccactcc
ctttggataccacattaccacagctctgcatcacttaactttcctcttatgtttttcacctt
ttttttttttttttttttgcattttatgtcctggggaatttccttaattcatttcatggttt
tactgttgatttttttaatattggccatcgcaacttttcttttcttttcctttcctttcctt
tcctttcctttccttttcttttcttttcttttcttttcttaattttcttttcttttcttttc
ttttctgttcttttcttttcttttcttttcttttctttcacacaggatcttggcgtgttgtc
caggctggcctcgaactcctgggctcaggtaatcctctcaccttggcctcccaaaatgccag
gattacaggcgtgcgccactgcatttggcggcaacttaatttttttatttttatttttcctt
ttagaggacacctagcactgagcattgcaacttttcatttccatgaacttttaagaaaactc
ttaaagacatgtttaattctgtacactttctattgttctttgattgctgtttttgaataaca
acaaggagtacgccttagcattttgatggtatcctcttaatagtcgcaataatagtcccctt
ggcgctctgtatactctcaagtcttaaatgttttgtatgcagctgtacgttgacagttgaat
ggtctcgctccaagtggatcagcaagaacataaagaatcatttaactggtacaggctgcggc
ttgtgaattccctattaacaccaaagaagacgtgtgagactccgtactgaaactaaagacga
cttgtgagttccacactgagatcaaataagtctttatgatggtgacagagagtggtgtcaac
gcctaaagttttggttaatctctctaaattgaggggctgaccaaaagggggaacttaactgt
attagacataattttgagaaacatgggtatgtggatggtaatggaggaaatgggtgtagatg
agattgcctagggagagtgagaagtaggttaggtctaagccttgatgagttcccaacatttc
caagggtagttgaggatactgaaaatgagtggccagtgagatagaggtaaagctagagactg
cccaggggagaggaattttcaacaatgaggaggtgtcaacattgtcaggtattgctgagagg
tcagataaaaccagaattgagcaaaatggccattggaagcctatggtgccctccgtaagagc
tgtttcgctgaagtgatagaaacggaaatcaggctgggcacagtggctcactcctgtaatcc
cagcactttgggaggccgaggtgggcggatcacctgaggttaggagttcgagaccagcctgg
ccaacatggtgaaaccctgtctctactaaaaatacaaaaagtagccaggtgtggtggcaggt
ccctgtaatcccagctactcaggaggctgaggcaggagaatcgcttgagccccagaggcgga
ggttgcagtgagcagagatcgagccactgcactccaacctgggtgacagagcaagactccgt
ttcaaaaaaaaaaaaaaaaaaagaaatggaaatcaggatggtttggcttttattttaataaa
atagctagagcagggaaatggggtactttttttcccccttttaagatgagacatagccaggt
gcagtggcttacacctgtaatcccaacactttgaaagggagggtcgcttgagctcaggagtt
tgagaccagcctaggcaacatagcaagaccttgtctctactaaaattcaaaaaaaattaact
gggcatgctggcacacacctctagtcccagctatttatgaagctgaggcaggaggatcacac
ttgagcccagatacgtggggctgcagtgagccctgataatgccattgcactccacgttgggc
aacagagcaagacttcgtctcaaaaataaataaataccctgtctcaaaaataaaaaataaat
atgggaggagagatttgacttagattcctcaaagggcaggaggaaagagaattccaaacagt
gattcacctttaatgggagaaagatcgcttaattttacatgaggaagaagaggattggtgga
gatacagtaggtgaacagtttttgtatgaggaagttgaacatgtgtcattctaatagcttcc
attctctgtgaagtagagggcaaggtcatctactgagagttggggaggtcaagagagataag
gggagattagaagagctcttctagcagagagtggaagaatgaattgctaagagagatgaagt
aggattgttaagtagttttgagggccctgttgagatgtgcttccagttgggtgtgattttct
ccagtagtgctttatttccctgggtacaggcagagagaaaaacaataaggctcatgtagggt
ttgtattttgttggacaagtcaaacagaaaagtcagaggacgagggagtttagaatgtttgc
aaaagagttattgaaacgatgaaccgcataatctaaggtggtaagtgggtgaatagataagg
aggatgtgaataggtaaggagaagaaagaaatatcagattattgattattgatggcgactct
ctaatacagctattatgccattttaaccgattaagaaactaaggctttagaaaattcataat
ttgccctaactgcacagctagtaagcagtggaaatgtgattggaaccagagttcttctgact
caatagactaaatggatgtaaggatgtagttgaaagaagggtgagctaaacgttgtggaacc
atgagctctttctctggttgatatccctctctgtaagtgataacatgggtcacgctggataa
aaccttgtggtgattggtgactttcctttgtccttcctcctgtgcctagtctggcgagtatc
tgcctttccctttcctttctcattgctgccacctaactttaggctcttccccttacatctgg
gtaactgaaataagatcacctttttgttccccttctgatttactttgacctaacattatctt
tactattttctttaaattaatgtttcattagtcttattctactcaggaactctgtagttccc
cattgcctacgaaaaaaagttaagcctcagccttatattcagtgactcttcaattggatatt
cagtccagttttactcctcctatgagccttctatgccagctccttgggtctcttgccctttc
attgtctcagctctgcacccttctttctcttttttattcttttttttttttgtacttttttg
gttttctttttggtttctttttttgttttatttattaaacctccatcacacttcatcctatg
gagttttgaaccacagcaaggtgcagtatcatcctggggctctggaggaagtggcagggagt
ccaaaatgtcaccttagcttcttatctggggccacatgtatttctgcatctgctgcttccca
cactcttgcccacaagtgtcgcttgtggaaataatttgagatttactgtctggctgacccta
gtttcaatctcttttccaccatttgctaatcattctaccttgggcaaaacatagaattaaaa
gaaaacttcagacaagttaaatttgatggagtttaattgagcaaagaaaaaaaatgatccac
aaattgggcagtctccagaatcaccgcagattcagagagactccaggggtgcctcgtggtca
gaacaaatttatagacagaaaaggtaaagtgacctacaggaatcagaattgagacatagaaa
cagtgagattggttacagctcggcgtttgccttatttgaacgcagtttgaacactcagcagt
ctatgagtggttgaagtatggccgctgggattggccaacactcagctgttattacagatgca
tactactaagttaggttttcgattttgtctgcctatttgagctaggttacagttcgtccaca
aggactcaaatataaaagtacggagtcctcttcgggccatatttagttcgctttaacaattc
ccccttttggtcagcccctcaatttagagagattgaccaaaactttaggcgttgacaccact
ctctgtcaccatcataaagacttatttggtctcagtgtggaactcacaagtcgtctttagtt
tcagtatggagtctcacacatcttctttggtgttaatagggaattcacaagttgcaactttg
taccagctaaatgattctttatgttcttgctgatccagttggagtaagaccattcaactgtc
aatgtacagctgcatacaaaacatttaagacttgagagtatacagtgcaccaaggggactat
tattatgactgttaagaggacaccgtcaaaatgctaaggtgtactccttaataaaagttctt
atgaaatgaactgaaccaaatcagccaagttaaggttcagacaatataagcagttcagcagt
attggggtctgattggtcagagtcttcagttggagtatgatagtgattaaggatcatagttc
gctgtaaagtagcttgacttaaagaggtgctcgttttcattgttaccttgttaatacaagtc
ataataacttgaaaacctgctagaagagatataaagattagaaacccttggaaaacccaagc
ttgccattcaccacttaggatgcctgcaaaccaactgttagttgctcctataaacatatcgt
gggttcctttctcttgagagatttctttattgtacttggtggcagtgtctaaggaaacagca
gtatcagccaccttttaaattaagctttttgtagtaacagaatcaggggagggattagtaca
aaattcagttttgtttaacaccaaacataggcctccagcttgagcaaaaagaagatctaaga
ctgcatgatcttccattaagtgttttcgttgaatatgtatgttgtcatgtgcctttctgaga
gtagcttctacccatctgaaaccctgggaggtctgattggctaccaaatccaagaattttcc
caatatacaaattagttttaaattccgtacaaatggtacttcactaccaccaagagtgagcc
cccaggaaccccagtggaatctttccccggtagaaactagcttatcctcgtctatttcgagg
ctagtgctaatttcagttattgatcattttggcctccaagtataagggctatcatgagaatt
ttcaggggaagcaattcgaaaggcaggagcaggccaggccagataacaagaaccaaaccaac
caaggaggcagaacagaatatgcagattctccacagacccaatagagaccctcaggggttgg
aaaagggggccacctagttgtatttgagcagggatcattcaggtttgttcgaccatgaatct
gtagctcctgaataacatccagtgggaaatttacttttctatggcccctttgtagtgtgttg
taagggtgtataaccacatctagtaaaaagagaccctactggatatacaagcaatcacttgt
actaacataagtaattcccaaatcttgagtatgtgatgcctgcaagcacaatatacgttttg
taggcatcatttggatttgttttttatatttggtgtgatcgactttatcagttgaaaaagag
tgttgtttttagtgagtgtaggaaagcaagtactagtgatgtttagagtatcaagaatagct
ttccattcttcccttggggtttcagggtgactcattgggaaacgtggaggggcactggcacc
cttggaatcatttcctgattttttggcattagcccacaaacccaacagttaccctggttttg
tgctagagcataagcttgagctgaagccatccactgattatggtcccatggattttcatgta
aggaaaaggaaaggattagggaaaaaaataaggaaaacagaaaaacacataaggctttcatg
gtggtagagaagtcttgatctgtgatctagggaaagctgtctgtaaccaggatgctgtctgc
ttctgggaagagatttccctggtcagctttaccttaaagtctccaacgggtatatagtacca
ggagtctgagggggcccttttgaattgtgagatgtggacccatggttcaaagccctgaagct
tctctgcactgtgggtggtaagaaggacttggtatggtcccatccaacgaggttcaagagtg
atcttcttctgatgtcatttccggaaggcccagtctccaaattccagaccatggagggtttg
attgtcctcagttggtggatcttgaaatgcttcctttacctggtggaagtatactttggcgt
aatacattaaagccttgcagtatttagtcatatcagagtttaagagagcaggagaagcatga
gatgctattattagggacatgggcctcccagtgactatttcataaggggtcaatttatgttt
tccaacaggattgaatctgattgccattaaaaccaaaaggtagtacctttggccaaggcaac
tcaattgattcagttaacttggacagtttcagttttcaaatgccatttgttctttcaagctt
tcctgaagactgagggtaataaggacaatggtaatgcaactgtgtcagtaacaccttattta
actgctttataacttgctcagtaaaatgagttcctctatcactggagacttttagagggatc
ccccataaaggaaaaacattttctaataatttcttagctatggtcacagcatcagctttcct
acatgggaaggcctttatccaaccagaaaacatgcaaactattacaagaacatactgatacc
ccattgagggtggtaactgaatgaagtccatctgtaaatgttcaaatggtccatcaggtggt
ggaaatatactgcctctagtttttctgggattatgagtttgacaagtcaaacattgattata
agccattttagtaatgtcagaatagtcaccccaccagtattttttcataatttggatcactt
tgtctgttccatgatgagctgtggagagctttcaataatggaagcttcaaagattcaggaag
gaccaggcggccgtccgggccctttgtgagtctttgcttcacgttaaatttacatcctttta
gataccagttttgtttttgcaaatcagatgcgttgcactgtttattaaataggtcatcgtaa
ggaaattggcttggattaatcttatggagttcattcagattgcgtatcttgatggttccagc
actagctgattgagcataaaaatctgctaaagcatttcactgatatttgggttcatttctac
aagtatgagcttcagtcttaataacagcaatctgcatttgtaacaggatagcagaaaggagc
tcatctgtttggagtccatttttgatggggatcccactagaggtgagaaaccttcgtagttt
ccatatcatgccaaaatcacgtactactccaaaagcatgtctactatccgtaaatatttact
gacttgtccttagctgtgtgacatgttcaggtaagggcagaaagttctgcaggttgggctga
cttgacttgaagagttcgcttctctattaactcattttgggtggtaacagcatatcctgact
gatatttttttttctgagtttttggcataggacccatcaacaaaaagtgttaattcaggatt
atccagtggagtatcttgtatagcaacacgaggggccactatttctgatactacactcacac
cgttgtggtcttcaccatcatcaggcagagataacagagtagcagcattaagtagattacag
ccttttagatgaagataagaaggagataggagaagtaattcataagatgttagtctactcac
tgaaaaatgctgggtttgattggaatttaatagactttccacagcgtgtgggacttgcaaat
taagttcatttcctaaaaccagatctgatgaagcttctaccagcttggctgctgctactgct
tttaaacaattaggatatgccttagagactgggcctaattgcaggctatagtatgcagtggt
cctatgtttagcaccgtgttcctgattattacattcatgaacaaacaaagtgaaaggtttag
tgtaatttggaagtcctaaagctgggggctgttgtaaggccaacttcatttggctaaaagcc
tgctcatgactgtcttcccaaggtaaaggctctggtacagcatttttagtgagctcatacag
tggtgaagctattaaggaaaaatttggaacccaggatctgcaatatcctgcaagcctaagaa
agccttttgtcttttggttgcaggtcgaggaaaactttaaataggttttatcctctcaggta
agagggaaatcccttcagcagccaagtcatgtcccaaatagtggactttttcttttgaaaat
tgaagttttggccaggcatggtggctaacgcctgtaatcccagcactttgggaggctgaggc
aggcggatcacctgaggtcgggagttcaaggacagcctgaccaacatggagaaaccctgtct
ctactaaaaatacaaaattagccaggcgtggtggtgcatgcctgtaatcccagctactcggg
aggctgaggcaggagaatcgcttgaacccaggaggcagaggttgtggtgagccagtatcaca
ccattgcactccagcctgggcaacaagagtgaaactccatctcaaaaaaaaaaaaaaagaaa
aaagaaaagaaaaaattgaagtttttccattgaagccctgtgacctttatatgcaagttgct
gtaaaaggtaaactgagtcaatttccgggcactccttaataggagagcataacaataagtta
tctacatactgaatgagagtagaattttgaggaaactgtagtgtcattaactcctgatgcag
tgcctggggaaaatatgaaggggcttcagtaaacccttgtggcattacactccaggtgtatt
gctgatttttccaagtaaaggcaaacaagtattgactttctttatggaatgctagagaaggc
tgagccaagatctattactgtggacaacttggaatcagtgggtacattaggttataaagtat
taggatttgggactacaggaaatcttggtattacaattttattaattgcctgtaaatctgga
acaaatctccagtctcatccattttgttttttaactggtaggattggagtgttacaggggct
ggtgcatggaattatgagtccttgtttaattaaatcttctacaattggtgagagcccttaaa
ttgcttcaggttttagtggatattgtggtaattaggcaaaggtttagaatgatctgttagta
cttttataggttctacacttttaattcttcctatatcagttgggaagaggcccataaacatt
aggtgttttcgaaagatcaggggtattacaggcttgagtttcgatcttatcaatttctgcct
gtagacagcataacaattctagttcaggagaatcaggaaaactcttaagattatttctgttt
ctgaggaaaattttaggtgcccttttagctttgaaagtaaatcttgccctaccaagtttact
ggaacagtatcacgtagtaaaaaactgtgtttttctgaaagggggctcagagttaattggat
gggttcagatatgggaacctctggaacttgatttgaaacccctgtcacagaaatgacctttt
tactctaagggatttgttggcttattaaggtggggtttatggtagatagagtagccctggta
tccataaggactatacacaactccctatttattttaacctctgtttccccatgttcctttaa
aggtattacggggagcaatccactggagaatcccttagagcctcctttaagttgaatattgt
caggaggactaaggtctcttgggctccctctagtggtgaaacagtttggcctagagggaggt
ttatcagccgacaatcccttttccagtgccctggttgtttgcaatacaggcagacatcttgg
ggtaaagaaattcttgttctgggacctcttgatttgatttttttaatatataattttaaaaa
tattttccaaagtgtgacttaaaaaaatttttttttattatactttaagttttagggtacat
gtgcacaacgtgcaggtttgttacatatgtatacatgtgccatgttggtgtgctgcacccat
taactcatcatttacattaggtatatctcctaatgctatccctcccccctcccccaacccca
caacaggccccagtgtgtgatgttccccttcctgtgtccaagtgttctcactgttcagttcc
cacctacgagtgagaacatgcggtgtttggttttttgtccttgtgatagtttgctgagaatg
atggtttccagcttcatccatgtccctacaaaggacattaactcatcattttttatggctcc
atagtattccatggtgtatatatgccacattttcttaatccagtctatcattgttggacatt
tgtgttggttccaagtctttgctattgtgaatagtgctgcaataaacatacgtgtgcatgtg
tctttatagcagcatgatttataatcctttgggtatatacccagtaatgggatggctgggtc
aaacggtatttctagttctagatccctgaggaattgccacactgacttccacaatggttgaa
ctagtttacagtcccaccaacagtgtaaaagtgttcctatttctccacatcctctccagcac
ctgttgtttcctgactttttaatgattgccattctaactggtgtgagttggtatctcattgt
ggttttgatttgcatttctctgatggccagtgatgatgagcattttttcatgtgtcttttgg
ctgcataaatgtcttcttttgagaagtgtctgttcatatccttcacccacttgttgatgggg
ttgtttgtttttctcttgtaagtttgtttgagttctttgtagattctggatattagcccttt
gtcagatgagaagtttcagaaattttctcccattctgtaggttgcctgttcactctgatggt
agtttcttttgctgtgcagaagctctttactttaatgagatcccatttgtcaattttggctt
ttgttgccattgcttttggtgttttagacatgaagtccttggccatgcctatgtcctgaatg
gtattgcctaggttttcttctaggatttttatggttttaggtctaaattaagtctttaatct
atcttgaattaatttttgtataaggtgtaaggaagggatccagtttcagctttctacatatg
gctagccagttttcccagcaccatttattaaatagggaatcgtttccccgtttcttgttttt
gtcaggtttgtcaaagatcagatagttgtagatatgcggcgttatttctgagggctctgttc
tgttccattggcctatatctctgttttggtaccagtaccatgctgttttggtgactgtagcc
ttgtatagtttgaagtcaggtagcgtgatgcctccagctttgttctttggcttaggattgac
ttggcaatgcaggctcttttttggttccatatgaactttaaagtagttttttccaattctgt
gaagaaagtctttggtagcttgatggggatggcattgaatctataaattaccctgggcagta
tggccattttcacgatattgattcttcctacccatgagcatggaatgttcttccatttgttt
gtatcctcttttatttccttgagcagtggtttgtagttctccttgaagaggtctttcacatc
ccttgtatgttggattcctaggtattttattctctttgaagcaattgtgaatgagagttcac
tcatgatttggctctctgtttgtctgttattggtatataagaatgctctcttttgttctttg
ttagtcttgctagcggtctatcaattttgttgatcttttcgaaaaaccagttactggattca
ttgattttttgaagggttttttgtgtctctatctccttcagttctgctctggtcttatttat
ttcttgccttctgctggcttttgaatgtgtttgctcttgcttctctagttcttttaattgtg
acgttagggtgtcaattttagatctttcctactttctcttgtgggcatttagtgctataaat
ttccctctacacactgctttgaatgtgtcccagagattctggtatgttgtgtctttgttctc
attggtttcaaagaacatctttacttctgccttcatttcgttatgtacccagtagtcattca
ggagcaggttgttcagtttccatgtagttgagcagttttgagtgagtttcttaatcctgagt
tctagtttgattccactgtggtctgagagacagtttgttataatttgtattcttttacattt
tctgaggagagctttatttccaactatgtggtcaattttggaataagtgcagtgtggtgcta
agaagaacgtatgttctgttgatttggggtggagagttctgtagatgtgtattaggtccgct
tggtgcagagctgagttgaattcctggatatccttgttaactttctgtctcgttggtctgtc
taatgttgacagtggggtgttaaagtctcccattattgttgtgtgggagtctgagtctcttt
gtaggtcactcagggcttgctttatgaatctgggtgctcctgtattggttgcatatatattt
aggatagttagctcttcttgttgaattgatccctttaccattatgtaatggccttctttgtc
tcttttgatctttgttggtttaaagtctgttttaccagagactaggattgaaacccctgcct
ttttttgttttccatttgcttggtagatcttcctccatccctttattttgagcctatgtgtg
actctgcacgtgagatgggtttcctgaatacagcacactgatgggtcttgactctttatcca
atttgccagtccgtgtcttttaattggagcatttagcccatttacatttaaggttaatattg
ttatgtgtgaatttgatcctgtcattctctcaacatttgcttgtctgtaaaggattttattt
ctccttcacttatgaagcttagtttggctggatatgaaattctgggttgaaaattcttttct
ttaagaatgttgaatattggcctccactctcttctggcgtgtagagtttctgccgagagatc
agctgttggtctgatgggcttccctttgtgggtaacctgacctttctctctagctgccatta
acattttttccttcatttcaactttggtgaatctgacaattatgtgtcttggagttgctctt
ttcgaggagtatctttgtggcattctctgtgtttcctgaatttgaatgttggcctgccttgc
tagattggggaagttctcctggataatatcctgcagagtgttttccaacttggttccattct
tcccgtcactttcaggtacaccaatcagacgtagatttggtcttttcacatagtcccatatt
tcttggaggctttgttcgtttctttttattcttttttctctaaacttctcttcccgcttcat
ttcattgatttgatcttccatcactgataccctttcttccagttgatcgaatcggctactga
ggcttgtgcatccgtcacgtagttctcgtgccttggttttcagctccatcaggtcctttaag
gacttctctgcattagttattctagttagccgttcgtcgaatttttttcaaggtttttaact
tctttgccatgggttcgaacttcctcctttagcttggatagtttgattgtctgaagtcttct
tctctcagctcgtcaaagtcattctctgtccagctttgttccgttgctggtgaggagctgca
ttcctttggaggaggagaggtgctctgatttttagaattttcagtatttttgctctgtttct
tccccatctttgtggttttgtctacctttggtctttgatgatggtgatgtacagatgggttt
ttggtgtggatgtcctttctgtttgttagttttccttctaacagtcaggaccctcagctgca
ggtctattggagtttgctggaggtccactccagaccatgtttgcctgggtatcagcagcgga
ggctgcagaacaacgaatattggtgaacagcagatgttgctgcctgatcgttcctctggaag
ttttgtctcagaggggtacccggccatgtgaggtgtcagtctgcccctactggggggtgcct
cccagttaggctattcgggggtcagggacccacttgaggaggcagtctgtctgttctcagat
ctcaagctgtgtgctgggagaaccactgctctcttccaagctgtcagacagggacatttaag
tctgcagaggtttctgctgccttttgttcggctatgccctgcctgcagaggtggagtctaca
gaggaaggcaggcctccttgagctgcagtgggctccacccagttcgagcttcccagctgctt
tttttacctgctcaagcctccgcaatggcgggcacccctcccccagcctcgctgccaccttg
cagtttgatctcagactgctgtgctagcaatgagcgaggctccatgggcataggacccgctg
agccaggcgcgggatatagtctcctggtgtgctgtttgctaagaccatcggaaaagcgcagt
attagggtgggagtgacccaattttccaggtgctgtctgtcacccctttccttggctaggaa
agggaattccctgaccccttgtgcttcctgggtgaggcgatgcctcgccctgctttggctca
tgctcggtgcgctgcacccactgtcctgcacccactgtctgacaatccccagtgagatgaac
ccagtacctcagttggaaatgcagaaatcacccgttttctgcgtcgctcaagctgggagctg
tagactggagctgttcctatttggccatcttggaaccgcccgattgtgatttaaaatgagaa
cgagatggtccctttggttcctggtccctgtaactgttgcaattgaaggggcataagcttat
tagccttttgaggttttttttgctctagagtcttctcaaaatgcttagctaggttgggcacg
atggctcacgcctgtaatcccagcactttggaaggccaaggtgggaggatcacgaggtcagg
agatcaagaccatcctggctaagatggtgaaatcccatctctactaaaaatacacagattag
ctgggcatggtggcacacgcctgtagtcgcagctactcgggaggctgaggcaagagaattgc
ttgaacctgggaggcagaggttgcagtgagccgagattgcgccactacactctagcctgggt
gacagagcaagactccacctcaaaaaaaaaaaaaaaaaaaaaaaaagttcagctaaggccac
caattcagtcacatctctaacttcccattgcaacttatgttttttagttaaactgctaagtt
caggatggagtccatttataagtaaagcagttaatgctgtttcagcccctgcagggaatact
ccttgctgtactttgagcccaggatgtttcacaaatatttctaagcgacttctgtaatctga
aactggttcatcttttcttttcttttttttttgcttacaagattgtatgatggaccaatttt
ttgtggaaaaattttaggaactgaatgttaaaaggttttcagcgatttttctagctattttt==
ggtccttcttgtgaggagctcttagagggccctttaaaatgtcctcctcaggtttgtcccat
tctgctgctgccatccatttctgagcttcaccagcccccagtatcatatgaataaattggta
aattcatgaagtcctggatcgtaagctcctattaggattctaaattcctcagtaaatttttg
agacttttcccttggaccagggaagtccttcacaatggggctaagctcagttttagaccatg
gagtgaaagtagttacagcaggcaggcctggctgatataaggtctcactttgtaagacatct
gtctaacttccttttttttttttttttttttttaaatcatcttcagggtgaaagtgtaattt
aacaaaaagtttagtggactcagagtatgtaggtagagatggacaaagaaggaacagtccga
gttagatcagtcaaagtacagtcctctttcttcatgtccttggtctgttgcttaagcttttc
atttggtttttgcaaagaatcttttaaggaggcactttttgattcacttagtcttttggagg
cctttgcgtatccatgagacaatacatcccactgtatttgtgggggctttgatccccttttt
ctaatatgccttgcaaacaattttatccaaattaaaacttctccattgtggccattttaatt
ctaagttttctttagtgaggttaacccattttactgaaaatgcacatgttctgggcccataa
tttttatacgtaaaattagctggagtccctgaagatggagtcccagactccttggattgaga
tgatcccattattaaataaggtacttatcagaggtctgaggcctctaactgaatccaatcca
gttaattatcaaatccaatttgatcttggatccagtccaggctaagtattgcttgagtaaac
tcggagagctcaaaacacaagttagtggagctcggaatctgagagaaaactcacccatgacc
tccagttacaatcaagagaccagtgagagcaacggcctcagtgggtacctcaccaggtcacc
tggtgttccagggggttgccagagtttttcttcaaatcccacttctgacaccagatctgtta
aaagaaaacttcagacaagttaaatttgatggagtttaattaagcaaggaaaataaacactt
tgcaaatcaggcagcctccagaattgaatgcagtttgaacacttagcagtctattagtgctt
gaagtatggccactgggattggccaacactcagctattattacagatgcatactactcaggt
tttccattttgtctgcctattgtgctaggttatggtttgtccacaagaacacaaatatagaa
gtatggagtccttctcaggccatatttagtttgctttaacaatacttaaaaaaaaaatttgt
aaaataaggatacttaaccttactcggtgtttctgagagttaacatttatatagttatgctg
tagtgaaaacagctagcgtaatgtctggtatgtataggaacacaagagataccgcttttccc
atatccccataccattcttcacagcattgctcctgtcttccttgattcctcctcctccttct
ttgttttttttttgtttgtttgtttgtttttttttggaggtggagtctcactctgttgccca
ggctggagtgcagtggtgtgatctcagcttactgcaacctctgcctcctgggttcaagtgat
tctcctgcctcagcctcctgaatagctgggattacaggcacacaccaacacactcagctaat
ttttgtatttttagtagggatggggtttcaccatgttggccaggctggtcttgaactcctga
cctcaggtgattcacccacctcagcctcccaaagtgctgggattacaggtgtgagccaccac
accctgcctccttcttaagaagtttccagtcccttgtaattaaaggaattaatattttttaa
ctacttagaatcagactggccctgattattagtaagcaactaatagtaagcaagcaactatg
tatgcaactatgagtgtatgttaagatatggttgttggtaacctttcattctcttcaggaag
aagaagagggtggagctctacagtcaatgtgtacatttaaattctgttccctttcgagcttt
tttgctactttcattcttctggggatccaggtgcttgagttgggattgattaacttccttaa
tttccacccctgtgctgtcaggatcgggagacatagatgaaggtgttctaaactgctagaaa
ttttgtttttgaaagcaaaagtttgcatgcatttttgttttcaacttttacttacagtgaat
agtagttaataaaataagtccctgccttttctctctttggtttcaattcctgagaccaggat
catagcccacatattagagtggagtcccactgctttggtttgaatcatgcctttgtttctta
tgtcagtgtgactttgggcaagttatttaagtctttgcaccacattttcctcatctgtaaaa
tgaggataatactagtactttctacatgggattgttagcaggattaaatgagatagcacata
ctgtaaccatgtctggcacatagtcaatggttagtaaatgtgaactattgtgtgacattgtg
gttagtcacgtatggggctgtgtttcctttagtatattgctcttttaatgtcatttcctttg
tactgttaccctctctgatctttcttccatattca
SEQ ID NO: 276
Organism: homo sapiens
Gene: OPA1
Intron: GRCh38/hg38:chr3 193618937 to 193626091
Intron Sequence:
gtaagtgtaaaagagaattgttcatgtaggtagtcttgaaagattttttaaagtttttactt
ctttggaagattttaaaatgataacatctgagaagcaaatacaaaaacatccaagtagagat
atcgttactaatcttagtgcaaagtacaaggtattacgtggcagttctggaaatataattga
gaagcccatttctttcacatatgtccagtgaagcattagtttcgagggttgtccccaagaaa
gagttgtgttgttaagtgtgtggggggagaaaggctcgtttagacaaggcaagcggacttct
tttctttccctaggacctctcatactgtaatatactcatgcgcattgtgaatttccaaggag
tcaaagcatacagtgttttcccaaattatttatcaacagaacccttttgctcatggaacgtc
gtatagggactagatttcactttggggaaactagaaagggaataggaattgggttattagga
aataaatcaattccctgatattgatagttaacaaagttatgtatggggttatttatggtatg
ttattttcaacacatattcattaacaaaatccatatgaaagttataggagaattgctgaggt
agaataacatactttgtttgtatttataatactcatatatttacctgacgttttctgagtct
tcacttttttcattcttttggaattggtaaaataactgattccttgaaagtttttttctaaa
taatacctagataatagatttatagaaaaaatattgtatgaatgttttaacattcatgtaat
atggaacatgtaatttttatactggaggttattatagttttaatacatcaaagaaataatgt
ttattttggaagcagaaagaagaaataatttctatgaataggttttcatctctttccttgtt
cttcaactttgaactttttatattccaaattttaattatatttcaaaagatttttttctttt
gccttttaattttatcttttggagaaaaatgtatgtcaaaatgtatgtacgtgtatttgtct
tttgatttgatcttttttgaccctcttttgcattgacattattttaaccaaaggacactctt
gattgttcatgctactgggggaaaaaaaaataagtagaaattagcctaatagttgtggctta
ttttgagtgaaggccttagcccttaaggcaattaaatttactgtggagagaagagctaatct
aatggggagaaggagcctttgttacaggtgtggtagtgtggttctttgagtgacaagatttc
tgtttgccagattggttaggagaagtctgtgtgtctgctttctctcttatggcctaggatca
ctgtggtgaatgaaaaacctgtctcagggcctgactcagataattcccttaaaacccggcta
aggtcatagatgaataatcagtaattgaacagaagctctgcaatagaaaagaagccagataa
ttatttttggaaatttaattatatttacagattttattttatacagtagacatggaattaaa
tttattacattatgttctaatttactctttgcttgttttgatttgcttgtttgacaatacat
gtccttgtaaactatttccttttaactttttctcaatttatggtgcttattttccccattaa
agacttaccaattttttttttaactatttgttacacatactgaatctagagttgtaattaag
ctactttcattactggttaagtcaaattatagcaaatgctactataaaaatttactatccaa
aaatgtgtctcaagccccaactgatggtttcaaattctgttattaataatatgcagcattgt
gtttgcaaagcttggctgttacttgtgatgcttgagaatgatgagtcactcagctaaactga
gtgattttgagacttgtgtacaaattgatggttgaatgtaagcatgcaaagagagaccttag
cttagcagtaccctttttgaaatcactctgacatcaagtttgaaaatgtgggcaataatcag
aggtggtaaggtggccaggctttagctgaatacttttttaactggttcagtctgagggctga
aagccccagatttaaacagtatttagaatttgaagcagtcaagtattagtttaatggttgtc
aggtttgtaacaaagtttctggctagacttctactagaaatgtaaaagtgcatgtgaatcag
ctttttaaaaaagtaataataattgaaaaacatttctacaactagaactaaagaaaagattt
gtcctttctaataggaaaacacatctggagaagtgctggcaactagcagaacagttaggacc
attcagaatcaactgaagtgaaagtgacggggagctgaggggaacacagatagtttgacttc
agtcagacagaataaacatgatgaaccgataacctgtgattcccagcctggggttactactg
gagttttaggtgtcctggaaagttataataccggtcttcaaaaagtctacagaaagcataga
tttccacataatgctgcacaggctaacgaattaatcaagtttctttggtttggcctggattt
atatccattcagtttgtggacactactgaattatttatgtcatgttgatcaaaagttctgat
atgatttgattaatgaaacattgaaaaaaatagtaaaaccaaccatttttaaccttacacta
ctatcttgaggtatgattgacatacattaaaaccacctcttaataaatgcttcttgttaatc
aaaaatttgaaaacgtatgtccactggaggaaaaaagacatagccctggatgtgaactgaat
attactgagactcggagaccttcagaactacctgaagatgaatcgaagtgctgcctacttta
gagaattggactaatttaatttgggagtcagcagattgctgtatatcagtcatcatatatac
cggtgacaagaccacttagttcattcccttttttagattctgtaagattattgtgttccagt
gaaattgatttgcaaaatgagacattttattttctgtgcttttgttctatcatgtttctgat
tggtcataagcatctcacagaagtaagaaatatggcgattcagaaggcaacaagcacattta
taatttatagaaaatatttgaaggactttttcatggcccaaatcatgaaaagtagtagtatt
gttttaagtataattattaaattataatacattaatgttctttcttgcaacatattactctc
attctttttttttttttttttttttgagacggagtctcactctgtcacccggctggagtaca
gtggtacgatcttggcccactgcaacctctgcctcccgggttcaagcgattctcctgcctca
gcctcccaagtagctgggattacaggctcctgccaccacgcctagctaatttttgtattttt
agtagagacagggtttcaccaggttggccaggatggtcttgatctcttgacctcatggtccg
tccacctctgcctcccaaagtgttgggattacaggcgtgagccacccagcagtctgattctt
aattttatagtttatgttgtacctccccagctgaagtatctcttttcttttttcccgcgtgt
ttagtgttcactcatctttatagcatagctcaattgtcacttcatgaagccttccataacct
ttgtagctccattaattatattcttctgagtgtttaaaacacttgccatatgaaacactatt
tactttggcttacattcttactatctaatcggccatttctgttactaaatctttttctcaga
gcacctgggatagtcttgtgtcttagtaaaatcagttgattgatttaactcggtagagtaga
ggctgattaaagtaaataaatctggttgatgccaacaaaattttggtcccctcaattttttg
ctctcattacctgcaaattctccctggccttcatatttggcaaccattgaggagaacaaggc
tgtaaaagtagttcatgtacttgatattctgaattggaattaagcagagttgcttaagtagg
acttgcttttctgggatttcttatgcaacaaataatgtagtaactggaaatccaagttcaag
acactggcagattcgatgtcttttgaggacccttggcttcatagatgatgccttctccctat
atccttacatagcaaaaggggccaggcagctctggcctttttttgtaaggccaataactcca
gaaacctcatgacctcatcacctcccaaaggccccacctctcaatactatcacattgtgagg
ctaggtttcaacatatgaattgtgggagacaaaaattcagaccatagtataatatttcaaga
ttacttaaactcttctctaccaaactcattaacttttaggttagcacagtattttcattgat
attttggtttctggagttattactaattttcttgatctgatgttataattaaaaaaaaacag
gactttgtacgtgaaatgagactgagataaggaagctgattcagagatggagatttaaaaaa
agagagatgagagattgagatctgcagtgtcaaactgacaatagccaggagtcaggagatat
taagagactatatcatctgtgattgttaatgattatttattgttatttataaatactactgt
attttatatattatatacattgttttaaaaattatttttgtaccatttcttgaaagaaaaat
gtctaagcttgggaaaatatttattgaaaaatgtggtttgtacatctgaggagtgtatcttg
cacagtaggtgcatagatttcttcctcttcctgttccacatggccttagcttagaggctgtg
tggccatcacttggtatttagggtaagactggtgcacaaaatcaaagacaggtaaccttggt
ataagtgtagtatcatgtaaatagcttttctatgtctaattcttgttttcttcctacttttt
caggaggtcaatttcagttcatttcaactatctttacataatagtgctttagtaacaggcat
ggaaggaaagagacatgtccctagagtgttttcttgaaatctaatagatgattggagtattt
accatgcagttgtgtatatacataagcagtgaattcgagaggaatttttaagctgtaaaaaa
aagcattgtgtgccttatagacgcgagtgagaaatgtggaatatggctgatccaaagggaat
gagttatctcaattgattaatcacagtcagttacagattgaactctttgttctactctttgc
ccccttctcactattgctcttgactagtcttaagaaagaaatgtggaatattttctcacggc
tttgggattttataaattagaatactagtggtatgtaaatacagcaggtacactactgtata
aaccaacataggaagccttctttaaagggaattgtttgagaaatttgaacacttggataatt
tgaataaaggattgtgataaatgatcaaatgaaagaaaataaatcaggttactcttctttct
gcttgataaagcaataattttttttaaaggtaaaaattatgagaatgatgaggatagtagtt
agcattgtctttctttgataggtttgttaatgatcataaaactgatttatttaaagacatgt
ctttttataactattttatactgttgtatctggaaacaaatattgaatttcatttgtcatgt
ggaagaaatcaactagttttaacctttgatttataataaatcaaccactttcatttattgtc
taatactggcaatgaacacagcctaatgtatcaaaactaacagaataaaaattctccaagtt
atatccagactttaagacactttctaattatataaaataaaatattttgggcagtcattttt
taactctgaaactatttaaaactcctaatttagaatatcttaataaatacccattttcctct
ttttatttttataacttggtaaaaattgagtccattgttttcccagaacgctgttcttaaac
aaatggttacctccttcattagaactttactttttttaggatttctaattaagaaaacatta
ggcttgtaacattgtcaaatcttggtggtctttcttccacgttttttgaggtcgattatcta
agaggccatcagttaataaagctatgcaggaaatgacatcatgccacatgtgaatatcctgt
attaaaaattgtatcaatatactattttataattatgaagtggaatgaattttagaaataga
aaaggtgattttttgtgcataggtccaaactgtgttttgttttcatttcagaatttcataat
aactatattgtctccatatcttaattgtgtttttttatagcacttttgtttagtaatttgta
tatgcttggctgtattctcagaggctgtttctatttaatgttgtcaaaacagctcataaaaa
gtgaaaattcggtcagactagttatttgatattatatatgaaatcaaaacaacctgaaacat
tatcttttaatttaaataaagaaccccaaattttaatcaaatgtatgcaaaggcacatagaa
tatatgacttaatgtacaacctttattaacttgatgatggaaacctgttcctagggaccttt
acttgaataaatgaaatatcaagaaaaaatactaacttaagaataataatttaataagtaag
taagctattatgatcttcaatcagtcctgagagaatcatggttgagaattagaaaatttaga
ccagtaagatcaacactgttaaaaaaaaaaaaaaatcagtattttttctccatattttttat
atatctggatcattttatttagcacttattattgcactttccttttcactttttaaactatg
ctgttttatttttctgagacatctgatttactgaggaggaaaatggaaatgcggtacagagc
ccaagggtatgacggctttaaatgagtttccatttctgttttaagttaaccatccctcccta
gcttacatctgttcctttgttgcacccttggtttaacattattctcctccccaatttcctct
tctcctcattgtgaactcgtggcag
SEQ ID NO: 277
Organism: homo sapiens
Gene: OPA1
Exon: GRCh38/hg38:chr3 193626092 to 193626202
Exon Sequence:
GGTCTGCTTGGTGAGCTCATTCTCTTACAACAACAAATTCAAGAGCATGAAGAGGAAGCGCG
CAGAGCCGCTGGCCAATATAGCACGAGCTATGCCCAACAGAAGCGCAAG
SEQ ID NO: 278
Organism: homo sapiens
Gene: OPA1
Intron: GRCh38/hg38:chr3 193626203 to 193631611
Intron Sequence:
gtgatggatggtttaagggggctaccgatacattcacactaatcagccatttctgccaagat
catgtcacctcaatctgttcatggactccaaatacaagaaattaatttgacaaagtgaaaat
ataaaagatgcatcatataaatatgtaacttttctggagtgggtagtataggtaaagccaaa
agaaacaaattcaagcagaggaattttggtttctgaaaattaggttgtctgtagggtccctg
tatttatacttagaacaaaattaggaatttctgtttatgtggtccagttattgagtcaccct
aagtttgtaggcatcttacctacctacttgctccccaagtttttatttctaaaatgaaaagc
attgctgtagatgaccagtttacactaaagaataacatttatttatttgttttagctaaagt
atatggacagggaacattcatattcttgtagaagaaaattattttgacttttgggcaaaagc
atgtagttcttatacactttgacaaactcattgcgtacatttttcacattaatcaaagtcag
cacaaataaattttcaccttggaccacggagggtttgaacactggaaatttgatataattct
ggttgctaaagaacaagttctaataaaagcttaagtgtataccaatatgtggctgttggtgc
aatcagcaggtccgtaaaaatatgattttaatggttaggtaatcccacaacggagatcccaa
agttcatgtttggaagagacttttgggtcaaagtgaaatcagtgtaatgaatttaaaattat
actctgagatcttgaaatcagctaattatgttacatcttattagctcagaaaagttttgaag
ttatatacaaatgctagtcaggaaaaaagattcagtcatgtaattcttgtacattctactat
ttaaatcaaccaatattatagattatgatttagtgcagtaattctgctggctaaccttatct
catttggtggtggttagtacttcagagtactcaccatagtttcatttatgttttcagcatca
cttcctggtttttctcaattccatggctgtggaatcaattcatatgtatatttagcttcggt
gagcaaaaacatagctagaaaaagaaaagaagtgagtttcctacctggttaaattaaagtcg
atgtgttaagccaaggaggacttcttttgaatggtactttaacaatccctgttctgtatact
gtgaatatatcatttaaatagcctaataaattggatgcttaggctgagccacctatacttta
gttttgttatggaaagaagggagaggagcaagtatgttcttatatgttacttagaaataaga
atgtagctgtagttacacattgttcttaagtttttttcgtaagacaacttgaaatgagtccc
ataggcctgctatttaacattctaagatatgacttaaggttaatgatgagcttttgaatctg
acaattcaagagatatccataatgaatactgattcattttctacattgctgaaagctaatgt
tcattttaagcctactttagtagcctttatttgggcttagagatgttattcctctttctgat
atttattgggttatctgtttaacccttttatatctccctttcccgatttgtaaattagagac
tggcaagactttttaccctgagtagagcaccaaacatggcttgtttctgcccacactgtagt
taccttgaggggaagtaaatgggactttaaaagcaatttatgctcttttatagtgaaattat
ccctcttactatcccgaaagactgttaccttacaatatcctccactcctttccccctgtagt
tactatagagatgacttttcggttcttcactgccataatgatcaaaatcctaattcatgaga
tttttatcattccaggcatgtgaggtttacttgatgcataaaaccgcaagtactttttgttg
ttttttaattgttttttctctcttatcttcttgaaagtctaagtagatcatcatttttgatg
tcttattagtagcaactaataaattttccctgtatcttctcagcaaaagaactcaagcagag
acagaagattagaactaccattggtagttttgcttcctatggatatgttcacatacatagaa
atttttacaatgacctttttatatatgtatttcagaatttcagaatggcctcaatgccttaa
taggaagaaatacttgaaatttttaaattagggcttggttttgtgaggagctagtaaaggtt
tttctctttcagctttagcttgtttctgcggaggattccgctctttctccatcagtttcata
gccctggaattgtagaaaagctctggtttcaagaccattgatatccatttctgtcagggtga
gttttaaatttatttcatgatgcaaacaatatattgaacaacaggacatgaacttgttcttg
ttgtaagtggctgaattttatcagtaaagcacatcaaaataaaatataccccaattgctagt
taagacctagagtgacagattgaaaatagcttgtgttattctcttaagaaaatatataaaaa
ttatcatctcatcaatctttaatgtttgttttataaatctaaatgtttttatattgtttcct
aggaaatattaggtctaattttttactttaccaccagctgtcttttattttactcttttttt
gagacggagtttcgctcttgttgcttaggctagagtgcagtggcactatctcagctcactgc
gacctctgcctcccgggttcaagcgattctcctgcctcagtctcccgagtagctgggattac
aggcacatgccactacaccaggctaattttgtatttttagtagagacggggtttcttcatgt
tggtcaggctggtctcgaactcccgacctcaggtgatccgcctgcctcggcctcccagagtg
ctgggattacaggcatgagccaccgcacctggccagctgtcttttaatataacattatgatt
aattgtgatgttccattaaactaagcggagaggaaacatgctggtaaaccatgtgtgagtta
ttcattgtaccagaaaggcaaatgatacattttatcctaaaattcaaatttataaacatctt
aacacttgtgatcattaaatactactaatctagcatataaattatatttgtaggcggggcac
ggtggctcacgcctgtaatcccagcactttgggaggctgaggtgggcagatcacgaggtcag
gagatcgagaccatcctggctaacatggtgaaaccccatctctactaaaaatacaaaaaaaa
ttagctgggtgtgctggcgggcacctgtagtcccagctacttgggaggctgaggcaggagaa
tggcgtgaccccaggaggcagagcttccagcctgggcgactccgtctcaaaaaaaaagaaaa
aagaaattatatttgtaatattctactaaccttatatcattttaactttttatataactttt
ttattttaccaaattaagttaaccttttatagcccttggcttatactaaacatcctaacttt
tttgtttaattgtattagtttttaagttattgccccagatgtcaagtaatgttggattttct
ataataatttaggatatattgcatgaagtcagttagtatttacatttaaaactaaaacaatt
tatactaatacagtttatacatttcatactaatttagctacagttggataaatatttaatgg
aacaaagtaaatcaaagtaccttttcaaatgaattggaaattaaatccacataacaattttt
tatgaccacactattacagtgtgatggcatgccaaatgatcataatgtggaattatgtattt
cttcattggctttcaagattctgttctttagtttgtgggctcctctccaacttgcttgtctc
ctcacagtttaggcgactgtttataattcttgtccatcctgcataaacacacacagtcaaaa
tgaaaaaaagcttctatcagcagatctgtgcttgctgtacagaaatgggaaaacaattgaag
tttgcattatcttttttctaattaccagatcgtttttggagctatttaggcatacgctttta
aggaaaaaagaaaaaaagagtgtaccttttgtttctaacaaaggttgttatctatattattg
aaataaaaaattggggatagttatgacaaagtatttagaaataggaattaaaatcttaaaat
aacttttcatagcatggacaagacttattaatgtctacctcaataagcaaatcatttaaaaa
tttttcatgtatatttgctgccatgatgtgttgtgattgcttaaataaccaatgaatgaaga
tcaacaaggatttaaatgaagaagaatatggatttaactattttctcctgtgaaataagttc
atatttacaagttttgattttcagaaattagacaattatttttaaaggctgggatgacaact
tctgcctcttaccaagaagtcaaagcacagttatgtgaattcatcataaatcacatcatttt
tattatattttgtatttataattgtattgtgactactttaaaacctgttataaaataaaatt
gttttttaatattttattttagaattattagcattaataacaatttgaagtagtttacacaa
tacctgtgagttttatttttgttttatattgaaattaattttagttgctttacttggcttca
ttgctatggatgcattctctgtgttacgagttagcagatctttccttggaactgaatttaaa
agcaagcatttggctccacttaaatctctgaaaatgcaacttgttctttgcatttattacat
aattcgctacttatggtacagaaatggatacaatacaaaaatatttccttataagatacact
gtgaccaatgagctttttaaatagctgtaatcagtaacatgtatttgacttttcaaaacaca
tttctggagggatatcagtgctttatttccccaaatatctgaatccctatgctttagtacaa
aacaacttctgaagaatttagtaaccatatgtgttgatctcttgtttttctaactagtcttt
cataagaaatgactagaatagcaacagggaaatgattgccttttaaggtttttgtttctcaa
tataaaattttggtgaaccatttttattgataaatacaggtatttttactttcttaaatcac
ttgatttaaaattactttgattaaatatgcatataaagtcagttgtttttaactctcaatac
ttatcaaaaaaatttaacttgctgtacattctgtataaacctaattctattcaactaaaatt
attttaaacatttag
SEQ ID NO: 279
Organism: homo sapiens
Gene: OPA1
NMD Exon: GRCh38/hg38:chr3 193628509 to 193628616
NMD Exon Sequence:
CTTTAGCTTGTTTCTGCGGAGGATTCCGCTCTTTCTCCATCAGTTTCATAGCCCTGGAATTG
TAGAAAAGCTCTGGTTTCAAGACCATTGATATCCATTTCTGTCAGG

### Example 2.2: Confirmation of NMD Exon Via Cycloheximide Treatment

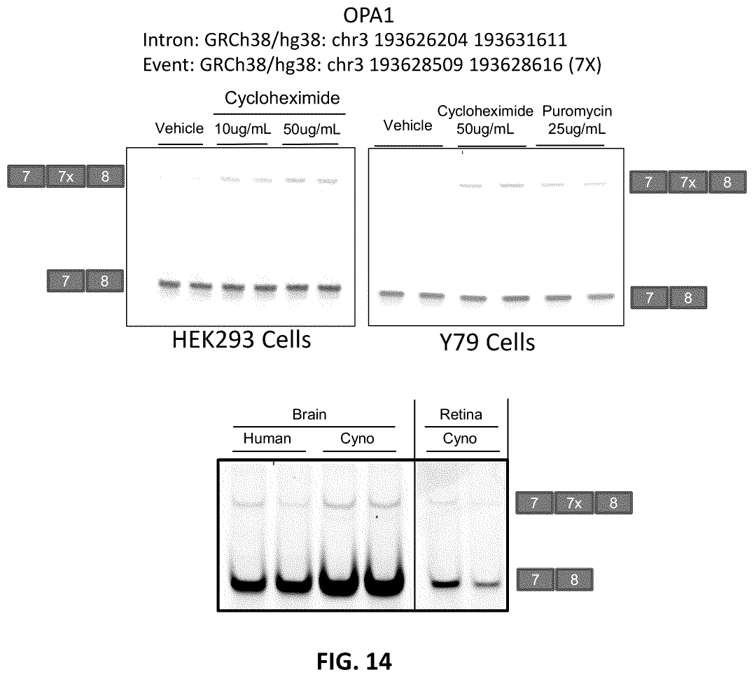

RT-PCR analysis using cytoplasmic RNA from DMSO-treated or puromycin or cycloheximide-treated human cells and primers in exons was used to confirm the presence of a band corresponding to an NMD-inducing exon. The identity of the product was confirmed by sequencing. Densitometry analysis of the bands was performed to calculate percent NMD exon inclusion of total transcript. Treatment of cells with cycloheximide or puromycin to inhibit NMD can lead to an increase of the product corresponding to the NMD-inducing exon in the cytoplasmic fraction. FIG. 14 depicts confirmation of exemplary NMD exons in OPA1 gene transcripts using cycloheximide or puromycin treatment, respectively.

### Example 2.3: NMD Exon Region ASO Walk

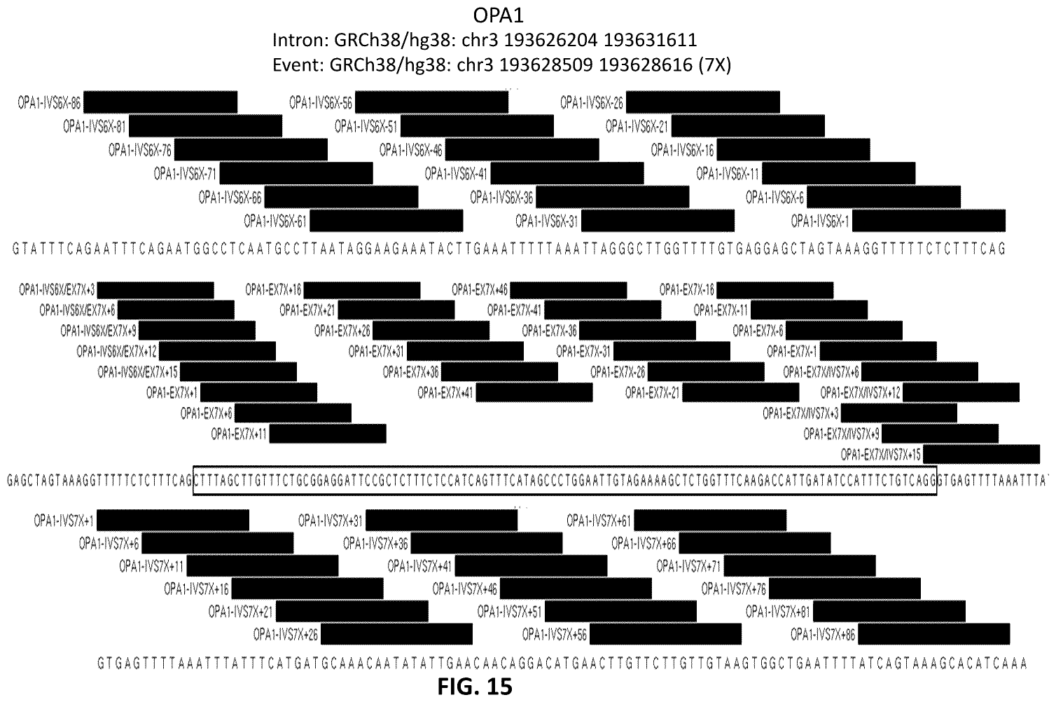

An ASO walk was performed for NMD exon region targeting sequences immediately upstream of the 3′ splice site, across the 3′splice site, the NMD exon, across the 5′ splice site, and downstream of the 5′ splice site using 2′-MOE ASOs, PS backbone. ASOs were designed to cover these regions by shifting 5 nucleotides at a time. FIG. 15 depicts an ASO walk for an exemplary OPA1 NMD exon region.

### Example 2.4: NMD Exon Region ASO Walk Evaluated by RT-PCR

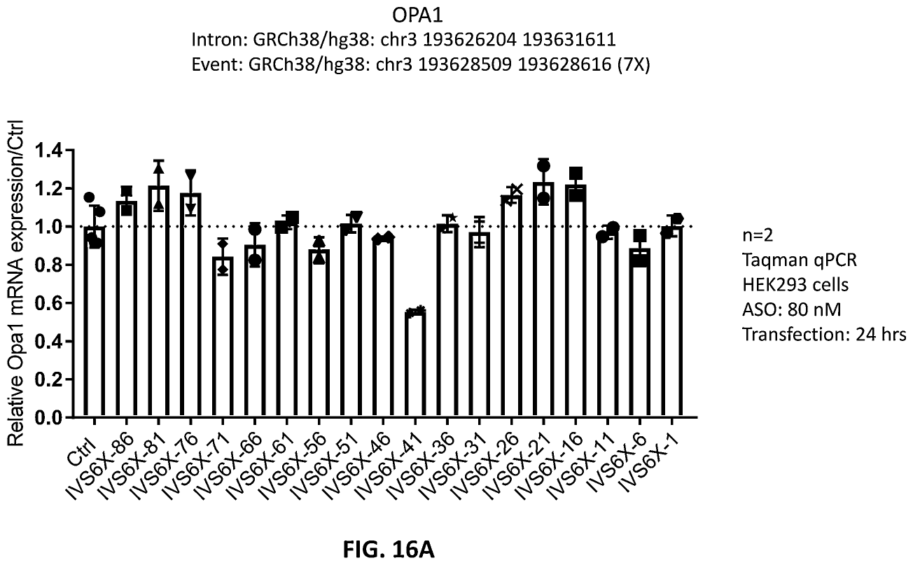

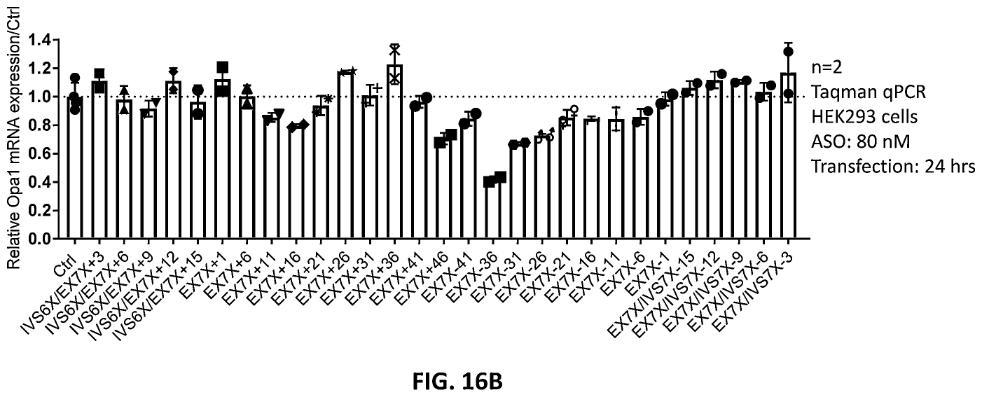

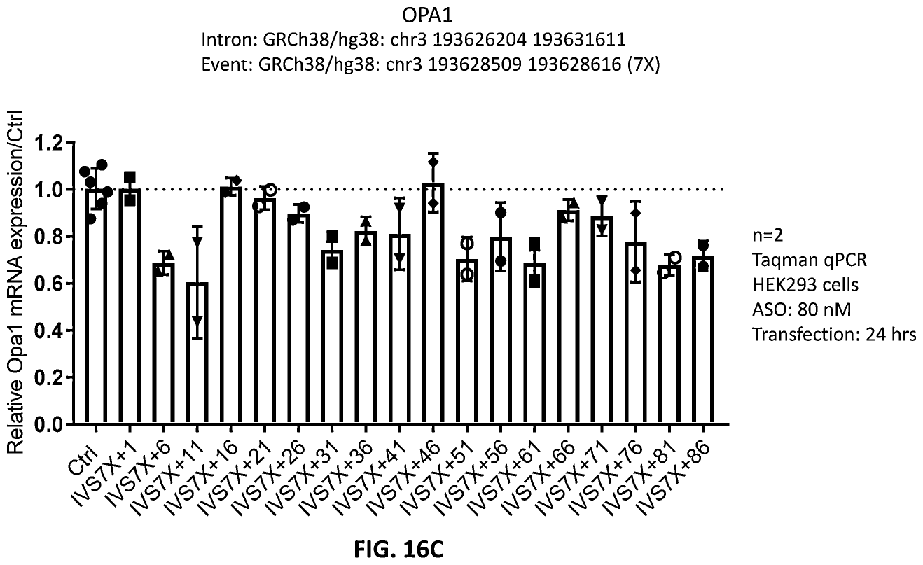

ASO walk sequences were evaluated by RT-PCR. HEK293 cells were transfected using Lipofectamine RNAiMax with control ASO treated (Ctrl), or with a 2′-MOE ASO targeting the OPA1 NMD exon regions as described herein. Products corresponding to OPA1 mRNA were quantified and normalized to RPL32 internal control, and fold-change relative to control was plotted. FIG. 16 depicts evaluation via TaqMan qPCR of various exemplary ASO walk along exemplary NMD exon regions. The measurement of the amount of OPA1 mRNA was carried out with HEK293 cells 24 hours after treatment with 80 nM of an exemplary ASO in the absence of cycloheximide, by TaqMan qPCR using probes spanning exon 7 and exon 8.

### Example 2.5: NMD Exon Region ASO Microwalk Evaluated by RT-qPCR

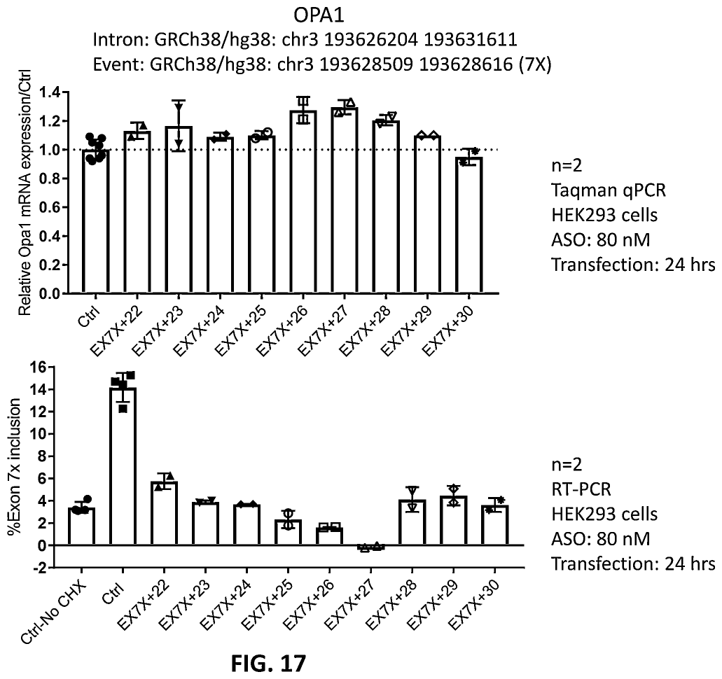

ASO microwalk sequences (across exon 7x) were evaluated by RT-PCR. HEK293 cells were transfected using Lipofectamine RNAiMax with control ASO treated (Ctrl), or with a 2′-MOE ASO targeting the OPA1 NMD exon regions as described herein. Products corresponding to NMD exon inclusion and full-length were quantified and percent NMD exon inclusion was plotted. FIG. 17 depicts evaluation of various exemplary ASO walk along exemplary NMD exon regions. The measurement of the amount of OPA1 mRNA was carried out with HEK293 cells 24 hours after transfection with 80 nM of an exemplary ASO in the absence of cycloheximide, by TaqMan qPCR using probes spanning exon 7 and exon 8 (top panel of FIG. 17). qPCR amplification results were normalized to RPL32 and plotted as fold change relative to control. The measurement of exon 7x inclusion was carried out by quantifying exon 7x inclusion based on RT-PCR using probes spanning exon 7 and exon 8 (bottom panel of FIG. 17).

### Example 2.6: Dose-Dependent Effect of Selected ASO in CHX-Treated Cells

PAGE can be used to show SYBR-safe-stained RT-PCR products of mock-treated (Sham, RNAiMAX alone), or treated with 2′-MOE ASOs targeting NMD exons at 30 nM, 80 nM, and 200 nM concentrations in mouse or human cells by RNAiMAX transfection. Products corresponding to NMD exon inclusion and full-length are quantified and percent NMD exon inclusion can be plotted. The full-length products can also be normalized to HPRT internal control and fold-change relative to Sham can be plotted.

### Example 2.7: Intravitreal (IVT) Injection of Selected ASOs

PAGEs of SYBR-safe-stained RT-PCR products of mice from PBS-injected (1 μL) (−) or ASOs or Cep290 (negative control ASO; Gerard et al, Mol. Ther. Nuc. Ac., 2015) 2′-MOE ASO-injected (1 μL) (+) at 10 mM concentration. Products corresponding to NMD exon inclusion and full-length (are quantified and percent NMD exon inclusion can be plotted Full-length products can be normalized to GAPDH internal control and fold-change of ASO-injected relative to PBS-injected can plotted.

### Example 2.8: Intracerebroventricular (ICV) Injection of Selected ASOs

PAGEs of SYBR-safe-stained RT-PCR products of mice from un-injected (−, no ASO control), or 300 μg of Cep290 (negative control ASO; Gerard et al, Mol. Ther. Nuc. Ac., 2015), 2′-MOE ASO-injected brains. Products corresponding to NMD exon inclusion and full-length can be quantified and percent NMD exon inclusion can be plotted. TaqMan PCR can be performed using two different probes spanning NMD exon junctions and the products can be normalized to GAPDH internal control and fold-change of ASO-injected relative to Cep290-injected brains can be plotted.

### Example 2.9: OPA1 Non-Productive Splicing Event Identification and Validation

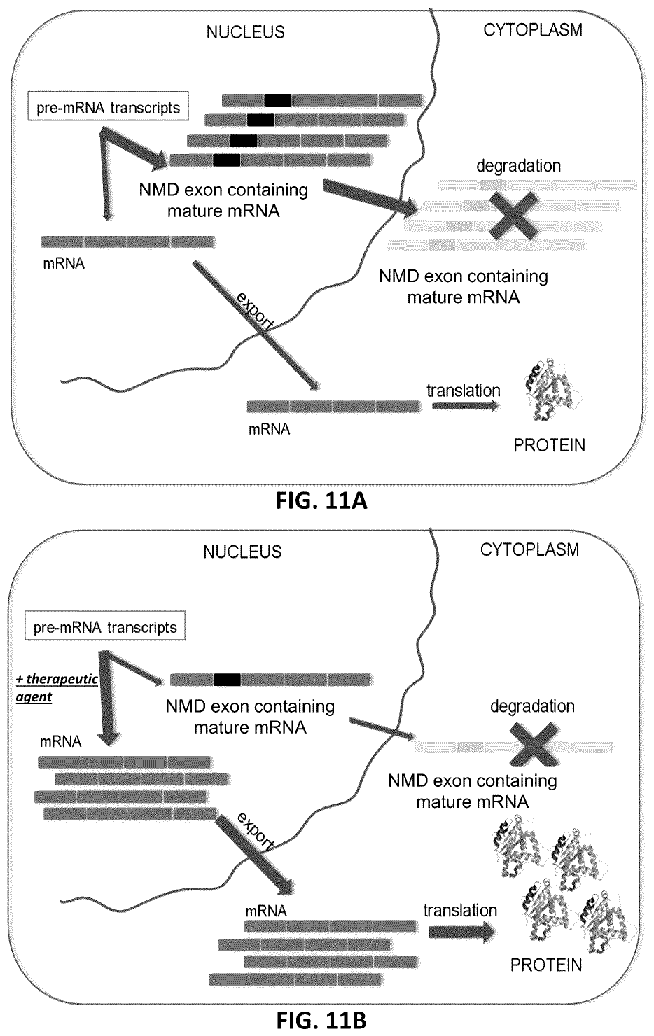

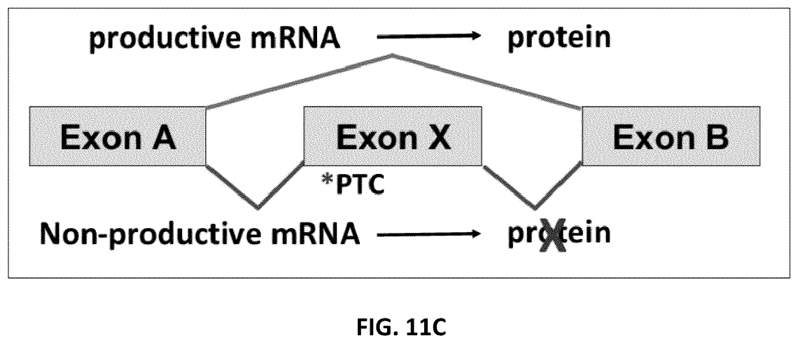

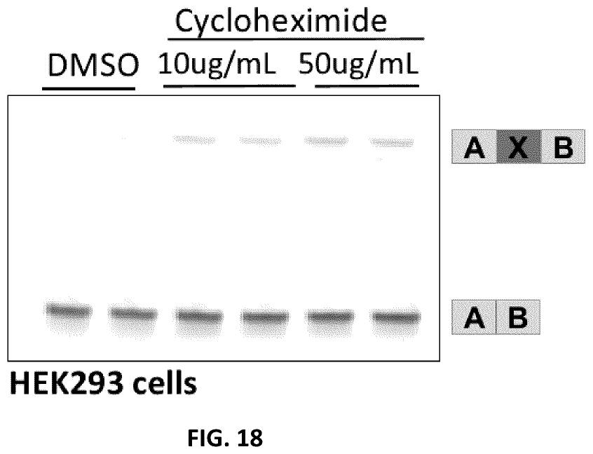

A novel nonsense-mediated decay (NMD) exon inclusion event (Exon X) was identified in the OPA1 gene which leads to the introduction of a premature termination codon (PTC) resulting in a non-productive mRNA transcript degraded by NMD, as diagramed in FIG. 11C. As NMD is a translation-dependent process, the protein synthesis inhibitor cycloheximide (CHX) was used to evaluate the true abundance of the event. FIG. 18 shows an increase in OPA1 transcripts containing the NMD exon in HEK293 cells with increasing CHX dose. Other ocular cell lines also validated for the presence of the NMD exon (ARPE-19, Y79).

### Example 2.10: OPA1 NMD Event is Conserved in Primate Eyes

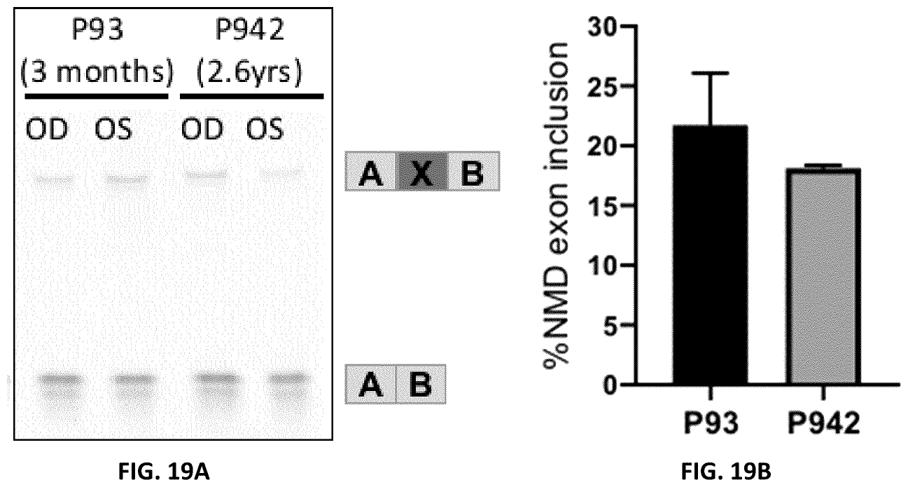

FIG. 19A shows reverse transcription PCR data from the posterior segment of the eye of Chlorocebus sabaeus (green monkey) at postnatal data P93 (3 months) and postnatal day P942 (2.6 years) for the right eye (OD) and left eye (OS). FIG. 19B shows quantification of the NMD exon abundance at 3 months and 2.6 years of age (N=1/age). Data represents average of right eye and left eye values for each animal. The abundance of the event may be higher in vivo, given that NMD is presumed active in the tissue.

### Example 2.11: OPA1 Antisense Oligonucleotides Reduce Non-Productive Splicing and Increase Productive OPA1 mRNA Levels In Vitro

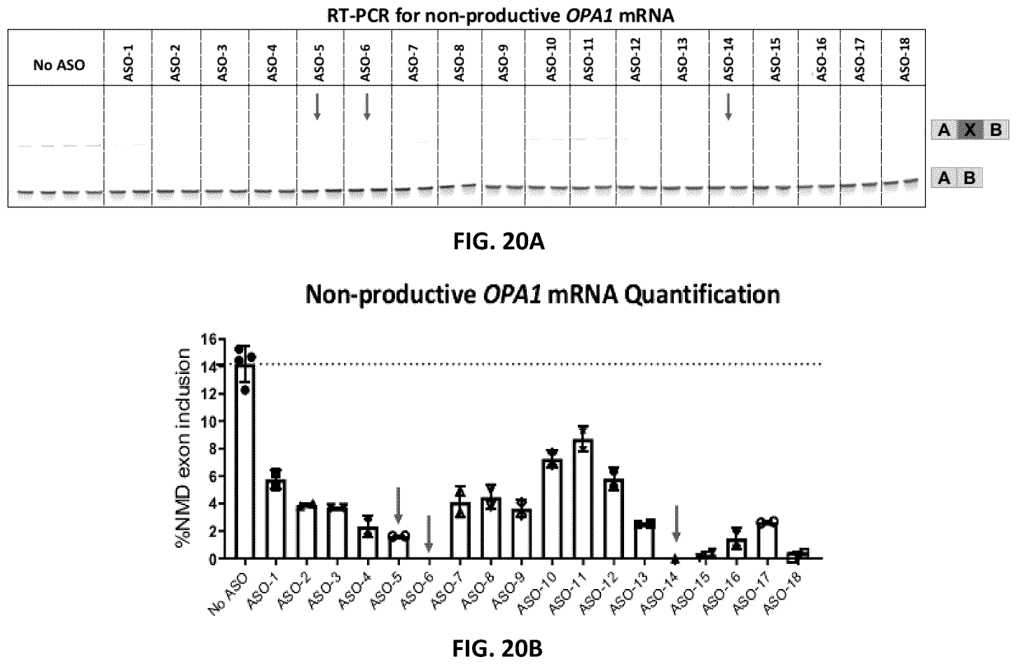

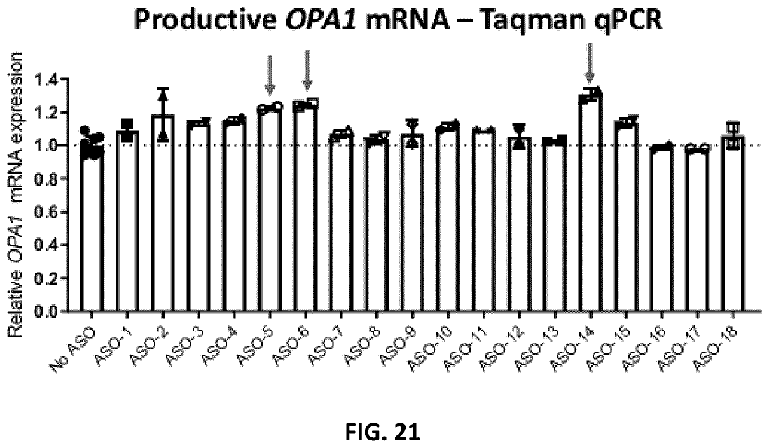

Exemplary antisense oligomers (ASOs) were transfected at 80 nM dose into HEK293 cells using Lipofectamine RNAiMax as a transfection agent. To assess the effect on the NMD exon, cells were treated with CHX (50 μg/ml, 3 hours) 21 hours after transfection. RNA was isolated for RT-PCR using probes spanning exon 7 and exon 8, as shown in FIG. 20A, and quantified in FIG. 20B. To assess levels of productive OPA1 mRNA expression, non-cycloheximide treated cells were used for TaqMan qPCR using probes spanning exon 23 and exon 24, and mRNA expression of OPA1 was normalized to RPL32, as shown in FIG. 21. Arrows highlight ASOs that reduce non-productive splicing and increase OPA1 mRNA expression by at least 20%. Among these, ASO-14 produces the most increase in OPA1 mRNA (30%).

### Example 2.12: ASO-14 Decreases Non-Productive OPA1 mRNA and Increases OPA1 Expression in a Dose-Dependent Manner In Vitro

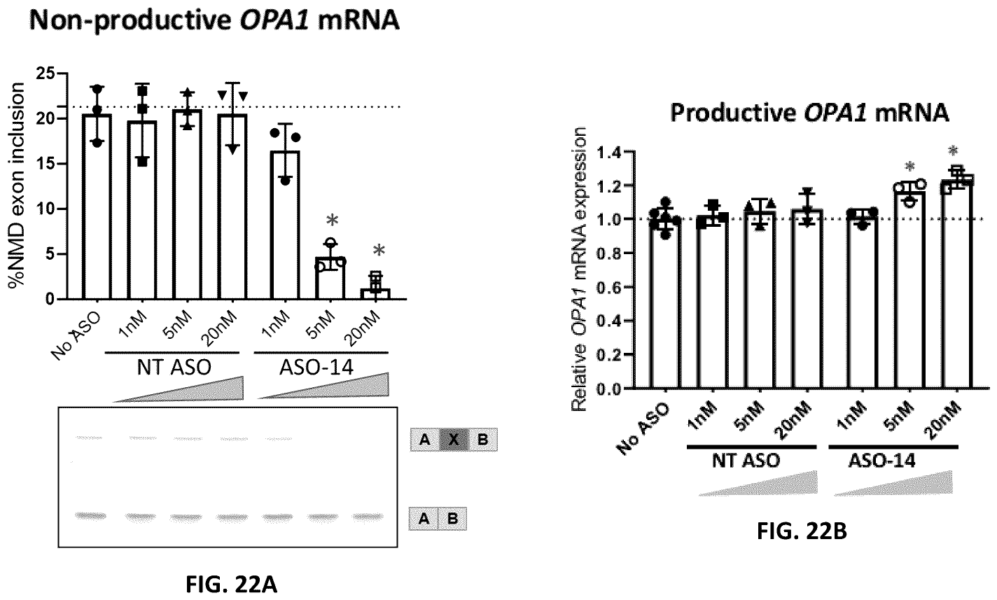

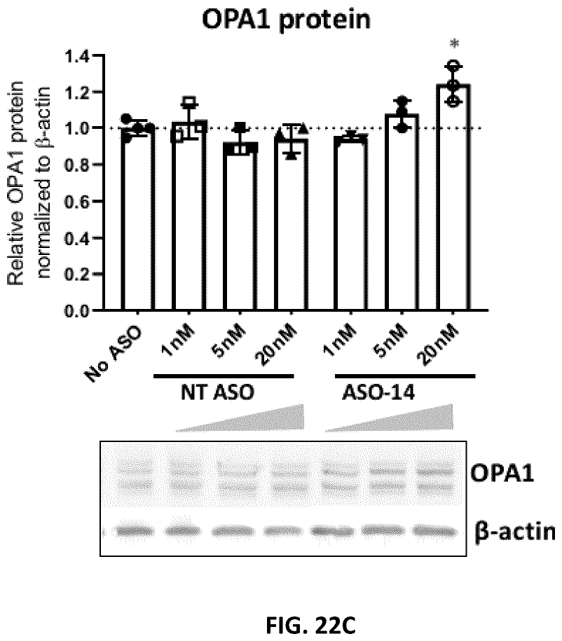

HEK293 cells were transfected with different doses of ASO-14 or non-targeting (NT) ASO. RNA was isolated 24 hours after transfection and analyzed for impact on non-productive OPA1 mRNA (FIG. 22A) and OPA1 mRNA expression (FIG. 22B) similarly to in Example 11. For protein analysis, cells were lysed with RIPA buffer 48 hours after transfection and western blots were probed with antibodies targeting OPA1 and β-actin, as shown in FIG. 22C. Multiple bands correspond to different isoforms of OPA1. Data represent the average of three independent experiments (* P<0.05 by one-way ANOVA compared to “NO ASO” group). The non-targeting ASO targets an unrelated gene.

### Example 2.13: ASO-14 Increases OPA1 Expression in an OPA1 Haploinsufficient (OPA1+/−) Cell Line

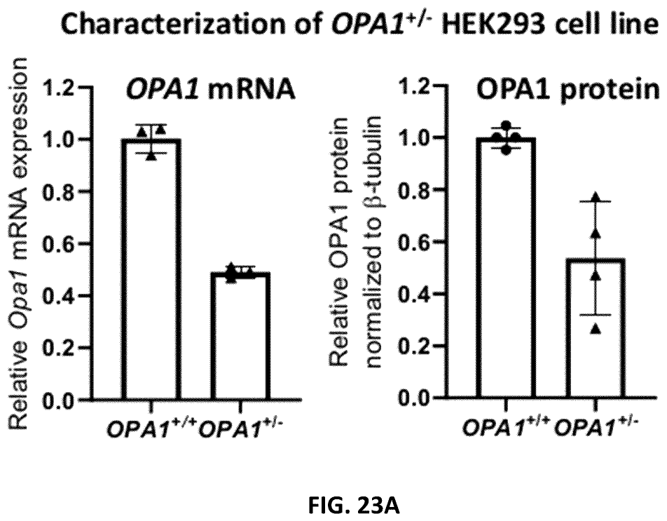

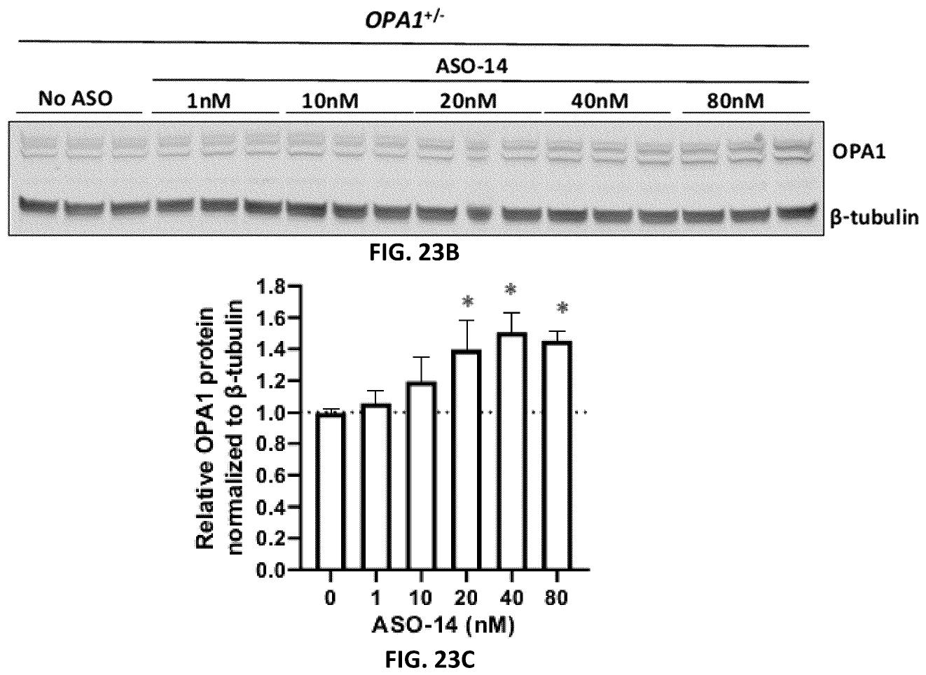

OPA1 haploinsufficient (OPA1+/−) HEK293 cells were generated using CRISPR-Cas9 gene editing. Similar to ADOA patient cells, OPA1+/−HEK293 cells show approximately 50% mRNA and protein levels of that observed in OPA1+/+ cells (FIG. 23A). The OPA1+/−HEK293 cells were transfected with different doses of ASO-14 as indicated in FIG. 23B, and total protein was isolated 72 hours after transfection. Western blots were probed with antibodies targeting OPA1 and β-tubulin, a representative blot is shown in FIG. 23B and quantification of two independent experiments is shown in FIG. 23C (* P<0.05 by one-way ANOVA compared to “No ASO” group). ASO-14 increases OPA1 protein levels in OPA1+/−HEK293 cells by 50%, which translates to 75% of wild-type levels.

### Example 2.14: Exemplary OPA1 ASOs Decrease Non-Productive Splicing and Increase OPA1 Expression in Wild-Type Rabbit Retinae Following Intravitreal Injection

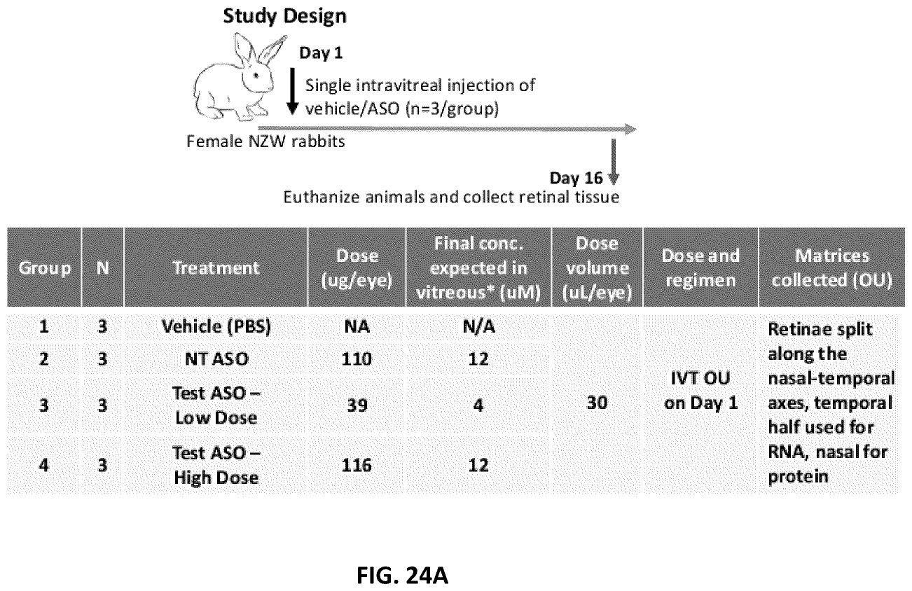

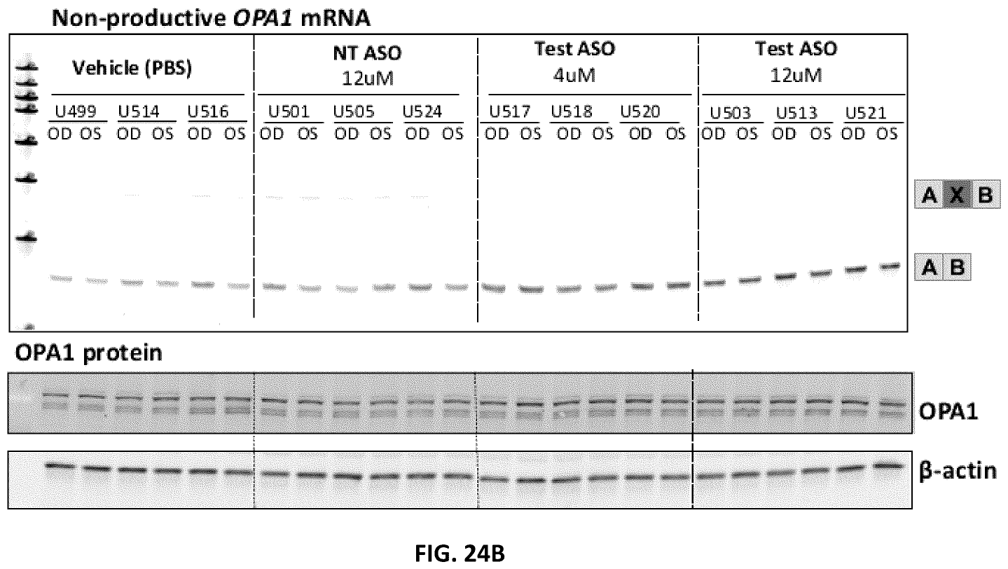

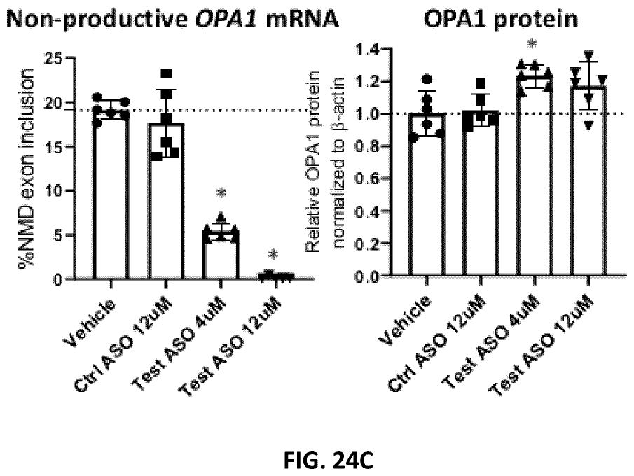

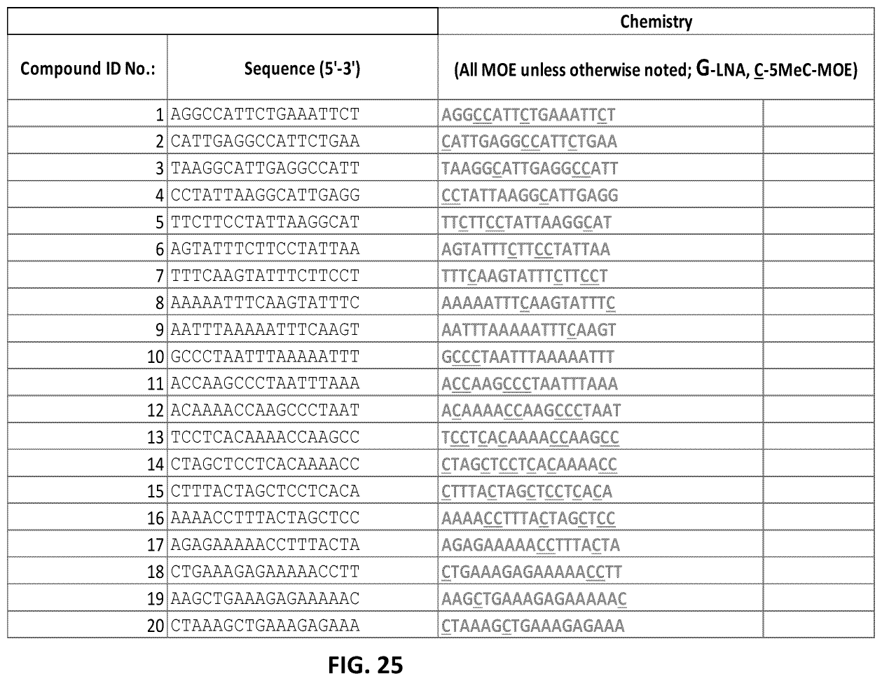

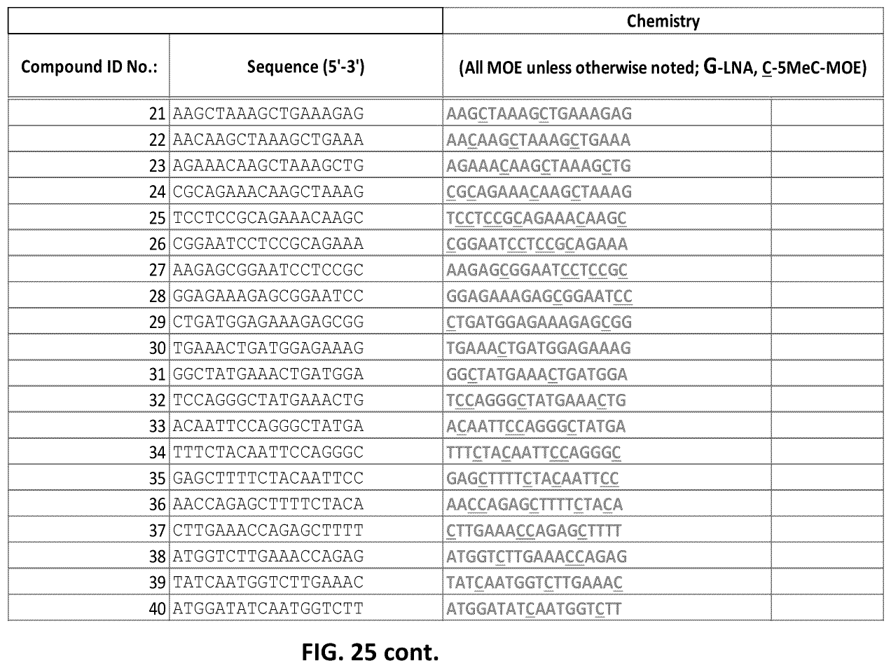

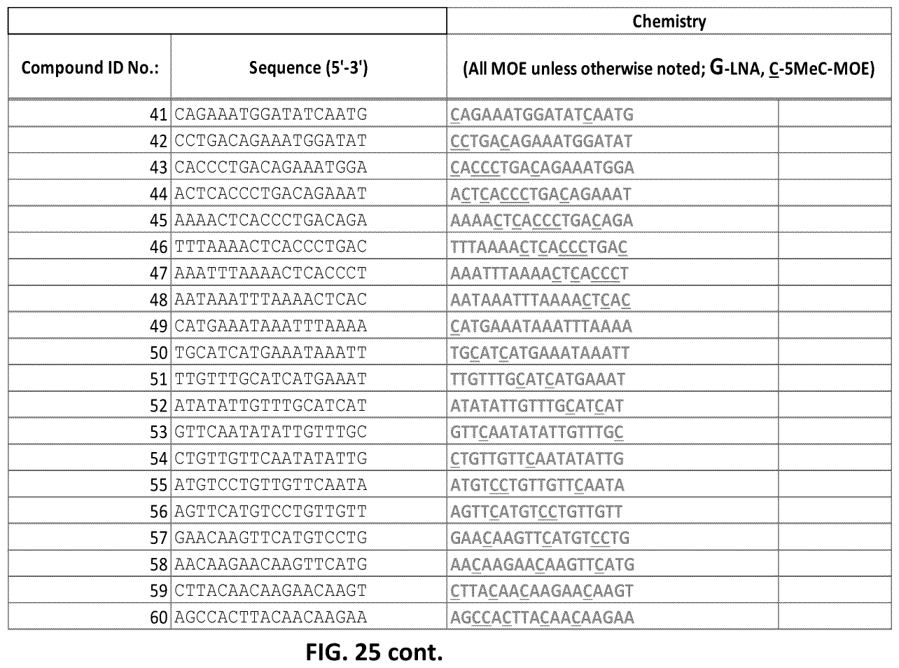

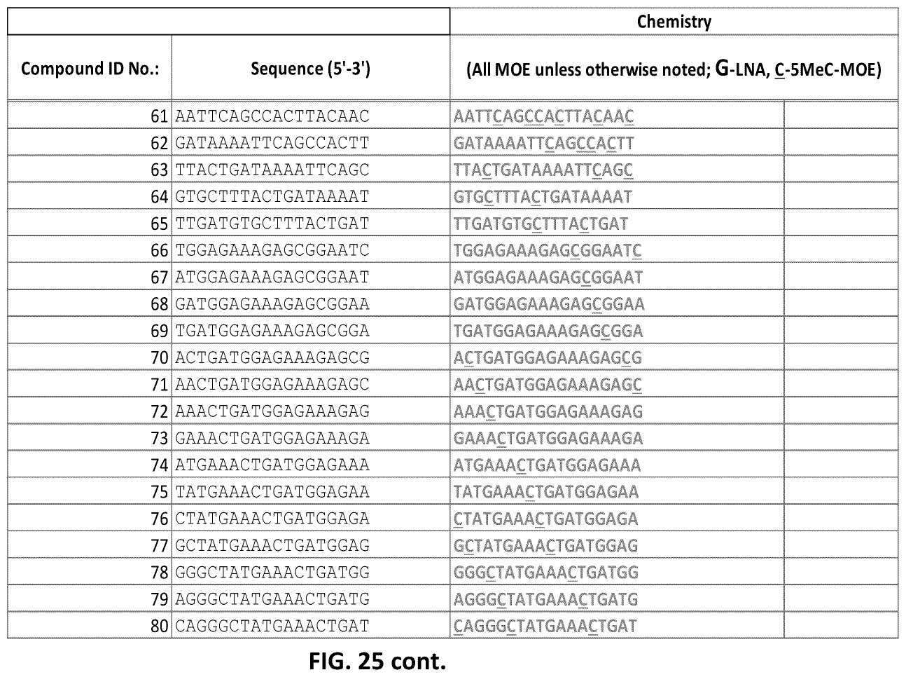

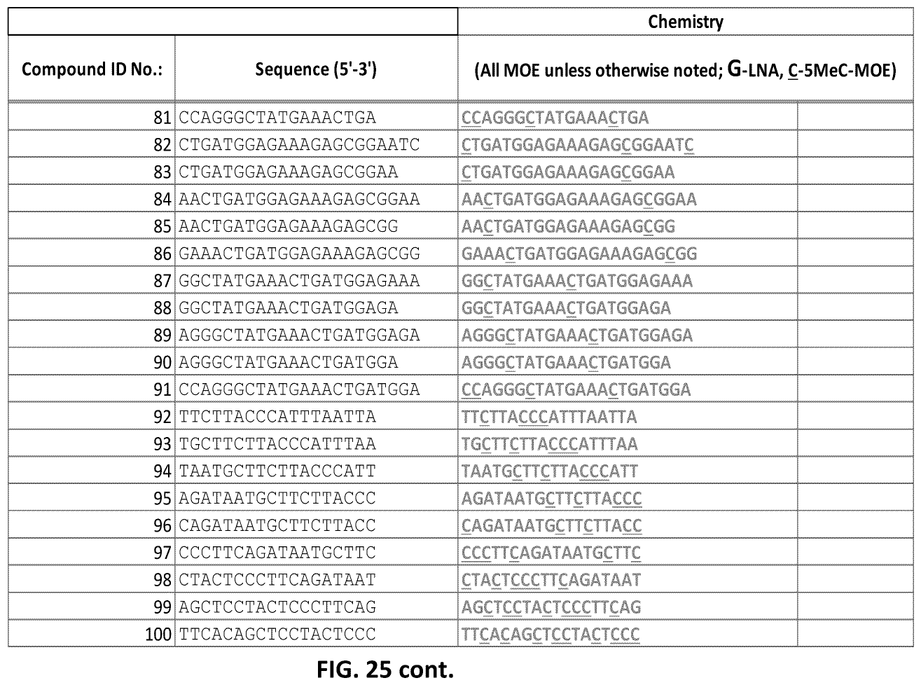

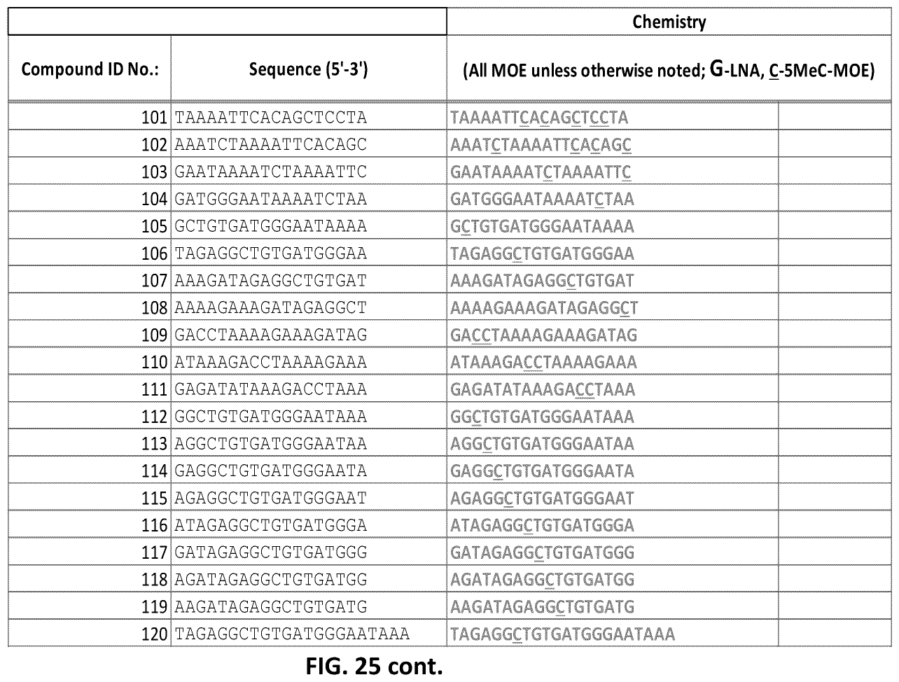

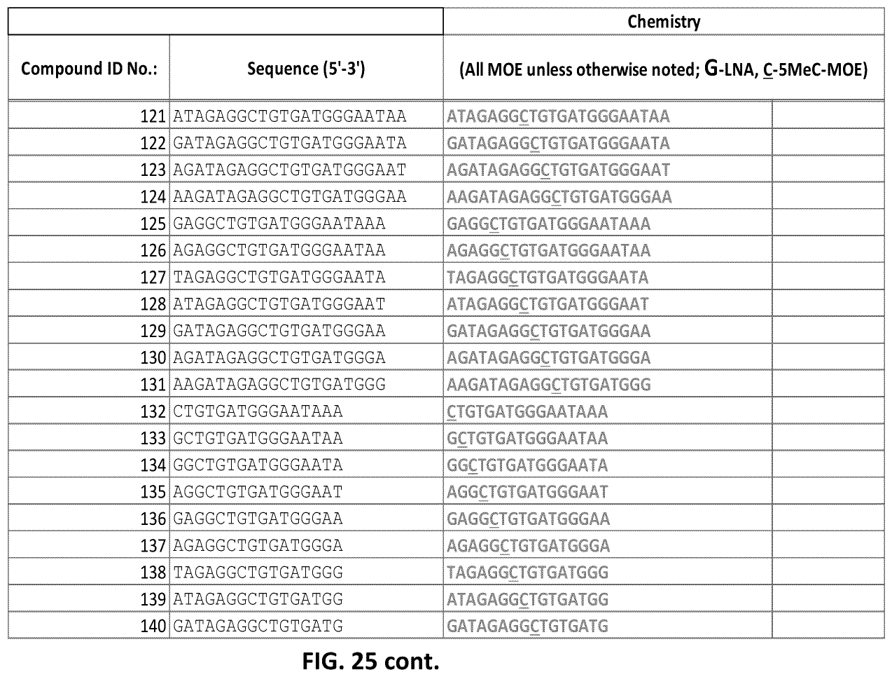

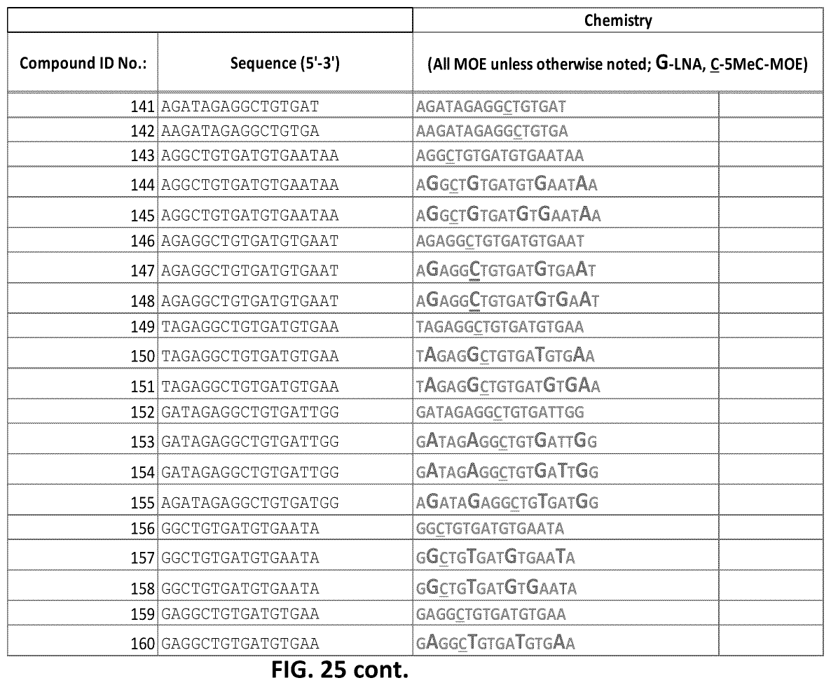

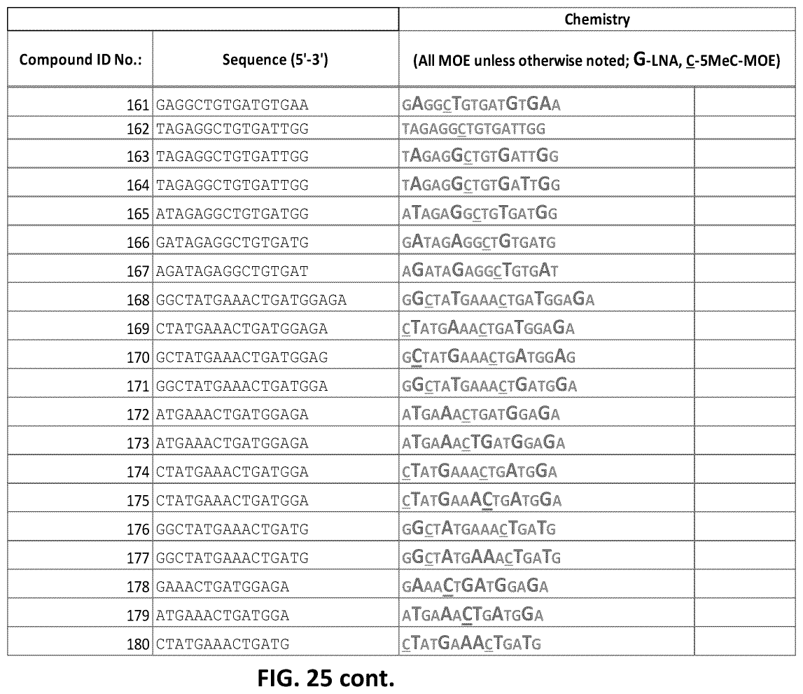

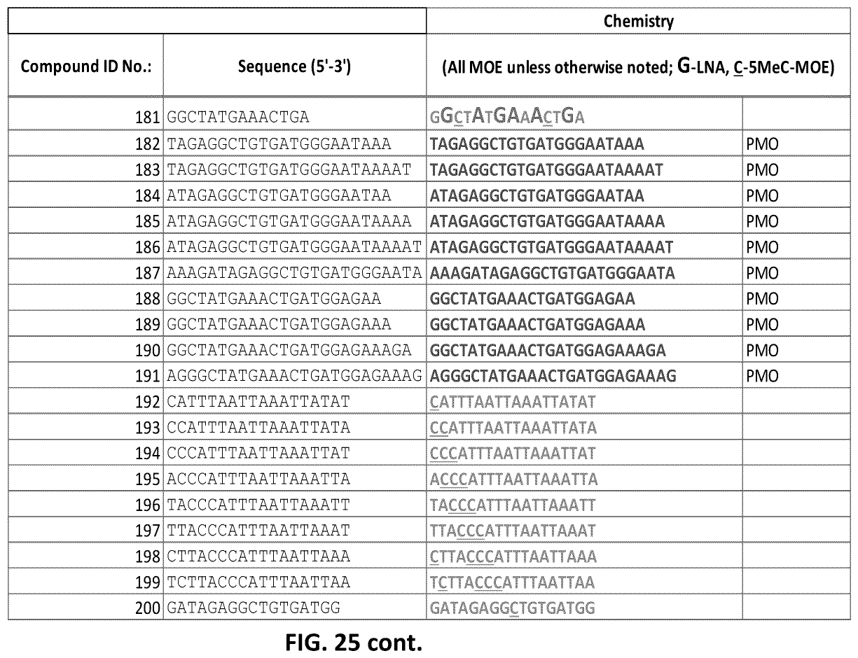

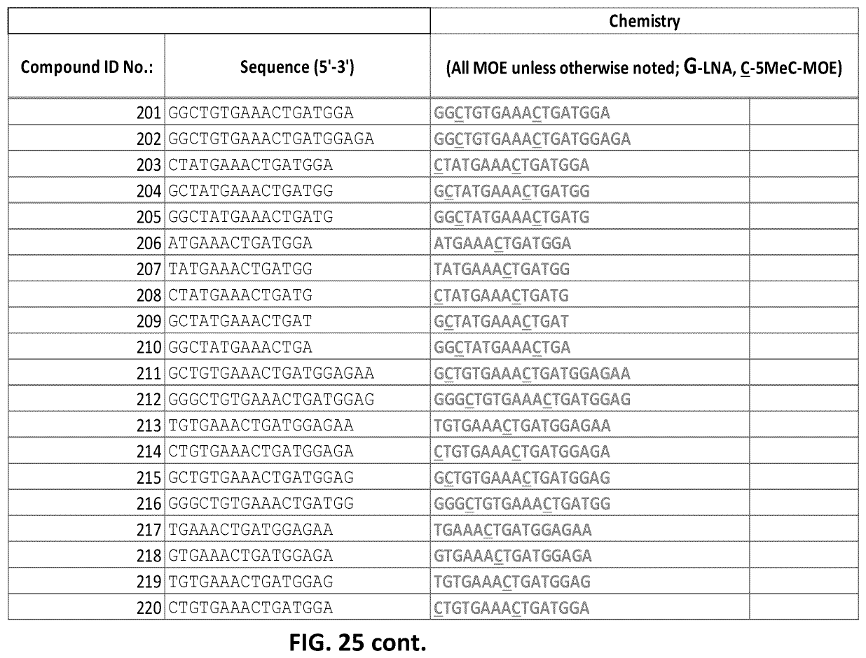

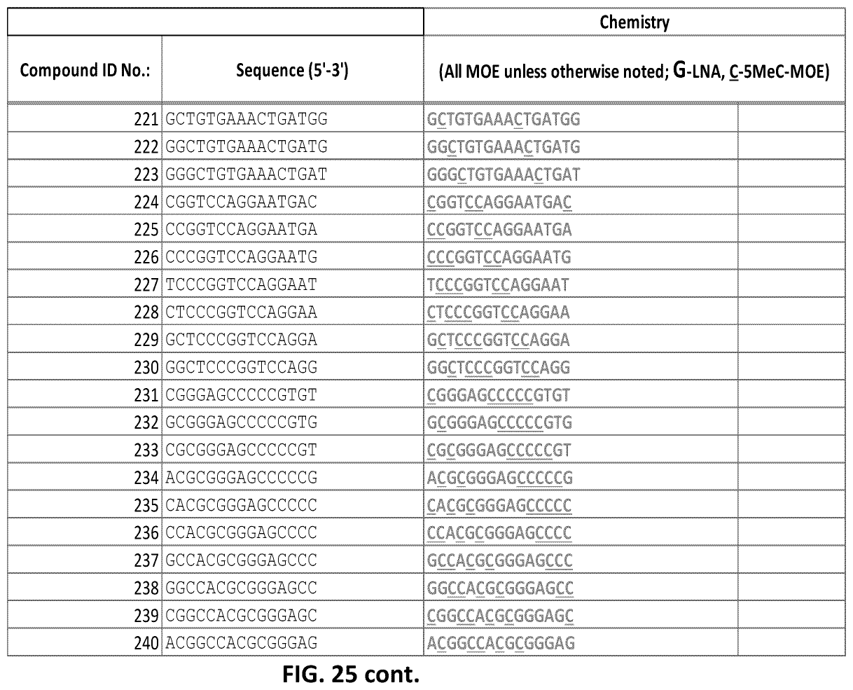

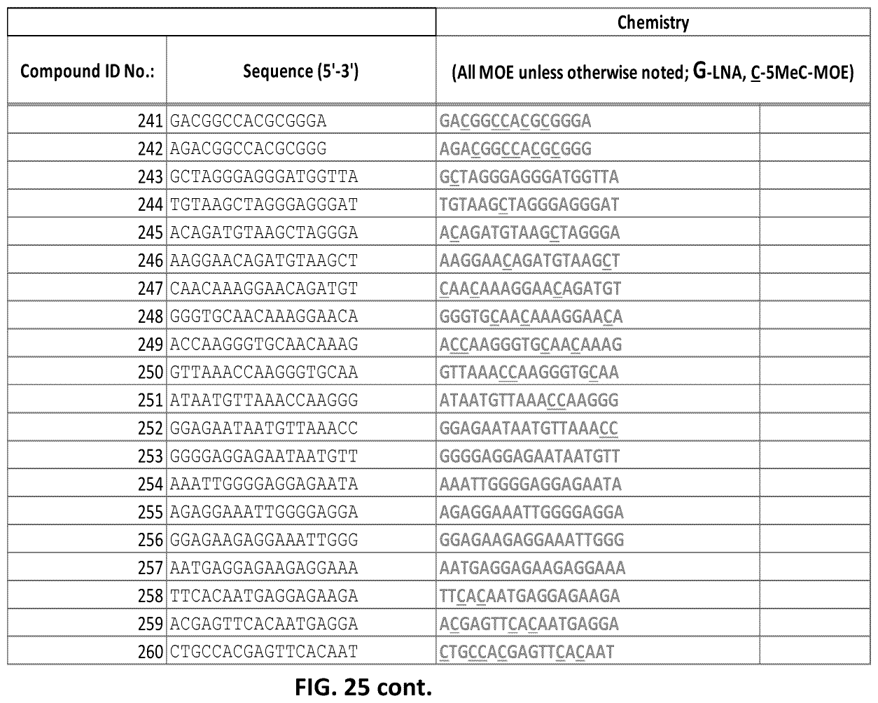

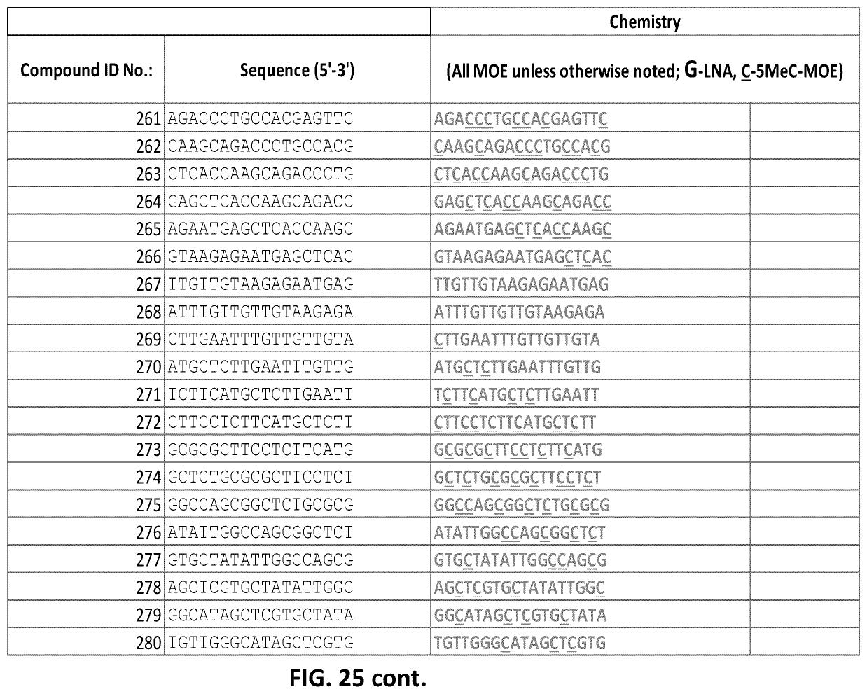

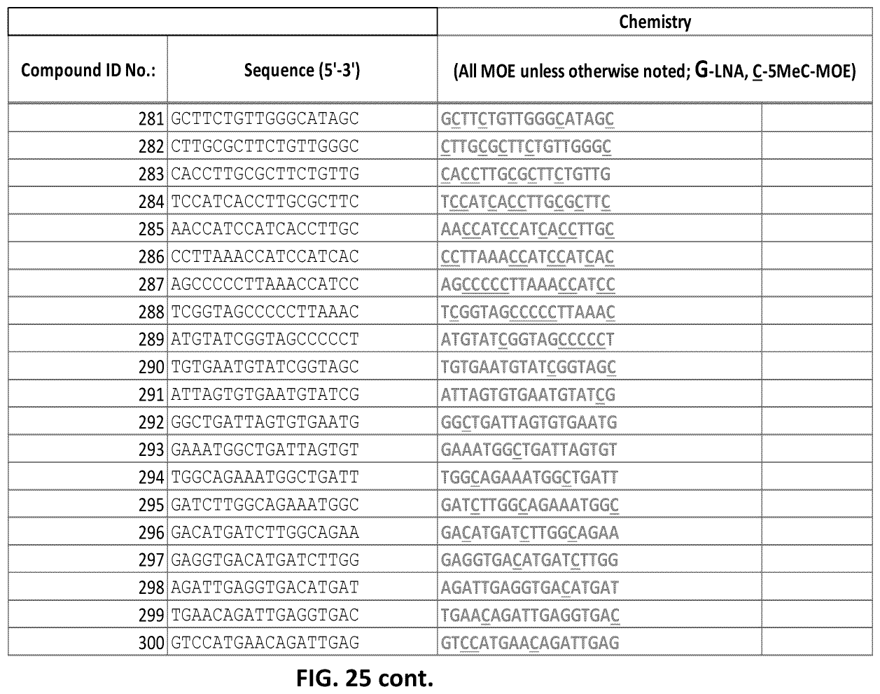

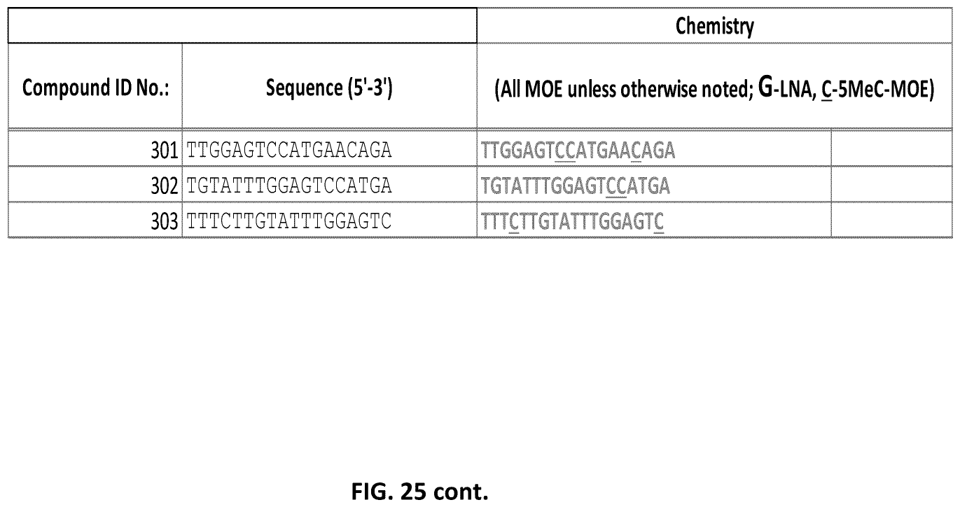

Female New Zealand White (NZW) adult rabbits were injected with either vehicle, non-targeting (NT), or test, antisense oligonucleotides. Animals were euthanized after 15 days to obtain retinal tissue. FIG. 24A outlines the study design, (*Final concentration in the vitreous was calculated assuming vitreal volume in the rabbit as 1.5 mL). FIG. 24B shows levels of productive and non-productive OPA1 mRNA and protein, and FIG. 24C shows quantification of this data (* P<0.05 by one-way ANOVA compared to Vehicle group). OD: oculus dexter (right eye), OS: oculus sinister (left eye).

It was also found that the antisense oligonucleotides were well-tolerated in wild-type rabbit for up to 28 days after intravitreal injection.

### Example 2.15: ASO-14 Modulates Inclusion of Both Exon 7 and Exon 7x in OPA1 mRNA Transcript

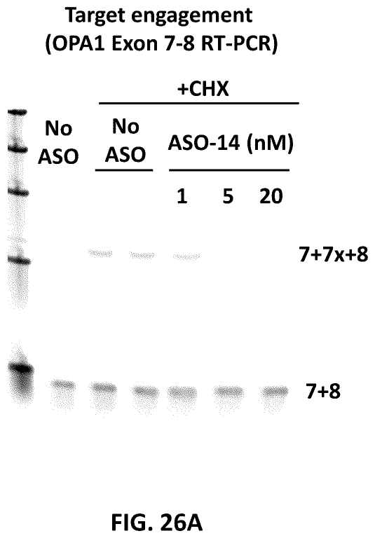

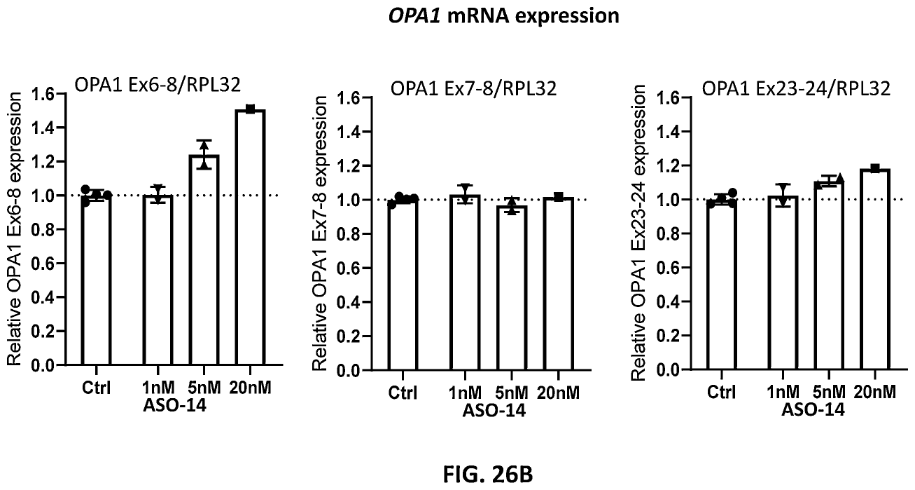

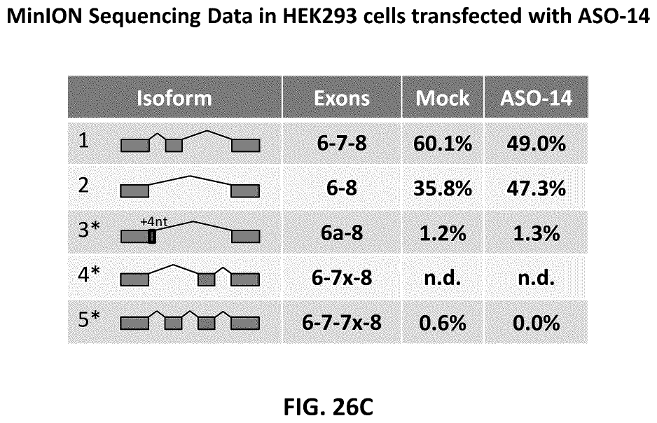

HEK293 cells were transfected with different doses of ASO-14 or no ASO, in the presence or absence of cycloheximide. RNA was isolated 24 hours after transfection and analyzed for impact on OPA1 mRNA splicing and OPA1 mRNA expression similarly to in Example 11. FIG. 26A shows a gel image of PCR products from RT-PCR reaction using probes spanning exon 7 and 8. As shown in the figure, the dose of ASO-14 increased from 1 nM, 5 nM, to 20 nM, the amount of transcripts having exon 7x between exons 7 and 8 (“7+7x+8”) gradually decreased, as compared to relatively stable amount of transcripts lacking exon 7x between exons 7 and 8 (“7+8”). FIG. 26B shows plots summarizing the relative amount of various OPA1 mRNA transcripts quantified by qPCR reactions using different pairs of probes: “Ex6-8,” probes spanning exons 6 and 8; “Ex7-8,” probes spanning exons 7 and 8; and “Ex23-24,” probes spanning exons 23 and 24. Results were normalized to RPL32 as an internal control. FIG. 26C shows a chart summarizing the quantification of various OPA1 mRNA transcripts based on sequencing of the RNA extracts from the treated HEK293 cells in the absence of cycloheximide. As suggested by the figures, ASO-14 appeared to induce reduction in OPA1 exon 7x inclusion, increase in OPA1 Ex6-8 transcripts (transcripts having exon 6 and exon 8 in tandem, thus lacking exon 7 and exon 7x), a modest decrease or no change in OPA1 Ex7-8 transcripts (transcripts having exon 7 and exon 8 in tandem, thus lacking exon 7x).

### Example 2.16: Exemplary OPA1 Antisense Oligomers Modulate Inclusion of Exon 7, Exon 7x, or Both in OPA1 mRNA Transcript

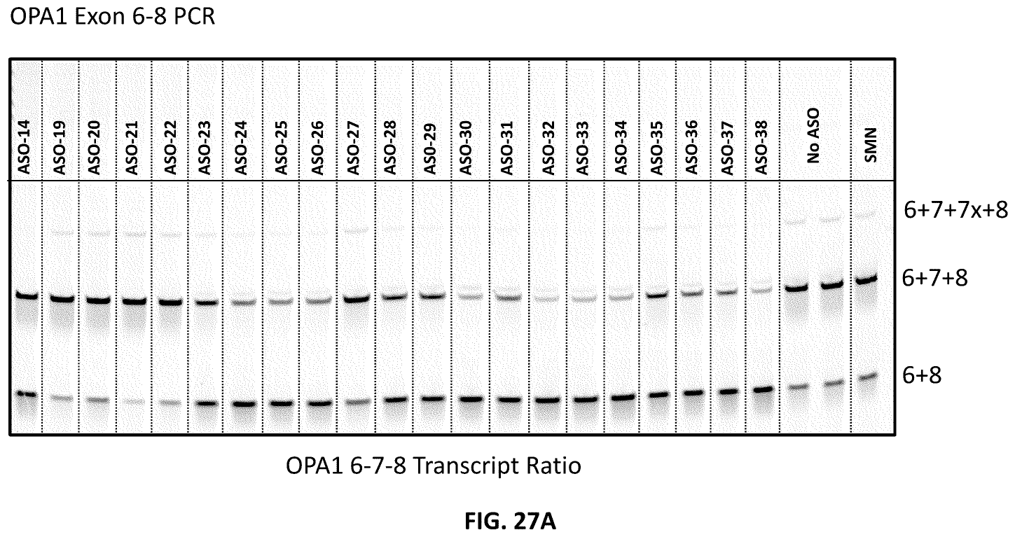

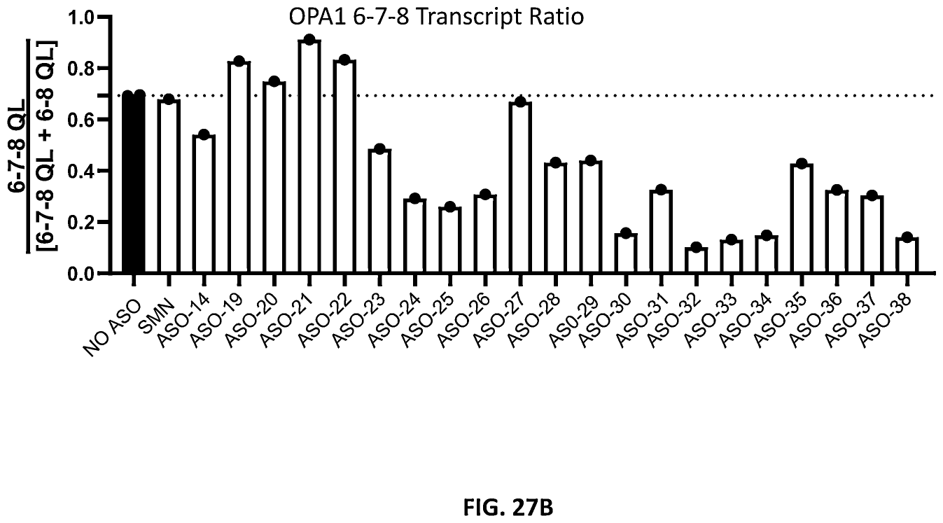

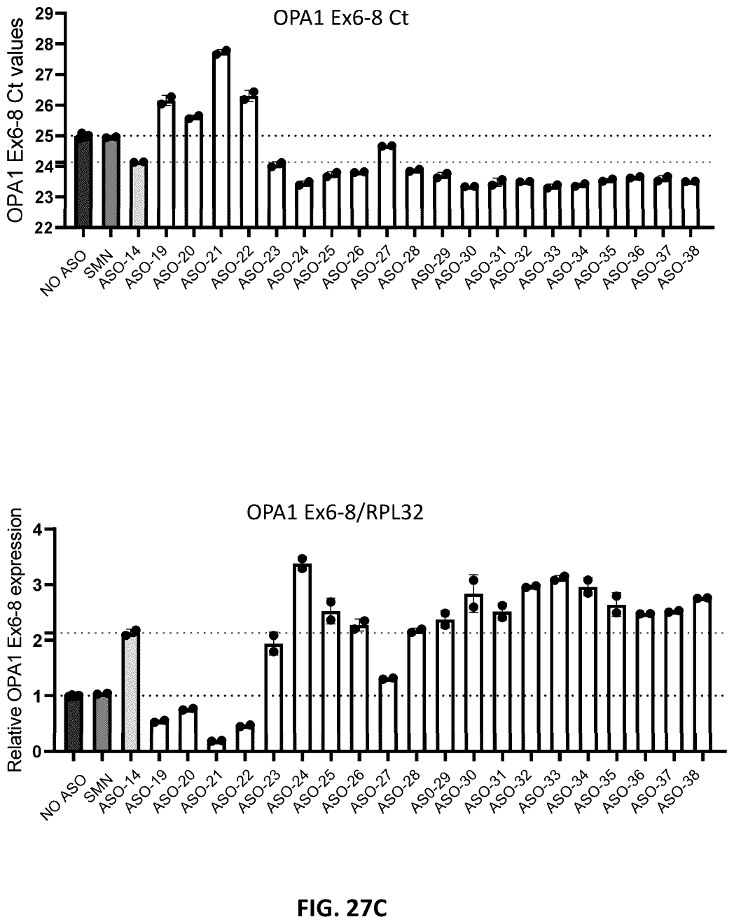

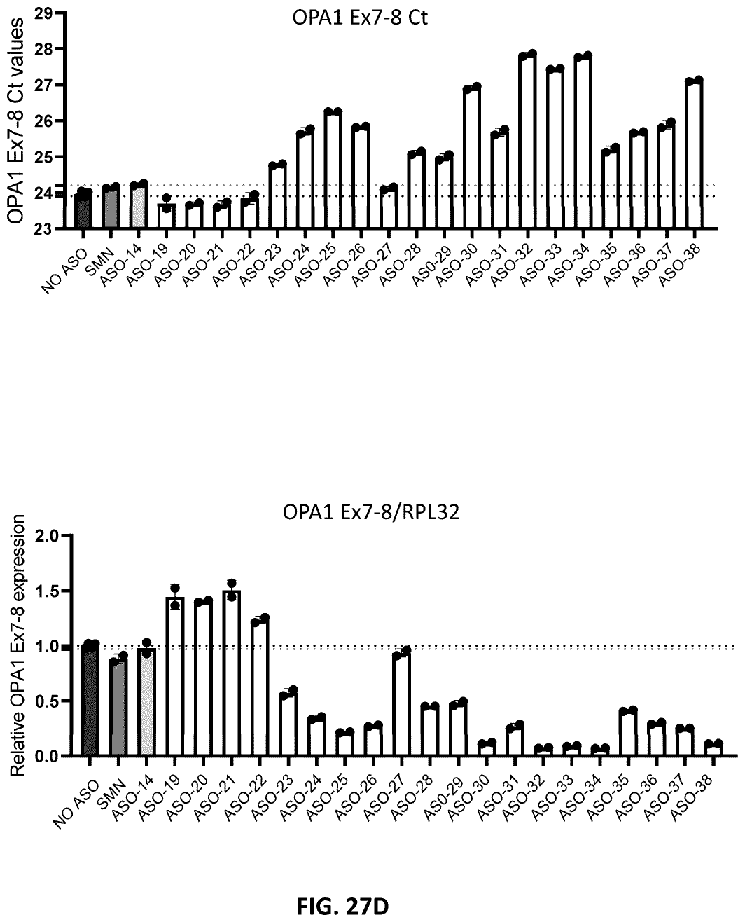

HEK293 cells were transfected with different exemplary OPA1 modified 2′MOE-PS (2′ methoxyethyl and phosphorothioate) ASOs. Each well of HEK 293 cells (about 100,000 cells/well) were treated with an exemplary ASO at 80 nM final concentration in the presence of 0.9 μL of Lipofectamine® RNAiMax in the absence of cycloheximide. The cells were harvested 24 hours after transfection and RNA was isolated and analyzed for impact on OPA1 mRNA splicing and OPA1 mRNA expression similarly to in Example 11. FIG. 27A shows a gel image of PCR products from RT-PCR reaction using probes spanning exon 6 and 8, and FIG. 27B is a plot summarizing the relative ratio of the amount of transcripts having exons 6, 7, and 8 in tandem (“6-7-8”) over the total amount of “6-7-8” transcripts and transcripts having exons 6 and 8 in tandem (“6-8”). As shown in the figures, certain ASOs, such as ASO-19, ASO-20, ASO-21, ASO-22, induced increase in the relative amount of “6-7-8” transcripts, suggesting an increase in the inclusion of exon 7 in mature OPA1 mRNA transcripts. Some ASOs, such as ASO-23, ASO-24, ASO-25, ASO-26, ASO-28, ASO-29, ASO-30, ASO-31, ASO-32, ASO-33, ASO-34, ASO-35, ASO-36, ASO-37, and ASO-38, in contrast, induced reduction in the relative amount of “6-7-8” transcripts, suggesting a reduction in the inclusion of exon 7 in mature OPA1 mRNA transcript. FIGS. 27C and 27D show the Ct values for the qPCR reaction (upper plots) for, and quantification of the relative amount (bottom plots) of, OPA1 transcripts having exons 6 and 8 (“Ex6-8”) and OPA1 transcripts having exons 7 and 8 (“Ex7-8”), respectively. Cells treated with ASO-29, ASO20, ASO-21, and ASO-22 showed reduced amount of “Ex6-8” transcripts and increased amount of “Ex7-8” transcripts, consistent with the suggestion that these ASOs promote the inclusion of exon 7 in OPA1 mature mRNA transcripts. Cells treated with ASO-23, ASO-24, ASO-25, ASO-26, ASO-28, ASO-29, ASO-30, ASO-31, ASO-32, ASO-33, ASO-34, ASO-35, ASO-36, ASO-37, and ASO-38 showed increase in the amount of “Ex6-8” transcripts and decrease in the amount of “Ex7-8” transcripts, consistent with the suggestion that these ASOs promote the exclusion of exon 7 from OPA1 mature mRNA transcripts.

### Example 2.17: Exemplary OPA1 Antisense Oligomers Modulate Inclusion of Exon 7, Exon 7x, or Both in OPA1 mRNA Transcript and Modulate Expression Level of OPA1 Protein

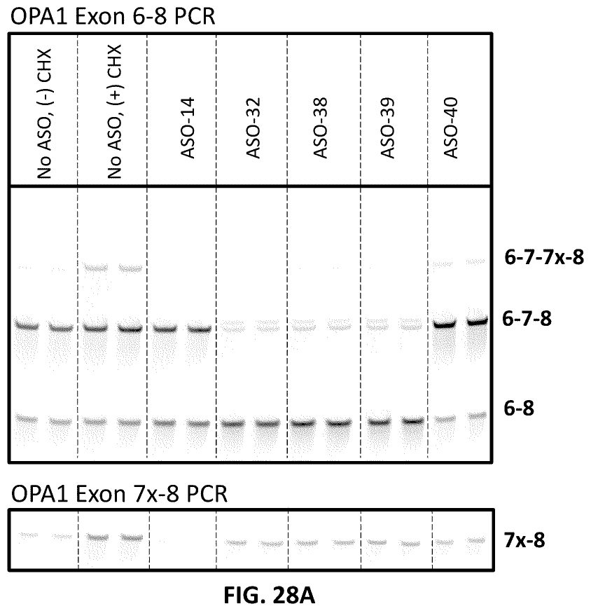

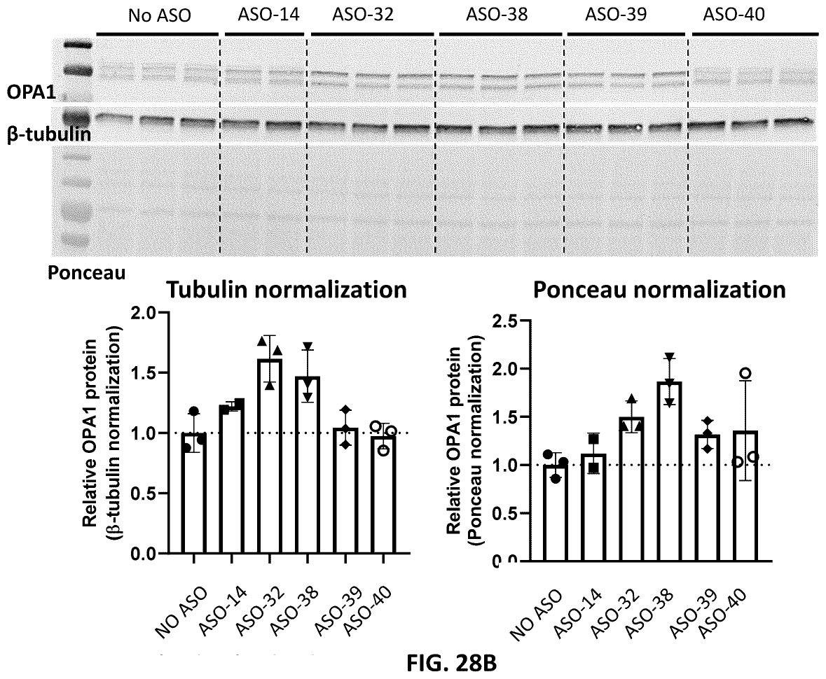

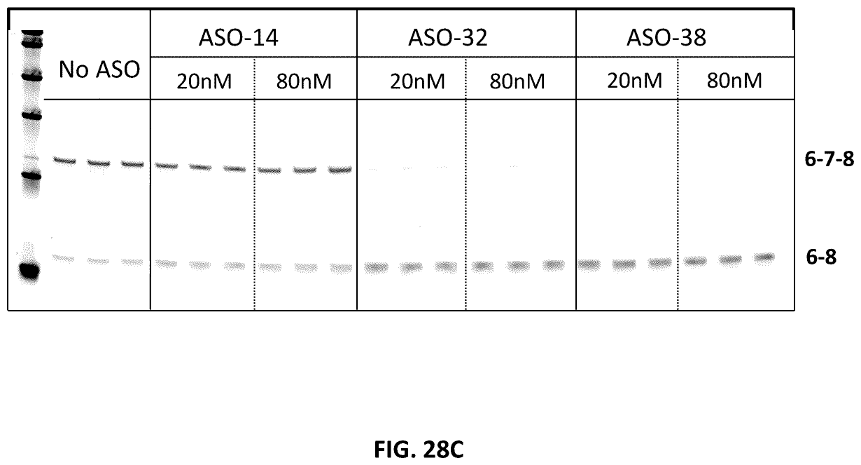

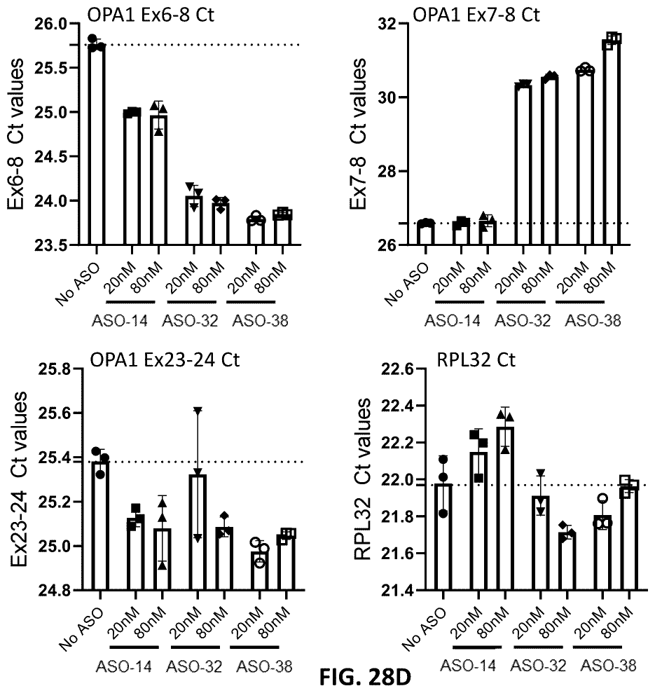

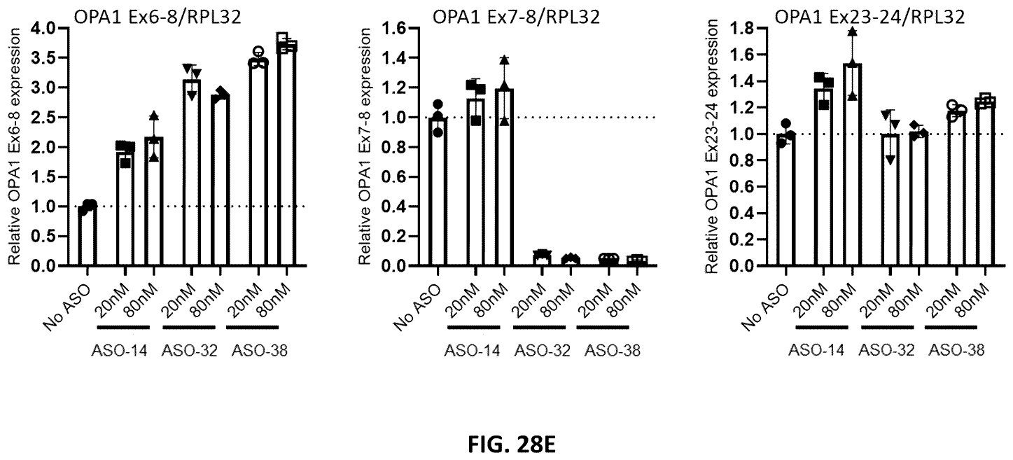

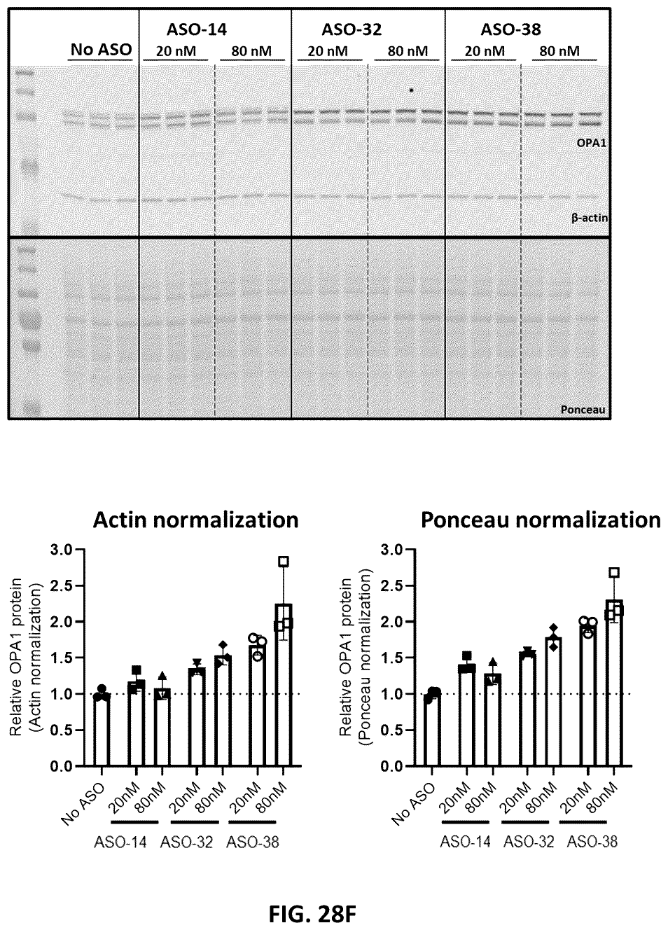

HEK293 cells were transfected with different exemplary OPA1 modified 2′MOE-PS (2′ methoxyethyl and phosphorothioate) ASOs. Each well of HEK293 cells (about 50,000 cells/well) was treated with an exemplary ASO at 80 nM final concentration in the presence of 0.9 μL of Lipofectamine® RNAiMax. Here, the cells were harvested 72 hours after transfection to test each ASO's effect on OPA1 mRNA and protein expression. The cells were treated with cycloheximide (50 μg/mL) for 3 hours prior to harvest for mRNA analysis. FIG. 28A shows a gel image of PCR products from RT-PCR reaction using probes spanning exon 6 and 8. As shown in the figure, ASO-14 induced reduction in the amount of transcripts having exons 6, 7, 7x, and 8 in tandem (“6-7-7x-8”). ASO-32, ASO-38, and ASO-39 induced significant reduction in the amount of “6-7-8” transcripts, and modest reduction in the amount of “6-7-7x-8” transcripts, whereas ASO-40 induced increase in the amount of “6-7-8” transcripts. These data suggest that ASO-14 promotes exclusion of exon 7x from OPA1 mRNA transcript, ASO-32, ASO-38, and ASO-39 promote exclusion of exon 7 from OPA1 mRNA transcript, and they also promote exclusion of exon 7x from OPA1 mRNA transcript. In contrast, the data suggest that ASO-40 promotes inclusion of exon 7 in OPA1 mRNA transcript.

FIG. 28B shows an image of a Western blot using antibody against OPA1 protein and antibody against β-tubulin protein in the cells after treatment with different ASOs or no ASO (control), as well as Ponceau staining image of the same blot. FIG. 28B also shows plots summarizing the amount of OPA1 protein under different treatment conditions as normalized relative to the amount of β-tubulin or Ponceau staining intensity. The data suggest that ASO-14, ASO-32, ASO-38, and ASO-39 all may induce increase in OPA1 protein expression, whereas ASO-40 may not significantly change the expression level of OPA1 protein.

Dose response of ASO-32 and ASO-38 were also tested along with ASO-14. ASO treatment, cell harvest, and RNA isolation and analysis were conducted similarly to the experiment above in this example. Each well of HEK293 cells (about 50,000 cells/well) were treated with either 20 nM or 80 nM of ASO-14, ASO-32, ASO-38, or no ASO. FIG. 28C shows a gel image of products from RT-PCR reaction using probes spanning exon 6 and 8. FIG. 28D shows quantification of qPCR Ct values for reactions under different experimental conditions using probes spanning exons and 8 (“Ex6-8”), probes spanning exons 7 and 8 (“Ex7-8”), and probes spanning exons 23 and 24 (“Ex23-24”), and FIG. 28E shows quantification of relative amount of the corresponding transcripts. The data show consistent observation that ASO-32 and ASO-38 promote exclusion of exon 7 from mature OPA1 mRNA transcripts. FIG. 28F shows the data on the OPA1 expression level after treatment of ASO-14, ASO-32, or ASO-38. Consistently, ASO-32 and ASO-38 increased OPA1 protein level.

### Example 2.18: ASO Microwalk Evaluated by RT-qPCR

In one experiment, microwalk was conducted to test ASOs that have sequences listed in Table 7. Briefly, about 30,000 HEK293 cells per well were treated gymnotically with 20 μM one of the 20 exemplary ASOs (free uptake) listed in Table 7 for 72 hours. After the treatment, the cells were harvested for analysis. RT-PCR reactions were conducted for products corresponding to Exon 7 or Exon 7x inclusion and full-length.

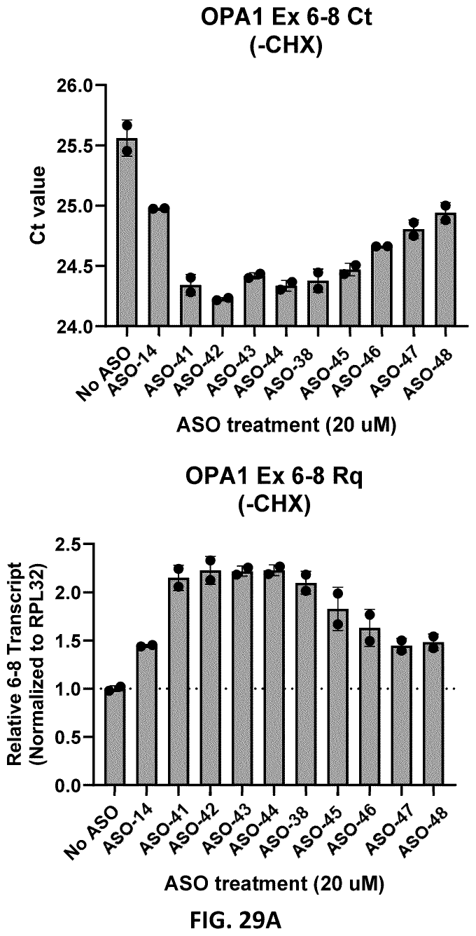

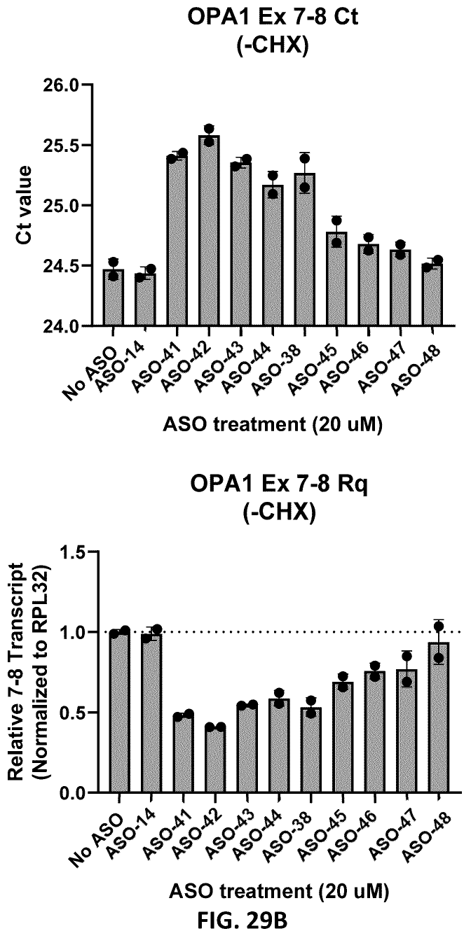

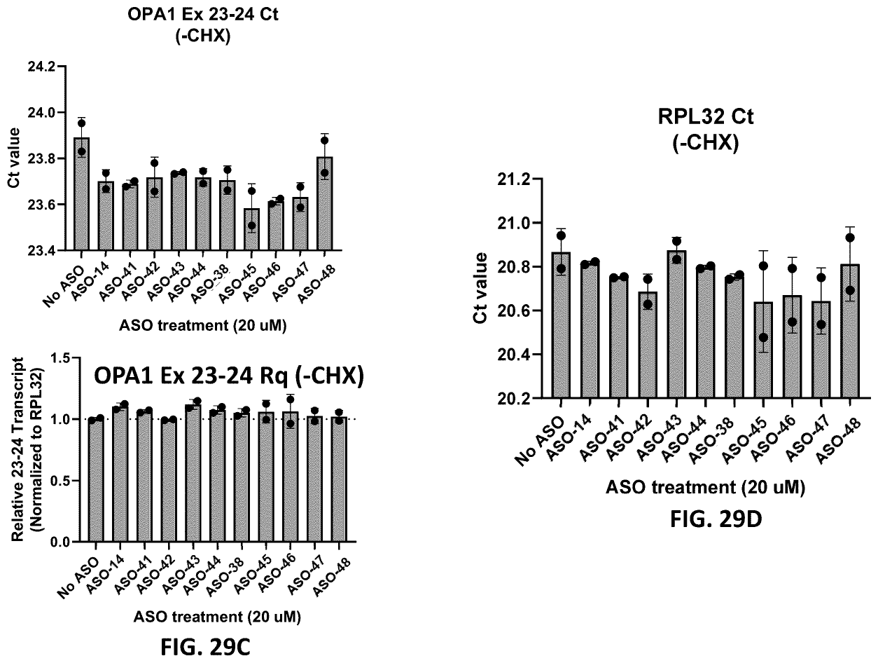

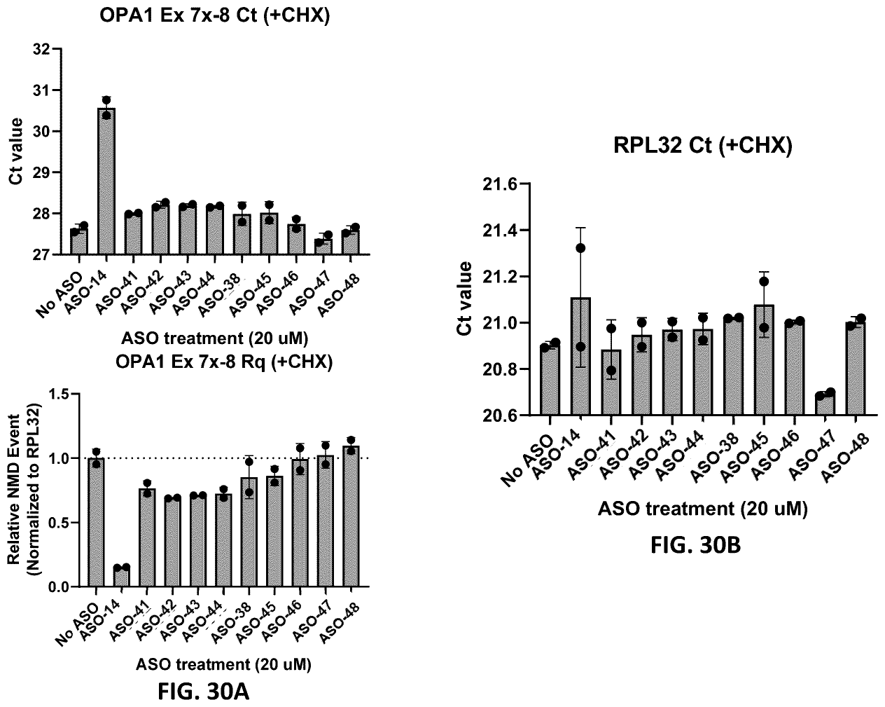

FIGS. 29A-30B demonstrate data from experiments with some of the 18-mers (named ASO-41 to ASO-48) listed in Table 7. FIGS. 29A-29B show the Ct values for the qPCR reaction (upper plots) for, and quantification of the relative amount (lower plots; normalized to Ct value of RPL32 qPCR product) of, OPA1 transcripts having exons 6 and 8 (“Ex6-8”) and OPA1 transcripts having exons 7 and 8 (“Ex7-8”), respectively. FIG. 29C shows the Ct values for the qPCR reaction (upper plots) for, and quantification of the relative amount (lower plots; normalized to Ct value of RPL32 qPCR product) of, OPA1 transcripts having exons 23 and 24 (“Ex23-24”), and FIG. 29D shows the Ct values for RPL32 transcripts as a loading control. These data demonstrate that cells treated with ASO-41 to ASO-47 all showed increased the amount of “Ex6-8” transcripts and decreased the amount of “Ex7-8” transcripts, suggesting these ASOs promote exclusion of Exon 7 from OPA1 transcripts. No cycloheximide was applied to the cells that were subject to these analyses for Exon 7 inclusion. FIG. 30A shows the Ct values for the qPCR reaction (upper plots) for, and quantification of the relative amount (lower plots; normalized to Ct value of RPL32 qPCR product) of, OPA1 transcripts having exons 7x and 8 (“Ex7x-8”), and FIG. 30B shows the Ct values for RPL32 transcripts as a loading control. These data demonstrate that cells treated with ASO-41 to ASO-44 all showed decreased amounts of “Ex7x-8” transcripts, suggesting these ASOs promote exclusion of Exon 7x from OPA1 transcripts. Cycloheximide was applied to the cells for these analyses for Exon 7x inclusion.

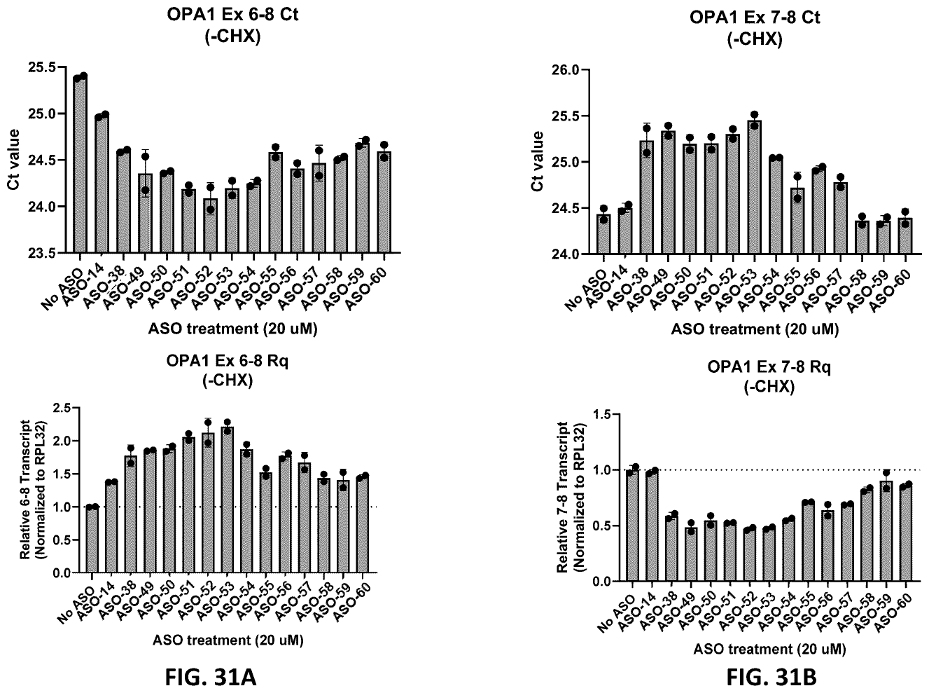

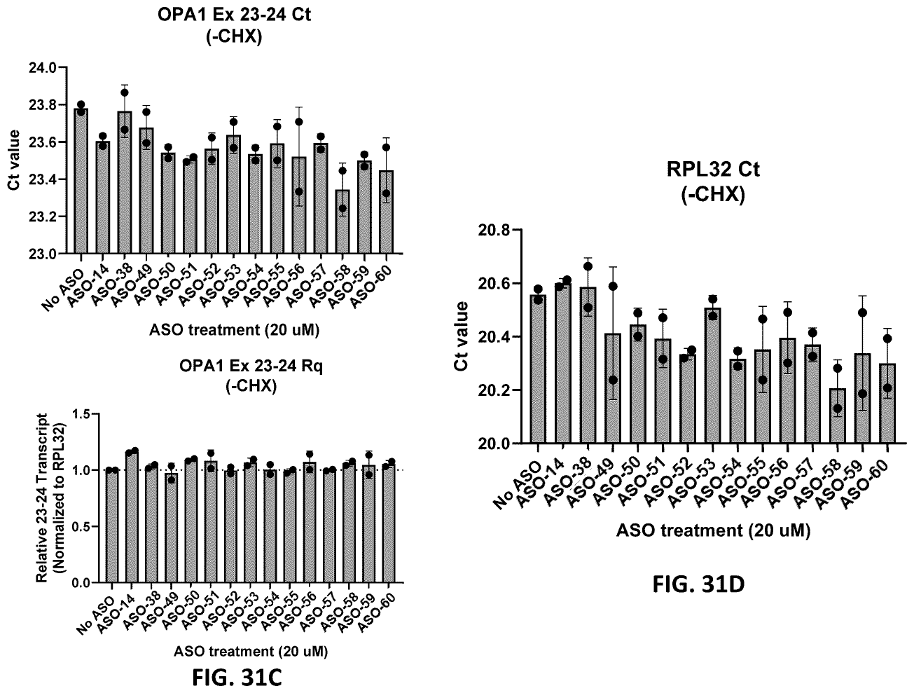

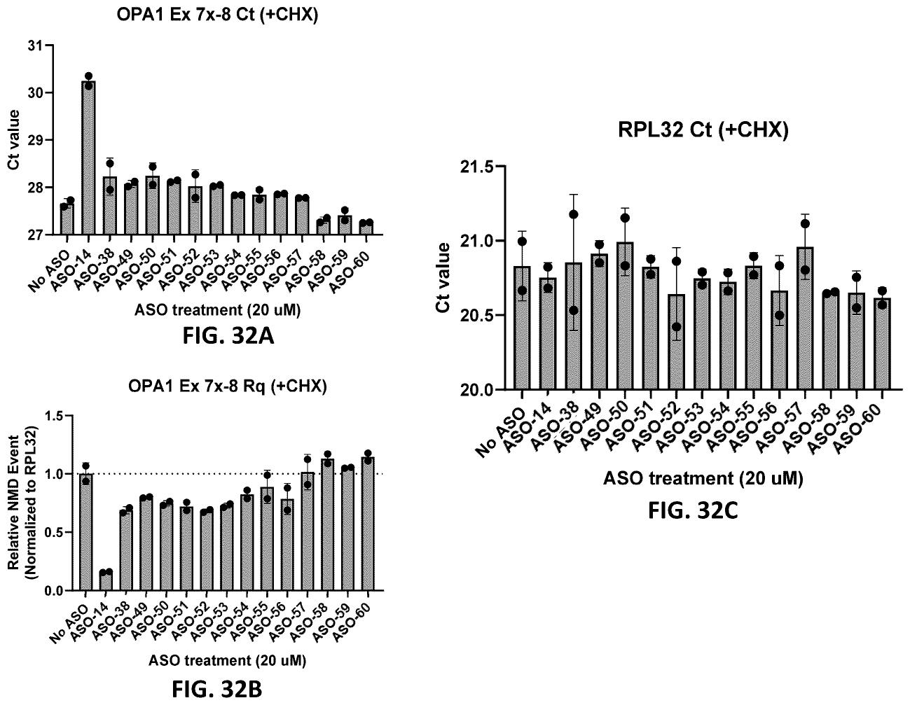

FIGS. 31A-32C demonstrate data from experiments with some of the 16-mers (named ASO-49 to ASO-60) listed in Table 7. FIGS. 31A-31B show the Ct values for the qPCR reaction (upper plots) for, and quantification of, the relative amount (lower plots; normalized to Ct value of RPL32 qPCR product) of OPA1 transcripts having exons 6 and 8 (“Ex6-8”) and OPA1 transcripts having exons 7 and 8 (“Ex7-8”), respectively. FIG. 31C shows the Ct values for the qPCR reaction (upper plots) for, and quantification of the relative amount (lower plots; normalized to Ct value of RPL32 qPCR product) of OPA1 transcripts having exons 23 and 24 (“Ex23-24”), and FIG. 31D shows the Ct values for RPL32 transcripts. These data demonstrate that cells treated with ASO-49 to ASO-60 all showed increased amounts of “Ex6-8” transcripts and decreased amounts of “Ex7-8” transcripts, suggesting these ASOs promote exclusion of Exon 7 from OPA1 transcripts. No cycloheximide was applied to the cells that were subject to these analyses for Exon 7 inclusion. FIG. 32A shows the Ct values for the qPCR reaction (upper plots) for, and quantification of, the relative amount (lower plots; normalized to Ct value of RPL32 qPCR product) of OPA1 transcripts having exons 7x and 8 (“Ex7x-8”), and FIG. 32C shows the Ct values for RPL32 transcripts as a loading control. These data demonstrate that cells treated with ASO-49 to ASO-56 all showed decreased amounts of “Ex7x-8” transcripts, suggesting these ASOs promote exclusion of Exon 7x from OPA1 transcripts. Cycloheximide was applied to the cells for these analyses for Exon 7x inclusion.

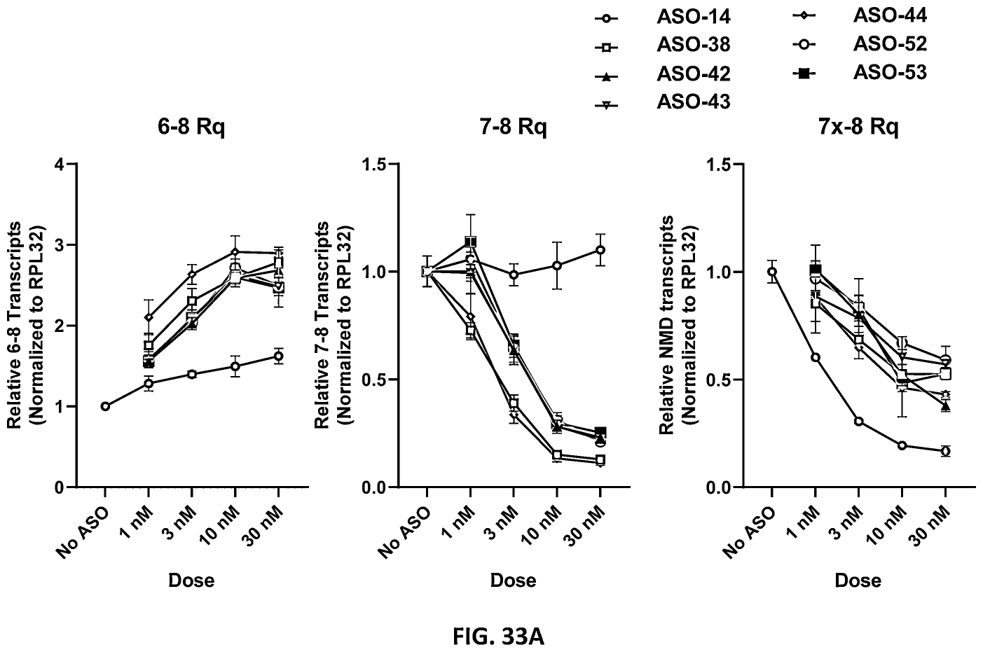

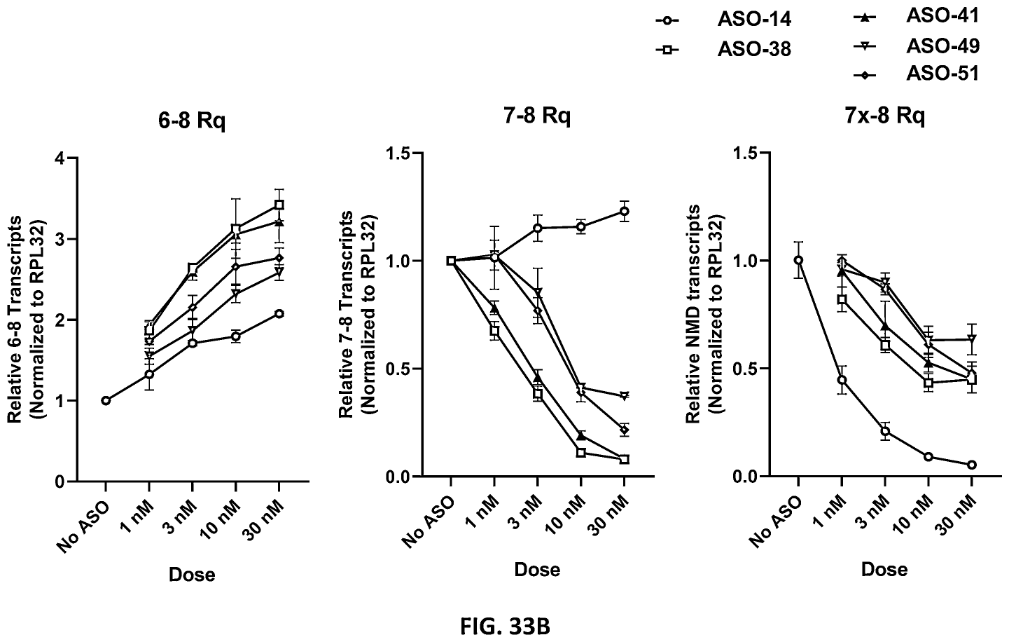

Another experiment was conducted to assess transfection dose response relationship with select ASOs among the ASOs tested above in the microwalk analyses. Briefly, 100,000 HEK293 cells per well were transfected with 1, 3, 10, or 30 nM of an exemplary ASO with 0.45 μL lipofectamine for 24 hours. Cells were later harvested for qPCR analysis as above. FIGS. 33A-33B show plots depicting the dose response curves of relative amounts of different OPA1 transcripts versus the transfection concentration of exemplary ASOs, ASO-14, 38, 41, 42, 43, 44, 49, 51, 52, and 53. The plots show that, as a general trend, in cells treated with ASOs like ASO-38, 41, 42, 43, 44, 49, 51, 52, or 53, the amount of OPA1 transcripts having Exon 6 and 8 (“6-8”) increased, while the amounts of OPA1 transcripts having Exon 7 and 8 (“7-8”) and OPA1 transcripts having Exon 7x and 8 (“7x-8”) decreased as the concentration of the exemplary ASO increased. In contrast, in cells treated with ASO-14, while “7x-8” decreased and “6-8” transcripts increased, “7-8” transcripts did not significantly change. These data suggest that ASO-38, 41, 42, 43, 44, 49, 51, 52, and 53 may all promote exclusion of both Exon 7 and Exon 7x, while ASO-14 may promote exclusion of Exon 7x.

TABLE 5
Exemplary OPA1 ASO sequences
(Organism: synthetic construct)

| | | Coordinates | |
|---|---|---|---|
| SEQ | | GRCh38/hg38: chr3 | |
| ID | | Oligo | Oligo |
| NO | Sequence (5′-3′) | Start | End |
| 6 | AGGCCATTCTGAAATTCT | 193628406 | 193628423 |
| 7 | CATTGAGGCCATTCTGAA | 193628411 | 193628428 |
| 8 | TAAGGCATTGAGGCCATT | 193628416 | 193628433 |
| 9 | CCTATTAAGGCATTGAGG | 193628421 | 193628438 |
| 10 | TTCTTCCTATTAAGGCAT | 193628426 | 193628443 |
| 11 | AGTATTTCTTCCTATTAA | 193628431 | 193628448 |
| 12 | TTTCAAGTATTTCTTCCT | 193628436 | 193628453 |
| 13 | AAAAATTTCAAGTATTTC | 193628441 | 193628458 |
| 14 | AATTTAAAAATTTCAAGT | 193628446 | 193628463 |
| 15 | GCCCTAATTTAAAAATTT | 193628451 | 193628468 |
| 16 | ACCAAGCCCTAATTTAAA | 193628456 | 193628473 |
| 17 | ACAAAACCAAGCCCTAAT | 193628461 | 193628478 |
| 18 | TCCTCACAAAACCAAGCC | 193628466 | 193628483 |
| 19 | CTAGCTCCTCACAAAACC | 193628471 | 193628488 |
| 20 | CTTTACTAGCTCCTCACA | 193628476 | 193628493 |
| 21 | AAAACCTTTACTAGCTCC | 193628481 | 193628498 |
| 22 | AGAGAAAAACCTTTACTA | 193628486 | 193628503 |
| 23 | CTGAAAGAGAAAAACCTT | 193628491 | 193628508 |
| 24 | AAGCTGAAAGAGAAAAAC | 193628494 | 193628511 |
| 25 | CTAAAGCTGAAAGAGAAA | 193628497 | 193628514 |
| 26 | AAGCTAAAGCTGAAAGAG | 193628500 | 193628517 |
| 27 | AACAAGCTAAAGCTGAAA | 193628503 | 193628520 |
| 28 | AGAAACAAGCTAAAGCTG | 193628506 | 193628523 |
| 29 | CGCAGAAACAAGCTAAAG | 193628509 | 193628526 |
| 30 | TCCTCCGCAGAAACAAGC | 193628514 | 193628531 |
| 31 | CGGAATCCTCCGCAGAAA | 193628519 | 193628536 |
| 32 | AAGAGCGGAATCCTCCGC | 193628524 | 193628541 |
| 33 | GGAGAAAGAGCGGAATCC | 193628529 | 193628546 |
| 34 | CTGATGGAGAAAGAGCGG | 193628534 | 193628551 |
| 35 | TGAAACTGATGGAGAAAG | 193628539 | 193628556 |
| 36 | GGCTATGAAACTGATGGA | 193628544 | 193628561 |
| 37 | TCCAGGGCTATGAAACTG | 193628549 | 193628566 |
| 38 | ACAATTCCAGGGCTATGA | 193628554 | 193628571 |
| 39 | TTTCTACAATTCCAGGGC | 193628559 | 193628576 |
| 40 | GAGCTTTTCTACAATTCC | 193628564 | 193628581 |
| 41 | AACCAGAGCTTTTCTACA | 193628569 | 193628586 |
| 42 | CTTGAAACCAGAGCTTTT | 193628574 | 193628591 |
| 43 | ATGGTCTTGAAACCAGAG | 193628579 | 193628596 |
| 44 | TATCAATGGTCTTGAAAC | 193628584 | 193628601 |
| 45 | ATGGATATCAATGGTCTT | 193628589 | 193628606 |
| 46 | CAGAAATGGATATCAATG | 193628594 | 193628611 |
| 47 | CCTGACAGAAATGGATAT | 193628599 | 193628616 |
| 48 | CACCCTGACAGAAATGGA | 193628602 | 193628619 |
| 49 | ACTCACCCTGACAGAAAT | 193628605 | 193628622 |
| 50 | AAAACTCACCCTGACAGA | 193628608 | 193628625 |
| 51 | TTTAAAACTCACCCTGAC | 193628611 | 193628628 |
| 52 | AAATTTAAAACTCACCCT | 193628614 | 193628631 |
| 53 | AATAAATTTAAAACTCAC | 193628617 | 193628634 |
| 54 | CATGAAATAAATTTAAAA | 193628622 | 193628639 |
| 55 | TGCATCATGAAATAAATT | 193628627 | 193628644 |
| 56 | TTGTTTGCATCATGAAAT | 193628632 | 193628649 |
| 57 | ATATATTGTTTGCATCAT | 193628637 | 193628654 |
| 58 | GTTCAATATATTGTTTGC | 193628642 | 193628659 |
| 59 | CTGTTGTTCAATATATTG | 193628647 | 193628664 |
| 60 | ATGTCCTGTTGTTCAATA | 193628652 | 193628669 |
| 61 | AGTTCATGTCCTGTTGTT | 193628657 | 193628674 |
| 62 | GAACAAGTTCATGTCCTG | 193628662 | 193628679 |
| 63 | AACAAGAACAAGTTCATG | 193628667 | 193628684 |
| 64 | CTTACAACAAGAACAAGT | 193628672 | 193628689 |
| 65 | AGCCACTTACAACAAGAA | 193628677 | 193628694 |
| 66 | AATTCAGCCACTTACAAC | 193628682 | 193628699 |
| 67 | GATAAAATTCAGCCACTT | 193628687 | 193628704 |
| 68 | TTACTGATAAAATTCAGC | 193628692 | 193628709 |
| 69 | GTGCTTTACTGATAAAAT | 193628697 | 193628714 |
| 70 | TTGATGTGCTTTACTGAT | 193628702 | 193628719 |
| 71 | TGGAGAAAGAGCGGAATC | 193628530 | 193628547 |
| 72 | ATGGAGAAAGAGCGGAAT | 193628531 | 193628548 |
| 73 | GATGGAGAAAGAGCGGAA | 193628532 | 193628549 |
| 74 | TGATGGAGAAAGAGCGGA | 193628533 | 193628550 |
| 75 | ACTGATGGAGAAAGAGCG | 193628535 | 193628552 |
| 76 | AACTGATGGAGAAAGAGC | 193628536 | 193628553 |
| 77 | AAACTGATGGAGAAAGAG | 193628537 | 193628554 |
| 78 | GAAACTGATGGAGAAAGA | 193628538 | 193628555 |
| 79 | ATGAAACTGATGGAGAAA | 193628540 | 193628557 |
| 80 | TATGAAACTGATGGAGAA | 193628541 | 193628558 |
| 81 | CTATGAAACTGATGGAGA | 193628542 | 193628559 |
| 82 | GCTATGAAACTGATGGAG | 193628543 | 193628560 |
| 83 | GGGCTATGAAACTGATGG | 193628545 | 193628562 |
| 84 | AGGGCTATGAAACTGATG | 193628546 | 193628563 |
| 85 | CAGGGCTATGAAACTGAT | 193628547 | 193628564 |
| 86 | CCAGGGCTATGAAACTGA | 193628548 | 193628565 |
| 87 | CTGATGGAGAAAGAGCGGAATC | 193628530 | 193628551 |
| 88 | CTGATGGAGAAAGAGCGGAA | 193628532 | 193628551 |
| 89 | AACTGATGGAGAAAGAGCGGAA | 193628532 | 193628553 |
| 90 | AACTGATGGAGAAAGAGCGG | 193628534 | 193628553 |
| 91 | GAAACTGATGGAGAAAGAGCGG | 193628534 | 193628555 |
| 92 | GGCTATGAAACTGATGGAGAAA | 193628540 | 193628561 |
| 93 | GGCTATGAAACTGATGGAGA | 193628542 | 193628561 |
| 94 | AGGGCTATGAAACTGATGGAGA | 193628542 | 193628563 |
| 95 | AGGGCTATGAAACTGATGGA | 193628544 | 193628563 |
| 96 | CCAGGGCTATGAAACTGATGGA | 193628544 | 193628565 |
| 97 | TTCTTACCCATTTAATTA | 193655041 | 193655059 |
| 98 | TGCTTCTTACCCATTTAA | 193655044 | 193655062 |
| 99 | TAATGCTTCTTACCCATT | 193655047 | 193655065 |
| 100 | AGATAATGCTTCTTACCC | 193655050 | 193655068 |
| 101 | CAGATAATGCTTCTTACC | 193655051 | 193655069 |
| 102 | CCCTTCAGATAATGCTTC | 193655056 | 193655074 |
| 103 | CTACTCCCTTCAGATAAT | 193655061 | 193655079 |
| 104 | AGCTCCTACTCCCTTCAG | 193655066 | 193655084 |
| 105 | TTCACAGCTCCTACTCCC | 193655071 | 193655089 |
| 106 | TAAAATTCACAGCTCCTA | 193655076 | 193655094 |
| 107 | AAATCTAAAATTCACAGC | 193655081 | 193655099 |
| 108 | GAATAAAATCTAAAATTC | 193655086 | 193655104 |
| 109 | GATGGGAATAAAATCTAA | 193655091 | 193655109 |
| 110 | GCTGTGATGGGAATAAAA | 193655096 | 193655114 |
| 111 | TAGAGGCTGTGATGGGAA | 193655101 | 193655119 |
| 112 | AAAGATAGAGGCTGTGAT | 193655106 | 193655124 |
| 113 | AAAAGAAAGATAGAGGCT | 193655111 | 193655129 |
| 114 | GACCTAAAAGAAAGATAG | 193655116 | 193655134 |
| 115 | ATAAAGACCTAAAAGAAA | 193655121 | 193655139 |
| 116 | GAGATATAAAGACCTAAA | 193655126 | 193655144 |
| 117 | GGCTGTGATGGGAATAAA | 193655097 | 193655115 |
| 118 | AGGCTGTGATGGGAATAA | 193655098 | 193655116 |
| 119 | GAGGCTGTGATGGGAATA | 193655099 | 193655117 |
| 120 | AGAGGCTGTGATGGGAAT | 193655100 | 193655118 |
| 121 | ATAGAGGCTGTGATGGGA | 193655102 | 193655120 |
| 122 | GATAGAGGCTGTGATGGG | 193655103 | 193655121 |
| 123 | AGATAGAGGCTGTGATGG | 193655104 | 193655122 |
| 124 | AAGATAGAGGCTGTGATG | 193655105 | 193655123 |
| 125 | TAGAGGCTGTGATGGGAATAAA | 193655097 | 193655119 |
| 126 | ATAGAGGCTGTGATGGGAATAA | 193655098 | 193655120 |
| 127 | GATAGAGGCTGTGATGGGAATA | 193655099 | 193655121 |
| 128 | AGATAGAGGCTGTGATGGGAAT | 193655100 | 193655122 |
| 129 | AAGATAGAGGCTGTGATGGGAA | 193655101 | 193655123 |
| 130 | GAGGCTGTGATGGGAATAAA | 193655097 | 193655117 |
| 131 | AGAGGCTGTGATGGGAATAA | 193655098 | 193655118 |
| 132 | TAGAGGCTGTGATGGGAATA | 193655099 | 193655119 |
| 133 | ATAGAGGCTGTGATGGGAAT | 193655100 | 193655120 |
| 134 | GATAGAGGCTGTGATGGGAA | 193655101 | 193655121 |
| 135 | AGATAGAGGCTGTGATGGGA | 193655102 | 193655122 |
| 136 | AAGATAGAGGCTGTGATGGG | 193655103 | 193655123 |
| 137 | CTGTGATGGGAATAAA | 193655097 | 193655113 |
| 138 | GCTGTGATGGGAATAA | 193655098 | 193655114 |
| 139 | GGCTGTGATGGGAATA | 193655099 | 193655115 |
| 140 | AGGCTGTGATGGGAAT | 193655100 | 193655116 |
| 141 | GAGGCTGTGATGGGAA | 193655101 | 193655117 |
| 142 | AGAGGCTGTGATGGGA | 193655102 | 193655118 |
| 143 | TAGAGGCTGTGATGGG | 193655103 | 193655119 |
| 144 | ATAGAGGCTGTGATGG | 193655104 | 193655120 |
| 145 | GATAGAGGCTGTGATG | 193655105 | 193655121 |
| 146 | AGATAGAGGCTGTGAT | 193655106 | 193655122 |
| 147 | AAGATAGAGGCTGTGA | 193655107 | 193655123 |
| 148 | AGGCTGTGATGTGAATAA | 193655099 | 193655116 |
| 149 | AGAGGCTGTGATGTGAAT | 193655101 | 193655118 |
| 150 | TAGAGGCTGTGATGTGAA | 193655102 | 193655119 |
| 151 | GATAGAGGCTGTGATTGG | 193655104 | 193655121 |
| 152 | GGCTGTGATGTGAATA | 193655100 | 193655115 |
| 153 | GAGGCTGTGATGTGAA | 193655102 | 193655117 |
| 154 | TAGAGGCTGTGATTGG | 193655104 | 193655119 |
| 155 | ATGAAACTGATGGAGA | 193628542 | 193628557 |
| 156 | CTATGAAACTGATGGA | 193628544 | 193628559 |
| 157 | GGCTATGAAACTGATG | 193628546 | 193628561 |
| 158 | GAAACTGATGGAGA | 193628542 | 193628555 |
| 159 | ATGAAACTGATGGA | 193628544 | 193628557 |
| 160 | CTATGAAACTGATG | 193628546 | 193628559 |
| 161 | GGCTATGAAACTGA | 193628548 | 193628561 |
| 162 | TAGAGGCTGTGATGGGAATAAAAT | 193655096 | 193655119 |
| 163 | ATAGAGGCTGTGATGGGAATAAAA | 193655097 | 193655120 |
| 164 | ATAGAGGCTGTGATGGGAATAAAAT | 193655096 | 193655120 |
| 165 | AAAGATAGAGGCTGTGATGGGAATA | 193655100 | 193655124 |
| 166 | GGCTATGAAACTGATGGAGAA | 193628541 | 193628561 |
| 167 | GGCTATGAAACTGATGGAGAAAGA | 193628538 | 193628561 |
| 168 | AGGGCTATGAAACTGATGGAGAAAG | 193628539 | 193628563 |
| 169 | CATTTAATTAAATTATAT | 193655033 | 193655051 |
| 170 | CCATTTAATTAAATTATA | 193655034 | 193655052 |
| 171 | CCCATTTAATTAAATTAT | 193655035 | 193655053 |
| 172 | ACCCATTTAATTAAATTA | 193655036 | 193655054 |
| 173 | TACCCATTTAATTAAATT | 193655037 | 193655055 |
| 174 | TTACCCATTTAATTAAAT | 193655038 | 193655056 |
| 175 | CTTACCCATTTAATTAAA | 193655039 | 193655057 |
| 176 | TCTTACCCATTTAATTAA | 193655040 | 193655058 |
| 177 | GATAGAGGCTGTGATGG | 193655104 | 193655122 |
| 178 | GGCTGTGAAACTGATGGA | 89481662 | 89481679 |
| 179 | GGCTGTGAAACTGATGGAGA | 89481660 | 89481679 |
| 180 | GCTATGAAACTGATGG | 193628545 | 193628560 |
| 181 | TATGAAACTGATGG | 193628545 | 193628558 |
| 182 | GCTATGAAACTGAT | 193628547 | 193628560 |
| 183 | GCTGTGAAACTGATGGAGAA | 89481659 | 89481678 |
| 184 | GGGCTGTGAAACTGATGGAG | 89481661 | 89481680 |
| 185 | TGTGAAACTGATGGAGAA | 89481659 | 89481676 |
| 186 | CTGTGAAACTGATGGAGA | 89481660 | 89481677 |
| 187 | GCTGTGAAACTGATGGAG | 89481661 | 89481678 |
| 188 | GGGCTGTGAAACTGATGG | 89481663 | 89481680 |
| 189 | TGAAACTGATGGAGAA | 89481659 | 89481674 |
| 190 | GTGAAACTGATGGAGA | 89481660 | 89481675 |
| 191 | TGTGAAACTGATGGAG | 89481661 | 89481676 |
| 192 | CTGTGAAACTGATGGA | 89481662 | 89481677 |
| 193 | GCTGTGAAACTGATGG | 89481663 | 89481678 |
| 194 | GGCTGTGAAACTGATG | 89481664 | 89481679 |
| 195 | GGGCTGTGAAACTGAT | 89481665 | 89481680 |
| 196 | CGGTCCAGGAATGAC | 193593285 | 193593303 |
| 197 | CCGGTCCAGGAATGA | 193593286 | 193593304 |
| 198 | CCCGGTCCAGGAATG | 193593287 | 193593305 |
| 199 | TCCCGGTCCAGGAAT | 193593288 | 193593306 |
| 200 | CTCCCGGTCCAGGAA | 193593289 | 193593307 |
| 201 | GCTCCCGGTCCAGGA | 193593290 | 193593308 |
| 202 | GGCTCCCGGTCCAGG | 193593291 | 193593309 |
| 203 | CGGGAGCCCCCGTGT | 193593318 | 193593336 |
| 204 | GCGGGAGCCCCCGTG | 193593319 | 193593337 |
| 205 | CGCGGGAGCCCCCGT | 193593320 | 193593338 |
| 206 | ACGCGGGAGCCCCCG | 193593321 | 193593339 |
| 207 | CACGCGGGAGCCCCC | 193593322 | 193593340 |
| 208 | CCACGCGGGAGCCCC | 193593323 | 193593341 |
| 209 | GCCACGCGGGAGCCC | 193593324 | 193593342 |
| 210 | GGCCACGCGGGAGCC | 193593325 | 193593343 |
| 211 | CGGCCACGCGGGAGC | 193593326 | 193593344 |
| 212 | ACGGCCACGCGGGAG | 193593327 | 193593345 |
| 213 | GACGGCCACGCGGGA | 193593328 | 193593346 |
| 214 | AGACGGCCACGCGGG | 193593329 | 193593347 |
| 215 | GCTAGGGAGGGATGGTTA | 193625988 | 193626006 |
| 216 | TGTAAGCTAGGGAGGGAT | 193625993 | 193626011 |
| 217 | ACAGATGTAAGCTAGGGA | 193625998 | 193626016 |
| 218 | AAGGAACAGATGTAAGCT | 193626003 | 193626021 |
| 219 | CAACAAAGGAACAGATGT | 193626008 | 193626026 |
| 220 | GGGTGCAACAAAGGAACA | 193626013 | 193626031 |
| 221 | ACCAAGGGTGCAACAAAG | 193626018 | 193626036 |
| 222 | GTTAAACCAAGGGTGCAA | 193626023 | 193626041 |
| 223 | ATAATGTTAAACCAAGGG | 193626028 | 193626046 |
| 224 | GGAGAATAATGTTAAACC | 193626033 | 193626051 |
| 225 | GGGGAGGAGAATAATGTT | 193626038 | 193626056 |
| 226 | AAATTGGGGAGGAGAATA | 193626043 | 193626061 |
| 227 | AGAGGAAATTGGGGAGGA | 193626048 | 193626066 |
| 228 | GGAGAAGAGGAAATTGGG | 193626053 | 193626071 |
| 229 | AATGAGGAGAAGAGGAAA | 193626058 | 193626076 |
| 230 | TTCACAATGAGGAGAAGA | 193626063 | 193626081 |
| 231 | ACGAGTTCACAATGAGGA | 193626068 | 193626086 |
| 232 | CTGCCACGAGTTCACAAT | 193626073 | 193626091 |
| 233 | AGACCCTGCCACGAGTTC | 193626078 | 193626096 |
| 234 | CAAGCAGACCCTGCCACG | 193626083 | 193626101 |
| 235 | CTCACCAAGCAGACCCTG | 193626088 | 193626106 |
| 236 | GAGCTCACCAAGCAGACC | 193626091 | 193626109 |
| 237 | AGAATGAGCTCACCAAGC | 193626096 | 193626114 |
| 238 | GTAAGAGAATGAGCTCAC | 193626101 | 193626119 |
| 239 | TTGTTGTAAGAGAATGAG | 193626106 | 193626124 |
| 240 | ATTTGTTGTTGTAAGAGA | 193626111 | 193626129 |
| 241 | CTTGAATTTGTTGTTGTA | 193626116 | 193626134 |
| 242 | ATGCTCTTGAATTTGTTG | 193626121 | 193626139 |
| 243 | TCTTCATGCTCTTGAATT | 193626126 | 193626144 |
| 244 | CTTCCTCTTCATGCTCTT | 193626131 | 193626149 |
| 245 | GCGCGCTTCCTCTTCATG | 193626136 | 193626154 |
| 246 | GCTCTGCGCGCTTCCTCT | 193626141 | 193626159 |
| 247 | GGCCAGCGGCTCTGCGCG | 193626149 | 193626167 |
| 248 | ATATTGGCCAGCGGCTCT | 193626154 | 193626172 |
| 249 | GTGCTATATTGGCCAGCG | 193626159 | 193626177 |
| 250 | AGCTCGTGCTATATTGGC | 193626164 | 193626182 |
| 251 | GGCATAGCTCGTGCTATA | 193626169 | 193626187 |
| 252 | TGTTGGGCATAGCTCGTG | 193626174 | 193626192 |
| 253 | GCTTCTGTTGGGCATAGC | 193626179 | 193626197 |
| 254 | CTTGCGCTTCTGTTGGGC | 193626184 | 193626202 |
| 255 | CACCTTGCGCTTCTGTTG | 193626187 | 193626205 |
| 256 | TCCATCACCTTGCGCTTC | 193626192 | 193626210 |
| 257 | AACCATCCATCACCTTGC | 193626197 | 193626215 |
| 258 | CCTTAAACCATCCATCAC | 193626202 | 193626220 |
| 259 | AGCCCCCTTAAACCATCC | 193626207 | 193626225 |
| 260 | TCGGTAGCCCCCTTAAAC | 193626212 | 193626230 |
| 261 | ATGTATCGGTAGCCCCCT | 193626217 | 193626235 |
| 262 | TGTGAATGTATCGGTAGC | 193626222 | 193626240 |
| 263 | ATTAGTGTGAATGTATCG | 193626227 | 193626245 |
| 264 | GGCTGATTAGTGTGAATG | 193626232 | 193626250 |
| 265 | GAAATGGCTGATTAGTGT | 193626237 | 193626255 |
| 266 | TGGCAGAAATGGCTGATT | 193626242 | 193626260 |
| 267 | GATCTTGGCAGAAATGGC | 193626247 | 193626265 |
| 268 | GACATGATCTTGGCAGAA | 193626252 | 193626270 |
| 269 | GAGGTGACATGATCTTGG | 193626257 | 193626275 |
| 270 | AGATTGAGGTGACATGAT | 193626262 | 193626280 |
| 271 | TGAACAGATTGAGGTGAC | 193626267 | 193626285 |
| 272 | GTCCATGAACAGATTGAG | 193626272 | 193626290 |
| 273 | TTGGAGTCCATGAACAGA | 193626277 | 193626295 |
| 274 | TGTATTTGGAGTCCATGA | 193626282 | 193626300 |
| 275 | TTTCTTGTATTTGGAGTC | 193626287 | 193626305 |

TABLE 6
Exemplary OPA1 ASO sequences (Organism: synthetic construct)

| | | | Coordinates: | |
|---|---|---|---|---|
| SEQ | | | GRCh38/hg38: chr3 | |
| ID NO | Region | Sequence (5′-3′) | Oligo Start | Oligo End |
| 215 | OPA1-IVS6-86 | GCTAGGGAGGGATGGTTA | 193625988 | 193626006 |
| 216 | OPA1-IVS6-81 | TGTAAGCTAGGGAGGGAT | 193625993 | 193626011 |
| 217 | OPA1-IVS6-76 | ACAGATGTAAGCTAGGGA | 193625998 | 193626016 |
| 218 | OPA1-IVS6-71 | AAGGAACAGATGTAAGCT | 193626003 | 193626021 |
| 227 | OPA1-IVS6-26 | AGAGGAAATTGGGGAGGA | 193626048 | 193626066 |
| 228 | OPA1-IVS6-21 | GGAGAAGAGGAAATTGGG | 193626053 | 193626071 |
| 229 | OPA1-IVS6-16 | AATGAGGAGAAGAGGAAA | 193626058 | 193626076 |
| 230 | OPA1-IVS6-11 | TTCACAATGAGGAGAAGA | 193626063 | 193626081 |
| 231 | OPA1-IVS6-6 | ACGAGTTCACAATGAGGA | 193626068 | 193626086 |
| 232 | OPA1-IVS6-1 | CTGCCACGAGTTCACAAT | 193626073 | 193626091 |
| 233 | OPA1-IVS6-EX7 + 5 | AGACCCTGCCACGAGTTC | 193626078 | 193626096 |
| 234 | OPA1-IVS6-EX7 + 10 | CAAGCAGACCCTGCCACG | 193626083 | 193626101 |
| 235 | OPA1-IVS6-EX7 + 15 | CTCACCAAGCAGACCCTG | 193626088 | 193626106 |
| 236 | OPA1-EX7 + 1 | GAGCTCACCAAGCAGACC | 193626091 | 193626109 |
| 237 | OPA1-EX7 + 6 | AGAATGAGCTCACCAAGC | 193626096 | 193626114 |
| 238 | OPA1-EX7 + 11 | GTAAGAGAATGAGCTCAC | 193626101 | 193626119 |
| 239 | OPA1-EX7 + 16 | TTGTTGTAAGAGAATGAG | 193626106 | 193626124 |
| 240 | OPA1-EX7 + 21 | ATTTGTTGTTGTAAGAGA | 193626111 | 193626129 |
| 241 | OPA1-EX7 + 26 | CTTGAATTTGTTGTTGTA | 193626116 | 193626134 |
| 242 | OPA1-EX7 + 31 | ATGCTCTTGAATTTGTTG | 193626121 | 193626139 |
| 250 | OPA1-EX7 − 21 | AGCTCGTGCTATATTGGC | 193626164 | 193626182 |
| 267 | OPA1-IVS7 + 46 | GATCTTGGCAGAAATGGC | 193626247 | 193626265 |

TABLE 7
Exemplary OPA1 ASO sequences (Organism: synthetic construct)

| | | | Coordinates: | |
|---|---|---|---|---|
| SEQ | | | GRCh38/hg38: chr3 | |
| ID NO | Region | Sequence (5′-3′) | Oligo Start | Oligo End |
| 280 | OPA1-EX7 + 27 | TCTTGAATTTGTTGTTGT | 193626117 | 193626135 |
| 281 | OPA1-EX7 + 28 | CTCTTGAATTTGTTGTTG | 193626118 | 193626136 |
| 282 | OPA1-EX7 + 29 | GCTCTTGAATTTGTTGTT | 193626119 | 193626137 |
| 283 | OPA1-EX7 + 30 | TGCTCTTGAATTTGTTGT | 193626120 | 193626138 |
| 284 | OPA1-EX7 + 32 | CATGCTCTTGAATTTGTT | 193626122 | 193626140 |
| 285 | OPA1-EX7 + 33 | TCATGCTCTTGAATTTGT | 193626123 | 193626141 |
| 286 | OPA1-EX7 + 34 | TTCATGCTCTTGAATTTG | 193626124 | 193626142 |
| 287 | OPA1-EX7 + 35 | CTTCATGCTCTTGAATTT | 193626125 | 193626143 |
| 288 | OPA1-EX7 + 26 | TGAATTTGTTGTTGTA | 193626116 | 193626132 |
| 289 | OPA1-EX7 + 27 | TTGAATTTGTTGTTGT | 193626117 | 193626133 |
| 290 | OPA1-EX7 + 28 | CTTGAATTTGTTGTTG | 193626118 | 193626134 |
| 291 | OPA1-EX7 + 29 | TCTTGAATTTGTTGTT | 193626119 | 193626135 |
| 292 | OPA1-EX7 + 30 | CTCTTGAATTTGTTGT | 193626120 | 193626136 |
| 293 | OPA1-EX7 + 31 | GCTCTTGAATTTGTTG | 193626121 | 193626137 |
| 294 | OPA1-EX7 + 32 | TGCTCTTGAATTTGTT | 193626122 | 193626138 |
| 295 | OPA1-EX7 + 33 | ATGCTCTTGAATTTGT | 193626123 | 193626139 |
| 296 | OPA1-EX7 + 34 | CATGCTCTTGAATTTG | 193626124 | 193626140 |
| 297 | OPA1-EX7 + 35 | TCATGCTCTTGAATTT | 193626125 | 193626141 |
| 298 | OPA1-EX7 + 36 | TTCATGCTCTTGAATT | 193626126 | 193626142 |
| 299 | OPA1-EX7 + 37 | CTTCATGCTCTTGAAT | 193626127 | 193626143 |

### Example 2.19: ASO-14 Mediates ATP Upregulation in OPA1 Haploinsufficient HEK293 Cell Line

The ATP levels generated through mitochondrial oxidative phosphorylation and the glycolytic pathway were measured in HEK293 cell lysates using a commercially available kit (Cat #ab83355, Abcam; USA) according to the manufacturer's instructions. Briefly, about 3×105 OPA1+/+(wildtype) and OPA1+/−HEK293 cells were plated in a T-25 flask and treated with 10 μM ASO-14. For the ATP test, 96-hours after treatment, cells were harvested, and two aliquots of cell suspension were prepared. One aliquot was processed for deproteinizing using commercially available kit (Cat #ab204708, Abcam; USA) to remove residual protein for executing ATP fluorescence assay to measure total ATP level. The second aliquot was used for BCA assay (Cat #23225, Thermo Fisher; USA) to measure total protein level. ATP level was then calculated by normalizing the measured total ATP level to the measured total protein level.

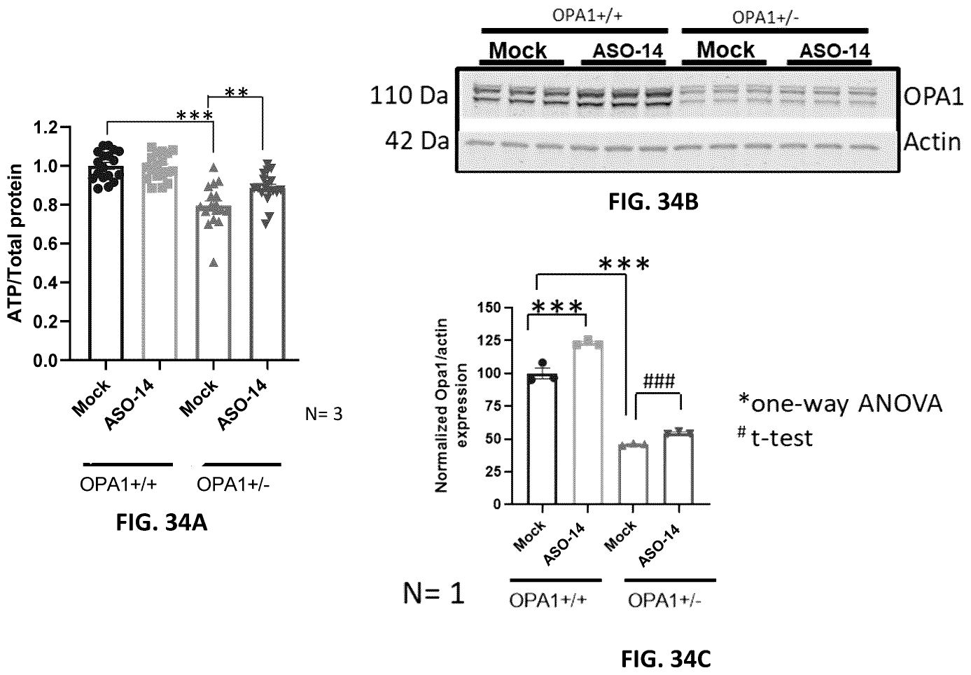

FIG. 34A summarizes the ATP level measured under each condition. In the mock group, untreated OPA1+/−HEK293 cells were found to have 0.79±0.02 ATP level as compared to untreated OPA1+/+HEK293 cells. There was about a 20% ATP deficit in OPA1+/−HEK293 cells. In comparison, OPA1+/−HEK293 cells treated with ASO-14 had ATP levels 0.88±0.01, significantly higher than the mock-treated OPA1+/−HEK293 cells, suggesting that treatment of ASO-14 reduced the deficit by about 50%. Data were collected from three independent experiments. (Statistics: Ordinary one-way ANOVA; ***P<0.0001; **P<0.0080).

FIGS. 34B-34C demonstrate the OPA1 protein under each condition. 96 hours after treatment with ASO-14 or no treatment (mock), cells were lysed with RIPA buffer and immunoblot blot was probed with antibodies targeting OPA1 and β-actin. The data show that treatment of ASO-14 upregulated about 18% OPA1 protein in OPA1+/− cells. FIG. 34B shows the immunoblot gel images. Multiple bands on the immunoblot image represent various isoforms of OPA1. FIG. 34C summarizes quantification of the immunoblot results. Untreated (mock) OPA1+/−HEK293 cells were found to have 46±0.5% OPA1 protein level as compared to untreated (mock) OPA1+/+HEK293 cells. OPA1+/+ cells treated with ASO-14 had OPA1 levels 123.2±1.3 of untreated OPA1+/+ cells. OPA1+/− cells treated with ASO-14 had OPA1 levels 54.54±0.6% of untreated OPA1+/+ cells. Statistics performed with corresponding mock. *** P<0.0001, by Ordinary one-Way ANOVA and ###P<0.0001, by Welch's t test. Data represent average of three technical replicates.

### Example 2.20: Exemplary Antisense Oligomers Restore OPA1 Expression in Cells with OPA1 Mutations from Diagnosed Patients

This example examines OPA1 mRNA and protein levels in cells with mutations in OPA1 gene from patients diagnosed with Autosomal dominant optic atrophy (ADOA), as well as effects of exemplary antisense oligomer ASO-14 on OPA1 mRNA and protein levels, and mitochondrial bioenergetics in the patient cells.

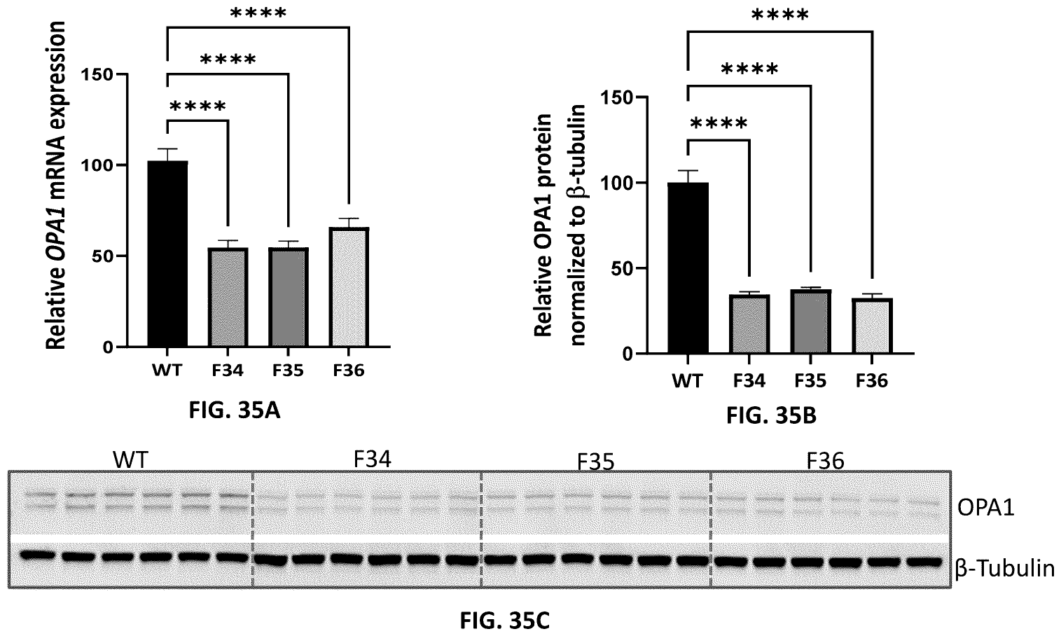

FIGS. 35A-35C summarize mRNA and protein expression of OPA1 gene in fibroblast cells from diagnosed patients that have a haploinsufficient mutation in the OPA1 gene: F34 (OPA1 canonical splice mutation at c.1608+1delGTGAGG); F35 (OPA1 frameshift mutation at c.2873_2876del); F36 (OPA1 frameshift mutation at c.635_636delAA). mRNA expression level of OPA1 gene in patient cells is about 50% to 60% of the mRNA level in wildtype (WT) cells (FIG. 35A); OPA1 protein level in patient cells is about 30% to about 40% of the protein level in WT cells (FIG. 35B). Histograms in FIGS. 35A-35B show mean±SEM of 3 independent experiments; one-way ANOVA compared to WT group (****P<0.0001). FIG. 35C shows a representative immunoblot image of OPA protein expression level in diseased fibroblast cells.

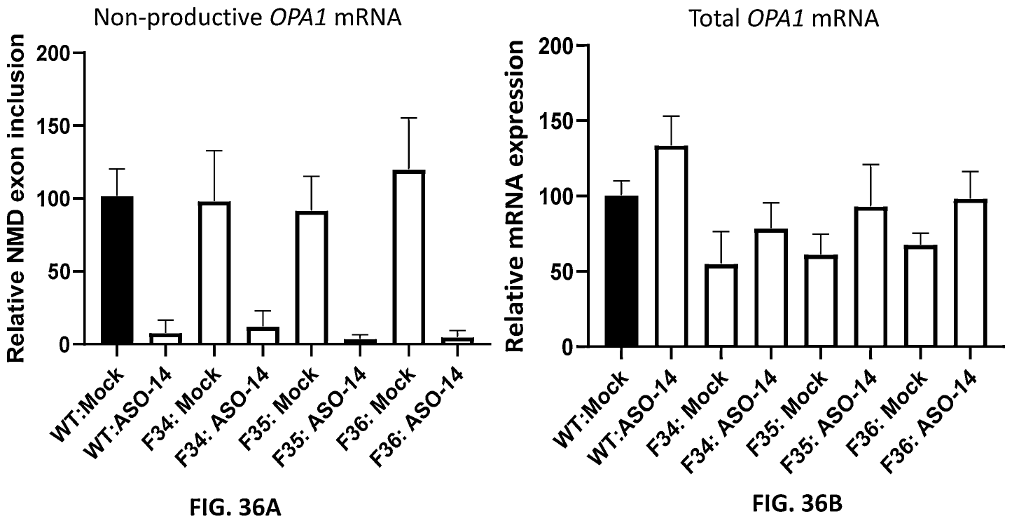

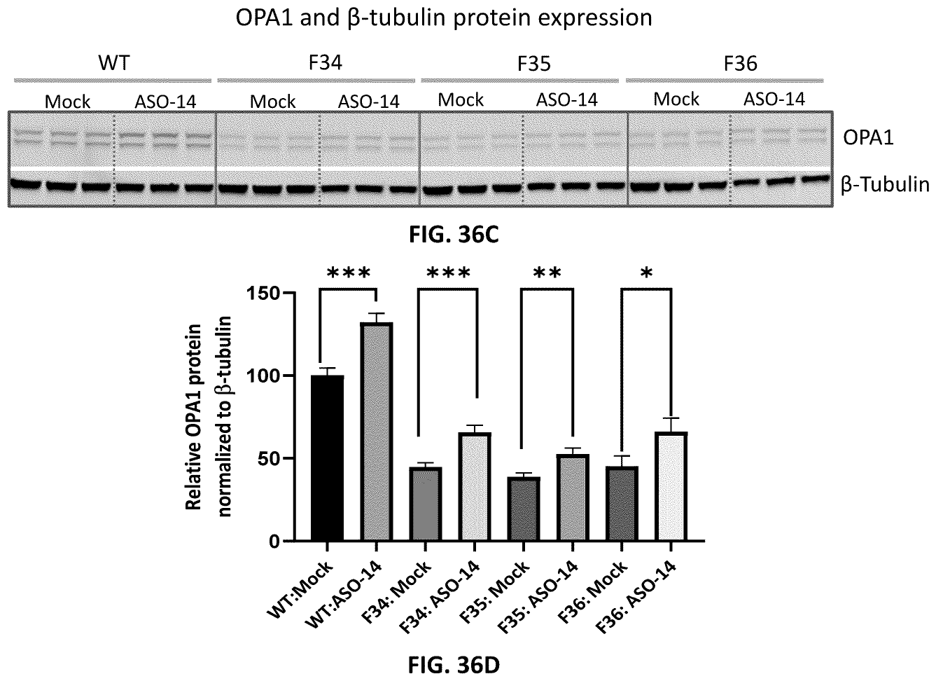

FIGS. 36A-36D demonstrate the effects of exemplary antisense oligomer, ASO-14, on OPA1 NMD exon inclusion, mRNA level, and protein level in wildtype (WT) fibroblast cells and fibroblast cells from diagnosed patients that have haploinsufficient mutation in OPA1 gene. The fibroblast cells were transfected with ASO-14 (40 nM), and RNA was isolated 24 hours after transfection and analyzed. For non-productive OPA1 mRNA measurement, cells were treated with cycloheximide (50 μg/mL) for 3 hours prior to RNA isolation. Immunoblot was performed 72 hours. post transfection with antibodies targeting OPA1 and β-tubulin. As shown in FIG. 36A, ASO-14 significantly decreased inclusion of NMD exon (exon 7x), measured by level of non-productive OPA1 mRNA, in WT cells and all diseased cells to lower than 20% of the level of the normalized level in WT cells. There was a trend of increase in total OPA1 mRNA level in all types of cells by the treatment of ASO-14 (FIG. 36B). Histograms in FIGS. 36A-36B show mean±SEM of 2-3 independent experiments; one-way ANOVA vs. Mock for respective cell line (*P<0.05; ***P<0.001; ****P<0.0001). Correspondingly, OPA1 protein level was significantly increased by the treatment of ASO-14 in all types of cells (FIGS. 36C-36D). FIG. 36C shows representative immunoblot images of OPA1 protein and loading control β-Tubulin under all types of conditions; FIG. 36D shows the statistical summary of the OPA1 protein levels, the histograms show mean SEM of 3 independent experiments; unpaired t-test vs. Mock for respective cell line (*P<0.05, ** P<0.01, ***<0.001).

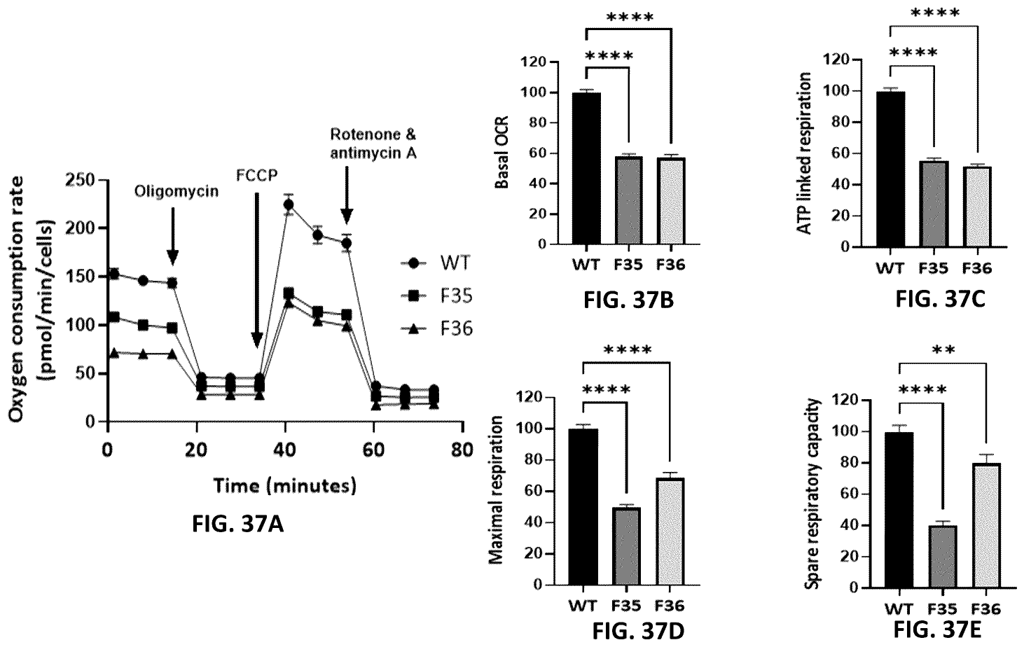

FIGS. 37A-37E demonstrate that patient fibroblast cells (cell lines F35 and F36) show deficiencies in mitochondrial bioenergetics. FIG. 37A shows representative time courses of the oxygen consumption rate of WT cells, F35 cells, and F36 cells at baseline level and when challenged sequentially with oligomycin, FCCP, rotenone, and antimycin A. Patient fibroblast cells, F35 and F36 cells, were found to have reduced basal oxygen consumption rate (FIG. 37B), ATP-linked respiration (FIG. 37C), maximal respiration (FIG. 37D), and spare respiratory capacity (FIG. 37E), as compared to WT fibroblast cells. Units in FIGS. 37B-37E are pmol/min/cells, data normalized to wild-type (WT). Histograms in FIGS. 37B-37E show mean SEM of >18 individual measurements from 2 independent experiments; one-way ANOVA vs. WT (** P<0.01; **** P<0.0001).

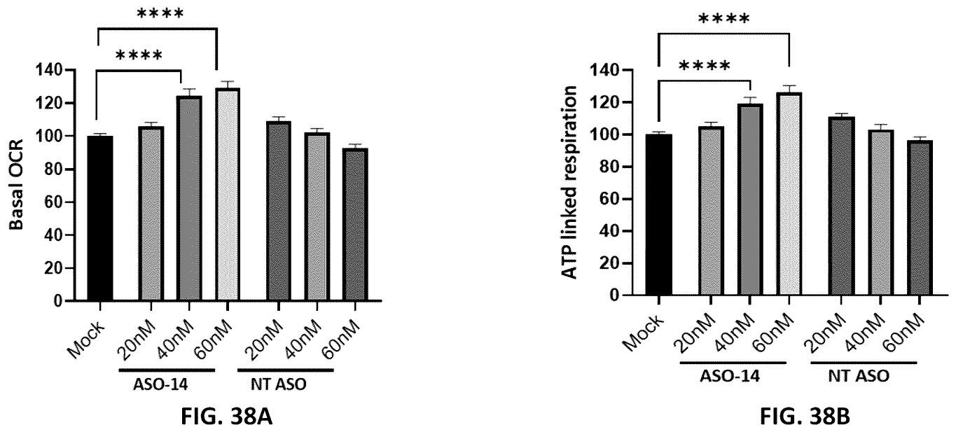

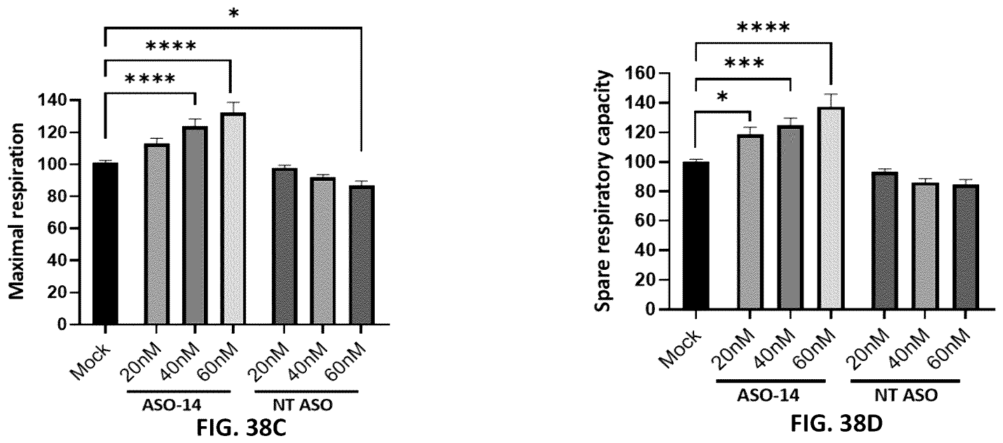

FIGS. 38A-38D demonstrate that ASO-14 increased mitochondrial energetics in F35 patient cell line. As shown in the figures, treatment with 40 nM or 60 nM ASO-14 increased basal oxygen consumption rate (FIG. 38A), ATP-linked respiration (FIG. 38B), maximal respiration (FIG. 38C), and spare respiratory capacity (FIG. 38D) of F35 patient cells in a dose-dependent manner. Treatment with 20 nM ASO-14 also significantly increased spare respiratory capacity (FIG. 38D). In contrast, non-targeting ASO (NT ASO, targeting an unrelated gene) did not significantly alter the parameters at any of the tested concentrations. Units in the figures are pmol/min/cells; the Oxygen Consumption Rates (OCR) are normalized to total cell count and plotted to Mock (No ASO). The histograms show mean±SEM of >20 individual measurements from at least 3 independent experiments; one-way ANOVA vs. Mock (*P<0.05; ***P<0.001; ****P<0.0001).

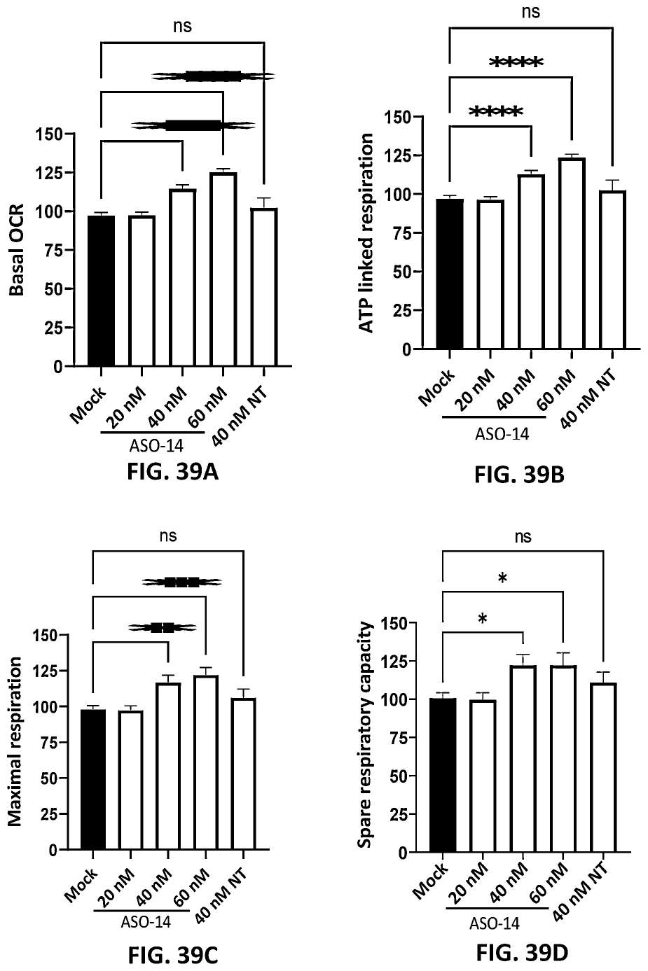

FIGS. 39A-39D demonstrate that ASO-14 increased mitochondrial energetics in F36 patient cell line. As shown in the figures, ASO-14 also increased basal oxygen consumption rate (FIG. 39A), ATP-linked respiration (FIG. 39B), maximal respiration (FIG. 39C), and spare respiratory capacity (FIG. 39D) of F36 patient cells in a dose-dependent manner from 20 nM, 40 nM, to 60 nM. In contrast, non-targeting ASO did not significantly alter the parameters at 40 nM. Units in the figures are pmol/min/cells; the Oxygen Consumption Rates (OCR) are normalized to total cell count and plotted to Mock (No ASO). The histograms show mean SEM of >20 individual measurements from 2-5 independent experiments; one-way ANOVA vs. Mock (*P<0.05; ** P<0.01; *** P<0.001 **** P<0.0001).

The experiments in F35 and F36 cells suggest that the dose-dependent improvement in mitochondrial bioenergetics by ASO-14 is mutation-independent.

The foregoing preclinical data support the TANGO disease modifying approach in ADOA. As demonstrated by the data, the exemplary antisense oligomer, ASO-14, reduced non-productive exon inclusion, increased total OPA1 mRNA and protein expression in all three patient fibroblast cell lines; increased ASO-14 dose increased mitochondrial respiration in two fibroblast cell lines. The data further suggest that the ASO mediated increase in OPA1 protein expression is disease modifying in ADOA in a mutation-independent manner.

TABLE 8
Exemplary ASOs Targeting 5′ UTR of OPA1 Mature
mRNA (Organism: synthetic construct)

| | | Coordinates: | |
|---|---|---|---|
| | SEQ | GRCh38/hg38: chr3 | |
| 16-mers | ID NO | Oligo Start | Oligo End |
| TCTAGGCGGGCAGCAC | 608 | 193593143 | 193593159 |
| TTCTAGGCGGGCAGCA | 609 | 193593144 | 193593160 |
| TTTCTAGGCGGGCAGC | 610 | 193593145 | 193593161 |
| CTTTCTAGGCGGGCAG | 611 | 193593146 | 193593162 |
| CCTTTCTAGGCGGGCA | 612 | 193593147 | 193593163 |
| CCCTTTCTAGGCGGGC | 613 | 193593148 | 193593164 |
| ACCCTTTCTAGGCGGG | 614 | 193593149 | 193593165 |
| CACCCTTTCTAGGCGG | 615 | 193593150 | 193593166 |
| TCACCCTTTCTAGGCG | 616 | 193593151 | 193593167 |
| TTCACCCTTTCTAGGC | 617 | 193593152 | 193593168 |
| CTTCACCCTTTCTAGG | 618 | 193593153 | 193593169 |
| ACTTCACCCTTTCTAG | 619 | 193593154 | 193593170 |
| CACTTCACCCTTTCTA | 620 | 193593155 | 193593171 |
| CCACTTCACCCTTTCT | 621 | 193593156 | 193593172 |
| ACCACTTCACCCTTTC | 622 | 193593157 | 193593173 |
| AACCACTTCACCCTTT | 623 | 193593158 | 193593174 |
| CAACCACTTCACCCTT | 624 | 193593159 | 193593175 |
| ACAACCACTTCACCCT | 625 | 193593160 | 193593176 |
| AACAACCACTTCACCC | 626 | 193593161 | 193593177 |
| AAACAACCACTTCACC | 627 | 193593162 | 193593178 |
| GAAACAACCACTTCAC | 628 | 193593163 | 193593179 |
| GGAAACAACCACTTCA | 629 | 193593164 | 193593180 |
| CGGAAACAACCACTTC | 630 | 193593165 | 193593181 |
| ACGGAAACAACCACTT | 631 | 193593166 | 193593182 |
| CACGGAAACAACCACT | 632 | 193593167 | 193593183 |
| TCACGGAAACAACCAC | 633 | 193593168 | 193593184 |
| GTCACGGAAACAACCA | 634 | 193593169 | 193593185 |
| CGTCACGGAAACAACC | 635 | 193593170 | 193593186 |
| CCGTCACGGAAACAAC | 636 | 193593171 | 193593187 |
| TCCGTCACGGAAACAA | 637 | 193593172 | 193593188 |
| GTCCGTCACGGAAACA | 638 | 193593173 | 193593189 |
| AGTCCGTCACGGAAAC | 639 | 193593174 | 193593190 |
| CAGTCCGTCACGGAAA | 640 | 193593175 | 193593191 |
| TCAGTCCGTCACGGAA | 641 | 193593176 | 193593192 |
| CTCAGTCCGTCACGGA | 642 | 193593177 | 193593193 |
| ACTCAGTCCGTCACGG | 643 | 193593178 | 193593194 |
| TACTCAGTCCGTCACG | 644 | 193593179 | 193593195 |
| GTACTCAGTCCGTCAC | 645 | 193593180 | 193593196 |
| CGTACTCAGTCCGTCA | 646 | 193593181 | 193593197 |
| CCGTACTCAGTCCGTC | 647 | 193593182 | 193593198 |
| CCCGTACTCAGTCCGT | 648 | 193593183 | 193593199 |
| ACCCGTACTCAGTCCG | 649 | 193593184 | 193593200 |
| CACCCGTACTCAGTCC | 650 | 193593185 | 193593201 |
| GCACCCGTACTCAGTC | 651 | 193593186 | 193593202 |
| GGCACCCGTACTCAGT | 652 | 193593187 | 193593203 |
| AGGCACCCGTACTCAG | 653 | 193593188 | 193593204 |
| CAGGCACCCGTACTCA | 654 | 193593189 | 193593205 |
| ACAGGCACCCGTACTC | 655 | 193593190 | 193593206 |
| GACAGGCACCCGTACT | 656 | 193593191 | 193593207 |
| TGACAGGCACCCGTAC | 657 | 193593192 | 193593208 |
| CTGACAGGCACCCGTA | 658 | 193593193 | 193593209 |
| CCTGACAGGCACCCGT | 659 | 193593194 | 193593210 |
| GCCTGACAGGCACCCG | 660 | 193593195 | 193593211 |
| AGCCTGACAGGCACCC | 661 | 193593196 | 193593212 |
| GAGCCTGACAGGCACC | 662 | 193593197 | 193593213 |
| AGAGCCTGACAGGCAC | 663 | 193593198 | 193593214 |
| AAGAGCCTGACAGGCA | 664 | 193593199 | 193593215 |
| CAAGAGCCTGACAGGC | 665 | 193593200 | 193593216 |
| GCAAGAGCCTGACAGG | 666 | 193593201 | 193593217 |
| CGCAAGAGCCTGACAG | 667 | 193593202 | 193593218 |
| CCGCAAGAGCCTGACA | 668 | 193593203 | 193593219 |
| TCCGCAAGAGCCTGAC | 669 | 193593204 | 193593220 |
| TTCCGCAAGAGCCTGA | 670 | 193593205 | 193593221 |
| CTTCCGCAAGAGCCTG | 671 | 193593206 | 193593222 |
| ACTTCCGCAAGAGCCT | 672 | 193593207 | 193593223 |
| GACTTCCGCAAGAGCC | 673 | 193593208 | 193593224 |
| GGACTTCCGCAAGAGC | 674 | 193593209 | 193593225 |
| TGGACTTCCGCAAGAG | 675 | 193593210 | 193593226 |
| ATGGACTTCCGCAAGA | 676 | 193593211 | 193593227 |
| CATGGACTTCCGCAAG | 677 | 193593212 | 193593228 |
| GCATGGACTTCCGCAA | 678 | 193593213 | 193593229 |
| CGCATGGACTTCCGCA | 679 | 193593214 | 193593230 |
| GCGCATGGACTTCCGC | 680 | 193593215 | 193593231 |
| GGCGCATGGACTTCCG | 681 | 193593216 | 193593232 |
| TGGCGCATGGACTTCC | 682 | 193593217 | 193593233 |
| ATGGCGCATGGACTTC | 683 | 193593218 | 193593234 |
| AATGGCGCATGGACTT | 684 | 193593219 | 193593235 |
| CAATGGCGCATGGACT | 685 | 193593220 | 193593236 |
| CCAATGGCGCATGGAC | 686 | 193593221 | 193593237 |
| CCCAATGGCGCATGGA | 687 | 193593222 | 193593238 |
| TCCCAATGGCGCATGG | 688 | 193593223 | 193593239 |
| CTCCCAATGGCGCATG | 689 | 193593224 | 193593240 |
| CCTCCCAATGGCGCAT | 690 | 193593225 | 193593241 |
| CCCTCCCAATGGCGCA | 691 | 193593226 | 193593242 |
| GCCCTCCCAATGGCGC | 692 | 193593227 | 193593243 |
| GGCCCTCCCAATGGCG | 693 | 193593228 | 193593244 |
| AGGCCCTCCCAATGGC | 694 | 193593229 | 193593245 |
| GAGGCCCTCCCAATGG | 695 | 193593230 | 193593246 |
| CGAGGCCCTCCCAATG | 696 | 193593231 | 193593247 |
| CCGAGGCCCTCCCAAT | 697 | 193593232 | 193593248 |
| GCCGAGGCCCTCCCAA | 698 | 193593233 | 193593249 |
| GGCCGAGGCCCTCCCA | 699 | 193593234 | 193593250 |
| CGGCCGAGGCCCTCCC | 700 | 193593235 | 193593251 |
| GCGGCCGAGGCCCTCC | 701 | 193593236 | 193593252 |
| CGCGGCCGAGGCCCTC | 702 | 193593237 | 193593253 |
| CCGCGGCCGAGGCCCT | 703 | 193593238 | 193593254 |
| GCCGCGGCCGAGGCCC | 704 | 193593239 | 193593255 |
| AGCCGCGGCCGAGGCC | 705 | 193593240 | 193593256 |
| GAGCCGCGGCCGAGGC | 706 | 193593241 | 193593257 |
| AGAGCCGCGGCCGAGG | 707 | 193593242 | 193593258 |
| CAGAGCCGCGGCCGAG | 708 | 193593243 | 193593259 |
| ACAGAGCCGCGGCCGA | 709 | 193593244 | 193593260 |
| CACAGAGCCGCGGCCG | 710 | 193593245 | 193593261 |
| GCACAGAGCCGCGGCC | 711 | 193593246 | 193593262 |
| GGCACAGAGCCGCGGC | 712 | 193593247 | 193593263 |
| GGGCACAGAGCCGCGG | 713 | 193593248 | 193593264 |
| AGGGCACAGAGCCGCG | 714 | 193593249 | 193593265 |
| AAGGGCACAGAGCCGC | 715 | 193593250 | 193593266 |
| CAAGGGCACAGAGCCG | 716 | 193593251 | 193593267 |
| GCAAGGGCACAGAGCC | 717 | 193593252 | 193593268 |
| AGCAAGGGCACAGAGC | 718 | 193593253 | 193593269 |
| CAGCAAGGGCACAGAG | 719 | 193593254 | 193593270 |
| GCAGCAAGGGCACAGA | 720 | 193593255 | 193593271 |
| AGCAGCAAGGGCACAG | 721 | 193593256 | 193593272 |
| CAGCAGCAAGGGCACA | 722 | 193593257 | 193593273 |
| TCAGCAGCAAGGGCAC | 723 | 193593258 | 193593274 |
| CTCAGCAGCAAGGGCA | 724 | 193593259 | 193593275 |
| CCTCAGCAGCAAGGGC | 725 | 193593260 | 193593276 |
| CCCTCAGCAGCAAGGG | 726 | 193593261 | 193593277 |
| GCCCTCAGCAGCAAGG | 727 | 193593262 | 193593278 |
| GGCCCTCAGCAGCAAG | 728 | 193593263 | 193593279 |
| TGGCCCTCAGCAGCAA | 729 | 193593264 | 193593280 |
| GTGGCCCTCAGCAGCA | 730 | 193593265 | 193593281 |
| AGTGGCCCTCAGCAGC | 731 | 193593266 | 193593282 |
| AAGTGGCCCTCAGCAG | 732 | 193593267 | 193593283 |
| GAAGTGGCCCTCAGCA | 733 | 193593268 | 193593284 |
| GGAAGTGGCCCTCAGC | 734 | 193593269 | 193593285 |
| AGGAAGTGGCCCTCAG | 735 | 193593270 | 193593286 |
| CAGGAAGTGGCCCTCA | 736 | 193593271 | 193593287 |
| CCAGGAAGTGGCCCTC | 737 | 193593272 | 193593288 |
| CCCAGGAAGTGGCCCT | 738 | 193593273 | 193593289 |
| ACCCAGGAAGTGGCCC | 739 | 193593274 | 193593290 |
| GACCCAGGAAGTGGCC | 740 | 193593275 | 193593291 |
| TGACCCAGGAAGTGGC | 741 | 193593276 | 193593292 |
| ATGACCCAGGAAGTGG | 742 | 193593277 | 193593293 |
| AATGACCCAGGAAGTG | 743 | 193593278 | 193593294 |
| GAATGACCCAGGAAGT | 744 | 193593279 | 193593295 |
| GGAATGACCCAGGAAG | 745 | 193593280 | 193593296 |
| AGGAATGACCCAGGAA | 746 | 193593281 | 193593297 |
| CAGGAATGACCCAGGA | 747 | 193593282 | 193593298 |
| CCAGGAATGACCCAGG | 748 | 193593283 | 193593299 |
| TCCAGGAATGACCCAG | 749 | 193593284 | 193593300 |
| GTCCAGGAATGACCCA | 750 | 193593285 | 193593301 |
| GGTCCAGGAATGACCC | 751 | 193593286 | 193593302 |
| CGGTCCAGGAATGACC | 752 | 193593287 | 193593303 |
| CCGGTCCAGGAATGAC | 753 | 193593288 | 193593304 |
| CCCGGTCCAGGAATGA | 754 | 193593289 | 193593305 |
| TCCCGGTCCAGGAATG | 755 | 193593290 | 193593306 |
| CTCCCGGTCCAGGAAT | 756 | 193593291 | 193593307 |
| GCTCCCGGTCCAGGAA | 757 | 193593292 | 193593308 |
| GGCTCCCGGTCCAGGA | 758 | 193593293 | 193593309 |
| CGGCTCCCGGTCCAGG | 759 | 193593294 | 193593310 |
| CCGGCTCCCGGTCCAG | 760 | 193593295 | 193593311 |
| CCCGGCTCCCGGTCCA | 761 | 193593296 | 193593312 |
| GCCCGGCTCCCGGTCC | 762 | 193593297 | 193593313 |
| AGCCCGGCTCCCGGTC | 763 | 193593298 | 193593314 |
| CAGCCCGGCTCCCGGT | 764 | 193593299 | 193593315 |
| CCAGCCCGGCTCCCGG | 765 | 193593300 | 193593316 |
| CCCAGCCCGGCTCCCG | 766 | 193593301 | 193593317 |
| CCCCAGCCCGGCTCCC | 767 | 193593302 | 193593318 |
| GCCCCAGCCCGGCTCC | 768 | 193593303 | 193593319 |
| AGCCCCAGCCCGGCTC | 769 | 193593304 | 193593320 |
| GAGCCCCAGCCCGGCT | 770 | 193593305 | 193593321 |
| TGAGCCCCAGCCCGGC | 771 | 193593306 | 193593322 |
| GTGAGCCCCAGCCCGG | 772 | 193593307 | 193593323 |
| TGTGAGCCCCAGCCCG | 773 | 193593308 | 193593324 |
| GTGTGAGCCCCAGCCC | 774 | 193593309 | 193593325 |
| CGTGTGAGCCCCAGCC | 775 | 193593310 | 193593326 |
| CCGTGTGAGCCCCAGC | 776 | 193593311 | 193593327 |
| CCCGTGTGAGCCCCAG | 777 | 193593312 | 193593328 |
| CCCCGTGTGAGCCCCA | 778 | 193593313 | 193593329 |
| CCCCCGTGTGAGCCCC | 779 | 193593314 | 193593330 |
| GCCCCCGTGTGAGCCC | 780 | 193593315 | 193593331 |
| AGCCCCCGTGTGAGCC | 781 | 193593316 | 193593332 |
| GAGCCCCCGTGTGAGC | 782 | 193593317 | 193593333 |
| GGAGCCCCCGTGTGAG | 783 | 193593318 | 193593334 |
| GGGAGCCCCCGTGTGA | 784 | 193593319 | 193593335 |
| CGGGAGCCCCCGTGTG | 785 | 193593320 | 193593336 |
| GCGGGAGCCCCCGTGT | 786 | 193593321 | 193593337 |
| CGCGGGAGCCCCCGTG | 787 | 193593322 | 193593338 |
| ACGCGGGAGCCCCCGT | 788 | 193593323 | 193593339 |
| CACGCGGGAGCCCCCG | 789 | 193593324 | 193593340 |
| CCACGCGGGAGCCCCC | 790 | 193593325 | 193593341 |
| GCCACGCGGGAGCCCC | 791 | 193593326 | 193593342 |
| GGCCACGCGGGAGCCC | 792 | 193593327 | 193593343 |
| CGGCCACGCGGGAGCC | 793 | 193593328 | 193593344 |
| ACGGCCACGCGGGAGC | 794 | 193593329 | 193593345 |
| GACGGCCACGCGGGAG | 795 | 193593330 | 193593346 |
| AGACGGCCACGCGGGA | 796 | 193593331 | 193593347 |
| GAGACGGCCACGCGGG | 797 | 193593332 | 193593348 |
| CGAGACGGCCACGCGG | 798 | 193593333 | 193593349 |
| CCGAGACGGCCACGCG | 799 | 193593334 | 193593350 |
| GCCGAGACGGCCACGC | 800 | 193593335 | 193593351 |
| CGCCGAGACGGCCACG | 801 | 193593336 | 193593352 |
| GCGCCGAGACGGCCAC | 802 | 193593337 | 193593353 |
| GGCGCCGAGACGGCCA | 803 | 193593338 | 193593354 |
| AGGCGCCGAGACGGCC | 804 | 193593339 | 193593355 |
| CAGGCGCCGAGACGGC | 805 | 193593340 | 193593356 |
| GCAGGCGCCGAGACGG | 806 | 193593341 | 193593357 |
| CGCAGGCGCCGAGACG | 807 | 193593342 | 193593358 |
| ACGCAGGCGCCGAGAC | 808 | 193593343 | 193593359 |
| CACGCAGGCGCCGAGA | 809 | 193593344 | 193593360 |
| TCACGCAGGCGCCGAG | 810 | 193593345 | 193593361 |
| GTCACGCAGGCGCCGA | 811 | 193593346 | 193593362 |
| GGTCACGCAGGCGCCG | 812 | 193593347 | 193593363 |
| AGGTCACGCAGGCGCC | 813 | 193593348 | 193593364 |
| GAGGTCACGCAGGCGC | 814 | 193593349 | 193593365 |
| GGAGGTCACGCAGGCG | 815 | 193593350 | 193593366 |
| GGGAGGTCACGCAGGC | 816 | 193593351 | 193593367 |
| GGGGAGGTCACGCAGG | 817 | 193593352 | 193593368 |
| CGGGGAGGTCACGCAG | 818 | 193593353 | 193593369 |
| GCGGGGAGGTCACGCA | 819 | 193593354 | 193593370 |
| GGCGGGGAGGTCACGC | 820 | 193593355 | 193593371 |
| CGGCGGGGAGGTCACG | 821 | 193593356 | 193593372 |
| CCGGCGGGGAGGTCAC | 822 | 193593357 | 193593373 |
| GCCGGCGGGGAGGTCA | 823 | 193593358 | 193593374 |
| CGCCGGCGGGGAGGTC | 824 | 193593359 | 193593375 |
| CCGCCGGCGGGGAGGT | 825 | 193593360 | 193593376 |
| CCCGCCGGCGGGGAGG | 826 | 193593361 | 193593377 |
| | | Coordinates: | |
| | SEQ | GRCh38/hg38: chr3 | |
| 18-mers | ID NO | Oligo Start | Oligo End |
| TTTCTAGGCGGGCAGCAC | 827 | 193593143 | 193593161 |
| CTTTCTAGGCGGGCAGCA | 828 | 193593144 | 193593162 |
| CCTTTCTAGGCGGGCAGC | 829 | 193593145 | 193593163 |
| CCCTTTCTAGGCGGGCAG | 830 | 193593146 | 193593164 |
| ACCCTTTCTAGGCGGGCA | 831 | 193593147 | 193593165 |
| CACCCTTTCTAGGCGGGC | 832 | 193593148 | 193593166 |
| TCACCCTTTCTAGGCGGG | 833 | 193593149 | 193593167 |
| TTCACCCTTTCTAGGCGG | 834 | 193593150 | 193593168 |
| CTTCACCCTTTCTAGGCG | 835 | 193593151 | 193593169 |
| ACTTCACCCTTTCTAGGC | 836 | 193593152 | 193593170 |
| CACTTCACCCTTTCTAGG | 837 | 193593153 | 193593171 |
| CCACTTCACCCTTTCTAG | 838 | 193593154 | 193593172 |
| ACCACTTCACCCTTTCTA | 839 | 193593155 | 193593173 |
| AACCACTTCACCCTTTCT | 840 | 193593156 | 193593174 |
| CAACCACTTCACCCTTTC | 841 | 193593157 | 193593175 |
| ACAACCACTTCACCCTTT | 842 | 193593158 | 193593176 |
| AACAACCACTTCACCCTT | 843 | 193593159 | 193593177 |
| AAACAACCACTTCACCCT | 844 | 193593160 | 193593178 |
| GAAACAACCACTTCACCC | 845 | 193593161 | 193593179 |
| GGAAACAACCACTTCACC | 846 | 193593162 | 193593180 |
| CGGAAACAACCACTTCAC | 847 | 193593163 | 193593181 |
| ACGGAAACAACCACTTCA | 848 | 193593164 | 193593182 |
| CACGGAAACAACCACTTC | 849 | 193593165 | 193593183 |
| TCACGGAAACAACCACTT | 850 | 193593166 | 193593184 |
| GTCACGGAAACAACCACT | 851 | 193593167 | 193593185 |
| CGTCACGGAAACAACCAC | 852 | 193593168 | 193593186 |
| CCGTCACGGAAACAACCA | 853 | 193593169 | 193593187 |
| TCCGTCACGGAAACAACC | 854 | 193593170 | 193593188 |
| GTCCGTCACGGAAACAAC | 855 | 193593171 | 193593189 |
| AGTCCGTCACGGAAACAA | 856 | 193593172 | 193593190 |
| CAGTCCGTCACGGAAACA | 857 | 193593173 | 193593191 |
| TCAGTCCGTCACGGAAAC | 858 | 193593174 | 193593192 |
| CTCAGTCCGTCACGGAAA | 859 | 193593175 | 193593193 |
| ACTCAGTCCGTCACGGAA | 860 | 193593176 | 193593194 |
| TACTCAGTCCGTCACGGA | 861 | 193593177 | 193593195 |
| GTACTCAGTCCGTCACGG | 862 | 193593178 | 193593196 |
| CGTACTCAGTCCGTCACG | 863 | 193593179 | 193593197 |
| CCGTACTCAGTCCGTCAC | 864 | 193593180 | 193593198 |
| CCCGTACTCAGTCCGTCA | 865 | 193593181 | 193593199 |
| ACCCGTACTCAGTCCGTC | 866 | 193593182 | 193593200 |
| CACCCGTACTCAGTCCGT | 867 | 193593183 | 193593201 |
| GCACCCGTACTCAGTCCG | 868 | 193593184 | 193593202 |
| GGCACCCGTACTCAGTCC | 869 | 193593185 | 193593203 |
| AGGCACCCGTACTCAGTC | 870 | 193593186 | 193593204 |
| CAGGCACCCGTACTCAGT | 871 | 193593187 | 193593205 |
| ACAGGCACCCGTACTCAG | 872 | 193593188 | 193593206 |
| GACAGGCACCCGTACTCA | 873 | 193593189 | 193593207 |
| TGACAGGCACCCGTACTC | 874 | 193593190 | 193593208 |
| CTGACAGGCACCCGTACT | 875 | 193593191 | 193593209 |
| CCTGACAGGCACCCGTAC | 876 | 193593192 | 193593210 |
| GCCTGACAGGCACCCGTA | 877 | 193593193 | 193593211 |
| AGCCTGACAGGCACCCGT | 878 | 193593194 | 193593212 |
| GAGCCTGACAGGCACCCG | 879 | 193593195 | 193593213 |
| AGAGCCTGACAGGCACCC | 880 | 193593196 | 193593214 |
| AAGAGCCTGACAGGCACC | 881 | 193593197 | 193593215 |
| CAAGAGCCTGACAGGCAC | 882 | 193593198 | 193593216 |
| GCAAGAGCCTGACAGGCA | 883 | 193593199 | 193593217 |
| CGCAAGAGCCTGACAGGC | 884 | 193593200 | 193593218 |
| CCGCAAGAGCCTGACAGG | 885 | 193593201 | 193593219 |
| TCCGCAAGAGCCTGACAG | 886 | 193593202 | 193593220 |
| TTCCGCAAGAGCCTGACA | 887 | 193593203 | 193593221 |
| CTTCCGCAAGAGCCTGAC | 888 | 193593204 | 193593222 |
| ACTTCCGCAAGAGCCTGA | 889 | 193593205 | 193593223 |
| GACTTCCGCAAGAGCCTG | 890 | 193593206 | 193593224 |
| GGACTTCCGCAAGAGCCT | 891 | 193593207 | 193593225 |
| TGGACTTCCGCAAGAGCC | 892 | 193593208 | 193593226 |
| ATGGACTTCCGCAAGAGC | 893 | 193593209 | 193593227 |
| CATGGACTTCCGCAAGAG | 894 | 193593210 | 193593228 |
| GCATGGACTTCCGCAAGA | 895 | 193593211 | 193593229 |
| CGCATGGACTTCCGCAAG | 896 | 193593212 | 193593230 |
| GCGCATGGACTTCCGCAA | 897 | 193593213 | 193593231 |
| GGCGCATGGACTTCCGCA | 898 | 193593214 | 193593232 |
| TGGCGCATGGACTTCCGC | 899 | 193593215 | 193593233 |
| ATGGCGCATGGACTTCCG | 900 | 193593216 | 193593234 |
| AATGGCGCATGGACTTCC | 901 | 193593217 | 193593235 |
| CAATGGCGCATGGACTTC | 902 | 193593218 | 193593236 |
| CCAATGGCGCATGGACTT | 903 | 193593219 | 193593237 |
| CCCAATGGCGCATGGACT | 904 | 193593220 | 193593238 |
| TCCCAATGGCGCATGGAC | 905 | 193593221 | 193593239 |
| CTCCCAATGGCGCATGGA | 906 | 193593222 | 193593240 |
| CCTCCCAATGGCGCATGG | 907 | 193593223 | 193593241 |
| CCCTCCCAATGGCGCATG | 908 | 193593224 | 193593242 |
| GCCCTCCCAATGGCGCAT | 909 | 193593225 | 193593243 |
| GGCCCTCCCAATGGCGCA | 910 | 193593226 | 193593244 |
| AGGCCCTCCCAATGGCGC | 911 | 193593227 | 193593245 |
| GAGGCCCTCCCAATGGCG | 912 | 193593228 | 193593246 |
| CGAGGCCCTCCCAATGGC | 913 | 193593229 | 193593247 |
| CCGAGGCCCTCCCAATGG | 914 | 193593230 | 193593248 |
| GCCGAGGCCCTCCCAATG | 915 | 193593231 | 193593249 |
| GGCCGAGGCCCTCCCAAT | 916 | 193593232 | 193593250 |
| CGGCCGAGGCCCTCCCAA | 917 | 193593233 | 193593251 |
| GCGGCCGAGGCCCTCCCA | 918 | 193593234 | 193593252 |
| CGCGGCCGAGGCCCTCCC | 919 | 193593235 | 193593253 |
| CCGCGGCCGAGGCCCTCC | 920 | 193593236 | 193593254 |
| GCCGCGGCCGAGGCCCTC | 921 | 193593237 | 193593255 |
| AGCCGCGGCCGAGGCCCT | 922 | 193593238 | 193593256 |
| GAGCCGCGGCCGAGGCCC | 923 | 193593239 | 193593257 |
| AGAGCCGCGGCCGAGGCC | 924 | 193593240 | 193593258 |
| CAGAGCCGCGGCCGAGGC | 925 | 193593241 | 193593259 |
| ACAGAGCCGCGGCCGAGG | 926 | 193593242 | 193593260 |
| CACAGAGCCGCGGCCGAG | 927 | 193593243 | 193593261 |
| GCACAGAGCCGCGGCCGA | 928 | 193593244 | 193593262 |
| GGCACAGAGCCGCGGCCG | 929 | 193593245 | 193593263 |
| GGGCACAGAGCCGCGGCC | 930 | 193593246 | 193593264 |
| AGGGCACAGAGCCGCGGC | 931 | 193593247 | 193593265 |
| AAGGGCACAGAGCCGCGG | 932 | 193593248 | 193593266 |
| CAAGGGCACAGAGCCGCG | 933 | 193593249 | 193593267 |
| GCAAGGGCACAGAGCCGC | 934 | 193593250 | 193593268 |
| AGCAAGGGCACAGAGCCG | 935 | 193593251 | 193593269 |
| CAGCAAGGGCACAGAGCC | 936 | 193593252 | 193593270 |
| GCAGCAAGGGCACAGAGC | 937 | 193593253 | 193593271 |
| AGCAGCAAGGGCACAGAG | 938 | 193593254 | 193593272 |
| CAGCAGCAAGGGCACAGA | 939 | 193593255 | 193593273 |
| TCAGCAGCAAGGGCACAG | 940 | 193593256 | 193593274 |
| CTCAGCAGCAAGGGCACA | 941 | 193593257 | 193593275 |
| CCTCAGCAGCAAGGGCAC | 942 | 193593258 | 193593276 |
| CCCTCAGCAGCAAGGGCA | 943 | 193593259 | 193593277 |
| GCCCTCAGCAGCAAGGGC | 944 | 193593260 | 193593278 |
| GGCCCTCAGCAGCAAGGG | 945 | 193593261 | 193593279 |
| TGGCCCTCAGCAGCAAGG | 946 | 193593262 | 193593280 |
| GTGGCCCTCAGCAGCAAG | 947 | 193593263 | 193593281 |
| AGTGGCCCTCAGCAGCAA | 948 | 193593264 | 193593282 |
| AAGTGGCCCTCAGCAGCA | 949 | 193593265 | 193593283 |
| GAAGTGGCCCTCAGCAGC | 950 | 193593266 | 193593284 |
| GGAAGTGGCCCTCAGCAG | 951 | 193593267 | 193593285 |
| AGGAAGTGGCCCTCAGCA | 952 | 193593268 | 193593286 |
| CAGGAAGTGGCCCTCAGC | 953 | 193593269 | 193593287 |
| CCAGGAAGTGGCCCTCAG | 954 | 193593270 | 193593288 |
| CCCAGGAAGTGGCCCTCA | 955 | 193593271 | 193593289 |
| ACCCAGGAAGTGGCCCTC | 956 | 193593272 | 193593290 |
| GACCCAGGAAGTGGCCCT | 957 | 193593273 | 193593291 |
| TGACCCAGGAAGTGGCCC | 958 | 193593274 | 193593292 |
| ATGACCCAGGAAGTGGCC | 959 | 193593275 | 193593293 |
| AATGACCCAGGAAGTGGC | 960 | 193593276 | 193593294 |
| GAATGACCCAGGAAGTGG | 961 | 193593277 | 193593295 |
| GGAATGACCCAGGAAGTG | 962 | 193593278 | 193593296 |
| AGGAATGACCCAGGAAGT | 963 | 193593279 | 193593297 |
| CAGGAATGACCCAGGAAG | 964 | 193593280 | 193593298 |
| CCAGGAATGACCCAGGAA | 965 | 193593281 | 193593299 |
| TCCAGGAATGACCCAGGA | 966 | 193593282 | 193593300 |
| GTCCAGGAATGACCCAGG | 967 | 193593283 | 193593301 |
| GGTCCAGGAATGACCCAG | 968 | 193593284 | 193593302 |
| CGGTCCAGGAATGACCCA | 969 | 193593285 | 193593303 |
| CCGGTCCAGGAATGACCC | 970 | 193593286 | 193593304 |
| CCCGGTCCAGGAATGACC | 971 | 193593287 | 193593305 |
| TCCCGGTCCAGGAATGAC | 972 | 193593288 | 193593306 |
| CTCCCGGTCCAGGAATGA | 973 | 193593289 | 193593307 |
| GCTCCCGGTCCAGGAATG | 974 | 193593290 | 193593308 |
| GGCTCCCGGTCCAGGAAT | 975 | 193593291 | 193593309 |
| CGGCTCCCGGTCCAGGAA | 976 | 193593292 | 193593310 |
| CCGGCTCCCGGTCCAGGA | 977 | 193593293 | 193593311 |
| CCCGGCTCCCGGTCCAGG | 978 | 193593294 | 193593312 |
| GCCCGGCTCCCGGTCCAG | 979 | 193593295 | 193593313 |
| AGCCCGGCTCCCGGTCCA | 980 | 193593296 | 193593314 |
| CAGCCCGGCTCCCGGTCC | 981 | 193593297 | 193593315 |
| CCAGCCCGGCTCCCGGTC | 982 | 193593298 | 193593316 |
| CCCAGCCCGGCTCCCGGT | 983 | 193593299 | 193593317 |
| CCCCAGCCCGGCTCCCGG | 984 | 193593300 | 193593318 |
| GCCCCAGCCCGGCTCCCG | 985 | 193593301 | 193593319 |
| AGCCCCAGCCCGGCTCCC | 986 | 193593302 | 193593320 |
| GAGCCCCAGCCCGGCTCC | 987 | 193593303 | 193593321 |
| TGAGCCCCAGCCCGGCTC | 988 | 193593304 | 193593322 |
| GTGAGCCCCAGCCCGGCT | 989 | 193593305 | 193593323 |
| TGTGAGCCCCAGCCCGGC | 990 | 193593306 | 193593324 |
| GTGTGAGCCCCAGCCCGG | 991 | 193593307 | 193593325 |
| CGTGTGAGCCCCAGCCCG | 992 | 193593308 | 193593326 |
| CCGTGTGAGCCCCAGCCC | 993 | 193593309 | 193593327 |
| CCCGTGTGAGCCCCAGCC | 994 | 193593310 | 193593328 |
| CCCCGTGTGAGCCCCAGC | 995 | 193593311 | 193593329 |
| CCCCCGTGTGAGCCCCAG | 996 | 193593312 | 193593330 |
| GCCCCCGTGTGAGCCCCA | 997 | 193593313 | 193593331 |
| AGCCCCCGTGTGAGCCCC | 998 | 193593314 | 193593332 |
| GAGCCCCCGTGTGAGCCC | 999 | 193593315 | 193593333 |
| GGAGCCCCCGTGTGAGCC | 1000 | 193593316 | 193593334 |
| GGGAGCCCCCGTGTGAGC | 1001 | 193593317 | 193593335 |
| CGGGAGCCCCCGTGTGAG | 1002 | 193593318 | 193593336 |
| GCGGGAGCCCCCGTGTGA | 1003 | 193593319 | 193593337 |
| CGCGGGAGCCCCCGTGTG | 1004 | 193593320 | 193593338 |
| ACGCGGGAGCCCCCGTGT | 1005 | 193593321 | 193593339 |
| CACGCGGGAGCCCCCGTG | 1006 | 193593322 | 193593340 |
| CCACGCGGGAGCCCCCGT | 1007 | 193593323 | 193593341 |
| GCCACGCGGGAGCCCCCG | 1008 | 193593324 | 193593342 |
| GGCCACGCGGGAGCCCCC | 1009 | 193593325 | 193593343 |
| CGGCCACGCGGGAGCCCC | 1010 | 193593326 | 193593344 |
| ACGGCCACGCGGGAGCCC | 1011 | 193593327 | 193593345 |
| GACGGCCACGCGGGAGCC | 1012 | 193593328 | 193593346 |
| AGACGGCCACGCGGGAGC | 1013 | 193593329 | 193593347 |
| GAGACGGCCACGCGGGAG | 1014 | 193593330 | 193593348 |
| CGAGACGGCCACGCGGGA | 1015 | 193593331 | 193593349 |
| CCGAGACGGCCACGCGGG | 1016 | 193593332 | 193593350 |
| GCCGAGACGGCCACGCGG | 1017 | 193593333 | 193593351 |
| CGCCGAGACGGCCACGCG | 1018 | 193593334 | 193593352 |
| GCGCCGAGACGGCCACGC | 1019 | 193593335 | 193593353 |
| GGCGCCGAGACGGCCACG | 1020 | 193593336 | 193593354 |
| AGGCGCCGAGACGGCCAC | 1021 | 193593337 | 193593355 |
| CAGGCGCCGAGACGGCCA | 1022 | 193593338 | 193593356 |
| GCAGGCGCCGAGACGGCC | 1023 | 193593339 | 193593357 |
| CGCAGGCGCCGAGACGGC | 1024 | 193593340 | 193593358 |
| ACGCAGGCGCCGAGACGG | 1025 | 193593341 | 193593359 |
| CACGCAGGCGCCGAGACG | 1026 | 193593342 | 193593360 |
| TCACGCAGGCGCCGAGAC | 1027 | 193593343 | 193593361 |
| GTCACGCAGGCGCCGAGA | 1028 | 193593344 | 193593362 |
| GGTCACGCAGGCGCCGAG | 1029 | 193593345 | 193593363 |
| AGGTCACGCAGGCGCCGA | 1030 | 193593346 | 193593364 |
| GAGGTCACGCAGGCGCCG | 1031 | 193593347 | 193593365 |
| GGAGGTCACGCAGGCGCC | 1032 | 193593348 | 193593366 |
| GGGAGGTCACGCAGGCGC | 1033 | 193593349 | 193593367 |
| GGGGAGGTCACGCAGGCG | 1034 | 193593350 | 193593368 |
| CGGGGAGGTCACGCAGGC | 1035 | 193593351 | 193593369 |
| GCGGGGAGGTCACGCAGG | 1036 | 193593352 | 193593370 |
| GGCGGGGAGGTCACGCAG | 1037 | 193593353 | 193593371 |
| CGGCGGGGAGGTCACGCA | 1038 | 193593354 | 193593372 |
| CCGGCGGGGAGGTCACGC | 1039 | 193593355 | 193593373 |
| GCCGGCGGGGAGGTCACG | 1040 | 193593356 | 193593374 |
| CGCCGGCGGGGAGGTCAC | 1041 | 193593357 | 193593375 |
| CCGCCGGCGGGGAGGTCA | 1042 | 193593358 | 193593376 |
| CCCGCCGGCGGGGAGGTC | 1043 | 193593359 | 193593377 |
| TTCACCCTTTCTAGGCGGGCAGCAC | 1044 | 193593143 | 193593168 |
| CTTCACCCTTTCTAGGCGGGCAGCA | 1045 | 193593144 | 193593169 |
| ACTTCACCCTTTCTAGGCGGGCAGC | 1046 | 193593145 | 193593170 |
| CACTTCACCCTTTCTAGGCGGGCAG | 1047 | 193593146 | 193593171 |
| CCACTTCACCCTTTCTAGGCGGGCA | 1048 | 193593147 | 193593172 |
| ACCACTTCACCCTTTCTAGGCGGGC | 1049 | 193593148 | 193593173 |
| AACCACTTCACCCTTTCTAGGCGGG | 1050 | 193593149 | 193593174 |
| CAACCACTTCACCCTTTCTAGGCGG | 1051 | 193593150 | 193593175 |
| ACAACCACTTCACCCTTTCTAGGCG | 1052 | 193593151 | 193593176 |
| AACAACCACTTCACCCTTTCTAGGC | 1053 | 193593152 | 193593177 |
| AAACAACCACTTCACCCTTTCTAGG | 1054 | 193593153 | 193593178 |
| GAAACAACCACTTCACCCTTTCTAG | 1055 | 193593154 | 193593179 |
| GGAAACAACCACTTCACCCTTTCTA | 1056 | 193593155 | 193593180 |
| CGGAAACAACCACTTCACCCTTTCT | 1057 | 193593156 | 193593181 |
| ACGGAAACAACCACTTCACCCTTTC | 1058 | 193593157 | 193593182 |
| CACGGAAACAACCACTTCACCCTTT | 1059 | 193593158 | 193593183 |
| TCACGGAAACAACCACTTCACCCTT | 1060 | 193593159 | 193593184 |
| GTCACGGAAACAACCACTTCACCCT | 1061 | 193593160 | 193593185 |
| CGTCACGGAAACAACCACTTCACCC | 1062 | 193593161 | 193593186 |
| CCGTCACGGAAACAACCACTTCACC | 1063 | 193593162 | 193593187 |
| TCCGTCACGGAAACAACCACTTCAC | 1064 | 193593163 | 193593188 |
| GTCCGTCACGGAAACAACCACTTCA | 1065 | 193593164 | 193593189 |
| AGTCCGTCACGGAAACAACCACTTC | 1066 | 193593165 | 193593190 |
| CAGTCCGTCACGGAAACAACCACTT | 1067 | 193593166 | 193593191 |
| TCAGTCCGTCACGGAAACAACCACT | 1068 | 193593167 | 193593192 |
| CTCAGTCCGTCACGGAAACAACCAC | 1069 | 193593168 | 193593193 |
| ACTCAGTCCGTCACGGAAACAACCA | 1070 | 193593169 | 193593194 |
| TACTCAGTCCGTCACGGAAACAACC | 1071 | 193593170 | 193593195 |
| GTACTCAGTCCGTCACGGAAACAAC | 1072 | 193593171 | 193593196 |
| CGTACTCAGTCCGTCACGGAAACAA | 1073 | 193593172 | 193593197 |
| CCGTACTCAGTCCGTCACGGAAACA | 1074 | 193593173 | 193593198 |
| CCCGTACTCAGTCCGTCACGGAAAC | 1075 | 193593174 | 193593199 |
| ACCCGTACTCAGTCCGTCACGGAAA | 1076 | 193593175 | 193593200 |
| CACCCGTACTCAGTCCGTCACGGAA | 1077 | 193593176 | 193593201 |
| GCACCCGTACTCAGTCCGTCACGGA | 1078 | 193593177 | 193593202 |
| GGCACCCGTACTCAGTCCGTCACGG | 1079 | 193593178 | 193593203 |
| AGGCACCCGTACTCAGTCCGTCACG | 1080 | 193593179 | 193593204 |
| CAGGCACCCGTACTCAGTCCGTCAC | 1081 | 193593180 | 193593205 |
| ACAGGCACCCGTACTCAGTCCGTCA | 1082 | 193593181 | 193593206 |
| GACAGGCACCCGTACTCAGTCCGTC | 1083 | 193593182 | 193593207 |
| TGACAGGCACCCGTACTCAGTCCGT | 1084 | 193593183 | 193593208 |
| CTGACAGGCACCCGTACTCAGTCCG | 1085 | 193593184 | 193593209 |
| CCTGACAGGCACCCGTACTCAGTCC | 1086 | 193593185 | 193593210 |
| GCCTGACAGGCACCCGTACTCAGTC | 1087 | 193593186 | 193593211 |
| AGCCTGACAGGCACCCGTACTCAGT | 1088 | 193593187 | 193593212 |
| GAGCCTGACAGGCACCCGTACTCAG | 1089 | 193593188 | 193593213 |
| AGAGCCTGACAGGCACCCGTACTCA | 1090 | 193593189 | 193593214 |
| AAGAGCCTGACAGGCACCCGTACTC | 1091 | 193593190 | 193593215 |
| CAAGAGCCTGACAGGCACCCGTACT | 1092 | 193593191 | 193593216 |
| GCAAGAGCCTGACAGGCACCCGTAC | 1093 | 193593192 | 193593217 |
| CGCAAGAGCCTGACAGGCACCCGTA | 1094 | 193593193 | 193593218 |
| CCGCAAGAGCCTGACAGGCACCCGT | 1095 | 193593194 | 193593219 |
| TCCGCAAGAGCCTGACAGGCACCCG | 1096 | 193593195 | 193593220 |
| TTCCGCAAGAGCCTGACAGGCACCC | 1097 | 193593196 | 193593221 |
| CTTCCGCAAGAGCCTGACAGGCACC | 1098 | 193593197 | 193593222 |
| ACTTCCGCAAGAGCCTGACAGGCAC | 1099 | 193593198 | 193593223 |
| GACTTCCGCAAGAGCCTGACAGGCA | 1100 | 193593199 | 193593224 |
| GGACTTCCGCAAGAGCCTGACAGGC | 1101 | 193593200 | 193593225 |
| TGGACTTCCGCAAGAGCCTGACAGG | 1102 | 193593201 | 193593226 |
| ATGGACTTCCGCAAGAGCCTGACAG | 1103 | 193593202 | 193593227 |
| CATGGACTTCCGCAAGAGCCTGACA | 1104 | 193593203 | 193593228 |
| GCATGGACTTCCGCAAGAGCCTGAC | 1105 | 193593204 | 193593229 |
| CGCATGGACTTCCGCAAGAGCCTGA | 1106 | 193593205 | 193593230 |
| GCGCATGGACTTCCGCAAGAGCCTG | 1107 | 193593206 | 193593231 |
| GGCGCATGGACTTCCGCAAGAGCCT | 1108 | 193593207 | 193593232 |
| TGGCGCATGGACTTCCGCAAGAGCC | 1109 | 193593208 | 193593233 |
| ATGGCGCATGGACTTCCGCAAGAGC | 1110 | 193593209 | 193593234 |
| AATGGCGCATGGACTTCCGCAAGAG | 1111 | 193593210 | 193593235 |
| CAATGGCGCATGGACTTCCGCAAGA | 1112 | 193593211 | 193593236 |
| CCAATGGCGCATGGACTTCCGCAAG | 1113 | 193593212 | 193593237 |
| CCCAATGGCGCATGGACTTCCGCAA | 1114 | 193593213 | 193593238 |
| TCCCAATGGCGCATGGACTTCCGCA | 1115 | 193593214 | 193593239 |
| CTCCCAATGGCGCATGGACTTCCGC | 1116 | 193593215 | 193593240 |
| CCTCCCAATGGCGCATGGACTTCCG | 1117 | 193593216 | 193593241 |
| CCCTCCCAATGGCGCATGGACTTCC | 1118 | 193593217 | 193593242 |
| GCCCTCCCAATGGCGCATGGACTTC | 1119 | 193593218 | 193593243 |
| GGCCCTCCCAATGGCGCATGGACTT | 1120 | 193593219 | 193593244 |
| AGGCCCTCCCAATGGCGCATGGACT | 1121 | 193593220 | 193593245 |
| GAGGCCCTCCCAATGGCGCATGGAC | 1122 | 193593221 | 193593246 |
| CGAGGCCCTCCCAATGGCGCATGGA | 1123 | 193593222 | 193593247 |
| CCGAGGCCCTCCCAATGGCGCATGG | 1124 | 193593223 | 193593248 |
| GCCGAGGCCCTCCCAATGGCGCATG | 1125 | 193593224 | 193593249 |
| GGCCGAGGCCCTCCCAATGGCGCAT | 1126 | 193593225 | 193593250 |
| CGGCCGAGGCCCTCCCAATGGCGCA | 1127 | 193593226 | 193593251 |
| GCGGCCGAGGCCCTCCCAATGGCGC | 1128 | 193593227 | 193593252 |
| CGCGGCCGAGGCCCTCCCAATGGCG | 1129 | 193593228 | 193593253 |
| CCGCGGCCGAGGCCCTCCCAATGGC | 1130 | 193593229 | 193593254 |
| GCCGCGGCCGAGGCCCTCCCAATGG | 1131 | 193593230 | 193593255 |
| AGCCGCGGCCGAGGCCCTCCCAATG | 1132 | 193593231 | 193593256 |
| GAGCCGCGGCCGAGGCCCTCCCAAT | 1133 | 193593232 | 193593257 |
| AGAGCCGCGGCCGAGGCCCTCCCAA | 1134 | 193593233 | 193593258 |
| CAGAGCCGCGGCCGAGGCCCTCCCA | 1135 | 193593234 | 193593259 |
| ACAGAGCCGCGGCCGAGGCCCTCCC | 1136 | 193593235 | 193593260 |
| CACAGAGCCGCGGCCGAGGCCCTCC | 1137 | 193593236 | 193593261 |
| GCACAGAGCCGCGGCCGAGGCCCTC | 1138 | 193593237 | 193593262 |
| GGCACAGAGCCGCGGCCGAGGCCCT | 1139 | 193593238 | 193593263 |
| GGGCACAGAGCCGCGGCCGAGGCCC | 1140 | 193593239 | 193593264 |
| AGGGCACAGAGCCGCGGCCGAGGCC | 1141 | 193593240 | 193593265 |
| AAGGGCACAGAGCCGCGGCCGAGGC | 1142 | 193593241 | 193593266 |
| CAAGGGCACAGAGCCGCGGCCGAGG | 1143 | 193593242 | 193593267 |
| GCAAGGGCACAGAGCCGCGGCCGAG | 1144 | 193593243 | 193593268 |
| AGCAAGGGCACAGAGCCGCGGCCGA | 1145 | 193593244 | 193593269 |
| CAGCAAGGGCACAGAGCCGCGGCCG | 1146 | 193593245 | 193593270 |
| GCAGCAAGGGCACAGAGCCGCGGCC | 1147 | 193593246 | 193593271 |
| AGCAGCAAGGGCACAGAGCCGCGGC | 1148 | 193593247 | 193593272 |
| CAGCAGCAAGGGCACAGAGCCGCGG | 1149 | 193593248 | 193593273 |
| TCAGCAGCAAGGGCACAGAGCCGCG | 1150 | 193593249 | 193593274 |
| CTCAGCAGCAAGGGCACAGAGCCGC | 1151 | 193593250 | 193593275 |
| CCTCAGCAGCAAGGGCACAGAGCCG | 1152 | 193593251 | 193593276 |
| CCCTCAGCAGCAAGGGCACAGAGCC | 1153 | 193593252 | 193593277 |
| GCCCTCAGCAGCAAGGGCACAGAGC | 1154 | 193593253 | 193593278 |
| GGCCCTCAGCAGCAAGGGCACAGAG | 1155 | 193593254 | 193593279 |
| TGGCCCTCAGCAGCAAGGGCACAGA | 1156 | 193593255 | 193593280 |
| GTGGCCCTCAGCAGCAAGGGCACAG | 1157 | 193593256 | 193593281 |
| AGTGGCCCTCAGCAGCAAGGGCACA | 1158 | 193593257 | 193593282 |
| AAGTGGCCCTCAGCAGCAAGGGCAC | 1159 | 193593258 | 193593283 |
| GAAGTGGCCCTCAGCAGCAAGGGCA | 1160 | 193593259 | 193593284 |
| GGAAGTGGCCCTCAGCAGCAAGGGC | 1161 | 193593260 | 193593285 |
| AGGAAGTGGCCCTCAGCAGCAAGGG | 1162 | 193593261 | 193593286 |
| CAGGAAGTGGCCCTCAGCAGCAAGG | 1163 | 193593262 | 193593287 |
| CCAGGAAGTGGCCCTCAGCAGCAAG | 1164 | 193593263 | 193593288 |
| CCCAGGAAGTGGCCCTCAGCAGCAA | 1165 | 193593264 | 193593289 |
| ACCCAGGAAGTGGCCCTCAGCAGCA | 1166 | 193593265 | 193593290 |
| GACCCAGGAAGTGGCCCTCAGCAGC | 1167 | 193593266 | 193593291 |
| TGACCCAGGAAGTGGCCCTCAGCAG | 1168 | 193593267 | 193593292 |
| ATGACCCAGGAAGTGGCCCTCAGCA | 1169 | 193593268 | 193593293 |
| AATGACCCAGGAAGTGGCCCTCAGC | 1170 | 193593269 | 193593294 |
| GAATGACCCAGGAAGTGGCCCTCAG | 1171 | 193593270 | 193593295 |
| GGAATGACCCAGGAAGTGGCCCTCA | 1172 | 193593271 | 193593296 |
| AGGAATGACCCAGGAAGTGGCCCTC | 1173 | 193593272 | 193593297 |
| CAGGAATGACCCAGGAAGTGGCCCT | 1174 | 193593273 | 193593298 |
| CCAGGAATGACCCAGGAAGTGGCCC | 1175 | 193593274 | 193593299 |
| TCCAGGAATGACCCAGGAAGTGGCC | 1176 | 193593275 | 193593300 |
| GTCCAGGAATGACCCAGGAAGTGGC | 1177 | 193593276 | 193593301 |
| GGTCCAGGAATGACCCAGGAAGTGG | 1178 | 193593277 | 193593302 |
| CGGTCCAGGAATGACCCAGGAAGTG | 1179 | 193593278 | 193593303 |
| CCGGTCCAGGAATGACCCAGGAAGT | 1180 | 193593279 | 193593304 |
| CCCGGTCCAGGAATGACCCAGGAAG | 1181 | 193593280 | 193593305 |
| TCCCGGTCCAGGAATGACCCAGGAA | 1182 | 193593281 | 193593306 |
| CTCCCGGTCCAGGAATGACCCAGGA | 1183 | 193593282 | 193593307 |
| GCTCCCGGTCCAGGAATGACCCAGG | 1184 | 193593283 | 193593308 |
| GGCTCCCGGTCCAGGAATGACCCAG | 1185 | 193593284 | 193593309 |
| CGGCTCCCGGTCCAGGAATGACCCA | 1186 | 193593285 | 193593310 |
| CCGGCTCCCGGTCCAGGAATGACCC | 1187 | 193593286 | 193593311 |
| CCCGGCTCCCGGTCCAGGAATGACC | 1188 | 193593287 | 193593312 |
| GCCCGGCTCCCGGTCCAGGAATGAC | 1189 | 193593288 | 193593313 |
| AGCCCGGCTCCCGGTCCAGGAATGA | 1190 | 193593289 | 193593314 |
| CAGCCCGGCTCCCGGTCCAGGAATG | 1191 | 193593290 | 193593315 |
| CCAGCCCGGCTCCCGGTCCAGGAAT | 1192 | 193593291 | 193593316 |
| CCCAGCCCGGCTCCCGGTCCAGGAA | 1193 | 193593292 | 193593317 |
| CCCCAGCCCGGCTCCCGGTCCAGGA | 1194 | 193593293 | 193593318 |
| GCCCCAGCCCGGCTCCCGGTCCAGG | 1195 | 193593294 | 193593319 |
| AGCCCCAGCCCGGCTCCCGGTCCAG | 1196 | 193593295 | 193593320 |
| GAGCCCCAGCCCGGCTCCCGGTCCA | 1197 | 193593296 | 193593321 |
| TGAGCCCCAGCCCGGCTCCCGGTCC | 1198 | 193593297 | 193593322 |
| GTGAGCCCCAGCCCGGCTCCCGGTC | 1199 | 193593298 | 193593323 |
| TGTGAGCCCCAGCCCGGCTCCCGGT | 1200 | 193593299 | 193593324 |
| GTGTGAGCCCCAGCCCGGCTCCCGG | 1201 | 193593300 | 193593325 |
| CGTGTGAGCCCCAGCCCGGCTCCCG | 1202 | 193593301 | 193593326 |
| CCGTGTGAGCCCCAGCCCGGCTCCC | 1203 | 193593302 | 193593327 |
| CCCGTGTGAGCCCCAGCCCGGCTCC | 1204 | 193593303 | 193593328 |
| CCCCGTGTGAGCCCCAGCCCGGCTC | 1205 | 193593304 | 193593329 |
| CCCCCGTGTGAGCCCCAGCCCGGCT | 1206 | 193593305 | 193593330 |
| GCCCCCGTGTGAGCCCCAGCCCGGC | 1207 | 193593306 | 193593331 |
| AGCCCCCGTGTGAGCCCCAGCCCGG | 1208 | 193593307 | 193593332 |
| GAGCCCCCGTGTGAGCCCCAGCCCG | 1209 | 193593308 | 193593333 |
| GGAGCCCCCGTGTGAGCCCCAGCCC | 1210 | 193593309 | 193593334 |
| GGGAGCCCCCGTGTGAGCCCCAGCC | 1211 | 193593310 | 193593335 |
| CGGGAGCCCCCGTGTGAGCCCCAGC | 1212 | 193593311 | 193593336 |
| GCGGGAGCCCCCGTGTGAGCCCCAG | 1213 | 193593312 | 193593337 |
| CGCGGGAGCCCCCGTGTGAGCCCCA | 1214 | 193593313 | 193593338 |
| ACGCGGGAGCCCCCGTGTGAGCCCC | 1215 | 193593314 | 193593339 |
| CACGCGGGAGCCCCCGTGTGAGCCC | 1216 | 193593315 | 193593340 |
| CCACGCGGGAGCCCCCGTGTGAGCC | 1217 | 193593316 | 193593341 |
| GCCACGCGGGAGCCCCCGTGTGAGC | 1218 | 193593317 | 193593342 |
| GGCCACGCGGGAGCCCCCGTGTGAG | 1219 | 193593318 | 193593343 |
| CGGCCACGCGGGAGCCCCCGTGTGA | 1220 | 193593319 | 193593344 |
| ACGGCCACGCGGGAGCCCCCGTGTG | 1221 | 193593320 | 193593345 |
| GACGGCCACGCGGGAGCCCCCGTGT | 1222 | 193593321 | 193593346 |
| AGACGGCCACGCGGGAGCCCCCGTG | 1223 | 193593322 | 193593347 |
| GAGACGGCCACGCGGGAGCCCCCGT | 1224 | 193593323 | 193593348 |
| CGAGACGGCCACGCGGGAGCCCCCG | 1225 | 193593324 | 193593349 |
| CCGAGACGGCCACGCGGGAGCCCCC | 1226 | 193593325 | 193593350 |
| GCCGAGACGGCCACGCGGGAGCCCC | 1227 | 193593326 | 193593351 |
| CGCCGAGACGGCCACGCGGGAGCCC | 1228 | 193593327 | 193593352 |
| GCGCCGAGACGGCCACGCGGGAGCC | 1229 | 193593328 | 193593353 |
| GGCGCCGAGACGGCCACGCGGGAGC | 1230 | 193593329 | 193593354 |
| AGGCGCCGAGACGGCCACGCGGGAG | 1231 | 193593330 | 193593355 |
| CAGGCGCCGAGACGGCCACGCGGGA | 1232 | 193593331 | 193593356 |
| GCAGGCGCCGAGACGGCCACGCGGG | 1233 | 193593332 | 193593357 |
| CGCAGGCGCCGAGACGGCCACGCGG | 1234 | 193593333 | 193593358 |
| ACGCAGGCGCCGAGACGGCCACGCG | 1235 | 193593334 | 193593359 |
| CACGCAGGCGCCGAGACGGCCACGC | 1236 | 193593335 | 193593360 |
| TCACGCAGGCGCCGAGACGGCCACG | 1237 | 193593336 | 193593361 |
| GTCACGCAGGCGCCGAGACGGCCAC | 1238 | 193593337 | 193593362 |
| GGTCACGCAGGCGCCGAGACGGCCA | 1239 | 193593338 | 193593363 |
| AGGTCACGCAGGCGCCGAGACGGCC | 1240 | 193593339 | 193593364 |
| GAGGTCACGCAGGCGCCGAGACGGC | 1241 | 193593340 | 193593365 |
| GGAGGTCACGCAGGCGCCGAGACGG | 1242 | 193593341 | 193593366 |
| GGGAGGTCACGCAGGCGCCGAGACG | 1243 | 193593342 | 193593367 |
| GGGGAGGTCACGCAGGCGCCGAGAC | 1244 | 193593343 | 193593368 |
| CGGGGAGGTCACGCAGGCGCCGAGA | 1245 | 193593344 | 193593369 |
| GCGGGGAGGTCACGCAGGCGCCGAG | 1246 | 193593345 | 193593370 |
| GGCGGGGAGGTCACGCAGGCGCCGA | 1247 | 193593346 | 193593371 |
| CGGCGGGGAGGTCACGCAGGCGCCG | 1248 | 193593347 | 193593372 |
| CCGGCGGGGAGGTCACGCAGGCGCC | 1249 | 193593348 | 193593373 |
| GCCGGCGGGGAGGTCACGCAGGCGC | 1250 | 193593349 | 193593374 |
| CGCCGGCGGGGAGGTCACGCAGGCG | 1251 | 193593350 | 193593375 |
| CCGCCGGCGGGGAGGTCACGCAGGC | 1252 | 193593351 | 193593376 |
| CCCGCCGGCGGGGAGGTCACGCAGG | 1253 | 193593352 | 193593377 |

TABLE 9
List of Exemplary Cell-penetrating peptide
Sequences (Organism: synthetic construct)

| SEQ ID NO | Sequence |
|---|---|
| 1281 | RRRRRRRRXB |
| 1282 | RXRRBRRXRRBRXB |
| 1283 | RXRRXRRXRRBRXB |
| 1284 | RBRBRBRBRBRXRBRXB |
| 1285 | RBRBRBRXRBRBRBRXX |
| 1286 | XRBRBRBRXRBRBRBRX |
| 1287 | RBRXRBRXRBRXRBRXB |
| 1288 | RBRBRBRBRXRXRXRXB |
| 1289 | RXRBRBRXRBRBRBRXX |
| 1290 | rXrrXrrXrrXrXB |
| 1291 | RXRRXRRXRRXRXB |
| 1292 | RBRBRBRBRBRBRBRBB |
| 1293 | RBRBRBRBRBRBRBRXB |
| 1294 | RBRBRBRBRBRBRBRBB |
| 1295 | RRSRTARAGRPGRNSSRPSAPR |
| 1296 | rrrrrrrrXB |
| 1297 | RRRRRRRRRXB |
| 1298 | RXRXRXRXRXRXRXRXB |
| 1299 | rXrXrXrXrXrXrXrXB |
| 1300 | RXRXRXRXRXRXRXB |
| 1301 | RXRXRXRXRXB |
| 1302 | RXRXRXB |
| 1303 | rXRrXRrXRrXRXB |
| 1304 | rXrrXrrXrrXrXB |
| 1305 | rBrBrBrBrBrBrBrBB |
| 1306 | RBRBRBRBRBRBRBB |
| 1307 | RBRBRBRBRBB |
| 1308 | RBRBRBB |
| 1309 | XRBRBRBRXRBRBRBRB |

Abbreviation:
B, β-alanine; r, d-arginine; R, 1-arginine; X, 6-aminohexanoic acid.
Sequences assigned to SEQ ID NOs do not include the linkage portion (X, B, or XB)

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the present disclosure may be employed in practicing the present disclosure. It is intended that the following claims define the scope of the present disclosure and that methods and structures within the scope of these claims and their equivalents be covered thereby.

1.-393. (canceled)

394. A method of increasing expression of an OPA1 protein in a cell having a processed mRNA that encodes the OPA1 protein and that comprises a translation regulatory element that inhibits translation of the processed mRNA, the method comprising:
contacting an agent or a vector encoding the agent to the cell, wherein the agent (a) binds to a targeted portion of a full-length mature mRNA or a wild-type mature mRNA, wherein the targeted portion does not bind to a canonical or non-canonical start site; and
(b) modulates formation of a secondary mRNA structure, wherein the secondary structure is either a guanine-quadruplex or a stem loop;
thereby increasing expression of the OPA1 protein in the cell.

395. The method of claim 394, wherein the secondary structure is a guanine quadruplex (G-quad) selected from the G-quads in FIG. 3.

396. The method of claim 395, wherein the secondary structure is in a 5′ UTR of a processed mRNA.

397. The method of claim 395, wherein the secondary structure involves base-pairing with at least one nucleotide of the main start codon of the processed mRNA.

398. The method of claim 397, wherein the agent inhibits the base-pairing with the at least one nucleotide of the main start codon of the processed mRNA.

399. The method of claim 394, wherein the targeted portion is at least 42 nucleotides upstream of the main start codon of the processed mRNA.

400. The method of claim 399, wherein the main start codon is defined by chromosomal coordinates GRCh38 chr3: 193,593,378-193,593,380.

401. The method of claim 399, wherein the targeted portion is within exon 1 of the full-length mature mRNA.

402. The method of claim 394, wherein the translation regulatory element comprises an upstream start codon.

403. The method of claim 402, wherein the agent promotes formation of a secondary mRNA structure that involves base-pairing with at least one nucleotide of the upstream start codon.

404. The method of claim 395, wherein the agent inhibits formation of the Guanine quadruplex formed by a G-rich sequence of the processed mRNA.

405. The method of claim 404, wherein the G-rich sequence comprises at least a portion of 5′ untranslated region (5′ UTR) of the processed mRNA.

406. The method of claim 394, wherein the agent increases the expression of the OPA1 protein in the cell by increasing the translation efficiency and/or rate of translation of a full-length mature mRNA or a wild-type mature mRNA.

407. The method of claim 394, wherein the translation efficiency and/or rate of translation of the processed mRNA that encodes the OPA1 protein in the cell contacted with the agent or the vector encoding the agent is increased by at least 1.1 fold.

408. The method of claim 394, wherein the agent is an antisense oligonucleotide.

409. The method of claim 408, wherein the antisense oligomer comprises a sequence with at least 90% sequence identity to a sequence selected from the group consisting of SEQ ID NOs: 608-1253.

410. The method of claim 408, wherein the antisense oligomer comprises a phosphorodiamidate morpholino, a locked nucleic acid, a peptide nucleic acid, a 2′-O-methyl moiety, a 2′-Fluoro moiety, or a 2′-O-m ethoxy ethyl moiety.

411. The method of claim 408, wherein the antisense oligomer comprises at least one modified sugar moiety.

412. The method of claim 408, wherein the antisense oligomer consists of from 8 to 25 nucleobases.

413. The method of claim 394, wherein the viral vector comprises an adenoviral vector, adeno-associated viral (AAV) vector, lentiviral vector, Herpes Simplex Virus (HSV) viral vector, or retroviral vector.
